SEQUENCE LISTING (91 sequences)

SEQ ID NO: 1 - DNA, 79 bp, synthetic construct
  source 1..79: mol_type = other DNA
aattcagcgggttcgtgcgaactgttgacaattaatactcggctcgtataatgtgtggaaggctagcaggaggaattca

SEQ ID NO: 2 - DNA, 961 bp, Pantoea agglomerans
  source 1..961: mol_type = genomic DNA
atgccactgctgtttgaaagcttaagccatcaaatttacctgtcagccccgctgtttatcctgatcctgctggggtactgcctgacgcgcctcgggaaatggcccgccagcatcagcgaagggatgaaccgctttgtgtttaacgttgccctgccctgcatgctgtttcgggtgatgagcaccttctcgcaaagtccgccggtggatgcgcgcctgttgctggcctttttcggcagttgcctgctggtgtttatcgtggggcgactgctggcaacaaggctgtttaatctggatggcgtagcggcgtcggtttttgcgctgggcggcatcttttccaataacgtgatgctggggatcccagtggccacggtactgctgggaccagaagccctgcccccggtggcgctggtgctggtgtttaacagcctgatcctctggaccttactgaccgtttcggtcgagtgggcaaaacagggcagcttctcgctggaaggactctggcgcaccctgctcggcgtgctgaagaatccgctgatcatcggtattctcagcggtacagcctggagctttctgcaacgtcccttgccgctgctggccgctgaaccgctaaagatgctggcctcactggccgcacctctgtcgctggtgacgctgggcatgagcctggcgcactatcgggtgcgggatggcttaaaagagagctataccatctgcctgctgaaactggtgctgcaaccgatgacggtatgggctatcgcgtggctgaccgatctgccgccgctggagagtaaagtggtggtgctgctgggatcgatggcggtcggggttaacgtctacctgatgtcgcagaagtttaatgttatcaccggcccggcagcggcgagcatgctcttttctaccgtgtttgcggcggtgacaacgccaatctggatgatgctgatgacgctggcgggctattaac

SEQ ID NO: 3 - DNA, 1944 bp, Arabidopsis thaliana
  source 1..1944: mol_type = genomic DNA
atgattacaggcaaagatatgtacgacgtgctggcagctatggtgcccctgtatgtggctatgatccttgcttacggctctgtccgctggtggggtatttttacacccgaccaatgctcaggaattaatcgttttgtcgctgtttttgccgttcctttgttatcatttcatttcattagctcaaatgacccttatgcaatgaattatcattttttagccgctgattccttacagaaagtggttatcttagcagccttgttcctgtggcaagccttctctcgtcgcgggagtttagaatggatgattacacttttttccctttctaccttgcccaatacgctggtaatgggtatcccccttttgcgcgcaatgtacggcgacttttctggtaaccttatggtccaaatcgtcgtcttacagagtattatttggtacactttgatgctgtttttgttcgagtttcgcggcgctaaacttctgattagtgaacaatttccggagactgcggggtcaatcactagttttcgtgttgatagtgatgtcatcagcttgaatgggcgcgagccattgcagaccgacgcggagatcggcgatgatgggaagcttcatgttgtagtacgccgtagcagtgctgcatccagtatgattagttcatttaacaagtcccatggagggggtcttaacagttcaatgattacaccacgtgctagtaatcttactggggttgagatttacagtgtgcagtctagtcgcgagcccaccccgcgtgcatccagtttcaaccaaacggacttttacgctatgttcaatgctagtaaggccccttcaccgcgccacggctatactaatagctacggaggcgcaggagcaggcccaggtggtgacgtgtacagccttcagtcttccaaaggggtcacaccgcgtacatctaatttcgatgaagaagttatgaaaactgcaaagaaagcaggtcgtggcggtcgctctatgtcaggggaattatacaacaacaattcggtgccaagttacccaccccctaacccgatgtttacaggatcaacgagtggtgcgtccggtgttaagaagaaggagtcaggcggagggggaagcgggggaggcgtcggggtaggtggccaaaacaaggaaatgaatatgttcgtctggtctagcagcgcctcacccgtcagcgaggcgaacgcaaagaatgccatgacgcgcggatctagtactgacgtgtcaaccgaccctaaagtgtccatcccaccccatgacaacttagcgaccaaggctatgcagaatcttattgaaaatatgtcgcctggtcgtaaaggccatgtagaaatggatcaggacgggaacaatggcgggaaatctccctacatgggtaagaaggggtctgatgtcgaggatggaggtcccggacctcgtaagcagcagatgcctccggcgagtgtaatgacacgcttgattcttattatggtctggcgtaagttaattcgcaatcctaacacatactcaagcctttttggtctggcctggtctttagtatcgtttaagtggaatattaaaatgccaactatcatgagcggtagcatttccattttgagcgacgcgggattgggcatggcaatgtttagtcttggcctgttcatggccttgcaaccaaaaattatcgcgtgcgggaagtctgtagccgggtttgcgatggcggttcgcttcctgactggaccagcggttattgctgccaccagtatcgctatcggtattcgcggggacctgttacacattgccatcgtacaggcggcactgccccagggaatcgtcccctttgtttttgccaaagaatacaatgtgcacccagatattctttctacggcagtgatttttggtatgcttgtggcgctgccggttaccgtgttatattatgttttacttggattataa

SEQ ID NO: 4 - DNA, 35 bp, synthetic construct
  source 1..35: mol_type = other DNA
ttgacagctagctcagtcctaggtataatgctagc

SEQ ID NO: 5 - DNA, 35 bp, synthetic construct
  source 1..35: mol_type = other DNA
ttgacggctagctcagtcctaggtacagtgctagc

SEQ ID NO: 6 - DNA, 35 bp, synthetic construct
  source 1..35: mol_type = other DNA
tttacagctagctcagtcctaggtattatgctagc

SEQ ID NO: 7 - DNA, 35 bp, synthetic construct
  source 1..35: mol_type = other DNA
ttgacagctagctcagtcctaggtactgtgctagc

SEQ ID NO: 8 - DNA, 35 bp, synthetic construct
  source 1..35: mol_type = other DNA
ctgatagctagctcagtcctagggattatgctagc

SEQ ID NO: 9 - DNA, 35 bp, synthetic construct
  source 1..35: mol_type = other DNA
ttgacagctagctcagtcctaggtattgtgctagc

SEQ ID NO: 10 - DNA, 35 bp, synthetic construct
  source 1..35: mol_type = other DNA
tttacggctagctcagtcctaggtactatgctagc

SEQ ID NO: 11 - DNA, 35 bp, synthetic construct
  source 1..35: mol_type = other DNA
tttacggctagctcagtcctaggtatagtgctagc

SEQ ID NO: 12 - DNA, 35 bp, synthetic construct
  source 1..35: mol_type = other DNA
tttacggctagctcagccctaggtattatgctagc

SEQ ID NO: 13 - DNA, 35 bp, synthetic construct
  source 1..35: mol_type = other DNA
ctgacagctagctcagtcctaggtataatgctagc

SEQ ID NO: 14 - DNA, 35 bp, synthetic construct
  source 1..35: mol_type = other DNA
tttacagctagctcagtcctagggactgtgctagc

SEQ ID NO: 15 - DNA, 35 bp, synthetic construct
  source 1..35: mol_type = other DNA
tttacggctagctcagtcctaggtacaatgctagc

SEQ ID NO: 16 - DNA, 35 bp, synthetic construct
  source 1..35: mol_type = other DNA
ttgacggctagctcagtcctaggtatagtgctagc

SEQ ID NO: 17 - DNA, 35 bp, synthetic construct; identical to SEQ ID NO: 8
  source 1..35: mol_type = other DNA

SEQ ID NO: 18 - DNA, 35 bp, synthetic construct
  source 1..35: mol_type = other DNA
ctgatggctagctcagtcctagggattatgctagc

SEQ ID NO: 19 - DNA, 35 bp, synthetic construct
  source 1..35: mol_type = other DNA
tttatggctagctcagtcctaggtacaatgctagc

SEQ ID NO: 20 - DNA, 35 bp, synthetic construct
  source 1..35: mol_type = other DNA
tttatagctagctcagcccttggtacaatgctagc

SEQ ID NO: 21 - DNA, 35 bp, synthetic construct
  source 1..35: mol_type = other DNA
ttgacagctagctcagtcctagggactatgctagc

SEQ ID NO: 22 - DNA, 35 bp, synthetic construct
  source 1..35: mol_type = other DNA
ttgacagctagctcagtcctagggattgtgctagc

SEQ ID NO: 23 - DNA, 35 bp, synthetic construct
  source 1..35: mol_type = other DNA
ttgacggctagctcagtcctaggtattgtgctagc

SEQ ID NO: 24 - DNA, 79 bp, synthetic construct
  source 1..79: mol_type = other DNA
aattcagcgggttcgtgcgaactgtagacaattaatactcggctcgtataatgtgtggaaggctagcaggaggaattca

SEQ ID NO: 25 - DNA, 20 bp, synthetic construct
  source 1..20: mol_type = other DNA
gcgtcaaggatgctctactc

SEQ ID NO: 26 - DNA, 20 bp, synthetic construct
  source 1..20: mol_type = other DNA
ttaattgccagccatcgcct

SEQ ID NO: 27 - DNA, 85 bp, synthetic construct
  source 1..85: mol_type = other DNA
gtgatctccaccggcttattagcgatagggattatctgtaccgtgccaattatggctcgcccttagttcctattccgaagttcct

SEQ ID NO: 28 - DNA, 87 bp, synthetic construct
  source 1..87: mol_type = other DNA
gtcgaaaatatcgagcagctcctgctctgacatagacgccacccgataccctttttgattcaccaccgccctgccactcatcgcagt

SEQ ID NO: 29 - DNA, 90 bp, synthetic construct
  source 1..90: mol_type = other DNA
tccgcacgtttatgatatgctatcgtactctttagcgagtacaaccgggggaggcattttgcttcccttagttcctattccgaagttcct

SEQ ID NO: 30 - DNA, 87 bp, synthetic construct
  source 1..87: mol_type = other DNA
cacgccgcatccggcattcggtgcacgatgcctgatgcgccacgtcttatcaggcctacaaaaccgccctgccactcatcgcagtac

SEQ ID NO: 31 - DNA, 21 bp, synthetic construct
  source 1..21: mol_type = other DNA
gacgctgccgcgtcttatcat

SEQ ID NO: 32 - DNA, 88 bp, synthetic construct
  source 1..88: mol_type = other DNA
tccgcacgtttatgatatgctatcgtactctttagcgagtacaaccgggggaggcattttgcttcttttgtaggcctgataagacgtg

SEQ ID NO: 33 - DNA, 87 bp, synthetic construct
  source 1..87: mol_type = other DNA
agaatagacaaaaactctgagtgtaataatgtagcctcgtgtcttgcgaggataagtgcattccttagttcctattccgaagttcct

SEQ ID NO: 34 - DNA, 87 bp, synthetic construct
  source 1..87: mol_type = other DNA
accataacaccccaaaatgcagagtgctttttttcagcttgatcagtcatgatgccacctttaccgccctgccactcatcgcagtac

SEQ ID NO: 35 - DNA, 26 bp, synthetic construct
  source 1..26: mol_type = other DNA
gtaataccgctgataatgttccaggt

SEQ ID NO: 36 - DNA, 89 bp, synthetic construct
  source 1..89: mol_type = other DNA
atagacaaaaactctgagtgtaataatgtagcctcgtgtcttgcgaggataagtgcattggtaaaggwggcatcatgactgatcaagct

SEQ ID NO: 37 - DNA, 84 bp, synthetic construct
  source 1..84: mol_type = other DNA
aattcagcgggttcgtgcgagctgtngrcaattaatactcggctcgtataatgtgtggaaggctagcaggaggaattcaatgat

SEQ ID NO: 38 - DNA, 173 bp, synthetic construct
  source 1..173: mol_type = other DNA
gttagaagtgatctccaccggcttattagcgatagggattatctgtaccgtgccaattatggctcgctggaaaaagctggtattgtggcaaaaaacacccgttcataatacgcgctgaattcagcgggttcgtgcgagctgttgacaattaatactcggctcgtataatgtgt

SEQ ID NO: 39 - DNA, 1191 bp, Escherichia coli
  source 1..1191: mol_type = genomic DNA
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaa

SEQ ID NO: 40 - DNA, 1176 bp, Arabidopsis thaliana
  source 1..1176: mol_type = genomic DNA
atggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaa

SEQ ID NO: 41 - DNA, 1653 bp, Pantoea agglomerans
  source 1..1653: mol_type = genomic DNA
atgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaa

SEQ ID NO: 42 - DNA, 1494 bp, Ustilago maydis
  source 1..1494: mol_type = genomic DNA
atgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaa

SEQ ID NO: 43 - DNA, 1086 bp, Escherichia coli
  source 1..1086: mol_type = genomic DNA
atggaaagtggtcatcgctttgatgctcagacgctgcacagttttattcaggctgtatttcgtcagatgggtagcgaggaacaagaagcgaaattagttgccgatcatttaatcgcggcaaacctggcagggcatgattcacatggtattggcatgatcccaagctatgtacgctcctggagtcaggggcacctgcaaattaaccatcatgccaaaaccgttaaagaggcgggggcggcggtcacgctcgatggcgatcgcgcatttggtcaggtcgcggcacatgaagcgatggcgctggggattgagaaagcgcatcagcacggtattgccgccgtggcgctacataactcgcatcatatcggccgtatcggttactgggcggagcagtgtgcagcggcggggtttgtctctatccactttgttagcgtggtcggtattccaatggtcgcgccgttccacggtcgcgacagccgctttggcaccaatccgttctgtgtggttttccctcgtaaagataatttcccgctgttgcttgattacgccaccagcgccattgcatttggcaaaacccgcgtcgcctggcataaaggcgtccccgtgccgccaggttgcctgattgacgttaacggcgtgccgacgaccaatccggcggtaatgcaggagtcgccgttgggttcgctgttgacctttgccgaacataaaggctacgcccttgcagcgatgtgtgaaattcttggcggggcgctttccggcggtaaaacgacgcatcaggaaacgttacaaaccagtcccgatgccattcttaactgcatgaccactatcatcatcaacccggaactcttcggcgcgccggattgtaacgcgcagaccgaagcctttgccgagtgggtgaaagcctcgccgcatgatgatgataagccgattttgctaccgggcgagtgggaagtgaacacgcgtcgcgaacggcagaagcaggggattccactggatgcgggaagctggcaggccatttgtgatgcagcgcggcagattggtatgccggaagagacgttgcaggctttctgtcagcagttagccagctaa

SEQ ID NO: 44 - DNA, 951 bp, Bifidobacterium longum
  source 1..951: mol_type = genomic DNA
atggtgaccatgaatcgtaataaagttgttatcgtgggtacgggccaggtcggcgccaccgctgcgttcggcattgttacgcatggtttgtgcaacgaactggtcttgatcgaccgttccgccgccaaggcattgggggaggctcgtgaccttgacgacggtagcgaatttcaggaccgtcacgttaaggtccgcgcgggagattacgccgattgcaaagacgctgacattgttgttattactgttgggcgtaagccaccagcgaacagtaaccgtatggcggaacttggttttacggtcggtttggtgggcgaagtagttgataatgttatggctagtggatttgatggagttattgttatggttagcaaccccgtcgatgttatggcttggtatgcttggaagcgttcgggactgccccgcacgcaagtccttggaacgggtaccgccttagatacctcccgccttaagacaattatcggagaagaaactggtttagatccacgtaacgtaggcggctttgttatgggggaacatggtgattctcagttcactgcttggagcactgtatcgttaggcggtaaaccgtttgctcgctttttagcagacaatcaagatcgcttcgcatcggtatcaactaccgagatcgaggaaaaaacgcgcacacgtggcgacgaaatcgtagcggccaagggcgggaccaattttggcattgcgtcaacagttgccggtatcgtacaaaccatcctgtgggacgagcgccgcatcgtaccagtaagcacgctgcttgatggagagtacggtgaacatgacgtatttttgggtgtcccaacggaactgcgtgcaaacggggcgaatgaaatcgtagaattggacttaagcgaagacgagcgcgctaagttacatcattcggccgaattggtgcgtgaccactgcaaaggattgctttga

SEQ ID NO: 45 - DNA, 996 bp, Clostridium sporogenes
  source 1..996: mol_type = genomic DNA
atgaaaatcttagcttattgtgtaagacctgatgaaattgattcatttaaaaatttttctgaaaaatatggacatactgttgatttaataccagattcttttggtccaagtgtggctcatttagctaaaggatatgatggcatttcaattttgggaaatgatacttgtaacagagaagcattagagaaaattaaagattgtggtataaaatatttagcaaccagaactgctggagtaaataatatagattttgatgctgcaaaagaatttggtataaatgtagctaatgttccagcttattctcctaattcagtttcagagtttactgtaggactcgcattatccctaacaaggaaaattccttttgcattaaaaagagtagagcttaacaattttgcattaggtggattaataggagtagaattaagaaacttaactttaggagttataggaactggaagaataggtttaaaagtaattgaaggcttttcaggctttggtatgaaaaagatgataggttacgatatttttgaaaatgaaaaggctaaagaatatattgaatataaatctttagatgaagtatataaagaagcagacataataacactacatgcacctttaacagatgataattaccatatgatagggaaagagagtattgctaaaatgaaagacggtgtattcataataaatgcagcacgaggagcattaattgatagtgaagctttaatagaaggactgaaatctggtaaaatagctggagcagccttagattcttatgaatatgaacaaggagttttccataacaataaaatgaatgaaatcatgaaagatgatacattagcaagacttaagagttttccaaatgtagttattacacctcatttaggattctatacagatgaagctgtatcaaatatggttgaaattactcttatgaatttacaagagtttgaattaaaaggaacttgtaaaaatcagagggtatgtaagtaa

SEQ ID NO: 46 - DNA, 4387 bp, Clostridium sporogenes
  source 1..4387: mol_type = genomic DNA
atggaaaacaatacaaatatgtttagtggagtaaaggttattgaattagcaaattttatagctgctccagcagcaggtagattttttgctgatggtggtgcagaggtaataaaaattgaatcacctgctggagatcctttaagatatactgctccttcagaaggaagaccattaagccaagaagaaaatactacttatgatttggaaaatgcaaataaaaaagcaatagtattaaatcttaaaagcgaaaaaggtaaaaagatattacatgaaatgttagcagaagcagatatattattaactaattggagaacaaaggctttagttaaacaaggattagactatgaaacactaaaagaaaaatatcctaaattagtttttgcacaaataactggttatggtgaaaaaggaccagataaagatcttccaggctttgattatactgcatttttcgctagaggcggtgtttcaggtactctttatgaaaaaggaactgtgcctccaaatgttgttccaggacttggagaccatcaagctgggatgtttttagcagcgggtatggcaggagctttatataaagcaaaaacaacaggacaaggagataaagtaacagtaagtttaatgcatagtgctatgtatggactaggtattatgatacaagctgctcaatataaagatcatggattagtatatccgataaatcgtaatgaaactccaaatccttttatagtttcatataaatctaaggatgattactttgttcaagtatgtatgccaccatatgatgttttctatgatagatttatgaccgctttaggaagagaagatttagttggagacgaaagatacaataaaatagaaaatttaaaagatggacgtgctaaggaagtatacagtataatcgaacaacaaatggttacaaagacaaaggatgaatgggataaaatatttagagatgcagacattccatttgctatcgcacaaacttgggaagatttattagaagatgaacaagcttgggcaaatgattatttgtataagatgaaatatccaacaggaaacgaaagagcattagtaagacttccagtattctttaaagaagcaggattaccagaatataatcaatcaccacaaatagcagaaaatactgtagaagttttaaaagaaatgggatatacagaacaagagattgaggaattagaaaaagataaagatataatggtaaggaaggaaaaataatggcagacatttatactatgggtgtagacataggttcaactgcatcaaaaacagtagtattaaaaaatggtaaagaaattgtaagtcaagcagtaataagtgtaggggccggaacaagtggccccaagagagctatagattctgtattaaaagatgctaaattatccattgaagatttagactatattgtatccactggatatggaagaaatagtttcgattttgctaacaaacaaatttctgaattaagttgtcatgcaaaaggggtctatttcgataacaataaagctagaacagttattgatataggcggacaagatattaaagtattaaaattagcggatagtggaagacttttaaactttataatgaatgataaatgtgctgcaggaacgggacgatttttagatgtaatgtctagagtaatagaagttccagttgatgagttaggaaaaaaagcattagaaagcaaaaatccttgtactattagttctacctgtacagtatttgcagagtcagaagtaatttctcaacttgcaagaggagttaaaactgaagatttgatagcaggaatttgtaaatctgtagcatcaagagtggctagccttgcaaagagaagtggtatagaagaattagtagttatgagtggaggagtagctaaaaatataggtgtagtaaaggcaatggaagcagaattgggaagagacatatatatatctaaaaattctcaattaaatggagcattgggagcaagtctatacgcttatgaaagttttcaaaaagaaaggagctaaaaacatgagtgatagaaataaggaagtaaaagaaaaaaaggcaaagcattatcttagagagattactgcaaagcattacaaagaagctctcgaagcaaaagaaaggggagaaaaggttggttggtgtgcatctaacttcccacaagaaatagctacaacattgggggtaaaagttgtttatccagaaaatcatgcagcagctgtagcagctagagggaatggacaaaatatgtgtgaacatgctgaggctatgggtttttctaatgatgtatgtggttatgcaagagtaaatttagctgttatggacataggtcatagtgaagatcaaccaatacctatgccagactttgtactttgctgtaataacatttgtaatcaaatgattaaatggtatgagcatatagcaaaaactttagatataccaatgattcttatagatataccatacaatacagaaaatactgtttcacaagatagaattaaatatattagagcacaatttgatgatgcaataaaacaattggaagaaataacaggcaaaaaatgggatgaaaataaatttgaagaagttatgaaaatatcccaagaaagtgcaaaacaatggttaagagcagcatcctatgcaaagtataaaccttcaccatttagcggatttgatttatttaatcatatggctgtagcagtttgtgcaagaggtacacaagaagctgcagatgcatttaagatgttagcagatgaatatgaggagaatgtaaaaactggaaaatccacttataggggagaagaaaaacaacgtatattatttgaagggattgcctgttggccatatttgagacataaattaactaagcttagtgaatatggtatgaacgtaactgcaactgtatacgcagaagcctttggtgttatatatgagaatatggatgaattaatggctgcttataataaagttcctaattcaattagttttgaaaacgcattaaaaatgagattaaatgctgttacaagcactaatacagaaggtgctgttattcatataaatagaagctgtaaattatggagtggatttttatatgagctagcaagaagattagaaaaggaaacaggaattcctgtagtatcatttgatggggaccaggcagacccaagaaatttctcagaagctcaatatgatactagaattcaaggacttaatgaagtaatggttgctaaaaaggaggctgaataagatgtcaaattcagataaattttttaatgactttaaggatattgtagaaaatcctaaaaaatatataatgaagcatatggaacaaactggacaaaaggctataggatgtatgccattatatactcctgaggaacttgtattagctgctggaatgtttccagtaggggtatggggaagcaatacagaactttcaaaagctaaaacatatttcccagcatttatttgttcaatattacaaacaacattggaaaatgcattaaatggagaatatgatatgttatctggtatgatgattacaaattattgtgattcattaaaatgcatgggacaaaattttaaactaaccgttgaaaatattgagtttatcccagtaacagttccacaaaatagaaaaatggaagctggaaaagagtttttaaaaagtcaatataaaatgaatattgagcaattagaaaagatttctggtaataaaataacagatgaatctttagaaaaagctatagaaatatatgatgaacacagaaaagtaatgaatgacttttcaatgttagcatcaaaatatccaggtataataacaccaactaaacgtaattatgttatgaaatctgcttattatatggataaaaaagaacatactgaaaaagttagacaattaatggatgaaattaaagctatagaaccaaaaccatttgaaggaaagagagttataactacaggtataattgcagattcagaagatttacttaaaatattagaagaaaataatatagctatagttggtgatgatatagcacatgaatctagacaatatagaacattgactccagaagcgaacacaccaatggataggttagctgagcaatttgctaatagagaatgtagtactttatatgatcctgaaaagaaaaggggtcaatatatagtagaaatggctaaagagagaaaagcagatggaattatatttttcatgacaaaattctgtgacccagaggaatatgattatccacaaatgaaaaaggattttgaagaagcaggcattccacatgtactaatagaaactgatatgcaaatgaaaaattatgaacaagctagaactgcaattcaggctttttcagaaacactttaa

SEQ ID NO: 47 - DNA, 1134 bp, Clostridium sporogenes
  source 1..1134: mol_type = genomic DNA
atgtttttcacagaacaacatgaacttattagaaaattagcaagagattttgcagagcaggaaatagagcctattgcagatgaagtagataaaactgccgagttccctaaagaaattgtgaaaaaaatggcccaaaatggtttttttggaataaaaatgcctaaagaatatggtggagctggagcagataatagagcctatgtaactataatggaagagatatcaagagcatctggtgtagctggtatatatttatcatcaccaaattcattgcttggcacaccgttcctactagttggtacagatgaacaaaaggaaaaatatctaaaaccaatgattagaggagaaaaaacattagcttttgctttaacagaaccaggtgcaggttcagacgcaggagcacttgcaacaactgctagagaagaaggggactattatatattaaatggaagaaaaacttttattacaggagctccaatatctgataatatcatagtgttcgcaaaaacagatatgagcaaaggaacaaagggaattactacatttatagtagatagtaaacaggaaggtgtatcttttggtaaacctgaagacaagatgggtatgattggttgccctactagtgatattattttagagaatgtaaaggttcataaatctgatatacttggagaagtaaataaaggctttataacagcaatgaagactttaagtgttggtcgtataggagttgcttctcaagctttaggaatagcacaagctgctgtagatgaagctgttaaatatgcaaaacaaagaaaacaatttaatagacctatagcaaaatttcaagctattcaatttaaattggctaacatggaaacaaaattaaatgcggcaaaattattagtgtataatgctgcttataaaatggattgtggagaaaaggcagataaagaagcttctatggcaaaatattttgctgctgaatctgctatacaaatcgtaaatgacgcactacaaatccatggtggttatggatacataaaagattataaaatagaaagattatatagagatgttagagtaattgctatatatgaaggaacttctgaagttcaacaaatggtaatcgcatcaaatcttttaaagtaa

SEQ ID NO: 48 - DNA, 10510 bp, synthetic construct
  source 1..10510: mol_type = other DNA
accgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgatcggcaaggtgttctggtcggcgcatagctgataacaattgagcaagaatcttcatcgaattaggggaattttcactcccctcagaacataacatagtaaatggattgaattatgaagaatggtttttatgcgacttaccgcagcaaaaataaagggaaagataagcgctcaataaacctgtctgttttccttaattctctgctggctgataatcatcacctgcaggttggctccaattatttgtatattcataaaatcgatggaaaaacttttctctttaccaaaacaaatgacaagagtctggttcagaagataaatcgctctaaagcttcagttgaagatattaagaacagcctcgcagatgacgaatcattgggattcccatcttttttgtttgttgaaggcgacaccattggttttgccagaactgttttcgggccgaccacatccgatctgacagattttttaatcgggaaaggaatgtcattaagcagtggagagcgcgttcagatagagccactgatgaggggaaccaccaaagacgatgttatgcatatgcatttcatcggccgaacaacggtgaaggtagaagccaagctacctgtatttggcgatatattaaaggtcttaggggcaacagatattgaaggggagctttttgactcattggatatagtcattaagccaaaatttaaaagggatataaaaaaggttgccaaggatattatttttaacccgtcacctcaattttcagacattagcctgcgggcaaaagatgaggccggagatattttaacagaacattatctatcagaaaaaggccatctctcagcgcctctgaacaaggtcaccaatgctgagatagctgaagagatggcatattgctacgcaagaatgaaaagtgatatactggaatgttttaaaaggcaggtgggcaaagttaaggattaattatcaggagtaattatgcggaacagaatcatgcctggtgtttacatagtaataattccttacgttatcgtaagcatttgctatctccttttccgccactacattcctggtgtttctttttcagctcatagagatggtcttggggcgacattgtcatcatatgcaggaaccatgattgcaatcctgattgctgccttgacgtttctaatcggaagcagaacgcgccgactggccaagattagagagtatgggtatatgacatcggtagttattgtctatgcccttagttttgttgagcttggagctttgtttttctgcgggttattgcttctttccagcataagcggctacatgatacccactatcgccatcggcattgcctctgcatcgttcattcatatatgcatccttgttttccaactatataatttgaccagagaacaagaataacccggcctcagcgccgggttttctttgcctcacgatcgcccccaaaacacataaccaattgtatttattgaaaaataaatagatacaactcactaaacatagcaattcagatctctcacctaccaaacaatgcccccctgcaaaaaataaattcatataaaaaacatacagataaccatctgcggtgataaattatctctggcggtgttgacataaataccactggcggtgatactgagcacatcagcaggacggtggtggaggtgtcccatgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaagattcagatagtaagattgataataatctagaccaggcagtggattaaagaggaggaaagtaatgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaaatatcaatgataaaggaggaaacaaatgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaaatccgaactatccgaactggggatcctctagagtcgaccaggaggcaaatcatgactgatcaagctgaaaaaaagcactctgcattttggggtgttatggttatagcaggtacagtaattggtggaggtatgtttgctttacctgttgatcttgccggtgcctggtttttctggggtgcctttatccttatcattgcctggttttcaatgcttcattccgggttattgttattagaagcaaatttaaattatcccgtcggctccagttttaacaccatcaccaaagatttaatcggtaacacctggaacattatcagcggtattaccgttgccttcgttctctatatcctcacttatgcctatatctctgctaatggtgcgatcattagtgaaacgatatcaatgaatttgggttatcacgctaatccacgtattgtcgggatctgcacagccattttcgttgccagcgtattgtggttaagttcgttagccgccagtcgtattacctcattgttcctcgggctgaagattatctcctttgtgatcgtgtttggttcttttttcttccaggtcgattactccattctgcgcgacgccaccagctccactgcgggaacgtcttacttcccgtatatctttatggctttgccggtgtgtctggcgtcatttggtttccacggcaatattcccagcctgattatttgctatggaaaacgcaaagataagttaatcaaaagcgtggtatttggttcgctgctggcgctggtgatttatctcttctggctctattgcaccatggggaatattccgcgagaaagctttaaggcgattatctcctcaggcggcaacgttgattcgctggtgaaatcgttcctcggcaccaaacagcacggcattatcgagttttgcctgctggtgttctctaacttagctgttgccagttcgttctttggtgtcacgctggggttgttcgattatctggcggacctgtttaagattgataactcccacggcgggcgtttcaaaaccgtgctgttaaccttcctgccacctgcgttgttgtatctgatcttcccgaacggctttatttacgggatcggcggtgccgggctgtgcgccaccatctgggcggtcattattcccgcagtgcttgcaatcaaagctcgcaagaagtttcccaatcagatgttcacggtctggggcggcaatcttattccggcgattgtcattctctttggtataaccgtgattttgtgctggttcggcaacgtctttaacgtgttacctaaatttggctaaatccgaactccaattattgaaggccgctaacgcggcctttttttgtttctggtctccctgaacgctctctactagagtcacactggctcaccttcgggtgggcctttctgcgtttataggtacccgttgaaaagggattataaaagctcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatgtatcaccgccagaggtaaaatagtcaacacgcacggtgttagatatttatcccttgcggtgatagatttaacgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatg

SEQ ID NO: 49 - DNA, 8098 bp, synthetic construct
  source 1..8098: mol_type = other DNA
cgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgggctagcaggaggaattcaatgatatgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaagattcagatagtaagattgataataatctagaccaggcagtggattaaagaggaggaaagtaatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaaatatcaatgataaaggaggaaacaaatgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaaaagaagaagtgaatgccactgctgtttgaaagcttaagccatcaaatttacctgtcagccccgctgtttatcctgatcctgctggggtactgcctgacgcgcctcgggaaatggcccgccagcatcagcgaagggatgaaccgctttgtgtttaacgttgccctgccctgcatgctgtttcgggtgatgagcaccttctcgcaaagtccgccggtggatgcgcgcctgttgctggcctttttcggcagttgcctgctggtgtttatcgtggggcgactgctggcaacaaggctgtttaatctggatggcgtagcggcgtcggtttttgcgctgggcggcatcttttccaataacgtgatgctggggatcccagtggccacggtactgctgggaccagaagccctgcccccggtggcgctggtgctggtgtttaacagcctgatcctctggaccttactgaccgtttcggtcgagtgggcaaaacagggcagcttctcgctggaaggactctggcgcaccctgctcggcgtgctgaagaatccgctgatcatcggtattctcagcggtacagcctggagctttctgcaacgtcccttgccgctgctggccgctgaaccgctaaagatgctggcctcactggccgcacctctgtcgctggtgacgctgggcatgagcctggcgcactatcgggtgcgggatggcttaaaagagagctataccatctgcctgctgaaactggtgctgcaaccgatgacggtatgggctatcgcgtggctgaccgatctgccgccgctggagagtaaagtggtggtgctgctgggatcgatggcggtcggggttaacgtctacctgatgtcgcagaagtttaatgttatcaccggcccggcagcggcgagcatgctcttttctaccgtgtttgcggcggtgacaacgccaatctggatgatgctgatgacgctggcgggctattaaatcatgatcaggagtcacaccatgaccattacgtctctggatggctatcgctggctgaagaacgatattattcgcggtaattttcaaccggatgaaaaattacgaatgagtttgctgacatcgcgttatgcacttggcgttgggccgttacgggaagctctttcgcaactggtggcggaacggctggtcacggtggtgaatcaaaaagggtatcgggtggcgtctatgtcagagcaggagctgctcgatattttcgacgcccgcgccaatatggaagcgatgttagtgagtctggcgattgcccgcggtggcgatgagtgggaggcagacgttctcgcaaaagcgcatctgctgagtaagcttgaggcctgtgacgccagcgagaaaatgcttgatgagtgggatctgcgtcatcaggcgtttcatacggcaattgtggcgggctgtggttctcactatttgctgcaaatgcgtgaacggttgtttgatctggcggcgcgttatcgatttatctggctgcggcgaacggtgctttcggtggaaatgctggaggataaacacgatcagcaccagaccctgactgcggcggtactggcgcgagataccgcgcgcgccagtgagttaatgcgccagcatttactgacgccaattcccattatccagcaggcgatggctggcaattaacgctctctactagagtcacactggctcaccttcgggtgggcctttctgcgggtctcacgctcttgatatgcaggtgctgcaggcttaatttgatgcctggcagtttatggcgggcgtcctgcccgccaccctccgggccgttgcttcctgcaggcacggcagcggctgcgcagggctcttctcgatcaatatcgttcactcatccaatctcactgacggttaggcgtgcctggtaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatg

SEQ ID NO: 50 - DNA, 6313 bp, synthetic construct
  source 1..6313: mol_type = other DNA
aaatatcaatgataaaggaggaaacaaatgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaaatccgaactccaattattgatgaaaagggattataaaagctcacttattcaggcgtagcacccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatgattcagcgggttcgtgcgagctgtagacaattaatactcggctcgtataatgtgtggaaggctagcaggaggaattcaatgatatgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaagattcagatagtaagattgataataatctagaccaggcagtggattaaagaggaggaaagtaatgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgt

SEQ ID NO: 51 - DNA, 7225 bp, synthetic construct
  source 1..7225: mol_type = other DNA
tccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatgattcagcgggttcgtgcgagctgtagacaattaatactcggctcgtataatgtgtggaaggctagcaggaggaattcaatgatatgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaagattcagatagtaagattgataataatctagaccaggcagtggattaaagaggaggaaagtaatgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaaatatcaatgataaaggaggaaacaaatgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaaatccgaactccaattattgatgaaaagggattataaaagcgttaatagcccgccagcgtcatcagcatcatccagattggcgttgtcaccgccgcaaacacggtagaaaagagcatgctcgccgctgccgggccggtgataacattaaacttctgcgacatcaggtagacgttaaccccgaccgccatcgatcccagcagcaccaccactttactctccagcggcggcagatcggtcagccacgcgatagcccataccgtcatcggttgcagcaccagtttcagcaggcagatggtatagctctcttttaagccatcccgcacccgatagtgcgccaggctcatgcccagcgtcaccagcgacagaggtgcggccagtgaggccagcatctttagcggttcagcggccagcagcggcaagggacgttgcagaaagctccaggctgtaccgctgagaataccgatgatcagcggattcttcagcacgccgagcagggtgcgccagagtccttccagcgagaagctgccctgttttgcccactcgaccgaaacggtcagtaaggtccagaggatcaggctgttaaacaccagcaccagcgccaccgggggcagggcttctggtcccagcagtaccgtggccactgggatccccagcatcacgttattggaaaagatgccgcccagcgcaaaaaccgacgccgctacgccatccagattaaacagccttgttgccagcagtcgccccacgataaacaccagcaggcaactgccgaaaaaggccagcaacaggcgcgcatccaccggcggactttgcgagaaggtgctcatcacccgaaacagcatgcagggcagggcaacgttaaacacaaagcggttcatcccttcgctgatgctggcgggccatttcccgaggcgcgtcaggcagtaccccagcaggatcaggataaacagcggggctgacaggtaaatttgatggcttaagctttcaaacagcagtggcattcacttcttcttacgcgccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcac

SEQ ID NO: 52 - DNA, 6298 bp, synthetic construct
  source 1..6298: mol_type = other DNA
atatcaatgataaaggaggaaacaaatgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaaatccgaactccaattattgatgaaaagggattataaaagctcacttattcaggcgtagcacccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatgattcagcgggttcgtgcgagctgtagacaattaatactcggctcgtataatgtgtggaaggctagcaggaggaattcaatgatatgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaagattcagatagtaagattgataataatctagaccaggcagtggattaaagaggaggaaagtaatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaa

SEQ ID NO: 53 - DNA, 7210 bp, synthetic construct
  source 1..7210: mol_type = other DNA
tccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatgattcagcgggttcgtgcgagctgtagacaattaatactcggctcgtataatgtgtggaaggctagcaggaggaattcaatgatatgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaagattcagatagtaagattgataataatctagaccaggcagtggattaaagaggaggaaagtaatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaaatatcaatgataaaggaggaaacaaatgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaaatccgaactccaattattgatgaaaagggattataaaagcgttaatagcccgccagcgtcatcagcatcatccagattggcgttgtcaccgccgcaaacacggtagaaaagagcatgctcgccgctgccgggccggtgataacattaaacttctgcgacatcaggtagacgttaaccccgaccgccatcgatcccagcagcaccaccactttactctccagcggcggcagatcggtcagccacgcgatagcccataccgtcatcggttgcagcaccagtttcagcaggcagatggtatagctctcttttaagccatcccgcacccgatagtgcgccaggctcatgcccagcgtcaccagcgacagaggtgcggccagtgaggccagcatctttagcggttcagcggccagcagcggcaagggacgttgcagaaagctccaggctgtaccgctgagaataccgatgatcagcggattcttcagcacgccgagcagggtgcgccagagtccttccagcgagaagctgccctgttttgcccactcgaccgaaacggtcagtaaggtccagaggatcaggctgttaaacaccagcaccagcgccaccgggggcagggcttctggtcccagcagtaccgtggccactgggatccccagcatcacgttattggaaaagatgccgcccagcgcaaaaaccgacgccgctacgccatccagattaaacagccttgttgccagcagtcgccccacgataaacaccagcaggcaactgccgaaaaaggccagcaacaggcgcgcatccaccggcggactttgcgagaaggtgctcatcacccgaaacagcatgcagggcagggcaacgttaaacacaaagcggttcatcccttcgctgatgctggcgggccatttcccgaggcgcgtcaggcagtaccccagcaggatcaggataaacagcggggctgacaggtaaatttgatggcttaagctttcaaacagcagtggcattcacttcttcttacgcgccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttc

SEQ ID NO: 54 - DNA, 9199 bp, synthetic construct
  source 1..9199: mol_type = other DNA
ggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaaatatcaatgataaaggaggaaacaaatgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaaatccgaactccaattattgaaggccgctaacgcggcctttttttgtttctggtctccctgaacgctctctactagagtcacactggctcaccttcgggtgggcctttctgcgtttataggtacccgttgaaaagggattataaaagctcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatgtatcaccgccagaggtaaaatagtcaacacgcacggtgttagatatttatcccttgcggtgatagatttaacgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgatcggcaaggtgttctggtcggcgcatagctgataacaattgagcaagaatcttcatcgaattaggggaattttcactcccctcagaacataacatagtaaatggattgaattatgaagaatggtttttatgcgacttaccgcagcaaaaataaagggaaagataagcgctcaataaacctgtctgttttccttaattctctgctggctgataatcatcacctgcaggttggctccaattatttgtatattcataaaatcgatggaaaaacttttctctttaccaaaacaaatgacaagagtctggttcagaagataaatcgctctaaagcttcagttgaagatattaagaacagcctcgcagatgacgaatcattgggattcccatcttttttgtttgttgaaggcgacaccattggttttgccagaactgttttcgggccgaccacatccgatctgacagattttttaatcgggaaaggaatgtcattaagcagtggagagcgcgttcagatagagccactgatgaggggaaccaccaaagacgatgttatgcatatgcatttcatcggccgaacaacggtgaaggtagaagccaagctacctgtatttggcgatatattaaaggtcttaggggcaacagatattgaaggggagctttttgactcattggatatagtcattaagccaaaatttaaaagggatataaaaaaggttgccaaggatattatttttaacccgtcacctcaattttcagacattagcctgcgggcaaaagatgaggccggagatattttaacagaacattatctatcagaaaaaggccatctctcagcgcctctgaacaaggtcaccaatgctgagatagctgaagagatggcatattgctacgcaagaatgaaaagtgatatactggaatgttttaaaaggcaggtgggcaaagttaaggattaattatcaggagtaattatgcggaacagaatcatgcctggtgtttacatagtaataattccttacgttatcgtaagcatttgctatctccttttccgccactacattcctggtgtttctttttcagctcatagagatggtcttggggcgacattgtcatcatatgcaggaaccatgattgcaatcctgattgctgccttgacgtttctaatcggaagcagaacgcgccgactggccaagattagagagtatgggtatatgacatcggtagttattgtctatgcccttagttttgttgagcttggagctttgtttttctgcgggttattgcttctttccagcataagcggctacatgatacccactatcgccatcggcattgcctctgcatcgttcattcatatatgcatccttgttttccaactatataatttgaccagagaacaagaataacccggcctcagcgccgggttttctttgcctcacgatcgcccccaaaacacataaccaattgtatttattgaaaaataaatagatacaactcactaaacatagcaattcagatctctcacctaccaaacaatgcccccctgcaaaaaataaattcatataaaaaacatacagataaccatctgcggtgataaattatctctggcggtgttgacataaataccactggcggtgatactgagcacatcagcaggacggtggtggaggtgtcccatgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaaaagattgataataatctagaccaggcagtggattaaagaggaggaaagtaatgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacac

SEQ ID NO: 55 - DNA, 11095 bp, synthetic construct
  source 1..11095: mol_type = other DNA
accgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgatcggcaaggtgttctggtcggcgcatagctgataacaattgagcaagaatcttcatcgaattaggggaattttcactcccctcagaacataacatagtaaatggattgaattatgaagaatggtttttatgcgacttaccgcagcaaaaataaagggaaagataagcgctcaataaacctgtctgttttccttaattctctgctggctgataatcatcacctgcaggttggctccaattatttgtatattcataaaatcgatggaaaaacttttctctttaccaaaacaaatgacaagagtctggttcagaagataaatcgctctaaagcttcagttgaagatattaagaacagcctcgcagatgacgaatcattgggattcccatcttttttgtttgttgaaggcgacaccattggttttgccagaactgttttcgggccgaccacatccgatctgacagattttttaatcgggaaaggaatgtcattaagcagtggagagcgcgttcagatagagccactgatgaggggaaccaccaaagacgatgttatgcatatgcatttcatcggccgaacaacggtgaaggtagaagccaagctacctgtatttggcgatatattaaaggtcttaggggcaacagatattgaaggggagctttttgactcattggatatagtcattaagccaaaatttaaaagggatataaaaaaggttgccaaggatattatttttaacccgtcacctcaattttcagacattagcctgcgggcaaaagatgaggccggagatattttaacagaacattatctatcagaaaaaggccatctctcagcgcctctgaacaaggtcaccaatgctgagatagctgaagagatggcatattgctacgcaagaatgaaaagtgatatactggaatgttttaaaaggcaggtgggcaaagttaaggattaattatcaggagtaattatgcggaacagaatcatgcctggtgtttacatagtaataattccttacgttatcgtaagcatttgctatctccttttccgccactacattcctggtgtttctttttcagctcatagagatggtcttggggcgacattgtcatcatatgcaggaaccatgattgcaatcctgattgctgccttgacgtttctaatcggaagcagaacgcgccgactggccaagattagagagtatgggtatatgacatcggtagttattgtctatgcccttagttttgttgagcttggagctttgtttttctgcgggttattgcttctttccagcataagcggctacatgatacccactatcgccatcggcattgcctctgcatcgttcattcatatatgcatccttgttttccaactatataatttgaccagagaacaagaataacccggcctcagcgccgggttttctttgcctcacgatcgcccccaaaacacataaccaattgtatttattgaaaaataaatagatacaactcactaaacatagcaattcagatctctcacctaccaaacaatgcccccctgcaaaaaataaattcatataaaaaacatacagataaccatctgcggtgataaattatctctggcggtgttgacataaataccactggcggtgatactgagcacatcagcaggacggtggtggaggtgtcccatgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaaaagattgataataatctagaccaggcagtggattaaagaggaggaaagtaatgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaaatatcaatgataaaggaggaaacaaatgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaaatccgaactccaattattgaaggccgctaacgcggcctttttttgtttctggtctccctgaacgctctctactagagtcacactggctcaccttcgggtgggcctttctgcgtttataggtacccgttgaaaagggattataaaagcttataatccaagtaaaacataatataacacggtaaccggcagcgccacaagcataccaaaaatcactgccgtagaaagaatatctgggtgcacattgtattctttggcaaaaacaaaggggacgattccctggggcagtgccgcctgtacgatggcaatgtgtaacaggtccccgcgaataccgatagcgatactggtggcagcaataaccgctggtccagtcaggaagcgaaccgccatcgcaaacccggctacagacttcccgcacgcgataatttttggttgcaaggccatgaacaggccaagactaaacattgccatgcccaatcccgcgtcgctcaaaatggaaatgctaccgctcatgatagttggcattttaatattccacttaaacgatactaaagaccaggccagaccaaaaaggcttgagtatgtgttaggattgcgaattaacttacgccagaccataataagaatcaagcgtgtcattacactcgccggaggcatctgctgcttacgaggtccgggacctccatcctcgacatcagaccccttcttacccatgtagggagatttcccgccattgttcccgtcctgatccatttctacatggcctttacgaccaggcgacatattttcaataagattctgcatagccttggtcgctaagttgtcatggggtgggatggacactttagggtcggttgacacgtcagtactagatccgcgcgtcatggcattctttgcgttcgcctcgctgacgggtgaggcgctgctagaccagacgaacatattcatttccttgttttggccacctaccccgacgcctcccccgcttccccctccgcctgactccttcttcttaacaccggacgcaccactcgttgatcctgtaaacatcgggttagggggtgggtaacttggcaccgaattgttgttgtataattcccctgacatagagcgaccgccacgacctgctttctttgcagttttcataacttcttcatcgaaattagatgtacgcggtgtgacccctttggaagactgaaggctgtacacgtcaccacctgggcctgctcctgcgcctccgtagctattagtatagccgtggcgcggtgaaggggccttactagcattgaacatagcgtaaaagtccgtttggttgaaactggatgcacgcggggtgggctcgcgactagactgcacactgtaaatctcaaccccagtaagattactagcacgtggtgtaatcattgaactgttaagaccccctccatgggacttgttaaatgaactaatcatactggatgcagcactgctacggcgtactacaacatgaagcttcccatcatcgccgatctccgcgtcggtctgcaatggctcgcgcccattcaagctgatgacatcactatcaacacgaaaactagtgattgaccccgcagtctccggaaattgttcactaatcagaagtttagcgccgcgaaactcgaacaaaaacagcatcaaagtgtaccaaataatactctgtaagacgacgatttggaccataaggttaccagaaaagtcgccgtacattgcgcgcaaaagggggatacccattaccagcgtattgggcaaggtagaaagggaaaaaagtgtaatcatccattctaaactcccgcgacgagagaaggcttgccacaggaacaaggctgctaagataaccactttctgtaaggaatcagcggctaaaaaatgataattcattgcataagggtcatttgagctaatgaaatgaaatgataacaaaggaacggcaaaaacagcgacaaaacgattaattcctgagcattggtcgggtgtaaaaataccccaccagcggacagagccgtaagcaaggatcatagccacatacaggggcaccatagctgccagcacgtcgtacatatctttgcctgtaatcattcacttcttcttacgcgccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatgtatcaccgccagaggtaaaatagtcaacacgcacggtgttagatatttatcccttgcggtgatagatttaacgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatg

SEQ ID NO: 56 - DNA, 10112 bp, synthetic construct
  source 1..10112: mol_type = other DNA
accgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgatcggcaaggtgttctggtcggcgcatagctgataacaattgagcaagaatcttcatcgaattaggggaattttcactcccctcagaacataacatagtaaatggattgaattatgaagaatggtttttatgcgacttaccgcagcaaaaataaagggaaagataagcgctcaataaacctgtctgttttccttaattctctgctggctgataatcatcacctgcaggttggctccaattatttgtatattcataaaatcgatggaaaaacttttctctttaccaaaacaaatgacaagagtctggttcagaagataaatcgctctaaagcttcagttgaagatattaagaacagcctcgcagatgacgaatcattgggattcccatcttttttgtttgttgaaggcgacaccattggttttgccagaactgttttcgggccgaccacatccgatctgacagattttttaatcgggaaaggaatgtcattaagcagtggagagcgcgttcagatagagccactgatgaggggaaccaccaaagacgatgttatgcatatgcatttcatcggccgaacaacggtgaaggtagaagccaagctacctgtatttggcgatatattaaaggtcttaggggcaacagatattgaaggggagctttttgactcattggatatagtcattaagccaaaatttaaaagggatataaaaaaggttgccaaggatattatttttaacccgtcacctcaattttcagacattagcctgcgggcaaaagatgaggccggagatattttaacagaacattatctatcagaaaaaggccatctctcagcgcctctgaacaaggtcaccaatgctgagatagctgaagagatggcatattgctacgcaagaatgaaaagtgatatactggaatgttttaaaaggcaggtgggcaaagttaaggattaattatcaggagtaattatgcggaacagaatcatgcctggtgtttacatagtaataattccttacgttatcgtaagcatttgctatctccttttccgccactacattcctggtgtttctttttcagctcatagagatggtcttggggcgacattgtcatcatatgcaggaaccatgattgcaatcctgattgctgccttgacgtttctaatcggaagcagaacgcgccgactggccaagattagagagtatgggtatatgacatcggtagttattgtctatgcccttagttttgttgagcttggagctttgtttttctgcgggttattgcttctttccagcataagcggctacatgatacccactatcgccatcggcattgcctctgcatcgttcattcatatatgcatccttgttttccaactatataatttgaccagagaacaagaataacccggcctcagcgccgggttttctttgcctcacgatcgcccccaaaacacataaccaattgtatttattgaaaaataaatagatacaactcactaaacatagcaattcagatctctcacctaccaaacaatgcccccctgcaaaaaataaattcatataaaaaacatacagataaccatctgcggtgataaattatctctggcggtgttgacataaataccactggcggtgatactgagcacatcagcaggacggtggtggaggtgtcccatgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaaaagattgataataatctagaccaggcagtggattaaagaggaggaaagtaatgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaaatatcaatgataaaggaggaaacaaatgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaaatccgaactccaattattgaaggccgctaacgcggcctttttttgtttctggtctccctgaacgctctctactagagtcacactggctcaccttcgggtgggcctttctgcgtttataggtacccgttgaaaagggattataaaagcgttaatagcccgccagcgtcatcagcatcatccagattggcgttgtcaccgccgcaaacacggtagaaaagagcatgctcgccgctgccgggccggtgataacattaaacttctgcgacatcaggtagacgttaaccccgaccgccatcgatcccagcagcaccaccactttactctccagcggcggcagatcggtcagccacgcgatagcccataccgtcatcggttgcagcaccagtttcagcaggcagatggtatagctctcttttaagccatcccgcacccgatagtgcgccaggctcatgcccagcgtcaccagcgacagaggtgcggccagtgaggccagcatctttagcggttcagcggccagcagcggcaagggacgttgcagaaagctccaggctgtaccgctgagaataccgatgatcagcggattcttcagcacgccgagcagggtgcgccagagtccttccagcgagaagctgccctgttttgcccactcgaccgaaacggtcagtaaggtccagaggatcaggctgttaaacaccagcaccagcgccaccgggggcagggcttctggtcccagcagtaccgtggccactgggatccccagcatcacgttattggaaaagatgccgcccagcgcaaaaaccgacgccgctacgccatccagattaaacagccttgttgccagcagtcgccccacgataaacaccagcaggcaactgccgaaaaaggccagcaacaggcgcgcatccaccggcggactttgcgagaaggtgctcatcacccgaaacagcatgcagggcagggcaacgttaaacacaaagcggttcatcccttcgctgatgctggcgggccatttcccgaggcgcgtcaggcagtaccccagcaggatcaggataaacagcggggctgacaggtaaatttgatggcttaagctttcaaacagcagtggcattcacttcttcttacgcgccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatgtatcaccgccagaggtaaaatagtcaacacgcacggtgttagatatttatcccttgcggtgatagatttaacgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatg

SEQ ID NO: 57 - DNA, 3064 bp, synthetic construct
  source 1..3064: mol_type = other DNA
attcagcgggttcgtgcgagctgtagacaattaatactcggctcgtataatgtgtggaaggctagcaggaggaattcaatgatatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaaatccgaactccaattattgatgaaaagggattataaaagctcacttattcaggcgtagcacccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatg

SEQ ID NO: 58 - DNA, 4083 bp, synthetic construct
  source 1..4083: mol_type = other DNA
attcagcgggttcgtgcgagctgtagacaattaatactcggctcgtataatgtgtggaaggctagcaggaggaattcaatgatatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaaatccgaactccaattattgaaggccgctaacgcggcctttttttgtttctggtctccctgaacgctctctactagagtcacactggctcaccttcgggtgggcctttctgcgtttataggtacccgttgaaaagggattataaaagcgttaatagcccgccagcgtcatcagcatcatccagattggcgttgtcaccgccgcaaacacggtagaaaagagcatgctcgccgctgccgggccggtgataacattaaacttctgcgacatcaggtagacgttaaccccgaccgccatcgatcccagcagcaccaccactttactctccagcggcggcagatcggtcagccacgcgatagcccataccgtcatcggttgcagcaccagtttcagcaggcagatggtatagctctcttttaagccatcccgcacccgatagtgcgccaggctcatgcccagcgtcaccagcgacagaggtgcggccagtgaggccagcatctttagcggttcagcggccagcagcggcaagggacgttgcagaaagctccaggctgtaccgctgagaataccgatgatcagcggattcttcagcacgccgagcagggtgcgccagagtccttccagcgagaagctgccctgttttgcccactcgaccgaaacggtcagtaaggtccagaggatcaggctgttaaacaccagcaccagcgccaccgggggcagggcttctggtcccagcagtaccgtggccactgggatccccagcatcacgttattggaaaagatgccgcccagcgcaaaaaccgacgccgctacgccatccagattaaacagccttgttgccagcagtcgccccacgataaacaccagcaggcaactgccgaaaaaggccagcaacaggcgcgcatccaccggcggactttgcgagaaggtgctcatcacccgaaacagcatgcagggcagggcaacgttaaacacaaagcggttcatcccttcgctgatgctggcgggccatttcccgaggcgcgtcaggcagtaccccagcaggatcaggataaacagcggggctgacaggtaaatttgatggcttaagctttcaaacagcagtggcattcacttcttcttacgcgccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatg

SEQ ID NO: 59 - DNA, 4678 bp, synthetic construct
  source 1..4678: mol_type = other DNA
attcagcgggttcgtgcgagctgtagacaattaatactcggctcgtataatgtgtggaaggctagcaggaggaattcaatgatatgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaaaagattgataataatctagaccaggcagtggattaaagaggaggaaagtaatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaatcacttcttcttacgcgccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatg

SEQ ID NO: 60 - DNA, 4175 bp, synthetic construct
  source 1..4175: mol_type = other DNA
attcagcgggttcgtgcgagctgtagacaattaatactcggctcgtataatgtgtggaaggctagcaggaggaattcaatgatatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaaatatcaatgataaaggaggaaacaaatggaaagtggtcatcgctttgatgctcagacgctgcacagttttattcaggctgtatttcgtcagatgggtagcgaggaacaagaagcgaaattagttgccgatcatttaatcgcggcaaacctggcagggcatgattcacatggtattggcatgatcccaagctatgtacgctcctggagtcaggggcacctgcaaattaaccatcatgccaaaaccgttaaagaggcgggggcggcggtcacgctcgatggcgatcgcgcatttggtcaggtcgcggcacatgaagcgatggcgctggggattgagaaagcgcatcagcacggtattgccgccgtggcgctacataactcgcatcatatcggccgtatcggttactgggcggagcagtgtgcagcggcggggtttgtctctatccactttgttagcgtggtcggtattccaatggtcgcgccgttccacggtcgcgacagccgctttggcaccaatccgttctgtgtggttttccctcgtaaagataatttcccgctgttgcttgattacgccaccagcgccattgcatttggcaaaacccgcgtcgcctggcataaaggcgtccccgtgccgccaggttgcctgattgacgttaacggcgtgccgacgaccaatccggcggtaatgcaggagtcgccgttgggttcgctgttgacctttgccgaacataaaggctacgcccttgcagcgatgtgtgaaattcttggcggggcgctttccggcggtaaaacgacgcatcaggaaacgttacaaaccagtcccgatgccattcttaactgcatgaccactatcatcatcaacccggaactcttcggcgcgccggattgtaacgcgcagaccgaagcctttgccgagtgggtgaaagcctcgccgcatgatgatgataagccgattttgctaccgggcgagtgggaagtgaacacgcgtcgcgaacggcagaagcaggggattccactggatgcgggaagctggcaggccatttgtgatgcagcgcggcagattggtatgccggaagagacgttgcaggctttctgtcagcagttagccagctaaatccgaactccaattattgatgaaaagggattataaaagctcacttattcaggcgtagcacccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatg

SEQ ID NO: 61 - DNA, 4039 bp, synthetic construct
  source 1..4039: mol_type = other DNA
ccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatgattcagcgggttcgtgcgagctgtagacaattaatactcggctcgtataatgtgtggaaggctagcaggaggaattcaatgatatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaaatatcaatgataaaggaggaaacaaatggtgaccatgaatcgtaataaagttgttatcgtgggtacgggccaggtcggcgccaccgctgcgttcggcattgttacgcatggtttgtgcaacgaactggtcttgatcgaccgttccgccgccaaggcattgggggaggctcgtgaccttgacgacggtagcgaatttcaggaccgtcacgttaaggtccgcgcgggagattacgccgattgcaaagacgctgacattgttgttattactgttgggcgtaagccaccagcgaacagtaaccgtatggcggaacttggttttacggtcggtttggtgggcgaagtagttgataatgttatggctagtggatttgatggagttattgttatggttagcaaccccgtcgatgttatggcttggtatgcttggaagcgttcgggactgccccgcacgcaagtccttggaacgggtaccgccttagatacctcccgccttaagacaattatcggagaagaaactggtttagatccacgtaacgtaggcggctttgttatgggggaacatggtgattctcagttcactgcttggagcactgtatcgttaggcggtaaaccgtttgctcgctttttagcagacaatcaagatcgcttcgcatcggtatcaactaccgagatcgaggaaaaaacgcgcacacgtggcgacgaaatcgtagcggccaagggcgggaccaattttggcattgcgtcaacagttgccggtatcgtacaaaccatcctgtgggacgagcgccgcatcgtaccagtaagcacgctgcttgatggagagtacggtgaacatgacgtatttttgggtgtcccaacggaactgcgtgcaaacggggcgaatgaaatcgtagaattggacttaagcgaagacgagcgcgctaagttacatcattcggccgaattggtgcgtgaccactgcaaaggattgctttgaatccgaactccaattattgatgaaaagggattataaaagctcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgct

SEQ ID NO: 62 - DNA, 4085 bp, synthetic construct
  source 1..4085: mol_type = other DNA
attcagcgggttcgtgcgagctgtagacaattaatactcggctcgtataatgtgtggaaggctagcaggaggaattcaatgatatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaaatatcaatgataaaggaggaaacaaatgaaaatcttagcttattgtgtaagacctgatgaaattgattcatttaaaaatttttctgaaaaatatggacatactgttgatttaataccagattcttttggtccaagtgtggctcatttagctaaaggatatgatggcatttcaattttgggaaatgatacttgtaacagagaagcattagagaaaattaaagattgtggtataaaatatttagcaaccagaactgctggagtaaataatatagattttgatgctgcaaaagaatttggtataaatgtagctaatgttccagcttattctcctaattcagtttcagagtttactgtaggactcgcattatccctaacaaggaaaattccttttgcattaaaaagagtagagcttaacaattttgcattaggtggattaataggagtagaattaagaaacttaactttaggagttataggaactggaagaataggtttaaaagtaattgaaggcttttcaggctttggtatgaaaaagatgataggttacgatatttttgaaaatgaaaaggctaaagaatatattgaatataaatctttagatgaagtatataaagaagcagacataataacactacatgcacctttaacagatgataattaccatatgatagggaaagagagtattgctaaaatgaaagacggtgtattcataataaatgcagcacgaggagcattaattgatagtgaagctttaatagaaggactgaaatctggtaaaatagctggagcagccttagattcttatgaatatgaacaaggagttttccataacaataaaatgaatgaaatcatgaaagatgatacattagcaagacttaagagttttccaaatgtagttattacacctcatttaggattctatacagatgaagctgtatcaaatatggttgaaattactcttatgaatttacaagagtttgaattaaaaggaacttgtaaaaatcagagggtatgtaagtaaatccgaactccaattattgatgaaaagggattataaaagctcacttattcaggcgtagcacccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatg

SEQ ID NO: 63 - DNA, 8486 bp, synthetic construct
  source 1..8486: mol_type = other DNA
attcagcgggttcgtgcgagctgtagacaattaatactcggctcgtataatgtgtggaaggctagcaggaggaattcaatgatatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaaatatcaatgataaaggaggaaacaaatgaaaatcttagcttattgtgtaagacctgatgaaattgattcatttaaaaatttttctgaaaaatatggacatactgttgatttaataccagattcttttggtccaagtgtggctcatttagctaaaggatatgatggcatttcaattttgggaaatgatacttgtaacagagaagcattagagaaaattaaagattgtggtataaaatatttagcaaccagaactgctggagtaaataatatagattttgatgctgcaaaagaatttggtataaatgtagctaatgttccagcttattctcctaattcagtttcagagtttactgtaggactcgcattatccctaacaaggaaaattccttttgcattaaaaagagtagagcttaacaattttgcattaggtggattaataggagtagaattaagaaacttaactttaggagttataggaactggaagaataggtttaaaagtaattgaaggcttttcaggctttggtatgaaaaagatgataggttacgatatttttgaaaatgaaaaggctaaagaatatattgaatataaatctttagatgaagtatataaagaagcagacataataacactacatgcacctttaacagatgataattaccatatgatagggaaagagagtattgctaaaatgaaagacggtgtattcataataaatgcagcacgaggagcattaattgatagtgaagctttaatagaaggactgaaatctggtaaaatagctggagcagccttagattcttatgaatatgaacaaggagttttccataacaataaaatgaatgaaatcatgaaagatgatacattagcaagacttaagagttttccaaatgtagttattacacctcatttaggattctatacagatgaagctgtatcaaatatggttgaaattactcttatgaatttacaagagtttgaattaaaaggaacttgtaaaaatcagagggtatgtaagtaaaaaggaggaaacaaatggaaaacaatacaaatatgtttagtggagtaaaggttattgaattagcaaattttatagctgctccagcagcaggtagattttttgctgatggtggtgcagaggtaataaaaattgaatcacctgctggagatcctttaagatatactgctccttcagaaggaagaccattaagccaagaagaaaatactacttatgatttggaaaatgcaaataaaaaagcaatagtattaaatcttaaaagcgaaaaaggtaaaaagatattacatgaaatgttagcagaagcagatatattattaactaattggagaacaaaggctttagttaaacaaggattagactatgaaacactaaaagaaaaatatcctaaattagtttttgcacaaataactggttatggtgaaaaaggaccagataaagatcttccaggctttgattatactgcatttttcgctagaggcggtgtttcaggtactctttatgaaaaaggaactgtgcctccaaatgttgttccaggacttggagaccatcaagctgggatgtttttagcagcgggtatggcaggagctttatataaagcaaaaacaacaggacaaggagataaagtaacagtaagtttaatgcatagtgctatgtatggactaggtattatgatacaagctgctcaatataaagatcatggattagtatatccgataaatcgtaatgaaactccaaatccttttatagtttcatataaatctaaggatgattactttgttcaagtatgtatgccaccatatgatgttttctatgatagatttatgaccgctttaggaagagaagatttagttggagacgaaagatacaataaaatagaaaatttaaaagatggacgtgctaaggaagtatacagtataatcgaacaacaaatggttacaaagacaaaggatgaatgggataaaatatttagagatgcagacattccatttgctatcgcacaaacttgggaagatttattagaagatgaacaagcttgggcaaatgattatttgtataagatgaaatatccaacaggaaacgaaagagcattagtaagacttccagtattctttaaagaagcaggattaccagaatataatcaatcaccacaaatagcagaaaatactgtagaagttttaaaagaaatgggatatacagaacaagagattgaggaattagaaaaagataaagatataatggtaaggaaggaaaaataatggcagacatttatactatgggtgtagacataggttcaactgcatcaaaaacagtagtattaaaaaatggtaaagaaattgtaagtcaagcagtaataagtgtaggggccggaacaagtggccccaagagagctatagattctgtattaaaagatgctaaattatccattgaagatttagactatattgtatccactggatatggaagaaatagtttcgattttgctaacaaacaaatttctgaattaagttgtcatgcaaaaggggtctatttcgataacaataaagctagaacagttattgatataggcggacaagatattaaagtattaaaattagcggatagtggaagacttttaaactttataatgaatgataaatgtgctgcaggaacgggacgatttttagatgtaatgtctagagtaatagaagttccagttgatgagttaggaaaaaaagcattagaaagcaaaaatccttgtactattagttctacctgtacagtatttgcagagtcagaagtaatttctcaacttgcaagaggagttaaaactgaagatttgatagcaggaatttgtaaatctgtagcatcaagagtggctagccttgcaaagagaagtggtatagaagaattagtagttatgagtggaggagtagctaaaaatataggtgtagtaaaggcaatggaagcagaattgggaagagacatatatatatctaaaaattctcaattaaatggagcattgggagcaagtctatacgcttatgaaagttttcaaaaagaaaggagctaaaaacatgagtgatagaaataaggaagtaaaagaaaaaaaggcaaagcattatcttagagagattactgcaaagcattacaaagaagctctcgaagcaaaagaaaggggagaaaaggttggttggtgtgcatctaacttcccacaagaaatagctacaacattgggggtaaaagttgtttatccagaaaatcatgcagcagctgtagcagctagagggaatggacaaaatatgtgtgaacatgctgaggctatgggtttttctaatgatgtatgtggttatgcaagagtaaatttagctgttatggacataggtcatagtgaagatcaaccaatacctatgccagactttgtactttgctgtaataacatttgtaatcaaatgattaaatggtatgagcatatagcaaaaactttagatataccaatgattcttatagatataccatacaatacagaaaatactgtttcacaagatagaattaaatatattagagcacaatttgatgatgcaataaaacaattggaagaaataacaggcaaaaaatgggatgaaaataaatttgaagaagttatgaaaatatcccaagaaagtgcaaaacaatggttaagagcagcatcctatgcaaagtataaaccttcaccatttagcggatttgatttatttaatcatatggctgtagcagtttgtgcaagaggtacacaagaagctgcagatgcatttaagatgttagcagatgaatatgaggagaatgtaaaaactggaaaatccacttataggggagaagaaaaacaacgtatattatttgaagggattgcctgttggccatatttgagacataaattaactaagcttagtgaatatggtatgaacgtaactgcaactgtatacgcagaagcctttggtgttatatatgagaatatggatgaattaatggctgcttataataaagttcctaattcaattagttttgaaaacgcattaaaaatgagattaaatgctgttacaagcactaatacagaaggtgctgttattcatataaatagaagctgtaaattatggagtggatttttatatgagctagcaagaagattagaaaaggaaacaggaattcctgtagtatcatttgatggggaccaggcagacccaagaaatttctcagaagctcaatatgatactagaattcaaggacttaatgaagtaatggttgctaaaaaggaggctgaataagatgtcaaattcagataaattttttaatgactttaaggatattgtagaaaatcctaaaaaatatataatgaagcatatggaacaaactggacaaaaggctataggatgtatgccattatatactcctgaggaacttgtattagctgctggaatgtttccagtaggggtatggggaagcaatacagaactttcaaaagctaaaacatatttcccagcatttatttgttcaatattacaaacaacattggaaaatgcattaaatggagaatatgatatgttatctggtatgatgattacaaattattgtgattcattaaaatgcatgggacaaaattttaaactaaccgttgaaaatattgagtttatcccagtaacagttccacaaaatagaaaaatggaagctggaaaagagtttttaaaaagtcaatataaaatgaatattgagcaattagaaaagatttctggtaataaaataacagatgaatctttagaaaaagctatagaaatatatgatgaacacagaaaagtaatgaatgacttttcaatgttagcatcaaaatatccaggtataataacaccaactaaacgtaattatgttatgaaatctgcttattatatggataaaaaagaacatactgaaaaagttagacaattaatggatgaaattaaagctatagaaccaaaaccatttgaaggaaagagagttataactacaggtataattgcagattcagaagatttacttaaaatattagaagaaaataatatagctatagttggtgatgatatagcacatgaatctagacaatatagaacattgactccagaagcgaacacaccaatggataggttagctgagcaatttgctaatagagaatgtagtactttatatgatcctgaaaagaaaaggggtcaatatatagtagaaatggctaaagagagaaaagcagatggaattatatttttcatgacaaaattctgtgacccagaggaatatgattatccacaaatgaaaaaggattttgaagaagcaggcattccacatgtactaatagaaactgatatgcaaatgaaaaattatgaacaagctagaactgcaattcaggctttttcagaaacactttaaatccgaactccaattattgatgaaaagggattataaaagctcacttattcaggcgtagcacccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatg

SEQ ID NO: 64 - DNA, 9634 bp, synthetic construct
  source 1..9634: mol_type = other DNA
attcagcgggttcgtgcgagctgtagacaattaatactcggctcgtataatgtgtggaaggctagcaggaggaattcaatgatatggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaaatatcaatgataaaggaggaaacaaatgaaaatcttagcttattgtgtaagacctgatgaaattgattcatttaaaaatttttctgaaaaatatggacatactgttgatttaataccagattcttttggtccaagtgtggctcatttagctaaaggatatgatggcatttcaattttgggaaatgatacttgtaacagagaagcattagagaaaattaaagattgtggtataaaatatttagcaaccagaactgctggagtaaataatatagattttgatgctgcaaaagaatttggtataaatgtagctaatgttccagcttattctcctaattcagtttcagagtttactgtaggactcgcattatccctaacaaggaaaattccttttgcattaaaaagagtagagcttaacaattttgcattaggtggattaataggagtagaattaagaaacttaactttaggagttataggaactggaagaataggtttaaaagtaattgaaggcttttcaggctttggtatgaaaaagatgataggttacgatatttttgaaaatgaaaaggctaaagaatatattgaatataaatctttagatgaagtatataaagaagcagacataataacactacatgcacctttaacagatgataattaccatatgatagggaaagagagtattgctaaaatgaaagacggtgtattcataataaatgcagcacgaggagcattaattgatagtgaagctttaatagaaggactgaaatctggtaaaatagctggagcagccttagattcttatgaatatgaacaaggagttttccataacaataaaatgaatgaaatcatgaaagatgatacattagcaagacttaagagttttccaaatgtagttattacacctcatttaggattctatacagatgaagctgtatcaaatatggttgaaattactcttatgaatttacaagagtttgaattaaaaggaacttgtaaaaatcagagggtatgtaagtaaaaaggaggaaacaaatgtttttcacagaacaacatgaacttattagaaaattagcaagagattttgcagagcaggaaatagagcctattgcagatgaagtagataaaactgccgagttccctaaagaaattgtgaaaaaaatggcccaaaatggtttttttggaataaaaatgcctaaagaatatggtggagctggagcagataatagagcctatgtaactataatggaagagatatcaagagcatctggtgtagctggtatatatttatcatcaccaaattcattgcttggcacaccgttcctactagttggtacagatgaacaaaaggaaaaatatctaaaaccaatgattagaggagaaaaaacattagcttttgctttaacagaaccaggtgcaggttcagacgcaggagcacttgcaacaactgctagagaagaaggggactattatatattaaatggaagaaaaacttttattacaggagctccaatatctgataatatcatagtgttcgcaaaaacagatatgagcaaaggaacaaagggaattactacatttatagtagatagtaaacaggaaggtgtatcttttggtaaacctgaagacaagatgggtatgattggttgccctactagtgatattattttagagaatgtaaaggttcataaatctgatatacttggagaagtaaataaaggctttataacagcaatgaagactttaagtgttggtcgtataggagttgcttctcaagctttaggaatagcacaagctgctgtagatgaagctgttaaatatgcaaaacaaagaaaacaatttaatagacctatagcaaaatttcaagctattcaatttaaattggctaacatggaaacaaaattaaatgcggcaaaattattagtgtataatgctgcttataaaatggattgtggagaaaaggcagataaagaagcttctatggcaaaatattttgctgctgaatctgctatacaaatcgtaaatgacgcactacaaatccatggtggttatggatacataaaagattataaaatagaaagattatatagagatgttagagtaattgctatatatgaaggaacttctgaagttcaacaaatggtaatcgcatcaaatcttttaaagtaaaaaggaggaaacaaatggaaaacaatacaaatatgtttagtggagtaaaggttattgaattagcaaattttatagctgctccagcagcaggtagattttttgctgatggtggtgcagaggtaataaaaattgaatcacctgctggagatcctttaagatatactgctccttcagaaggaagaccattaagccaagaagaaaatactacttatgatttggaaaatgcaaataaaaaagcaatagtattaaatcttaaaagcgaaaaaggtaaaaagatattacatgaaatgttagcagaagcagatatattattaactaattggagaacaaaggctttagttaaacaaggattagactatgaaacactaaaagaaaaatatcctaaattagtttttgcacaaataactggttatggtgaaaaaggaccagataaagatcttccaggctttgattatactgcatttttcgctagaggcggtgtttcaggtactctttatgaaaaaggaactgtgcctccaaatgttgttccaggacttggagaccatcaagctgggatgtttttagcagcgggtatggcaggagctttatataaagcaaaaacaacaggacaaggagataaagtaacagtaagtttaatgcatagtgctatgtatggactaggtattatgatacaagctgctcaatataaagatcatggattagtatatccgataaatcgtaatgaaactccaaatccttttatagtttcatataaatctaaggatgattactttgttcaagtatgtatgccaccatatgatgttttctatgatagatttatgaccgctttaggaagagaagatttagttggagacgaaagatacaataaaatagaaaatttaaaagatggacgtgctaaggaagtatacagtataatcgaacaacaaatggttacaaagacaaaggatgaatgggataaaatatttagagatgcagacattccatttgctatcgcacaaacttgggaagatttattagaagatgaacaagcttgggcaaatgattatttgtataagatgaaatatccaacaggaaacgaaagagcattagtaagacttccagtattctttaaagaagcaggattaccagaatataatcaatcaccacaaatagcagaaaatactgtagaagttttaaaagaaatgggatatacagaacaagagattgaggaattagaaaaagataaagatataatggtaaggaaggaaaaataatggcagacatttatactatgggtgtagacataggttcaactgcatcaaaaacagtagtattaaaaaatggtaaagaaattgtaagtcaagcagtaataagtgtaggggccggaacaagtggccccaagagagctatagattctgtattaaaagatgctaaattatccattgaagatttagactatattgtatccactggatatggaagaaatagtttcgattttgctaacaaacaaatttctgaattaagttgtcatgcaaaaggggtctatttcgataacaataaagctagaacagttattgatataggcggacaagatattaaagtattaaaattagcggatagtggaagacttttaaactttataatgaatgataaatgtgctgcaggaacgggacgatttttagatgtaatgtctagagtaatagaagttccagttgatgagttaggaaaaaaagcattagaaagcaaaaatccttgtactattagttctacctgtacagtatttgcagagtcagaagtaatttctcaacttgcaagaggagttaaaactgaagatttgatagcaggaatttgtaaatctgtagcatcaagagtggctagccttgcaaagagaagtggtatagaagaattagtagttatgagtggaggagtagctaaaaatataggtgtagtaaaggcaatggaagcagaattgggaagagacatatatatatctaaaaattctcaattaaatggagcattgggagcaagtctatacgcttatgaaagttttcaaaaagaaaggagctaaaaacatgagtgatagaaataaggaagtaaaagaaaaaaaggcaaagcattatcttagagagattactgcaaagcattacaaagaagctctcgaagcaaaagaaaggggagaaaaggttggttggtgtgcatctaacttcccacaagaaatagctacaacattgggggtaaaagttgtttatccagaaaatcatgcagcagctgtagcagctagagggaatggacaaaatatgtgtgaacatgctgaggctatgggtttttctaatgatgtatgtggttatgcaagagtaaatttagctgttatggacataggtcatagtgaagatcaaccaatacctatgccagactttgtactttgctgtaataacatttgtaatcaaatgattaaatggtatgagcatatagcaaaaactttagatataccaatgattcttatagatataccatacaatacagaaaatactgtttcacaagatagaattaaatatattagagcacaatttgatgatgcaataaaacaattggaagaaataacaggcaaaaaatgggatgaaaataaatttgaagaagttatgaaaatatcccaagaaagtgcaaaacaatggttaagagcagcatcctatgcaaagtataaaccttcaccatttagcggatttgatttatttaatcatatggctgtagcagtttgtgcaagaggtacacaagaagctgcagatgcatttaagatgttagcagatgaatatgaggagaatgtaaaaactggaaaatccacttataggggagaagaaaaacaacgtatattatttgaagggattgcctgttggccatatttgagacataaattaactaagcttagtgaatatggtatgaacgtaactgcaactgtatacgcagaagcctttggtgttatatatgagaatatggatgaattaatggctgcttataataaagttcctaattcaattagttttgaaaacgcattaaaaatgagattaaatgctgttacaagcactaatacagaaggtgctgttattcatataaatagaagctgtaaattatggagtggatttttatatgagctagcaagaagattagaaaaggaaacaggaattcctgtagtatcatttgatggggaccaggcagacccaagaaatttctcagaagctcaatatgatactagaattcaaggacttaatgaagtaatggttgctaaaaaggaggctgaataagatgtcaaattcagataaattttttaatgactttaaggatattgtagaaaatcctaaaaaatatataatgaagcatatggaacaaactggacaaaaggctataggatgtatgccattatatactcctgaggaacttgtattagctgctggaatgtttccagtaggggtatggggaagcaatacagaactttcaaaagctaaaacatatttcccagcatttatttgttcaatattacaaacaacattggaaaatgcattaaatggagaatatgatatgttatctggtatgatgattacaaattattgtgattcattaaaatgcatgggacaaaattttaaactaaccgttgaaaatattgagtttatcccagtaacagttccacaaaatagaaaaatggaagctggaaaagagtttttaaaaagtcaatataaaatgaatattgagcaattagaaaagatttctggtaataaaataacagatgaatctttagaaaaagctatagaaatatatgatgaacacagaaaagtaatgaatgacttttcaatgttagcatcaaaatatccaggtataataacaccaactaaacgtaattatgttatgaaatctgcttattatatggataaaaaagaacatactgaaaaagttagacaattaatggatgaaattaaagctatagaaccaaaaccatttgaaggaaagagagttataactacaggtataattgcagattcagaagatttacttaaaatattagaagaaaataatatagctatagttggtgatgatatagcacatgaatctagacaatatagaacattgactccagaagcgaacacaccaatggataggttagctgagcaatttgctaatagagaatgtagtactttatatgatcctgaaaagaaaaggggtcaatatatagtagaaatggctaaagagagaaaagcagatggaattatatttttcatgacaaaattctgtgacccagaggaatatgattatccacaaatgaaaaaggattttgaagaagcaggcattccacatgtactaatagaaactgatatgcaaatgaaaaattatgaacaagctagaactgcaattcaggctttttcagaaacactttaaatccgaactccaattattgatgaaaagggattataaaagctcacttattcaggcgtagcacccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaagtggaggttgtataatg

SEQ ID NO: 65 - DNA, 85 bp, Escherichia coli
  source 1..85: mol_type = unassigned DNA
cgactactttctgacttccttcgtgacttgccctaagcatgttgtagtgcgatacttgtaatgacatttgtaattacaagaggtg

SEQ ID NO: 66 - DNA, 85 bp, Escherichia coli
  source 1..85: mol_type = unassigned DNA
aacctaaatatttgttgttaagctgcaatggaaacggtaaaagcggctagtatttaaagggatggatgacatctcagcgttgtcg

SEQ ID NO: 67 - DNA, 85 bp, Escherichia coli
  source 1..85: mol_type = unassigned DNA
aaaagataaatccacacagtttgtattgttttgtgcaaaagtttcactacgctttattaacaatactttctggcgacgtgcgcca

SEQ ID NO: 68 - DNA, 85 bp, Escherichia coli
  source 1..85: mol_type = unassigned DNA
agccagagcatgccctgacttcaccccgctgtgtctgcttttcccgactattcttaatgagcttcgatgcaattcacgatcccgc

SEQ ID NO: 69 - DNA, 85 bp, Escherichia coli
  source 1..85: mol_type = unassigned DNA
acggtacaacatttacgccactttacgcctgaataataaaagcgtgttatactctttccctgcaatgggttccgtagcagggaaa

SEQ ID NO: 70 - DNA, 85 bp, Escherichia coli; identical to SEQ ID NO: 69
  source 1..85: mol_type = unassigned DNA

SEQ ID NO: 71 - DNA, 85 bp, Escherichia coli
  source 1..85: mol_type = unassigned DNA
tgcgtaatttattcacaagcttgcattgaacttgtggataaaatcacggtctgataaaacagtgaatgataacctcgttgctctt

SEQ ID NO: 72 - DNA, 85 bp, Escherichia coli
  source 1..85: mol_type = unassigned DNA
tttattcacaagcttgcattgaacttgtggataaaatcacggtctgataaaacagtgaatgataacctcgttgctcttaagctct

SEQ ID NO: 73 - DNA, 85 bp, Escherichia coli
  source 1..85: mol_type = unassigned DNA
tgcattgaacttgtggataaaatcacggtctgataaaacagtgaatgataacctcgttgctcttaagctctggcacagttgttgc

SEQ ID NO: 74 - DNA, 85 bp, Escherichia coli
  source 1..85: mol_type = unassigned DNA
aacagtgaatgataacctcgttgctcttaagctctggcacagttgttgctaccactgaagcgccagaagatatcgattgagagga

SEQ ID NO: 75 - DNA, 216 bp, Escherichia coli
  source 1..216: mol_type = unassigned DNA
atgcagaataaacctacacctgaagaagtaaagaatgcgcgggttgcggcaggtcttactcttaaagaagctgctgatatttttggttatcaactgaattcctggcagatgaaagaaagtgcaggtaaggccagtcgttctttatctattggtgaatatcagtatttattgttattagcaaatatgcatccgtcttacaggctggtaaaaaaataa

SEQ ID NO: 76 - DNA, 90 bp, synthetic construct
  source 1..90: mol_type = other DNA
tacggctgcatgcccgaggcagacagcctcaagcacccgcagctattctacagtaaaaactcgcgcttctctagactatattaccctgtt

SEQ ID NO: 77 - DNA, 90 bp, synthetic construct
  source 1..90: mol_type = other DNA
agcggtacggaagtttcccccgacaccatgatggcggttagttgcgtcagaaatagttacgtatgcaactaatcagtcctgctcctcggc

SEQ ID NO: 78 - DNA, 90 bp, synthetic construct
  source 1..90: mol_type = other DNA
gtggtttgccacaaaacagtgcagtcacacatgacaggagaagatatgagccgggtaccgcggctctgagactcagtcctgctcctcggc

SEQ ID NO: 79 - DNA, 90 bp, synthetic construct
  source 1..90: mol_type = other DNA
ggtgcaggaaataaaaataatcatttctttatatgatctttttatcaatggagatagtatcatttatactctagactatattaccctgtt

SEQ ID NO: 80 - DNA, 85 bp, synthetic construct; identical to SEQ ID NO: 27
  source 1..85: mol_type = other DNA

SEQ ID NO: 81 - DNA, 87 bp, synthetic construct; identical to SEQ ID NO: 28
  source 1..87: mol_type = other DNA

SEQ ID NO: 82 - DNA, 21 bp, synthetic construct
  source 1..21: mol_type = other DNA
gtgatcaccagataatgttgc

SEQ ID NO: 83 - DNA, 18 bp, synthetic construct
  source 1..18: mol_type = other DNA
gcttcatcaccactgacc

SEQ ID NO: 84 - DNA, 87 bp, synthetic construct
  source 1..87: mol_type = other DNA
ccggtatcgggttaaagagtgggaaaaagttgccggtgttcctgttgcagaagtattttgactcacttccctgttaagtatcttcct

SEQ ID NO: 85 - DNA, 87 bp, synthetic construct
  source 1..87: mol_type = other DNA
aaaagaaaaagccggttcagagaaaaccggctgacggattatcgtctaacagttgatagctaccattacgccccgccctgccactca

SEQ ID NO: 86 - DNA, 89 bp, synthetic construct
  source 1..89: mol_type = other DNA
cgcagacaataaataatgatcatccccagaaatcatgtggagcacaaccaacaactgacgaaaaatactgatgatcggcacgtaagagg

SEQ ID NO: 87 - DNA, 90 bp, synthetic construct
  source 1..90: mol_type = other DNA
accccggacttagtgaacagggttttacagtctgattgacagatatggtcaccagaaaatatttgtattcctaattcagcgggttcgtgc

SEQ ID NO: 88 - DNA, 20 bp, synthetic construct
  source 1..20: mol_type = other DNA
cggcacgtaagaggctaacc

SEQ ID NO: 89 - DNA, 90 bp, synthetic construct
  source 1..90: mol_type = other DNA
ggaagtttcccccgacaccatgatggcggttagttgcgtcagaaatagttacgtatgcaactaaaagcgcgagtttttactgtagaatag

SEQ ID NO: 90 - DNA, 22 bp, synthetic construct
  source 1..22: mol_type = other DNA
cctgtatttttctgctcaatgg

SEQ ID NO: 91 - DNA, 90 bp, synthetic construct
  source 1..90: mol_type = other DNA
aaacagtgcagtcacacatgacaggagaagatatgagccgggtaccgcggctctgagactataaatgatactatctccattgataaaaag

(19) United States
(12) Patent Application Publication
(10) Pub. No.: US 2026/0115234 A1
(43) Pub. Date: Apr. 30, 2026

(54) PRODUCTION & SECRETION OF AUXIN-LIKE MOLECULES IN BACTERIA

(71) Applicant: SNIPR Biome ApS, Copenhagen (DK)

(72) Inventors: Szabolcs SEMSEY, Copenhagen (DK); Emilie SØNDBERG, Copenhagen (DK); Christian MUNCK, Copenhagen (DK); Andreas KOULOUKTSIS, Copenhagen (DK)

(21) Appl. No.: 19/431,700

(22) Filed: Dec. 23, 2025

Related U.S. Application Data
(63) Continuation of application No. PCT/EP2024/068944, filed on Jul. 4, 2024.

(30) Foreign Application Priority Data
Jul. 4, 2023 (GB) 2310227.0

(51) Int. Cl. A61K 35/741; A61K 31/405; A61K 31/407; A61K 31/4178; A61K 31/427; A61K 31/43; A61K 31/431; A61K 31/4709; A61K 31/675; A61K 31/7036; A61K 31/7048; A61K 35/00; A61K 38/12; A61P 1/14; C12N 15/52; C12N 15/70

(52) U.S. Cl. CPC A61K 35/741; A61K 31/405; A61K 31/407; A61K 31/4178; A61K 31/427; A61K 31/43; A61K 31/431; A61K 31/4709; A61K 31/675; A61K 31/7036; A61K 31/7048; A61K 38/12; A61P 1/14; C12N 15/52; C12N 15/70; A61K 2035/115; C12N 2800/101

(57) ABSTRACT

The present invention relates to modified bacteria and plasmids (e.g. conjugative plasmids) which are engineered to express auxins and auxin-like molecules (ALMs), to pharmaceutical compositions containing them and their use in the treatment of various metabolic and cardiovascular diseases.

### CROSS REFERENCE TO RELATED APPLICATIONS

This application is a continuation of International Application No. PCT/EP2024/068944, filed internationally on Jul. 4, 2024, which claims priority to Great Britain Application No. 2310227.0, filed on Jul. 4, 2023, the disclosures of each of which are hereby incorporated herein by reference in their entirety.

### REFERENCE TO AN ELECTRONIC SEQUENCE LISTING

The content of the electronic sequence listing (786212002501SeqList.xml; Size: 218,841 bytes; and Date of Creation: Dec. 22, 2025) is herein incorporated by reference in its entirety.

### BACKGROUND

Metabolic disorders have gradually become public health-threatening problems with an estimated incidence of around ¼ worldwide. Strategies and therapeutic attention are demanded to prevent metabolic disorders, particularly type 2 diabetes (T2D), obesity, and non-alcoholic fatty liver disease (NAFLD).

Gut microbiome dysbiosis has been associated with these metabolic disorders, resulting in an inflammatory state due to increased permeability of the intestinal epithelium (Taleb, Frontiers in Immunology, 10:2113, 2019). The intestinal epithelium is maintained by the presence of tight and adherence junctions in the epithelial cells and serves as a protective barrier against external substances. However, in inflammatory diseases, this barrier can become dysfunctional, leading to increased permeability and the movement of microbial components such as lipopolysaccharide (LPS) into the bloodstream. This can result in observed inflammation, which is implicated in the development of cardiometabolic diseases.

Recent evidence has linked indole-3-acetic acid (IAA), a gut microbiota-derived metabolite from dietary tryptophan, with resistance to liver damage and steatosis in mice (Ji et al., Nutrient 11:2062, 2019; Li et al., Frontiers in Pharmacology, 12:769501, 2021), and improved epithelial barrier function in mice (Laurans et al., Nature Medicine, 24:1113, 2018) and piglets (Liang et al., Frontiers in Microbiology, 9:1736, 2018). In humans, decreased levels of tryptophan and IAA, and increased levels of kynurenine, have been observed in the fecal samples from patients with obesity and T2D compared to healthy subjects, and similarly a change in tryptophan metabolism towards more kynurenine and less IAA in people with obesity and T2D (Laurans et al., 2018, supra; Natividad et al., Cell Metabolism, 28:737, 2018).

IAA and other tryptophan-derived compounds have previously been characterized as aryl hydrocarbon receptor (AHR) agonists, establishing a direct effect of the gut microbiome and its metabolites on intestinal homeostasis. In vitro data demonstrated that IAA induces activation of the aryl hydrocarbon receptor (AHR) in intestinal immune cells, increasing the production of anti-inflammatory interleukins like IL-17 and IL-22, which are important for antimicrobial immunity and mucosal barrier integrity (Laurans et al., 2018, supra). Moreover, IAA has been shown to attenuate lipogenesis in hepatocytes induced by cytokine and free fatty acids (Krishnan et al., Cell Reports, 23:1099, 2018).

Whilst IAA is a key auxin, in the treatment of cardiometabolic diseases, other auxin-like molecules may have therapeutic benefits. Other tryptophan-derived metabolites have been shown to regulate intestinal barrier function, and contributing to an anti-inflammatory environment in the gut epithelium. Those include indole-3-propionic acid (IPA) (Lanis et al., Mucosal Immunology 10:1133, 2017), indole-3-acetaldehyde (IAAld), or indole-3-pyruvate (IPyA) (Scott et al., PNAS, 117:19376, 2020). Specifically, IPyA has been shown to prevent chronic inflammation in the colon by activating AHR (Aoki et al., The Journal of Immunology, 15 (201), 3683, 2018) and IPA protected against indomethacin-induced intestinal injury (Venkatesh et al., Immunity, 41:296, 2014). Indole-3-butyric acid (IBA) significantly inhibited the LPS-induced upregulation of IL-4 and IL-6 mRNA (Zhen et al., Journal of Asthma and Allergy, 15:117, 2022).

Romasi & Lee, J. Microbiol. Biotechnol., 23 (12), 1726-1736, 2013, doi: http://dx.doi.org/10.4014/jmb.1308.08082 discloses IAA-producing E. coli comprising ipdC, aspC and iad1 genes.

WO2017/123418 (Synlogic, Inc) discloses inter alia genetic circuits for inclusion in bacteria for the production of indole metabolites and derivatives.

WO2021/242897 (Synlogic, Inc) discloses gene cassettes for producing IAA which are operably linked to an inducible promoter.

There remains a need for efficient in situ production of auxins and auxin like molecules which have a beneficial effect in vivo, for example in the gut microbiome.

### SUMMARY

The inventors have advantageously realised that increased levels of secretion of Auxin-Like Molecules (ALMs) can be achieved by modifying bacteria to include various heterologous exporters. As is known in the art, diffusion of molecules through bacterial membrane(s) (both gram-negative and gram-positive bacteria) can limit the amount of a particular molecule which diffuses to the local environment. Without being bound by theory, in general, bacterial membrane(s) are hydrophobic, so the more hydrophilic the small molecule is, the less likely it is to be able to cross the membrane. However, certain molecules which are detrimental to the cell (for example toxins, bacteriocins, etc) are exported more often, along with certain molecules which provide specific extra-cellular functions (such as molecules associated with quorum sensing, iron acquisition, etc). In addition, some molecules are actively transported through the membrane(s) by dedicated exporters (for example, excess amino acids may be exported from the cell to maintain homeostasis, such as the alaE exporter of alanine, and the leuE exporter of leucine both found in E. coli), or exporters which export a certain class of molecule (for example, the setA transporter found in E. coli exports various sugar molecules). However, the majority of ALM transporters to date have been found in plant species. Thus, in many bacteria, secretion of ALMs are limited by the rate of diffusion. The inventors have engineered bacteria to express heterologous exporters which are capable of exporting the ALMs across the membrane(s) of the bacterium. Unexpectedly, heterologous exporters are able to form and function within the bacterial membrane. This leads to an increased secretion of the desired ALMs into the local environment, and contributes to the reduced fitness disadvantage of the expression of ALMs within the bacterium, because (without being bound by theory) the ALMs are removed from the cytoplasm and/or periplasm into the local environment.

Furthermore, there remains a need for consistent and high-level biosynthesis of ALMs from bacteria, in particular for therapeutic applications. This is achieved by the present invention in several ways:

Firstly, previous attempts to include heterologous genes for the biosynthesis of ALMs on plasmids has required the control of inducible and/or weak promoters. This has been necessitated because, when constitutive promoters are used to control heterologous genes expression ALMs on a plasmid, it results in plasmid instability and high levels of genetic mutation within the heterologous genes. These mutations and instability ultimately result in gene inactivation and can even result in bacterial cell death. The present inventors unexpectedly realised that the provision of the heterologous genes directly into the chromosome of the bacterium leads to stable expression of the heterologous genes, without the associated plasmid instability and detrimental genetic mutations.

Furthermore, the inventors are able to use constitutive promoters to control the expression of the heterologous genes comprised by the chromosome of the bacterium, which allows for a higher and more consistent expression of the ALMs. Unexpectedly, this higher production appears to come at little-to-no fitness cost to the bacteria as may be expected, in contrast to plasmid production.

The inventors have also chosen combinations of specific heterologous genes for the production and extracellular export of the auxin indole-3-acetic acid (IAA), IPyA, IAAld, ILA, IA and IPA (as described in FIGS. 8, 11, 13 and 18), which may provide advantages over other genes in terms of one or more of: production stability, production levels, pathway efficiency and optimal temperature range for production in vivo.

The present invention therefore provides the following:

### In a First Configuration:

There is provided a modified bacterium, or a plasmid (e.g. a conjugative plasmid), for producing and secreting one or more Auxin Like Molecules (ALMs), wherein the bacterium or plasmid comprises one or more heterologous genes for the biosynthesis of said one or more ALMs, and wherein:
a. the bacterium further comprises a heterologous gene encoding an exporter which is capable of exporting said one or more ALMs out of the bacterium; or
b. the plasmid further comprises a heterologous gene encoding an exporter which is capable of exporting said one or more ALMs out of a bacterium which comprises said plasmid and produces and secretes said one or more ALMs.

### In a Second Configuration:

There is provided a modified bacterium, or a plasmid (e.g. a conjugative plasmid), for secreting one or more Auxin Like Molecules (ALMs), wherein:
a. the bacterium comprises one or more genes for the production of said one or more ALMs and further comprises a heterologous gene encoding an exporter which is capable of exporting said one or more ALMs out of the bacterium; or
b. the plasmid comprises a heterologous gene encoding an exporter which is capable of exporting said one or more ALMs out of a bacterium which comprises said plasmid, and the bacterium which comprises said plasmid comprises one or more genes for the production of said one or more ALMs

### In a Third Configuration:

There is provided a modified bacterium, or a plasmid (e.g. a conjugative plasmid), for the biosynthesis of an Auxin Like Molecule (ALM) which is indole-3-acetic acid (IAA), comprising heterologous genes for the biosynthesis of IAA, which heterologous genes encode:
i. Indole-3-pyruvate decarboxylase (ipdC) from a Pantoea species, e.g. from Pantoea agglomerans,
ii. Tryptophan-pyruvate aminotransferase 1 (taa1) from an Arabidopsis species, e.g. from Arabidopsis thaliana, and
iii. Indole-3-acetaldehyde dehydrogenase (iad1) from an Ustilago species, e.g. from Ustilago maydis,
wherein heterologous genes i. to iii. are each or all under the control of one or more constitutive promoter(s).

### In a Fourth Configuration:

There is provided a host (e.g. donor) cell comprising a plasmid (e.g. a conjugative plasmid) as described herein.

### In a Fifth Configuration:

There is provided a pharmaceutical composition comprising a modified bacterium as described herein, or comprising a host (donor) cell comprising the plasmid (e.g. a conjugative plasmid) as described herein, and a pharmaceutically acceptable excipient or carrier.

### In a Sixth Configuration:

There is provided a method of producing an ALM (e.g. IAA) in the gut of a subject, comprising administering to said subject a modified bacterium as described herein, a host (donor) cell as described herein, or a pharmaceutical composition as described herein.

### In a Seventh Configuration:

There is provided a method of treating a metabolic disease, such as a cardiovascular metabolic disease, comprising administering to a subject in need thereof a modified bacterium as described herein, a host (donor) cell as described herein, or a pharmaceutical composition as described herein.

### In an Eighth Configuration:

There is provided a modified bacterium as described herein, a host (donor) cell as described herein, or a pharmaceutical composition as described herein for use as a medicament.

### In a Ninth Configuration:

There is provided a method of producing a modified bacterium as described herein, wherein the heterologous genes are comprised by the chromosome of said bacterium, said method comprising the use of recombineering to introduce the heterologous genes into the chromosome of the bacterium.

### BRIEF DESCRIPTION OF THE FIGURES

FIG. 1: Schematic diagram showing the biosynthetic pathway for the conversion of indole to indole-3-acetic acid (IAA). Note that in the absence of a specific gene for the conversion of IAA to IBA, it is believed (without being bound by theory) to be an equilibrium reaction which spontaneously converts IAA to IBA.

FIG. 2: Schematic diagram showing the biosynthetic pathway for the conversion of indole to indole-3-propionic acid (IPA).

FIG. 3: Schematic diagram showing the biosynthetic pathway for the conversion of indole to indole-3-pyruvic acid (IPyA).

FIG. 4: Schematic diagram showing the biosynthetic pathway for the conversion of indole to indole-3-acetaldehyde (IAAld) and indole-3-ethanol. Note that there is no specific gene for the conversion of IAAld to indole-3-ethanol, but an equilibrium exists between the two molecules.

FIG. 5: Schematic diagram showing the biosynthetic pathway for the conversion of indole to indole-3-lactic acid (ILA).

FIG. 6: Schematic diagram showing the biosynthetic pathway for the conversion of indole to indole-3-acrylic acid (IAID).

FIG. 7: Schematic diagram showing the biosynthetic pathway for the conversion of indole to Indole-3-carboxylic acid (ICA).

FIG. 8: Layout of the IAA biosynthetic pathway. Plasmid p1915 contains ipdC, aspC, iad1 and tnaB under control of a cI based temperature-sensitive promoter, Pts. Plasmid p1138 contains a purple pigment, amilCP, under control of the same Pts Both plasmids carry a chloramphenicol resistance gene and use a p15A origin.

FIG. 9: IAA production in vitro with b5674 (E. coli MG1655 harbouring p1915). IAA production was measured in bacterial supernatants by Salkowski assay following over-night culture in M9 media containing 1 mM tryptophan. Strain b5674 harbours the p1915 plasmid which is engineered to produce IAA, while strain b5673 harbors the control plasmid lacking the IAA pathway.

FIG. 10: Pathway stability after in vivo passage. Production of IAA was quantified in all colonies recovered from faecal samples from active group (b5674) up to 54 h post-inoculation after a single inoculation.

FIG. 11: Layout of the plasmids with temperature inducible IAA biosynthetic pathway with exporters. Plasmids p1880, p1881 and p1882 contain ipdC, aspC and iad1 under control of a cI based temperature-sensitive promoter, Pts. All plasmids carry a chloramphenicol resistance gene and use a p15A origin. In addition, plasmids p1881 and 1882 carry the exporters pin2 and aec respectively, expressed from the promoter that drives expression of the resistance marker.

FIG. 12: IAA production in vitro with and without exporters. IAA production was measured in bacterial supernatants by Salkowski assay following over-night culture in M9 media containing 5 mM tryptophan. Strains b5626, b5627 and b5629 harbor plasmids p1880, p1881 and p1882 respectively, carrying the IAA pathway (Pts-ipdC-aspC-iad1) without exporter, with exporter pin2 and with exporter aec respectively.

FIG. 13: Layout of constructs containing constitutively expressed IAA biosynthetic pathway with exporters All plasmids contain a chloramphenicol resistance marker and a cloDF based origin with a copy number of 10-20. Plasmid p2526 contains ipdC, aspC and iad1 under control of promoter Pc-aga. Plasmid p2527 additionally contains the aec exporter expressed from the promoter that drives expression of the resistance marker. Plasmids p2528 and p2529 are like above, with taa1 replacing aspC. The IAA pathway inserted into the chromosome of b6131 contains ipdC, taa1, iad1 and aec under control of promoter Pc-tga.

FIG. 14: Pathway optimization with taa1 and aec lead to improved IAA secretion. IAA production was measured in bacterial supernatants by Salkowski assay following over-night culture in M9 media containing 1 mM tryptophan. Engineered plasmids were integrated into the same backbone strain carrying either of the two aminotransferases (aspqC/taa1) with and without the aec exporter. Strain b7196 (aspC); b7197 (aspC, aec); b7198 (taa1); b7199 (taa1, aec)

FIG. 15: Chromosomal integration of optimized pathway. IAA production was measured in bacterial supernatants by Salkowski assay following over-night culture in M9 media containing 1 mM tryptophan. Production is compared between strain b6131 (chromosomal Ptga-ipdC-taa1-iad1-aec in symbioflor G6/7 ΔtnaCA, ΔtrpR) and strain b7199 (containing plasmid p1882 with Paga-ipdC-taa1-iad1, aec, in symbioflor G6/7 ΔtnaCA, ΔtrpR). Strain b6570 (symbioflor G6/7 ΔtnaCA, ΔtrpR) serves as a negative control. There is no significant difference between IAA production levels between strains b7199 and b6131.

FIG. 16: Pathway stability after in vivo passage. Production of IAA was quantified in all colonies recovered from faecal samples from active group (strain b6131) up to 7 days post-inoculation (day 8). IAA production was measured in bacterial supernatants by Salkowski assay following over-night culture in M9 media containing 1 mM tryptophan. The production capability remained at the level of the b6131 strain, which had not been dosed in vivo and could be used as control.

FIG. 17: IAA production relative to growth. IAA production was measured in bacterial supernatants by Salkowski assay following over-night culture in M9 media containing 1 mM tryptophan. IAA production remained stable at both 30° C. and 37° C. Strain b6570 was included as a control.

FIG. 18: Layout of plasmids with biosynthetic pathways for the production of alternative tryptophan metabolites All plasmids contain a chloramphenicol resistance marker and a cloDF based origin with a copy number of 10-20. All genes shown are under control of promoter Pc-aga, except for aec which is expressed from the promoter that drives expression of the resistance marker. The plasmids contain the pathways needed to produce indole-3-pyruvic acid (IPyA), indole-3-acetaldehyde (IAAld), indole-3-lactic acid (IIA), indole-3-acrylic acid (IA) and indole-3-propionic acid (IPA), as shown.

FIGS. 19A-19C: Results of the LC-MS analyses. Substances quantified were tryptophan (TRP), indole-3-acetic acid (IAA), indole (IND) and indole-3-ethanol (HEI). FIG. 19A) and FIG. 19B) Performance of the different chromosomal deletions was assessed in the presence and absence of the plasmid borne IAA production pathway. FIG. 19C) The pathway was integrated into the chromosome of the double mutant strain (b6570, ΔtrpRΔtnaCA) and the resulting strain (b6131, ΔtrpRΔtnaCA IAA by csiR) was studied at different substrate combinations.

FIG. 20: Quantification of IAA in the supernatant of ON cultures grown in M9 media with 1 mM tryptophan. b6570 (no IAA) acts as negative control and b6131 (pathway at csiR) as benchmark. Strain b7848 (pathway at aslA) and b7829 (pathway at uspG) produces IAA at levels comparable to b6131.

FIG. 21: Quantification of IAA in the supernatant of ON cultures grown anaerobically in M9 media with 1 mM tryptophan. b6570 (no IAA pathway) acts as negative control and b6131 (pathway at csiR) as benchmark. An unpaired, two-tailed t-test comparing the two groups yields a p-value of 1.2-10.

FIG. 22: Determination of CFU in feces showing the levels for the individual animals in groups 5 and 6, and further with a geometric mean displayed (black line).

FIG. 23: IAA detected in nmol/g feces by LC-MS for group 5 and 6 having shown stable colonization.

FIG. 24: Tryptophan detected in nmol/g feces by LC-MS for groups 5 and 6 having shown stable colonization. At 24 hours all analysis had shown levels below limit of detection in the group 5 animals.

FIG. 25: Indole detected in nmol/g feces by LC-MS for groups 5 and 6 having shown stable colonization.

FIG. 26: Schematic representation of the modifications introduced into the conjugative β10 plasmid. The inserted DNA contains the three genes necessary to produce IAA from tryptophan (ipdC, taa1 and iad1) as well as a transporter aec that moves IAA out of the cell. Additionally, a chloramphenicol resistance marker is added for selection.

FIG. 27: IAA produced following overnight aerobic growth in M9 supplemented with 1 mM tryptophan. The IAA pathway is integrated into a conjugative vector (CGV) and conjugated into symbioflor G 6/7 (SBF) wildtype E. coli and E. hormaechei strains. The level of IAA produced from strains carrying the CGV is as high observed from SBF, wt E. coli and E. hormaechei, as it is from “SBF lead candidate” which contains the pathway in its chromosome.

FIG. 28: CFU counts in feces (CFU/g) during the in vivo study. Day 1 sample is taken before the first dosing, and the day 30 sample (includes day 29 and 30) is taken just before termination of the mice. The data reveals an initial colonization at approx. 2×10⁹ CFU/g feces on day 3 and shows a slow decline to 4×10⁵ CFU/g feces by day 19 and seems to remain stable around this level for the remaining duration of the study.

FIG. 29: IAA (nmol/g) quantification in feces of mice. Day 1 showed levels in the mice before dose initiation. The first timepoint on day 3 shows a mean of 26 nmol/g feces which declines to approx. 5.5 nmol/g feces on day 10 and 2.5 nmol/g feces on day of termination.

FIG. 30: Bar chart showing the total number of recipients (R) and transconjugants (T) after conjugation of plasmids p2464 (B10, control) and p2806 (B10 with IAA pathway), from b8524 (donor) into b5652 (recipient E. coli).

FIG. 31: shows the CFU level in feces. A pre-sample was taken on day 1 and followed by a single peroral dose on day 1. The animals were given a prebiotic in the drinking water until day 11. The prebiotic did not affect colonization of strain b8344, as the colonization remained relatively stable after removal. Colonization was shown to have stabilized at approx. 1×10⁷ CFU/g feces on day 16.

FIG. 32: shows levels fecal levels of IAA in mice before the single peroral dose on day 1, and then following the dose on days 4, 11 and 16 as measured by LCMS. The pre-sample was at a mean below 1 nmol/g feces, and the IAA was then increasing by day 4 and topped on day 11 with 54 nmol/g feces. On day 16 the fecal samples contained a mean of 42 nmol/g feces.

### DETAILED DESCRIPTION

### Definitions

As used herein, an “auxotroph” describes a mutation in a bacterium, or in a bacterium which comprises a plasmid described herein and which produces and secretes one or more ALMs (i.e. a recipient bacterium when the plasmid is a conjugative plasmid) which results in the bacterium, or results in in the bacterium which comprises the plasmid (i.e. results in the recipient bacterium) being incapable of producing a compound which is required for its growth. Thus, auxotrophic bacteria, and auxotrophic bacteria which comprise a plasmid described herein and which produce and secrete one or more ALMs (i.e. auxotrophic recipient bacteria when the plasmid is a conjugative plasmid) require an additional nutrient for cell proliferation and ultimately survival, as compared to the un-mutated bacterium or the un-mutated bacterium which comprises a plasmid described herein and which produces and secretes one or more ALMs (i.e. the un-mutated recipient bacterium when the plasmid is a conjugative plasmid).

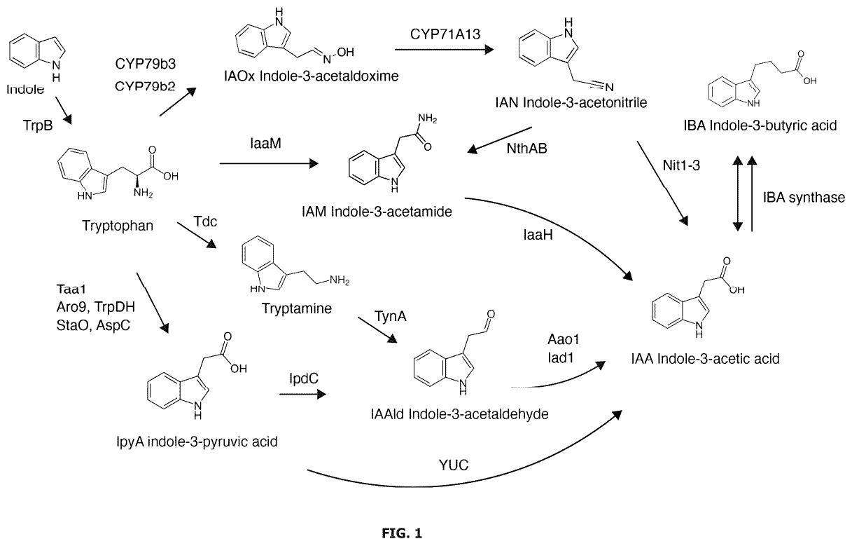

As used herein, an “Auxin-Like Molecules (ALMs)” includes natural auxins, synthetic auxin analogues (such as 1-naphthaleneacetic acid), various indole-based derivates, and intermediates in the metabolic pathways for the production of auxins. In one embodiment, the ALM is an auxin. In another embodiment, the ALM is an indole derivate selected from indole-3-pyruvic acid (IPyA), indole-3-acetaldehyde (IAAld), indole-3-lactic acid (ILA), indole-3-acrylic acid (IA), indole-3-carboxylic acid (ICA), indole-3-ethanol, tryptamine, indole-3-methanol (IM) and indole-3-carboxaldehyde (IAID). In another embodiment, the ALM is an indole derivate selected from indole-3-pyruvic acid (IPyA), indole-3-acetaldehyde (IAAld), indole-3-lactic acid (ILA), indole-3-acrylic acid (IA), indole-3-carboxylic acid (ICA) and indole-3-ethanol. In one embodiment, the ALM is an indole derivate selected from indole-3-pyruvic acid (IPyA), indole-3-acetaldehyde (IAAld), indole-3-lactic acid (ILA), indole-3-acrylic acid (IA) and indole-3-carboxylic acid (ICA). In another embodiment, the ALM is selected from tryptamine, indole-3-methanol (IM) and indole-3-acetaldehyde (IAID). In another embodiment, the ALM is any intermediate molecule from any of the pathways shown in FIGS. 1 to 7.

As used herein an “auxin” refers to a group of naturally occurring plant hormones that regulate growth, particularly by stimulating cell elongation in stems. Auxins play a role in cell division and differentiation, in fruit development, in the formation of roots from cuttings, in the inhibition of lateral branching (apical dominance), and in leaf fall (abscission). The most important naturally occurring auxin is indole-3-acetic acid (IAA), which is formed either from the amino acid tryptophan or from the breakdown of glycosides. Other naturally occurring auxins include 4-chloroindole-3-acetic acid (4-Cl-IAA), 2-phenylactic acid (PAA), indole-3-butyric acid (IBA) and indole-3-propionic acid (IPA). In one embodiment, the auxin is selected from IPA, IBA and IAA. In one embodiment, the auxin is selected from IAA and IPA. In one embodiment, the auxin is IAA.

As used herein, a “conjugative plasmid” is a plasmid which, when comprised within a bacterial cell (“host” or “donor” cell, used interchangeably herein) is able to be transferred to another bacterium (“recipient” cell) through the mechanism of bacterial conjugation. Bacterial conjugation is the unidirectional and horizontal transmission of genetic information from one bacterium to another. Conjugative plasmids generally fall into two classes: mobilizable plasmids and self-transmissible plasmids. Mobilizable plasmids comprise at least an origin of transfer (oriT), a relaxase and other genetic information on the plasmid which is transferred to the recipient cell, and require helper functions provided by e.g. a second plasmid or the chromosome of the donor cell to effect the plasmid transfer. On the other hand, self-transmissible plasmids, in addition to the genetic information on the plasmid which is transferred to the recipient cell, also contain all the molecular machinery needed for self-transfer (e.g. for pilus formation and initiation of gene transfer) on the same plasmid. Both mobilizable plasmids and self-transmissible plasmids comprise relaxase genes which recognises the origin of transfer (oriT) and catalyses both the initial cleavage of oriT in the donor, to produce the DNA strand from the plasmid that will be transferred, as well as the final ligation of the transported DNA in the recipient cell that reconstitutes the conjugated plasmid. Thus, in one embodiment, the conjugative plasmid is a mobilizable plasmid. In another embodiment, the conjugative plasmid is a self-transmissible plasmid. In another embodiment, the conjugative plasmid includes an origin of transfer (oriT). Plasmid mobility, mechanisms and structures are described in more detail in Smillie et al., Microbiol. Mol. Biol. Rev., 74 (3): 434-452, 2010 doi: 10.1128/MMBR.00020-10, which is incorporated herein in its entirety.

Bacterial cells possessing a conjugative self-transmissible plasmid contain a surface structure (pilus) encoded by the conjugative machinery on the plasmid that is involved in the coupling of donor and recipient cells, and the transfer of the genetic information contained within the plasmid. Conjugation involves contact between cells, and the transfer of genetic traits can be mediated by many plasmids. Among all natural transfer mechanisms, conjugation is the most efficient. For example, F plasmid of E. coli, pCFIO plasmid of Enterococcus faecalis and pXO16 plasmid of Bacillus thuringiensis employ different mechanisms for the establishment of mating pairs, the sizes of mating aggregates are different, and they have different host ranges within gram-negative (F) as well as gram-positive (pCFIO and pXO16) bacteria. Their plasmid sizes are also different; 54, 100 and 200 kb, respectively. Remarkably, however despite differences in origin and size, those conjugation systems are able to sustain efficient conjugative transfer in liquid medium. The conjugative process permits the protection of plasmid DNA against environmental nucleases, and thus efficient delivery of plasmid DNA into a recipient cell can be obtained. Conjugation functions are naturally plasmid encoded. Numerous conjugative plasmids (and transposons) are known, which can transfer associated genes within one species (narrow host range) or between many species (broad host range). Transmissible plasmids are widespread across the domain of bacteria and similar systems of horizontal gene transfer through pilus structures have recently been described for Archaea. Engineered conjugative plasmids are described in more detail in WO2021/037732 (SNIPR Biome ApS), which is incorporated herein in its entirety. The features of such conjugative plasmids and bacterial cells comprising them as described in the claims as filed in WO2021/037732 are also incorporated herein by reference.

“Constitutive promoter” refers to a promoter that is capable of facilitating continuous transcription of a coding sequence or gene under its control and/or to which it is operably linked. Constitutive promoters and variants are well known in the art and are described elsewhere herein.

As used herein an “exporter” refers to a membrane-integrated protein which is capable of exporting ALMs out of the bacterial cell. Particular exporters for use in the disclosure are described elsewhere herein. As discussed elsewhere herein, in some bacteria there are no endogenous exporters of ALMs, and thus the cell must rely on diffusion of the ALMs to remove the ALMs from the cell. Without being bound by theory, the export of ALMs in gram-negative bacteria may be particularly problematic, due to the presence of both an inner and outer membrane. Thus, the addition of a heterologous exporter may provide further benefits in this type of bacterial cell. The ability of any given protein to act as an exporter can be easily measured by those skilled in the art, and methods are provided in the Examples hereinbelow. Identification of putative exporters can be achieved through literature searches as well as by using protein and genetic databases, such as GenBank, which are well-known to those skilled in the art.

“Heterologous” refers to a nucleotide sequence that is not normally found in a given cell in nature. As used herein, a heterologous sequence encompasses a nucleic acid sequence that is exogenously introduced into a given cell. A heterologous gene includes a native gene, or fragment thereof, that has been introduced into the cell. For example, a heterologous gene may include a native coding sequence that is a portion of a chimeric gene to include a native coding sequence that is a portion of a chimeric gene to include non-native regulatory regions that is reintroduced into the cell. A heterologous gene may also include a native gene, or fragment thereof, introduced into a non-native cell. Thus. a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. For plasmids, such as conjugative plasmid, a heterologous gene is one which has been engineered to be included on such plasmid (e.g. conjugative plasmid).

An “inducible promoter” refers to a promoter which transcribes a coding sequence or gene under its control and/or to which it is operably linked in the presence of an inducer of said promoter. The inducer may be one or more environmental condition(s) and/or one or more inducing molecule(s).

A “kill switch” refers to a biocontainment system which is included in the bacterium or in the plasmid (e.g. the conjugative plasmid) and is designed to destroy the bacterium, or in the case of a plasmid (e.g. a conjugative plasmid), either the plasmid itself only, or the plasmid and the bacterium comprising the plasmid together, when no longer contained within its desired environment (e.g. within a microbiome, such as a gut microbiome, within a subject). Such means are well-known in the art, and are regulatable, for example by the addition of non-naturally occurring substances (e.g. synthetic amino acids), temperature and the like.

A “microbiome,” as used herein, refers to the totality of microbes in a particular environment (e.g. in/on an organism, in a marine environment (e.g. ocean), and/or in a terrestrial environment (e.g. soil)). In some embodiments, a microbiome may refer to the totality of microbes that reside, or are stably maintained, for example, on the surface and in deep layers of the skin, in the saliva and oral mucosa, in the conjunctiva, and in the gastrointestinal tracts of an organism. The microbiome may exist within any of the organs described elsewhere herein.

“Preventing” as used herein in relation to transcription or expression of a gene or protein refers to a complete ablation of transcription or expression.

“Reducing” as used herein in relation to transcription or expression of a gene or protein refers to a reduction in gene transcription or expression, such as at least a 50% reduction of transcription or expression. In one embodiment, the reduction is at least a 60%, at least a 70%, at least an 80% reduction. In one embodiment, the reduction is at least an 85% reduction. In one embodiment, the reduction is at least a 90% reduction. In one embodiment, the reduction is at least a 95% reduction. In one embodiment, the reduction is at least a 97% reduction. In one embodiment, the reduction is at least a 99% reduction. In one embodiment, the reduction is a 100% reduction.

### Genes for Use in the Biosynthetic Pathways of ALMs

The genes (including for production of ALMs, the heterologous exporters, any kill switch genes) which are inserted into the modified bacteria and plasmids (e.g. conjugative plasmids) as described herein may be from various sources, e.g. plant, mammal, bacterial. In one embodiment, the one or more heterologous gene(s) (including for production of ALMs, the heterologous exporters, any kill switch genes) is from a prokaryotic origin. In one embodiment, the one or more heterologous gene(s) (including for production of ALMs, the heterologous exporters, any kill switch genes) is from a bacterial origin where such gene exists (e.g. from any of the bacterial strains described elsewhere herein, for example any of the strains listed in Table 3).

Particular sources of heterologous genes for the production and secretion of ALMs used in the various pathways described herein are described below.

As will be apparent to those skilled in the art, the below list may not encompass all known genes which catalyse the stated reaction. Further genes may be identified, for example, through literature searching, using known databases (such as GenBank, Kegg, EMBL or protein databases such as Uniprot and the like), either through keyword searching, putative annotations and/or sequence homology to a known enzyme (protein and/or DNA sequence). A list of databases can be found here: https://en.wikipedia.org/wiki/List_of_biological_databases.

A gene for the conversion of indole to tryptophan includes, but is not limited to trpB. Tryptophan synthase β-subunit (trpB) may be from the genus Escherichia, e.g E. coli. trpB includes paralogs TrpB1 and TrpB2.

A gene for the conversion of tryptophan to tryptamine includes but is not limited to tdc. Tryptophan decarboxylase (tdc) may be from the genus Catharanthus, e.g. Catharanthus roseus or from the genus Clostridium, e.g. Clostridium sporogenes.

A gene for the conversion of tryptamine to IAAld includes but is not limited to tynA. Monoamine oxidase (tynA) may be from the genus Escherichia, e.g. E. coli.

Genes for the conversion of IAAld to IAA include but are not limited to iad1 and aao1. Indole-3-acetaldehyde dehydrogenase (iad1) may be from the genus Ustilago, e.g. Ustilago maydis. Indole-3-acetaldehyde oxidase (aao1) may be from the genus Arabidopsis, e.g. Arabidopsis thaliana. In one embodiment, the iad1 comprises the nucleotide sequence of Seq ID No: 42. In one embodiment, the gene has at least 80% identity (such as 85%, 90% or 95%, in particular 90% identity) to Seq ID No: 42, and converts IAAld to IAA.

Genes for the conversion of tryptophan to IPyA include, but are not limited to staO, aro9, aspC, taa1 and trpDH. L-tryptophan oxidase (staO) may be from the genus streptomycese, e.g. streptomyces sp. TP-A0274. L-tryptophan aminotransferase (aro9), may be from S. cerevisae. Aspartate aminotransferase (aspC) may be from E. coli. L-tryptophan-pyruvate aminotransferase (taa1) may be from the genus Arabidopsis, e.g. Arabidopsis thaliana. Tryptophan dehydrogenase (trpDH) may be from the genus Nostoc, e.g. a strain of Nostoc punctiforme, such as Nostoc punctiforme NIES-2108. In one embodiment, the aspC comprises the nucleotide sequence of Seq ID No: 39. In one embodiment, the gene has at least 80% identity (such as 85%, 90% or 95%, in particular 90% identity) to Seq ID No: 39, and converts tryptophan to IPyA. In one embodiment, the taa1 comprises the nucleotide sequence of Seq ID No: 40. In one embodiment, the gene has at least 80% identity (such as 85%, 90% or 95%, in particular 90% identity) to Seq ID No: 40, and converts tryptophan to IPyA.

trpDH may be disadvantageous, due to its reported instability, see Matsui et al., J. Biotechnol., 196-197:27-32, 2015, doi: 10.1016/j.jbiotec.2015.01.010, which is incorporated herein in its entirety. Even at low temperatures (4° C.), trpDH (EC1.4.1.19) showed >80% reduction in activity after 24 hours. Depending on the desired application, aspC may be less attractive, due to its range of substrates/catalytic reactions. aspC has been identified as a multifunctional enzyme, that catalyses the synthesis of aspartate, phenylalanine, tyrosine and other compounds via a transamination reaction. In contrast, whilst taa1 may catalyse reactions with tryptophan, phenylalanine and tyrosine, taa1 has a much higher affinity for tryptophan than its other substrate, and thus is likely to provide higher levels of conversion of tryptophan to IPyA than aspC.

A gene for the conversion of IPyA to IAAld includes, but is not limited to IpdC. Indole-3-pyruvate decarboxylase (IpdC) may be from the genus Enterobacter, e.g. Enterobacter cloacae or from the genus Pantoea, e.g. Pantoea agglomerans, in particular Pantoea agglomerans. In one embodiment, the IpdC comprises the nucleotide sequence of Seq ID No: 41. In one embodiment, the gene has at least 80% identity (such as 85%, 90% or 95%, in particular 90% identity) to Seq ID No: 41, and converts IPyA to IAAld.

A gene for the conversion of IPyA to IAA includes, but is not limited to YUC genes. Indole-3-pyruvate monooxygenase (YUC) genes may be from the genus Arabidopsis, e.g. Arabidopsis thaliana. YUC genes may also be from the genus Escherichia, e.g. E. coli, such as YUC2 and YUC6.

A gene for the conversion of tryptophan to IAM includes, but is not limited to iaaM. Tryptophan 2-monooxygenase (iaaM) may be from the genus Pseudomonas, e.g. Pseudomonas savastanoi.

A gene for the conversion of IAM to IAA includes, but is not limited to iaaH. Indoleacetamide hydrolase (iaaH) may be from the genus Pseudomonas, e.g. Pseudomonas savastanoi.

Genes for the conversion of tryptophan to IAOx include, but are not limited CYP79B2 and CYP79B3. Tryptophan N-monooxygenase (CYP79B2) may be from the genus Arabidopsis, e.g. Arabidopsis thaliana. Tryptophan N-monooxygenase (CYP79B3) may be from the genus Arabidopsis, e.g. Arabidopsis thaliana.

A gene for the conversion of IAOx to IAN includes but is not limited to CYP71A13. Indoleacetaldoxime dehydratase (CYP71A13) may be from the genus Arabidopsis, e.g. Arabidopis thaliana. The enzyme is described in more detail in Nafisi et al., The Plant Cell, 19 (6), 2039-2052, 2007, which is incorporated herein in its entirety.

Genes for the conversion of IAN to IAA include but are not limited to nit1, nit2 and nit3. Nitrilases may be from the genus Arabidopsis, e.g. Arabidopsis thaliana.

A gene for the conversion of IAN to IAID includes but it not limited to CYP71B6. Cytochrome P450 monooxygenase (CYP71B6) may be from the genus Arabidopsis, e.g. Arabidopsis thaliana. The enzyme is described in more detail in Böttcher et al., Plant Physiology, 165 (2), 2014, doi: 10.1104/pp.114.235630, which is incorporated herein in its entirety.

A gene for the conversion of IAN to IAM includes but it not limited to nthAB. Nitrile hydratase (nthAB) may be from the genus Arabidopsis, e.g. Arabidopsis thaliana. nthAB may be from a Pseudomonas strain, e.g. Pseudomonas sp Strain UW4. The enzyme is described in more detail in Duca et al., Applied and Environmental Microbiology 80 (15), 2014, doi: 10.1128/AEM.00649-14 which is incorporated herein in its entirety.

Genes for the conversion of IPyA to ILA include but are not limited to hcxB, Idh4 and fldH. hcxB may be from the genus Escherichia, e.g. E. coli. Idh4 may be from the genus Bifidobacterium, e.g. Bifidobacterium longum. Indole-3-lactate dehydrogenase (fldH) may be from the genus Clostridium, e.g. Clostridium sporogenes. In one embodiment, the hcxB comprises the nucleotide sequence of Seq ID No: 43. In one embodiment, the gene has at least 80% identity (such as 85%, 90% or 95%, in particular 90% identity) to Seq ID No: 43, and converts IPyA to ILA. In one embodiment, the Idh4 comprises the nucleotide sequence of Seq ID No: 44. In one embodiment, the gene has at least 80% identity (such as 85%, 90% or 95%, in particular 90% identity) to Seq ID No: 44, and converts IPyA to ILA. In one embodiment, the fldH comprises the nucleotide sequence of Seq ID No: 45. In one embodiment, the gene has at least 80% identity (such as 85%, 90% or 95%, in particular 90% identity) to Seq ID No: 45, and converts IPyA to ILA.

Genes for the conversion of ILA to IA include, but are not limited to all of fldA, fldI, fldB and fldC (together referred to as fldAIBC). fldAIBC may be from the genus Clostridium, e.g. Clostridium sporogenes. In one embodiment, the fldAIBC comprises the nucleotide sequence of Seq ID No: 46. In one embodiment, the gene has at least 80% identity (such as 85%, 90% or 95%, in particular 90% identity) to Seq ID No: 46, and converts ILA to IA. This gene is described in more detail in Dodd et al., Nature Letters, 551, 648, 2017, doi: 10.1038/nature24661, which is incorporated herein by reference in its entirety.

A gene for the conversion of IA to IPA includes, but is not limited to acdA. Acyl-CoA dehydrogenase (acdA) may be from the genus Clostridium, e.g. Clostridium sporogenes. In one embodiment, the acdA comprises the nucleotide sequence of Seq ID No: 47. In one embodiment, the gene has at least 80% identity (such as 85%, 90% or 95%, in particular 90% identity) to Seq ID No: 47, and converts IA to IPA.

A gene for the conversion of IAA to skatole includes, but is not limited to IAD. Indoleacetate decarboxylase (IAD) may be from the genus Clostridium, e.g. Clostridium scatologenes. This enzyme is described in more detail in Liu et al., Nat Commun., 9:224, 2018, doi: 10.1038/s41467-018-06627-x, which is incorporated herein in its entirety.

A gene for the conversion of skatole to IM includes, but is not limited to TSO. Tryptophan side chain oxidase may be from the genus Pseudomonas, e.g. Pseudomonas spp. (ATCC 29574). The enzyme is described in more detail in Ushiro et al., J. Biol. Chem., 253 (24), 9002-9008, 1978 and Takai et al., J. Biol. Chem., 252 (8), 2648-2656, 1977, both of which are incorporated herein in their entirety.

A gene for the conversion of IM to IAID includes, but is not limited to TSO. Tryptophan side chain oxidase may be from the genus Pseudomonas, e.g. Pseudomonas spp. (ATCC 29574). The enzyme is described in more detail in Ushiro et al., supra and Takai et al., supra.

A gene for the conversion of skatole to indole-3-acetaldehyde includes, but is not limited to TSO. Tryptophan side chain oxidase may be from the genus Pseudomonas, e.g. Pseudomonas spp. (ATCC 29574). The enzyme is described in more detail in Ushiro et al., supra and Takai et al., supra.

A gene for the conversion of tryptophan to indole-3-acrylic acid includes, but is not limited to WAL. Tryptophan ammonia lyase (WAL) may be from the genus Rubrivivax, e.g. Rubrivivax benzoatilyticus.

A gene for the conversion of chorismate to anthranilic acid includes, but is not limited to trpE. Anthranilate synthase (trpE) may be from the genus Escherichia, e.g. E. coli.

A gene for the conversion of anthranilic acid to N-(5-phosphoribosyl)-anthranilate includes, but is not limited to trpD. Phosphoribosyl transferase (trpD) may be from the genus Escherichia, e.g. E. coli.

A gene for the conversion of N-(5-phosphoribosyl)-anthranilate to 1-(o-carboxyphynylamino-1-deoxyribulose-5-phosphate includes, but is not limited to trpC. InGP synthase (trpC)) may be from the genus Escherichia, e.g. E. coli.

A gene for the conversion of 1-(o-carboxyphynylamino-1-deoxyribulose-5-phosphate to indole-3-glycerol phosphate includes, but is not limited to trpA. Tryptophan synthase (trpA)) may be from the genus Escherichia, e.g. E. coli.

### Production of Auxin-Like Molecules (ALMs)

As discussed elsewhere herein, ALMs are therapeutically useful molecules and there remains a need for efficient in situ production of ALMs (including auxins, e.g. IAA).

There is provided a modified bacterium for secreting one or more Auxin Like Molecules (ALMs, e.g. any of the ALMs described herein, such as IAA), wherein the bacterium comprises one or more genes for the biosynthesis of said one or more ALMs, and further comprises a heterologous gene encoding an exporter which is capable of exporting said one or more ALMs out of the bacterium.

There is also provided a plasmid (e.g. a conjugative plasmid) for secreting one or more Auxin Like Molecules (ALMs, e.g. any of the ALMs described herein, such as IAA), wherein the plasmid comprises a heterologous gene encoding an exporter which is capable of exporting said one or more ALMs out of a bacterium which comprises said plasmid (i.e. a recipient bacterium when the plasmid is a conjugative plasmid), and the bacterium which comprises said plasmid comprises one or more genes for the production of said one or more ALMs.

In some embodiments, the bacterium or the bacterium which comprises a plasmid (e.g. a conjugative plasmid) comprises one or more endogenous genes for the production of an ALM, e.g. any of the ALMs described herein, such as IAA. In these embodiments, the addition of a heterologous gene encoding an exporter as described herein is expected to improve the secretion of the ALM into the external environment of the bacterial cell or bacterial cell which comprise the plasmid (e.g. the conjugative plasmid).

There is provided a modified bacterium for producing and secreting one or more Auxin Like Molecules (ALMs, e.g. any of the ALMs described herein, such as IAA), wherein the bacterium comprises one or more heterologous genes for the biosynthesis of said one or more ALMs, and further comprises a heterologous gene encoding an exporter which is capable of exporting said one or more ALMs out of the bacterium.

There is also provided a plasmid (e.g. a conjugative plasmid) for producing and secreting one or more Auxin Like Molecules (ALMs, e.g. any of the ALMs described herein, such as IAA), wherein the plasmid comprises one or more heterologous genes for the biosynthesis of said one or more ALMs, and further comprises a heterologous gene encoding an exporter which is capable of exporting said one or more ALMs out of a bacterium which comprises said plasmid and produces and which bacterium secretes said one or more ALMs (i.e. a recipient bacterium when the plasmid is a conjugative plasmid).

In any embodiment described herein relating to a modified bacterium, the bacterium for producing and secreting one or more ALMs further comprises a modification in endogenous tryptophanase (tnaA). The TnaA protein is responsible for conversion of tryptophan into indole. When active in the bacteria described herein, TnaA activity reduces the amount of tryptophan which is available to be used in other pathways for the production of desired ALMs (see FIGS. 1 to 7). Thus, inactivation of the tnaA gene will increase the amount of tryptophan which is available to be used in the biosynthesis of the desired ALM. In the examples herein (see e.g. Example 2.1), the inventors use a deletion of tnaCA, which, in addition to the effect of increasing the amount of tryptophan available by reducing its conversion into indole, further increases the amount of tryptophan in the cell through deletion of the tnaC leader peptide and the intergenic region found immediately upstream of tnaA. Bacterial cells may include genes (tnaB) which encode an importer of tryptophan, known as TnaB, located just downstream of tnaA. Deletion of the leader sequence including the TnaC peptide removes the tryptophan dependent regulation of the TnaB importer of tryptophan, thereby further increasing the amount of tryptophan available for the biosynthesis of ALMs.

Thus, in any embodiment, the bacterium for producing and secreting one or more ALMs further comprises a modification in an endogenous tryptophanase (tnaA) and/or tnaC, which reduces expression (or prevents expression) of said tnaA and/or tnaC. In any embodiment, the modification in an endogenous tnaA and/or tnaC is a deletion of one or more nucleotides in an endogenous tnaA and/or tnaC which prevents or reduces (e.g. prevents) transcription or expression of said tnaA. In another embodiment, the modification is a modification in endogenous tnaC which is a deletion which comprises deletion of at least the nucleotides which, when transcribed, express a TnaC peptide. In another embodiment, the deletion both prevents the transcription or expression of tnaA and of tnaC.

In any embodiment described herein relating to a modified bacterium, the bacterium for producing and secreting one or more ALMs includes a modification in endogenous tryptophan transcriptional repressor (trpR). The trpR gene, when transcribed, forms a homodimer, which binds to tryptophan. Upon tryptophan binding, TrpR binds to the DNA of the promoter of the trp operon and sterically blocks RNA polymerase from binding and initiating transcription. Thus, the mutation in endogenous trpR results in increased transcription of the trp operon. The effect is threefold: (i) to de-repress synthesis of chorismate (thus increasing the amount of chorismate in the cell), (ii) to de-repress trpEDCA, which converts chorismate to indole (thus increasing the amount of indole in the cell), and (iii) to de-repress the mtr proton symporter of tryptophan and indole into the cell (thus increasing the amount of tryptophan and indole in the cell).

Thus, in any embodiment, the bacterium for producing and secreting one or more ALMs further comprises a modification in an endogenous tryptophan transcriptional repressor (trpR) gene which reduces expression (or prevents expression) of said trpR. In any embodiment, the modification in endogenous trpR is a deletion of one or more nucleotides in an endogenous trpR gene which prevents or reduces (e.g. prevents) transcription or expression of trpR. In another embodiment, the modification of endogenous trpR results in TrpR no longer being able to form a homodimer (e.g. the modification is a deletion or mutation of one or more amino acids in the dimerization interface, which prevents dimerization of TrpR). In another embodiment, the modification of endogenous trpR results in TrpR no longer being able to bind to tryptophan (e.g. the modification is a deletion or mutation of one or more amino acids in the tryptophan binding site, which prevents the conformational change in the TrpR dimer upon tryptophan binding).

The crystal structure of TrpR has been solved (see for example, Schevitz et al., Nature, 317 (6040), 782-786, 198, doi: 10.1038/317782a0, incorporated herein by reference in its entirety) and several studies on its dimerization and effect of mutations have been published (see, for example, Pal et al., Structure, 25, 867-877, 2017, http://dx.doi.org/10.1016/j.str.2017.04.015 and Sprenger et al., Acta Crystallogr. F. Struct. Biol. Commun., 77 (Pt 7), 215-225, 2021, doi: 10.1107/S2053230X21006142, both incorporated herein by reference in their entirety). Using this information and similar publications, a skilled person can identify potential sites for modification (e.g. deletion or substitution).

In any embodiment herein, further genes may be included in the bacterium or the plasmid (e.g. the conjugative plasmid) to increase the production of tryptophan.

Thus, in any embodiment, the bacterium, or the plasmid (e.g. a conjugative plasmid) as described herein comprises one or more heterologous gene(s) for the biosynthesis of tryptophan from chorismate ((3R,4R)-3-[(1-carboxyvinyl)oxy]-4-hydroxycyclohexa-1,5-diene-1-carboxylic acid).

In one embodiment, the one or more heterologous gene(s) for the biosynthesis of tryptophan comprise (or consists of) a gene for the conversion of chorismate to anthranilic acid (optionally a gene which is anthranilate synthase (trpE)), a gene for the conversion of anthranilic acid to N-(5-phosphoribosyl)-anthranilate (optionally a gene which is phosphoribosyl transferase (trpD)), a gene for the conversion of N-(5-phosphoribosyl)-anthranilate to 1-(o-carboxyphynylamino-1-deoxyribulose-5-phosphate (optionally a gene which is (trpF)), a gene for the conversion of 1-(o-carboxyphynylamino-1-deoxyribulose-5-phosphate to indole-3-glycerol phosphate (optionally a gene which is InGP synthase (trpC)), and a gene for the conversion of indole-3-glycerol phosphate to indole (optionally a gene which is tryptophan synthase (trpA)).

In any embodiment, the bacterium or the plasmid (e.g. the conjugative plasmid) as described herein comprises one or more heterologous gene(s) for the biosynthesis of tryptophan from anthranilic acid.

In one embodiment, the one or more heterologous gene(s) for the biosynthesis of tryptophan comprise (or consists of) a gene for the conversion of anthranilic acid to N-(5-phosphoribosyl)-anthranilate (optionally a gene which is phosphoribosyl transferase (trpD)), a gene for the conversion of N-(5-phosphoribosyl)-anthranilate to 1-(o-carboxyphynylamino-1-deoxyribulose-5-phosphate (optionally a gene which is (trpf)), a gene for the conversion of 1-(o-carboxyphynylamino-1-deoxyribulose-5-phosphate to indole-3-glycerol phosphate (optionally a gene which is InGP synthase (trpC)), and a gene for the conversion of indole-3-glycerol phosphate to indole (optionally a gene which is tryptophan synthase (trpA)).

In any embodiment, the bacterium or the plasmid (e.g. the conjugative plasmid) as described herein comprises one or more heterologous gene(s) for the biosynthesis of tryptophan from N-(5-phosphoribosyl)-anthranilate.

In one embodiment, the one or more heterologous gene(s) for the biosynthesis of tryptophan comprise (or consists of) a gene for the conversion of N-(5-phosphoribosyl)-anthranilate to 1-(o-carboxyphynylamino-1-deoxyribulose-5-phosphate (optionally a gene which is (trpf)), a gene for the conversion of 1-(o-carboxyphynylamino-1-deoxyribulose-5-phosphate to indole-3-glycerol phosphate (optionally a gene which is InGP synthase (trpC)), and a gene for the conversion of indole-3-glycerol phosphate to indole (optionally a gene which is tryptophan synthase (trpA)).

In any embodiment, the bacterium or the plasmid (e.g. the conjugative plasmid) as described herein comprises one or more heterologous gene(s) for the biosynthesis of tryptophan from 1-(o-carboxyphynylamino-1-deoxyribulose-5-phosphate.

In one embodiment, the one or more heterologous gene(s) for the biosynthesis of tryptophan comprise (or consists of) a gene for the conversion of 1-(o-carboxyphynylamino-1-deoxyribulose-5-phosphate to indole-3-glycerol phosphate (optionally a gene which is InGP synthase (trpC)), and a gene for the conversion of indole-3-glycerol phosphate to indole (optionally a gene which is tryptophan synthase (trpA)).

In any embodiment, the bacterium or the plasmid (e.g. the conjugative plasmid) as described herein comprises one or more heterologous gene(s) for the biosynthesis of tryptophan from indole-3-glycerol phosphate.

In one embodiment, the one or more heterologous gene(s) for the biosynthesis of tryptophan comprise (or consists of) a gene for the conversion of indole-3-glycerol phosphate to indole (optionally a gene which is tryptophan synthase (trpA)).

In an alternative, in any embodiment described herein, the modified bacterium, or the plasmid (e.g. the conjugative plasmid), does not comprise any heterologous genes for the biosynthesis of tryptophan.

Thus, in any embodiment described herein, the modified bacterium, or the plasmid (e.g. the conjugative plasmid), does not comprise genes encoding one or more genes selected from trpA, trpB, trpC, trpD and trpE. In one embodiment, the modified bacterium, or the plasmid (e.g. the conjugative plasmid), does not comprise genes encoding trpA, trpC, and trpD. In one embodiment, the modified bacterium, or the plasmid (e.g. the conjugative plasmid), does not comprise genes encoding trpA, trpC, and trpE. In one embodiment, the modified bacterium, or the plasmid (e.g. the conjugative plasmid), does not comprise genes encoding trpC, trpD, and trpE.

In any embodiment described herein, the modified bacterium, or the plasmid (e.g. the conjugative plasmid), does not comprise the genes consisting of trpA, trpB, trpC, trpD and trpE.

In any embodiment described herein, the modified bacterium, or the plasmid (e.g. the conjugative plasmid), does not comprise the genes consisting of trpA, trpC, trpD and trpE.

In an alternative, in any embodiment described herein, the modified bacterium, or the plasmid (e.g. the conjugative plasmid), comprises no other heterologous genes other than the one or more heterologous gene(s) for the biosynthesis of said one or more ALMs (e.g. any of the ALMs described herein, such as IAA).

In an alternative, in any embodiment described herein, the modified bacterium, or the plasmid (e.g. the conjugative plasmid), comprises no other heterologous genes other than the one or more heterologous gene(s) for the biosynthesis of said one or more ALMs (e.g. any of the ALMs described herein, such as IAA) and any kill switch genes.

In an alternative, in any embodiment described herein relating to a modified bacterium, the bacterium comprises no other heterologous genes other than the one or more heterologous gene(s) for the biosynthesis of said one or more ALMs (e.g. any of the ALMs described herein, such as IAA), and any heterologous or modified auxotrophy genes.

In an alternative, in any embodiment described herein relating to a modified bacterium, the bacterium comprises no other heterologous genes other than the one or more heterologous gene(s) for the biosynthesis of said one or more ALMs (e.g. any of the ALMs described herein, such as IAA), any kill switch genes and any heterologous or modified auxotrophy genes.

In an alternative, in any embodiment described herein, the modified bacterium, or a plasmid (e.g. a conjugative plasmid), comprises no other heterologous genes other than the one or more heterologous gene(s) for the biosynthesis of said one or more ALMs (e.g. any of the ALMs described herein, such as IAA) and the heterologous gene encoding an exporter which is capable of exporting said one or more ALMs.

In an alternative, in any embodiment described herein, the modified bacterium, or the plasmid (e.g. the conjugative plasmid), comprises no other heterologous genes other than the one or more heterologous gene(s) for the biosynthesis of said one or more ALMs (e.g. any of the ALMs described herein, such as IAA) and the heterologous gene encoding an exporter which is capable of exporting said one or more ALMs and any kill switch genes.

In an alternative, in any embodiment described herein relating to a modified bacterium, the bacterium comprises no other heterologous genes other than the one or more heterologous gene(s) for the biosynthesis of said one or more ALMs (e.g. any of the ALMs described herein, such as IAA) and the heterologous gene encoding an exporter which is capable of exporting said one or more ALMs, and any heterologous or modified auxotrophy genes.

In an alternative, in any embodiment described herein relating to a modified bacterium, the bacterium comprises no other heterologous genes other than the one or more heterologous gene(s) for the biosynthesis of said one or more ALMs (e.g. any of the ALMs described herein, such as IAA) and the heterologous gene encoding an exporter which is capable of exporting said one or more ALMs, any kill switch genes and any heterologous or modified auxotrophy genes.

In an alternative, in any embodiment described herein where the bacterium, or the bacterium comprising the plasmid (e.g. the conjugative plasmid) comprises one or more endogenous genes for the production of an ALM (e.g. any of the ALMs described herein, such as IAA), the modified bacterium, or the plasmid (e.g. the conjugative plasmid), comprises no other heterologous genes other than the heterologous gene encoding an exporter which is capable of exporting said one or more ALMs.

In an alternative, in any embodiment described herein where the bacterium, or the bacterium comprising the plasmid (e.g. the conjugative plasmid), comprises one or more endogenous genes for the production of an ALM (e.g. any of the ALMs described herein, such as IAA), the modified bacterium, or the plasmid (e.g. the conjugative plasmid), comprises no other heterologous genes other than the heterologous gene encoding an exporter which is capable of exporting said one or more ALMs and any kill switch genes.

In an alternative, in any embodiment described herein relating to a modified bacterium which comprises one or more endogenous genes for the production of an ALM (e.g. any of the ALMs described herein, such as IAA), the bacterium comprises no other heterologous genes other than the heterologous gene encoding an exporter which is capable of exporting said one or more ALMs, and any heterologous or modified auxotrophy genes.

In an alternative, in any embodiment described herein relating to a modified bacterium which comprises one or more endogenous genes for the production of an ALM (e.g. any of the ALMs described herein, such as IAA), the bacterium comprises no other heterologous genes other than the heterologous gene encoding an exporter which is capable of exporting said one or more ALMs, any kill switch genes and any heterologous or modified auxotrophy genes.

In any of the embodiments herein, the one or more genes for the biosynthesis of said one or more ALMs (e.g. any of the ALMs described herein, such as IAA) are each or all under the control of one or more promoter(s) as described elsewhere herein. In one embodiment, the one or more promoter(s) is one or more constitutive promoter(s) (e.g. any of the constitutive promoters described elsewhere herein).

In any of the embodiments herein, the one or more genes for the biosynthesis of said one or more ALMs (e.g. any of the ALMs described herein, such as IAA) are comprised within an operon under the control of a single promoter as described elsewhere herein. In one embodiment, the promoter is a constitutive promoter (e.g. any of the constitutive promoters described elsewhere herein).

In any of the embodiments herein, the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), is under the control of a promoter as described elsewhere herein. In one embodiment, the promoter is a constitutive promoter (e.g. any of the constitutive promoters described elsewhere herein).

In any of the embodiments herein, the one or more genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), are each or all under the control of one or more promoter(s) as described elsewhere herein. In one embodiment, the promoter is a constitutive promoter (e.g. any of the constitutive promoters described elsewhere herein).

In any of the embodiments herein, the one or more genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), are all under the control of a single promoter as described elsewhere herein. In one embodiment, the promoter is a constitutive promoter (e.g. any of the constitutive promoters described elsewhere herein).

In any of the embodiments herein, the one or more genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), are comprised within an operon under the control of a single promoter as described elsewhere herein. In one embodiment, the promoter is a constitutive promoter (e.g. any of the constitutive promoters described elsewhere herein).

In any embodiment herein relating to a modified bacterium, one or more genes for the biosynthesis of said one or more ALMs (e.g. any of the ALMs described herein, such as IAA) are comprised by the chromosome of the bacterium.

In any embodiment herein relating to a modified bacterium, the one or more genes for the biosynthesis of said one or more ALMs (e.g. any of the ALMs described herein, such as IAA) are all comprised by the chromosome of the bacterium.

In any embodiment herein relating to a modified bacterium, the gene encoding an exporter which is capable of exporting said one or more ALMs (e.g. any of the ALMs described herein, such as IAA) is comprised by the chromosome of the bacterium.

In any embodiment herein relating to a modified bacterium, the one or more genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium are comprised by the chromosome of the bacterium.

In any embodiment herein relating to a modified bacterium, the one or more genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium are comprised by an operon which is comprised the chromosome of the bacterium.

In any embodiment herein relating to a modified bacterium, the one or more genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium are comprised by an operon under the control of a single promoter as described elsewhere herein, which operon is comprised the chromosome of the bacterium. In one embodiment, the promoter is a constitutive promoter (e.g. any of the constitutive promoters described elsewhere herein).

Positions for recombineering heterologous genes into the chromosome of a bacterium are known to those in the art. For example, the genes may be placed into a desert region in the chromosome or may replace a gene (or portion or a gene, or one or more nucleotides) which is being deleted to provide another effect, such as to provide an auxotrophy.

### Production of Indole-3-Acetic Acid (IAA)

Recent evidence has linked indole-3-acetic acid (IAA, a gut microbiota-derived metabolite from dietary tryptophan and an auxin), with resistance to liver damage and steatosis in mice (Ji et al., supra; Li et al., supra), and improved epithelial barrier function in mice (Laurans et al., supra) and piglets (Liang et al., supra). In humans, decreased levels of tryptophan and IAA, and increased levels of kynurenine, have been observed in the fecal samples from patients with obesity and T2D compared to healthy subjects, and similarly a change in tryptophan metabolism towards more kynurenine and less IAA in people with obesity and T2D (Laurans et al., 2018, supra, Natividad et al., supra).

In vitro data demonstrated that IAA induces activation of the aryl hydrocarbon receptor (AHR) in intestinal immune cells, increasing the production of anti-inflammatory interleukins like IL-17 and IL-22, which are important for antimicrobial immunity and mucosal barrier integrity (Laurans et al., 2018, supra). Moreover, IAA has been shown to attenuate lipogenesis in hepatocytes induced by cytokine and free fatty acids (Krishnan et al., supra). Ji et al., supra showed that activation of AHR by IAA alleviates high-fat diet (HFD)-induced hepatotoxicity in mice. Other indications mediated by IAA are described herein.

There are various pathways for the production of IAA, which are shown schematically in FIG. 1. Each step of the pathway is governed by one or more heterologous genes. Any of the genes of the pathways (or part pathways, if it is desirable to start production of IAA from an intermediate molecule) shown in FIG. 1 could be inserted into a bacterium or into a plasmid (e.g. into a conjugative plasmid) described herein to produce IAA.

There is provided a modified bacterium for producing and secreting an Auxin Like Molecule (ALM) which is indole-3-acetic acid (IAA), wherein the bacterium comprises one or more heterologous gene(s) for the biosynthesis of IAA, and further comprises a heterologous gene encoding an exporter which is capable of exporting IAA out of the bacterium.

There is provided a plasmid (e.g. a conjugative plasmid) for producing and secreting an Auxin Like Molecule (ALM) which is indole-3-acetic acid (IAA), wherein the plasmid comprises one or more heterologous gene(s) for the biosynthesis of IAA, and further comprises a heterologous gene encoding an exporter which is capable of exporting IAA out of a bacterium which comprises said plasmid and produces and secretes IAA (i.e. out of a recipient bacterium when the plasmid is a conjugative plasmid).

There is provided a host (e.g. a donor) cell (as described elsewhere herein) comprising a plasmid (e.g. a conjugative plasmid) as described herein for producing and secreting an Auxin Like Molecule (ALM) which is indole-3-acetic acid (IAA).

In one embodiment, the bacterium, or the plasmid (e.g. the conjugative plasmid), comprises one or more heterologous gene(s) for the biosynthesis of IAA.

In one embodiment, the bacterium, or the plasmid (e.g. the conjugative plasmid), comprises one or more heterologous gene(s) for the biosynthesis of IAA from indole.

In one embodiment, the one or more heterologous gene(s) for the biosynthesis of IAA comprise (or consists of) a gene for the conversion of indole to tryptophan, a gene for the conversion of tryptophan to tryptamine, a gene for the conversion of tryptamine to indole-3-acetaldehyde (IAAld), and a gene for the conversion of IAAld to IAA.

In one embodiment, the one or more heterologous gene(s) for the biosynthesis of IAA comprise (or consists of) a gene for the conversion of indole to tryptophan, a gene for the conversion of tryptophan to indole-3-pyruvic acid (IPyA), a gene for the conversion of IPyA to IAAld, and a gene for the conversion of IAAld to IAA.

In one embodiment, the one or more heterologous gene(s) for the biosynthesis of IAA comprise (or consists of) a gene for the conversion of indole to tryptophan, a gene for the conversion of tryptophan to indole-3-pyruvic acid (IPyA), and a gene for the conversion of IPyA to IAA.

In one embodiment, the one or more heterologous gene(s) for the biosynthesis of IAA comprise (or consists of) a gene for the conversion of indole to tryptophan, a gene for the conversion of tryptophan to indole-3-acetamide (IAM), and a gene for the conversion of IAM to IAA.

In one embodiment, the one or more heterologous gene(s) for the biosynthesis of IAA comprise (or consists of) a gene for the conversion of indole to tryptophan, a gene for the conversion of tryptophan to indole-3-acetaldoximine (IAOx), a gene for the conversion of IAOx to indole-3-acetonitrile (IAN), and a gene for the conversion of IAN to IAA.

In one embodiment, the one or more heterologous gene(s) for the biosynthesis of IAA comprise (or consists of) a gene for the conversion of indole to tryptophan, a gene for the conversion of tryptophan to indole-3-acetaldoximine (IAOx), a gene for the conversion of IAOx to indole-3-acetonitrile (IAN), a gene for the conversion of IAN to IAM, and a gene for the conversion of IAM to IAA.

In one embodiment, the one or more heterologous gene(s) for the biosynthesis of IAA are such that production of IAA is comparable (e.g. statistically similar, such as within statistical error) at 30° C. and at 37° C. in vitro. In vitro methods are described below in Example 2.5.

In one embodiment, the bacterium, or the plasmid (e.g. the conjugative plasmid), comprises one or more heterologous gene(s) for the biosynthesis of IAA from tryptophan.

In one embodiment, the one or more heterologous gene(s) for the biosynthesis of IAA comprise (or consists of) a gene for the conversion of tryptophan to tryptamine, a gene for the conversion of tryptamine to indole-3-acetaldehyde (IAAld), and a gene for the conversion of IAAld to IAA.

In one embodiment, the one or more heterologous gene(s) for the biosynthesis of IAA comprise (or consists of) a gene for the conversion of tryptophan to indole-3-pyruvic acid (IPyA), a gene for the conversion of IPyA to IAAld, and a gene for the conversion of IAAld to IAA.

In one embodiment, the one or more heterologous gene(s) for the biosynthesis of IAA comprise (or consists of) a gene for the conversion of tryptophan to indole-3-pyruvic acid (IPyA), and a gene for the conversion of IPyA to IAA.

In one embodiment, the one or more heterologous gene(s) for the biosynthesis of IAA comprise (or consists of) a gene for the conversion of tryptophan to indole-3-acetamide (IAM), and a gene for the conversion of IAM to IAA.

In one embodiment, the one or more heterologous gene(s) for the biosynthesis of IAA comprise (or consists of) a gene for the conversion of tryptophan to indole-3-acetaldoximine (IAOx), a gene for the conversion of IAOx to indole-3-acetonitrile (IAN), and a gene for the conversion of IAN to IAA.

In one embodiment, the one or more heterologous gene(s) for the biosynthesis of IAA comprise (or consists of) a gene for the conversion of tryptophan to indole-3-acetaldoximine (IAOx), a gene for the conversion of IAOx to indole-3-acetonitrile (IAN), a gene for the conversion of IAN to IAM, and a gene for the conversion of IAM to IAA.

In another embodiment, the bacterium, or the plasmid (e.g. the conjugative plasmid) as described herein comprises one or more heterologous gene(s) for the biosynthesis of IAA from tryptamine.

In one embodiment, the one or more heterologous gene(s) for the biosynthesis of IAA comprise (or consists of) a gene for the conversion of tryptamine to IAAld and a gene for the conversion of IAAld to IAA.

In another embodiment, the bacterium, or the plasmid (e.g. the conjugative plasmid) as described herein comprises one or more heterologous gene(s) for the biosynthesis of IAA from IAAld.

In one embodiment, the one or more heterologous gene(s) for the biosynthesis of IAA comprises (or consists of) a gene for the conversion of IAAld to IAA.

In another embodiment, the bacterium, or the plasmid (e.g. the conjugative plasmid) as described herein comprises one or more heterologous gene(s) for the biosynthesis of IAA from IPyA.

In one embodiment, the one or more heterologous gene(s) for the biosynthesis of IAA comprises (or consists of) a gene for the conversion of IPyA to IAA.

In one embodiment, the one or more heterologous gene(s) for the biosynthesis of IAA comprises (or consists of) a gene for the conversion of IPyA to IAAld, and a gene for the conversion of IAAld to IAA.

In another embodiment, the bacterium, or the plasmid (e.g. the conjugative plasmid) as described herein comprises one or more heterologous gene(s) for the biosynthesis of IAA from IAM.

In one embodiment, the one or more heterologous gene(s) for the biosynthesis of IAA comprises (or consists of) a gene for the conversion of IAM to IAA.

In another embodiment, the bacterium, or the plasmid (e.g. the conjugative plasmid) as described herein comprises one or more heterologous gene(s) for the biosynthesis of IAA from IAOx.

In one embodiment, the one or more heterologous gene(s) for the biosynthesis of IAA comprises (or consists of) a gene for the conversion of IAOx to IAN, and a gene for the conversion of IAN to IAA.

In one embodiment, the one or more heterologous gene(s) for the biosynthesis of IAA comprises (or consists of) a gene for the conversion of IAOx to IAN, a gene for the conversion of IAN to IAM, and a gene for the conversion of IAM to IAA.

In another embodiment, the bacterium, or the plasmid (e.g. the conjugative plasmid) as described herein comprises one or more heterologous gene(s) for the biosynthesis of IAA from IAN. In one embodiment, the one or more heterologous gene(s) for the biosynthesis of IAA comprises (or consists of) a gene for the conversion of IAN to IAM, and a gene for the conversion of IAM to IAA.

In one embodiment, the one or more heterologous gene(s) for the biosynthesis of IAA comprises (or consists of) a gene for the conversion of IAN to IAA.

In another embodiment, the bacterium, the or plasmid (e.g. the conjugative plasmid) may comprise genes from more than one of the pathways described hereinabove.

In one embodiment, heterologous gene(s) for the biosynthesis of IAA comprise:
i. Indole-3-pyruvate decarboxylase (ipdC);
ii. Tryptophan-pyruvate aminotransferase 1 (taa1); and
iii. Indole-3-acetaldehyde dehydrogenase (iad1).

In one embodiment, heterologous gene(s) for the biosynthesis of IAA comprise:
i. Indole-3-pyruvate decarboxylase (ipdC) from a Pantoea species;
ii. Tryptophan-pyruvate aminotransferase 1 (taa1) from an Arabidopsis species; and
iii. Indole-3-acetaldehyde dehydrogenase (iad1) from an Ustilago species.

In one embodiment, heterologous gene(s) for the biosynthesis of IAA comprise:
i. Indole-3-pyruvate decarboxylase (ipdC) from Pantoea agglomerans,
ii. Tryptophan-pyruvate aminotransferase 1 (taa1) from Arabidopsis thaliana, and
iii. Indole-3-acetaldehyde dehydrogenase (iad1) from Ustilago maydis.

In some embodiments, the modified bacterium, or the plasmid (e.g. the conjugative plasmid), may not comprise a heterologous gene encoding an exporter which is capable of exporting IAA. However, in any of the following embodiments, the bacterium may also further comprise a heterologous gene encoding an exporter which is capable of exporting IAA out of the bacterium. Equally, in any of the following embodiments, the plasmid (e.g. the conjugative plasmid) may further comprise a heterologous gene encoding an exporter which is capable of exporting IAA out of a bacterium which comprises said plasmid and produces and secretes IAA (i.e. out of a recipient bacterium when the plasmid is a conjugative plasmid).

Further, in any of the following embodiments, the heterologous genes for the biosynthesis of IAA and/or the heterologous gene encoding an exporter which is capable of exporting IAA may be in a single operon. The single operon may be under the control of a constitutive promoter.

There is provided a modified bacterium, or a plasmid (e.g. a conjugative plasmid), for the biosynthesis of an Auxin Like Molecule (ALM) which is indole-3-acetic acid (IAA), comprising heterologous genes for the biosynthesis of IAA, which heterologous genes encode (optionally in downstream order):
i. Indole-3-pyruvate decarboxylase (ipdC) from a Pantoea species, e.g. from Pantoea agglomerans,
ii. Tryptophan-pyruvate aminotransferase 1 (taa1) from an Arabidopsis species, e.g. from Arabidopsis thaliana, and
iii. Indole-3-acetaldehyde dehydrogenase (iad1) from an Ustilago species, e.g. from Ustilago maydis, wherein heterologous genes i. to iii. are each or all under the control of one or more constitutive promoter(s).

There is provided a modified bacterium, or a plasmid (e.g. a conjugative plasmid), for the biosynthesis of an Auxin Like Molecule (ALM) which is indole-3-acetic acid (IAA), comprising heterologous genes for the biosynthesis of IAA, which heterologous genes encode (optionally in downstream order):
i. Indole-3-pyruvate decarboxylase (ipdC) from a Pantoea species, e.g. from Pantoea agglomerans,
ii. Tryptophan-pyruvate aminotransferase 1 (taa1) from an Arabidopsis species, e.g. from Arabidopsis thaliana, and
iii. Indole-3-acetaldehyde dehydrogenase (iad1) from an Ustilago species, e.g. from Ustilago maydis, wherein heterologous genes i. to iii. are all under the control of single a constitutive promoter.

There is provided a modified bacterium for the biosynthesis of an Auxin Like Molecule (ALM) which is indole-3-acetic acid (IAA), comprising heterologous genes for the biosynthesis of IAA, which heterologous genes encode (optionally in downstream order):
i. Indole-3-pyruvate decarboxylase (ipdC) from Pantoea agglomerans,
ii. Tryptophan-pyruvate aminotransferase 1 (taa1) from Arabidopsis thaliana, and
iii. Indole-3-acetaldehyde dehydrogenase (iad1) from Ustilago maydis,
wherein heterologous genes i. to iii. are all under the control of single a constitutive promoter.

There is provided a plasmid (e.g. a conjugative plasmid) for the biosynthesis of an Auxin Like Molecule (ALM) which is indole-3-acetic acid (IAA), comprising heterologous genes for the biosynthesis of IAA, which heterologous genes encode (optionally in downstream order):
i. Indole-3-pyruvate decarboxylase (ipdC) from Pantoea agglomerans,
ii. Tryptophan-pyruvate aminotransferase 1 (taa1) from Arabidopsis thaliana, and
iii. Indole-3-acetaldehyde dehydrogenase (iad1) from Ustilago maydis, wherein heterologous genes i. to iii, are all under the control of single a constitutive promoter.

There is provided a modified bacterium for the biosynthesis of an Auxin Like Molecule (ALM) which is indole-3-acetic acid (IAA), comprising heterologous genes for the biosynthesis of IAA, which heterologous genes encode:
i. Indole-3-pyruvate decarboxylase (ipdC) from Pantoea agglomerans;
ii. Tryptophan-pyruvate aminotransferase 1 (taa1) from Arabidopsis thaliana, and
iii. Indole-3-acetaldehyde dehydrogenase (iad1) from Ustilago maydis,
wherein heterologous genes i. to iii. are all under the control of single a constitutive promoter, and
wherein the bacterium further comprises a heterologous gene encoding an exporter which is capable of exporting IAA out of the bacterium.

There is provided a plasmid (e.g. a conjugative plasmid) for the biosynthesis of an Auxin Like Molecule (ALM) which is indole-3-acetic acid (IAA), comprising heterologous genes for the biosynthesis of IAA, which heterologous genes encode:
i. Indole-3-pyruvate decarboxylase (ipdC) from Pantoea agglomerans,
ii. Tryptophan-pyruvate aminotransferase 1 (taa1) from Arabidopsis thaliana, and
iii. Indole-3-acetaldehyde dehydrogenase (iad1) from Ustilago maydis,
wherein heterologous genes i. to iii. are all under the control of single a constitutive promoter, and
wherein the plasmid further comprises a heterologous gene encoding an exporter which is capable of exporting IAA out of a bacterium which comprises said plasmid and produces and secretes IAA (i.e. out of a recipient bacterium when the plasmid is a conjugative plasmid).

In one embodiment, the heterologous gene encoding an exporter which is capable of exporting said one or more ALMs (e.g. IAA) comprised within an operon which comprises the following genes in downstream order:
i. Indole-3-pyruvate decarboxylase (ipdC), optionally from a Pantoea species, e.g. from Pantoea agglomerans,
ii. Tryptophan-pyruvate aminotransferase 1 (taa1), optionally from an Arabidopsis species, e.g. from Arabidopsis thaliana, and
iii. Indole-3-acetaldehyde dehydrogenase (iad1), optionally an Ustilago species, e.g. from Ustilago maydis, and
iv. the heterologous gene encoding an exporter of IAA.
optionally wherein the operon is comprised by the chromosome of the modified bacterium.

In one embodiment, the heterologous gene encoding an exporter which is capable of exporting said one or more ALMs (e.g. IAA) comprised within an operon which comprises the following genes in downstream order:
1. Indole-3-pyruvate decarboxylase (ipdC), optionally from a Pantoea species, e.g. from Pantoea agglomerans,
ii. Tryptophan-pyruvate aminotransferase 1 (taa1), optionally from an Arabidopsis species, e.g. from Arabidopsis thaliana, and
iii. Indole-3-acetaldehyde dehydrogenase (iad1), optionally an Ustilago species, e.g. from Ustilago maydis, and
iv. the heterologous gene encoding an exporter of IAA, and
wherein the operon is comprised by the chromosome of the modified bacterium

### Production of Indole-3-Butyric Acid (IBA)

The ALM indole-3-butyric acid (IBA) significantly inhibited the LPS-induced upregulation of IL-4 and IL-6 mRNA (Zhen et al., supra). Thus, it may be expected that IBA can provide a therapeutic effect in various settings.

There are various pathways for the production of IBA, which are shown schematically in FIG. 1. Each step of the pathway is governed by one or more heterologous genes. Any of the genes of the pathways (or part pathways, if it is desirable to start production of IBA from an intermediate molecule) shown in FIG. 1 could be inserted into a bacterium or into a plasmid (e.g. into a conjugative plasmid) described herein to produce IBA. Without the addition of a specific gene for the conversion of IAA into IBA, an equilibrium exists between the two products, and it is expected that a certain amount of IBA will inevitably be produced when producing IAA. However, specific genes exist which catalyse the conversion of IAA to IBA and these can be included in the bacterium or in the plasmid (e.g. in the conjugative plasmid).

There is provided a modified bacterium for producing and secreting an Auxin Like Molecule (ALM) which is indole-3-butyric acid (IBA), wherein the bacterium comprises one or more heterologous gene(s) for the biosynthesis of IBA, and further comprises a heterologous gene encoding an exporter which is capable of exporting IBA out of the bacterium.

There is provided a plasmid (e.g. a conjugative plasmid) for producing and secreting an Auxin Like Molecule (ALM) which is indole-3-butyric acid (IBA), wherein the plasmid comprises one or more heterologous gene(s) for the biosynthesis of IBA, and further comprises a heterologous gene encoding an exporter which is capable of exporting IBA out of a bacterium which comprises said plasmid and produces and secretes IBA (i.e. out of a recipient bacterium when the plasmid is a conjugative plasmid).

There is provided a host (e.g. a donor) cell (as described elsewhere herein) comprising a plasmid (e.g. conjugative plasmid) as described herein for producing and secreting an Auxin Like Molecule (ALM) which is indole-3-butyric acid (IBA).

In one embodiment, the bacterium, or the plasmid (e.g. the conjugative plasmid) comprises one or more heterologous gene(s) for the biosynthesis of IBA.

In one embodiment, the bacterium, or the plasmid (e.g. the conjugative plasmid) comprises one or more heterologous gene(s) for the biosynthesis of IBA from indole. These include any of the genes described herein for the biosynthesis of IAA from indole, wherein the bacterium, or the plasmid (e.g. the conjugative plasmid), further comprises a gene for the conversion of IAA to IBA.

In one embodiment, the bacterium, or the plasmid (e.g. the conjugative plasmid), comprises one or more heterologous gene(s) for the biosynthesis of IBA from tryptophan. These include any of the genes described herein for the biosynthesis of IAA from tryptophan, wherein the bacterium, or the plasmid (e.g. the conjugative plasmid) further comprises a gene for the conversion of IAA to IBA.

In another embodiment, the bacterium, or the plasmid (e.g. the conjugative plasmid) as described herein comprises one or more heterologous gene(s) for the biosynthesis of IBA from tryptamine. These include any of the genes described herein for the biosynthesis of IAA from tryptamine, wherein the bacterium or the plasmid (e.g. the conjugative plasmid) further comprises a gene for the conversion of IAA to IBA.

In another embodiment, the bacterium, or the plasmid (e.g. conjugative plasmid) as described herein comprises one or more heterologous gene(s) for the biosynthesis of IBA from IAAld. These include any of the genes described herein for the biosynthesis of IAA from IAAld, wherein the bacterium, or the plasmid (e.g. the conjugative plasmid), further comprises a gene for the conversion of IAA to IBA.

In another embodiment, the bacterium, or the plasmid (e.g. the conjugative plasmid) as described herein comprises one or more heterologous gene(s) for the biosynthesis of IBA from IPyA. These include any of the genes described herein for the biosynthesis of IAA from IPyA, wherein the bacterium, or the plasmid (e.g. the conjugative plasmid), further comprises a gene for the conversion of IAA to IBA.

In another embodiment, the bacterium, or the plasmid (e.g. the conjugative plasmid) as described herein comprises one or more heterologous gene(s) for the biosynthesis of IBA from IAM. These include any of the genes described herein for the biosynthesis of IAA from IAAld, wherein the bacterium, or the plasmid (e.g. the conjugative plasmid), further comprises a gene for the conversion of IAA to IBA.

In another embodiment, the bacterium, or the plasmid (e.g. the conjugative plasmid) as described herein comprises one or more heterologous gene(s) for the biosynthesis of IBA from IAOx. These include any of the genes described herein for the biosynthesis of IAA from IAOx, wherein the bacterium, or the plasmid (e.g. the conjugative plasmid), further comprises a gene for the conversion of IAA to IBA.

In another embodiment, the bacterium, or the plasmid (e.g. the conjugative plasmid) as described herein comprises one or more heterologous gene(s) for the biosynthesis of IBA from IAN. These include any of the genes described herein for the biosynthesis of IAA from IAN, wherein the bacterium, or the plasmid (e.g. the conjugative plasmid), further comprises a gene for the conversion of IAA to IBA.

In another embodiment, the bacterium, or the plasmid (e.g. conjugative plasmid), may comprise genes from more than one of the pathways described hereinabove.

### Production of Indole-3-Propionic Acid (IPA)

The tryptophan-derived metabolite and ALM indole-3-propionic acid (IPA) has been shown to regulate intestinal barrier function, and contribute to an anti-inflammatory environment in the gut epithelium (Lanis et al., supra). IPA produced by the gut microbiota has a significant positive effect on barrier integrity. IPA does not signal through AhR, but rather through a different receptor (PXR), and has been shown to protect against indomethacin-induced intestinal injury (Venkatesh et al., Immunity, 41, 296-310, 2014). In one study, IPA reduced the increased intestinal permeability observed in HFD-fed mice (see Jennis et al., Neurogastroenterology & Motility, e13178, 2017, DOI: 10.1111/nmo.13178). IPA has been shown to promote secretion of IL-10 and inhibit TNF production. Due to these effects, which include anti-inflammatory properties, biosynthesis of IPA may be therapeutically useful.

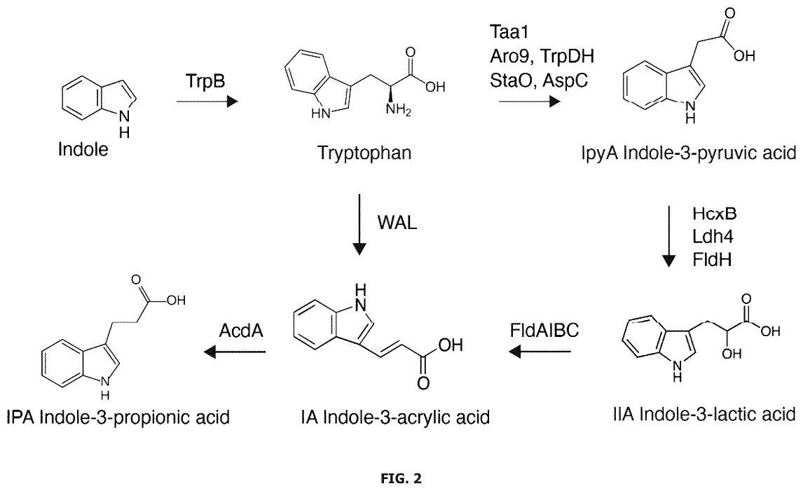

There are various pathways for the production of IPA, which are shown schematically in FIG. 2. Each step of the pathway is governed by one or more heterologous genes. Any of the genes of the pathways (or part pathways, if it is desirable to start production of IPA from an intermediate molecule) shown in FIG. 2 could be inserted into a bacterium, or into a plasmid (e.g. a conjugative plasmid), described herein to produce IPA.

There is provided a modified bacterium for producing and secreting an Auxin Like Molecule (ALM) which is indole-3-propionic acid (IPA), wherein the bacterium comprises one or more heterologous gene(s) for the biosynthesis of IPA, and further comprises a heterologous gene encoding an exporter which is capable of exporting IPA out of the bacterium.

There is provided a plasmid (e.g. a conjugative plasmid) for producing and secreting an Auxin Like Molecule (ALM) which is indole-3-propionic acid (IPA), wherein the plasmid comprises one or more heterologous gene(s) for the biosynthesis of IPA, and further comprises a heterologous gene encoding an exporter which is capable of exporting IPA out of a bacterium which comprises said plasmid and produces and secretes IPA (i.e. out of a recipient bacterium when the plasmid is a conjugative plasmid).

There is provided a host (e.g. a donor) cell (as described elsewhere herein) comprising a plasmid (e.g. conjugative plasmid) as described herein for producing and secreting an Auxin Like Molecule (ALM) which is indole-3-propionic acid (IPA).

In one embodiment, the bacterium, or the plasmid (e.g. the conjugative plasmid), comprises one or more heterologous gene(s) for the biosynthesis of IPA.

In one embodiment, the bacterium. or the plasmid (e.g. the conjugative plasmid), comprises one or more heterologous gene(s) for the biosynthesis of IPA from indole.

In one embodiment, the one or more heterologous gene(s) for the biosynthesis of IPA comprise (or consists of) a gene for the conversion of indole to tryptophan, a gene for the conversion of tryptophan to IPyA, a gene for the conversion of IPyA to ILA, a gene for the conversion of ILA to IA, and a gene for the conversion of IA to IPA.

In one embodiment, the one or more heterologous gene(s) for the biosynthesis of IPA comprise (or consists of) a gene for the conversion of indole to tryptophan, a gene for the conversion of tryptophan to IA; and a gene for the conversion of IA to IPA.

In one embodiment, the bacterium, or the plasmid (e.g. the conjugative plasmid), comprises one or more heterologous gene(s) for the biosynthesis of IPA from tryptophan.

In one embodiment, the one or more heterologous gene(s) for the biosynthesis of IPA comprise (or consists of) a gene for the conversion of tryptophan to IPyA, a gene for the conversion of IPyA to indole-3-lactic acid (ILA), a gene for the conversion of ILA to indole-3-acrylic acid (IA), and a gene for the conversion of IA to IPA.

In one embodiment, the one or more heterologous gene(s) for the biosynthesis of IPA comprise (or consists of) a gene for the conversion of tryptophan to IA, and a gene for the conversion of IA to IPA.

In another aspect, the bacterium, or the plasmid (e.g. the conjugative plasmid) as described herein comprises one or more heterologous gene(s) for the biosynthesis of IPA from IPyA.

In one embodiment, the one or more heterologous gene(s) for the biosynthesis of IPA comprise (or consists of) a gene for the conversion of IPyA to indole-3-lactic acid (ILA), a gene for the conversion of ILA to indole-3-acrylic acid (IA), and a gene for the conversion of IA to IPA.

In another aspect, the bacterium, or the plasmid (e.g. the conjugative plasmid) as described herein comprises one or more heterologous gene(s) for the biosynthesis of IPA from ILA.

In one embodiment, the one or more heterologous gene(s) for the biosynthesis of IPA comprise (or consists of) a gene for the conversion of ILA to indole-3-acrylic acid (IA), and a gene for the conversion of IA to IPA.

In another aspect, the bacterium, or the plasmid (e.g. the conjugative plasmid) as described herein comprises one or more heterologous gene(s) for the biosynthesis of IPA from IA.

In one embodiment, the one or more heterologous gene(s) for the biosynthesis of IPA comprise (or consists of) a gene for the conversion of IA to IPA.

In another embodiment, the bacterium, or the plasmid (e.g. the conjugative plasmid), may comprise genes from more than one of the pathways described hereinabove.

### Production of Indole-3-Pyruvic Acid (IPyA)

The tryptophan-derived metabolite and ALM IPyA has been shown to prevent chronic inflammation in the colon by activating AHR (Aoki et al., supra). Scott et al. showed that IPyA regulated intestinal barrier function in mice (Scott et al., supra). Due to these effects, biosynthesis of IPyA may be therapeutically useful.

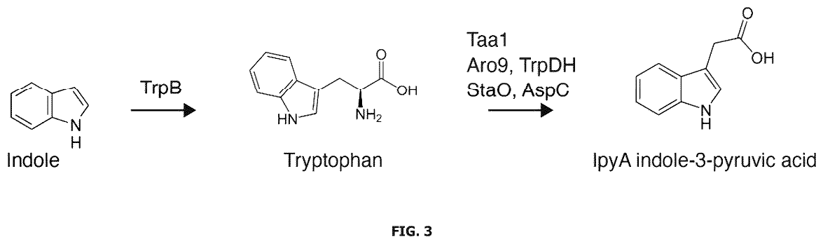

There are various pathways for the production of IPyA, which are shown schematically in FIG. 3. Each step of the pathway is governed by one or more heterologous genes. Any of the genes of the pathways (or part pathways, if it is desirable to start production of IPyA from an intermediate molecule) shown in FIG. 3 could be inserted into a bacterium, or into a plasmid (e.g. into a conjugative plasmid), described herein to produce IPyA.

There is provided a modified bacterium for producing and secreting an Auxin Like Molecule (ALM) which is indole-3-pyruvic acid (IPyA), wherein the bacterium comprises one or more heterologous gene(s) for the biosynthesis of IPyA, and further comprises a heterologous gene encoding an exporter which is capable of exporting IPyA out of the bacterium.

There is provided a plasmid (e.g. a conjugative plasmid) for producing and secreting an Auxin Like Molecule (ALM) which is indole-3-pyruvic acid (IPyA), wherein the plasmid comprises one or more heterologous gene(s) for the biosynthesis of IPyA, and further comprises a heterologous gene encoding an exporter which is capable of exporting IPyA out of a bacterium which comprises said plasmid and produces and secretes IPyA (i.e. out of a recipient bacterium when the plasmid is a conjugative plasmid).

There is provided a host (e.g. a donor) cell (as described elsewhere herein) comprising a plasmid (e.g. a conjugative plasmid) as described herein for producing and secreting an Auxin Like Molecule (ALM) which is indole-3-pyruvic acid (IPyA).

In one embodiment, the bacterium, or the plasmid (e.g. the conjugative plasmid), comprises one or more heterologous gene(s) for the biosynthesis of IPyA.

In one embodiment, the bacterium, or the plasmid (e.g. the conjugative plasmid), comprises one or more heterologous gene(s) for the biosynthesis of IPyA from indole.

In one embodiment, the one or more heterologous gene(s) for the biosynthesis of IPyA comprise (or consists of) a gene for the conversion of indole to tryptophan (optionally a gene which is trpB), and a gene for the conversion of tryptophan to IPyA.

In one embodiment, the bacterium, or the plasmid (e.g. the conjugative plasmid), comprises one or more heterologous gene(s) for the biosynthesis of IPyA from tryptophan.

In one embodiment, the one or more heterologous gene(s) for the biosynthesis of IPyA comprise (or consists of) a gene for the conversion of tryptophan to IPyA.

### Production of Indole-3-Acetaldehyde (IAAld)

The tryptophan-derived metabolite and ALM, indole-3-acetaldehyde (IAAld) has been shown to regulate intestinal barrier function (Scott et al., supra). It increases proliferation of epithelial cells and promotes goblet cell differentiation, reversing an effect of aging as well as acting via the xenobiotic aryl hydrocarbon receptor to increase expression of the cytokine IL-10 (see Powell et al., PNAS, 117 (35), 21519-21526, 2020, https://doi.org/10.1073/pnas.2003004117). Tryptophan degradation to indole derivatives, such as IAAld activated AhR, for production of IL-22, and the AhR-IL-22 acis has been shown to provide antifungal resistance and mucosal protection (see Zelante et al., Immunity, 39 (2), 372-385, 2013, DOI: https://doi.org/10.1016/j.immuni.2013.08.003). Due to these effects, biosynthesis of IAAld may be therapeutically useful.

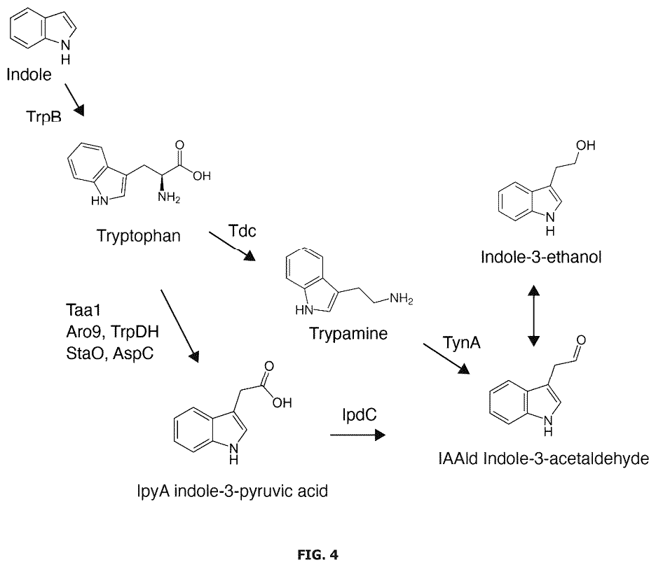

There are various pathways for the production of IAAld, which are shown schematically in FIG. 4. Each step of the pathway is governed by one or more heterologous genes. Any of the genes of the pathways (or part pathways, if it is desirable to start production of IAAld from an intermediate molecule) shown in FIG. 4 could be inserted into a bacterium, or into a plasmid (e.g. into a conjugative plasmid) described herein to produce IAAld.

There is provided a modified bacterium for producing and secreting an Auxin Like Molecule (ALM) which is indole-3-acetaldehyde (IAAld), wherein the bacterium comprises one or more heterologous gene(s) for the biosynthesis of IAAld, and further comprises a heterologous gene encoding an exporter which is capable of exporting IAAld out of the bacterium.

There is provided a plasmid (e.g. a conjugative plasmid) for producing and secreting an Auxin Like Molecule (ALM) which is indole-3-acetaldehyde (IAAld), wherein the plasmid comprises one or more heterologous gene(s) for the biosynthesis of IAAld, and further comprises a heterologous gene encoding an exporter which is capable of exporting IAAld out of a bacterium which comprises said plasmid and produces and secretes IAAld (i.e. out of a recipient bacterium when the plasmid is a conjugative plasmid).

There is provided a host (e.g. a donor) cell (as described elsewhere herein) comprising a plasmid (e.g. a conjugative plasmid) as described herein for producing and secreting an Auxin Like Molecule (ALM) which is indole-3-acetaldehyde (IAAld).

In one embodiment, the bacterium, or the plasmid (e.g. the conjugative plasmid), comprises one or more heterologous gene(s) for the biosynthesis of IAAld.

In one embodiment, the bacterium, or the plasmid (e.g. the conjugative plasmid), comprises one or more heterologous gene(s) for the biosynthesis of IAAld from indole.

In one embodiment, the one or more heterologous gene(s) for the biosynthesis of IAAld comprise (or consists of) a gene for the conversion of indole to tryptophan, a gene for the conversion of tryptophan to tryptamine, and a gene for the conversion of tryptamine to IAAld.

In one embodiment, the one or more heterologous gene(s) for the biosynthesis of IAAld comprise (or consists of) a gene for the conversion of indole to tryptophan, a gene for the conversion of tryptophan to IPyA and a gene for the conversion of IPyA to IAAld.

In one embodiment, the bacterium, or the plasmid (e.g. the conjugative plasmid), comprises one or more heterologous gene(s) for the biosynthesis of IAAld from tryptophan.

In one embodiment, the one or more heterologous gene(s) for the biosynthesis of IAAld comprise (or consists of) a gene for the conversion of tryptophan to tryptamine and a gene for the conversion of tryptamine to IAAld.

In one embodiment, the one or more heterologous gene(s) for the biosynthesis of IAAld comprise (or consists of) a gene for the conversion of tryptophan to IPyA, and a gene for the conversion of IPyA to IAAld.

In another embodiment, the bacterium, or the plasmid (e.g. the conjugative plasmid) as described herein comprises one or more heterologous gene(s) for the biosynthesis of IAAld from tryptamine.

In one embodiment, the one or more heterologous gene(s) for the biosynthesis of IAAld comprise (or consists of) a gene for the conversion of tryptamine to IAAld.

In another embodiment, the bacterium, or the plasmid (e.g. the conjugative plasmid) as described herein comprises one or more heterologous gene(s) for the biosynthesis of IAAld from IPyA.

In one embodiment, the one or more heterologous gene(s) for the biosynthesis of IAAld comprise (or consists of) a gene for the conversion of IPyA to IAAld.

In another embodiment, the bacterium, or the plasmid (e.g. the conjugative plasmid), may comprise genes from more than one of the pathways described hereinabove.

### Production of Indole-3-Ethanol

The tryptophan-derived metabolite and ALM indole-3-ethanol has been shown to regulated intestinal barrier function in mice (Scott et al., supra). Due to these effects, biosynthesis of indole-3-ethanol may be therapeutically useful.

There are various pathways for the production of indole-3-ethanol, which are shown schematically in FIG. 4. Each step of the pathway is governed by one or more heterologous genes. Any of the genes of the pathways (or part pathways, if it is desirable to start production of indole-3-ethanol from an intermediate molecule) shown in FIG. 4 could be inserted into a bacterium or into a plasmid (e.g. into a conjugative plasmid) described herein to produce indole-3-ethanol. An equilibrium exists between IAAld and indole-3-ethanol, and it is expected that a certain amount of indole-3-ethanol will inevitably be produced when producing IAAld.

### Production of Indole-3-Lactic Acid (ILA)

The tryptophan-derived metabolite and ALM, indole-3-lactic acid (ILA) has been shown to be capable in vitro of activating the AhR, a receptor important for controlling intestinal homoeostasis and immune responses. Ex vivo, it modulates immune responses of human CD4⁺ T cells and monocytes in a dose-dependent manner by acting as an agonist of AhR (see Roager et al., Nature Microbiology, 6, 1367-1382, 2021. Due to these effects, biosynthesis of ILA may be therapeutically useful.

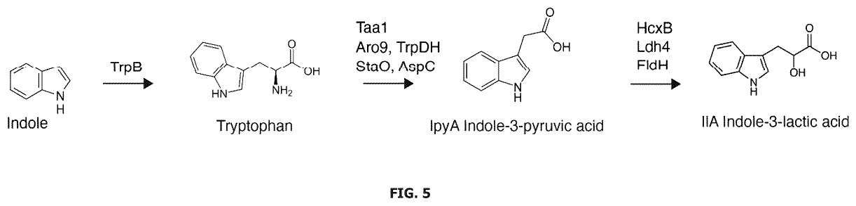

There are various pathways for the production of ILA, which are shown schematically in FIG. 5. Each step of the pathway is governed by one or more heterologous genes. Any of the genes of the pathways (or part pathways, if it is desirable to start production of ILA from an intermediate molecule) shown in FIG. 5 could be inserted into a bacterium or into a plasmid (e.g. into a conjugative plasmid) described herein to produce ILA.

There is provided a modified bacterium for producing and secreting an Auxin Like Molecule (ALM) which is indole-3-lactic acid (ILA), wherein the bacterium comprises one or more heterologous gene(s) for the biosynthesis of ILA, and further comprises a heterologous gene encoding an exporter which is capable of exporting ILA out of the bacterium.

There is provided a plasmid (e.g. a conjugative plasmid) for producing and secreting an Auxin Like Molecule (ALM) which is indole-3-lactic acid (ILA), wherein the plasmid comprises one or more heterologous gene(s) for the biosynthesis of ILA, and further comprises a heterologous gene encoding an exporter which is capable of exporting ILA out of a bacterium which comprises said plasmid and produces and secretes ILA (i.e. out of a recipient bacterium when the plasmid is a conjugative plasmid).

There is provided a host (e.g. a donor) cell (as described elsewhere herein) comprising a plasmid (e.g. a conjugative plasmid) as described herein for producing and secreting an Auxin Like Molecule (ALM) which is indole-3-lactic acid (ILA).

In one embodiment, the bacterium, or the plasmid (e.g. the conjugative plasmid), comprises one or more heterologous gene(s) for the biosynthesis of ILA.

In one embodiment, the bacterium, or the plasmid (e.g. the conjugative plasmid), comprises one or more heterologous gene(s) for the biosynthesis of ILA from indole.

In one embodiment, the one or more heterologous gene(s) for the biosynthesis of ILA comprise (or consists of) a gene for the conversion of indole to tryptophan, a gene for the conversion of tryptophan to IPyA, and a gene for the conversion of IPyA to ILA.

In one embodiment, the bacterium, or the plasmid (e.g. the conjugative plasmid), comprises one or more heterologous gene(s) for the biosynthesis of ILA from tryptophan.

In one embodiment, the one or more heterologous gene(s) for the biosynthesis of ILA comprise (or consists of) a gene for the conversion of tryptophan to IPyA, and a gene for the conversion of IPyA to ILA.

In another embodiment, the bacterium, or the plasmid (e.g. the conjugative plasmid) as described herein comprises one or more heterologous gene(s) for the biosynthesis of ILA from IPyA.

In one embodiment, the one or more heterologous gene(s) for the biosynthesis of ILA comprise (or consists of) a gene for the conversion of IPyA to ILA.

In another embodiment, the bacterium, or the plasmid (e.g. the conjugative plasmid), may comprise genes from more than one of the pathways described hereinabove.

### Production of Indole-3-Acrylic Acid (IA)

The tryptophan-derived metabolite and ALM, indole-3-acrylic acid (IA) promotes intestinal epithelial barrier function and mitigates inflammatory responses. It is a potent activator of AhR and upregulates Muc2 gene expression. IA enhances gut barrier function and tight junctions, and provides anti-inflammatory effects. Treatment of human PBMCs with IA led to reduced IL-6 and IL-1B secretion in response to LPS stimulation and activation of the NRF2-ARE pathway, in addition to AhR activation, suggesting that IA may have an anti-oxidative and anti-inflammatory functions in humans (see Wlodarska et al., Cell Host Microbe, 22 (1), 25-37, e6, 2017, doi: 10.1016/j.chom.2017.06.007). Due to these effects, biosynthesis of IA may be therapeutically useful.

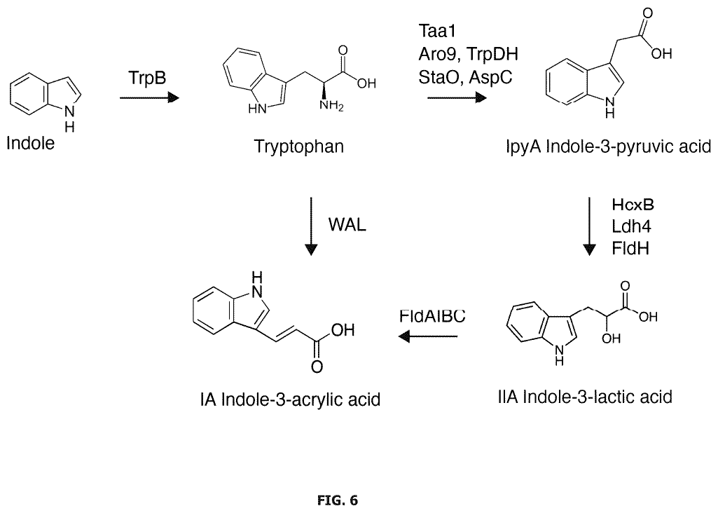

There are various pathways for the production of IA, which are shown schematically in FIG. 6. Each step of the pathway is governed by one or more heterologous genes. Any of the genes of the pathways (or part pathways, if it is desirable to start production of IA from an intermediate molecule) shown in FIG. 6 could be inserted into a bacterium or into a plasmid (e.g. into a conjugative plasmid) described herein to produce IA.

There is provided a modified bacterium for producing and secreting an Auxin Like Molecule (ALM) which is indole-3-acrylic acid (IA), wherein the bacterium comprises one or more heterologous gene(s) for the biosynthesis of IA, and further comprises a heterologous gene encoding an exporter which is capable of exporting IA out of the bacterium.

There is provided a plasmid (e.g. a conjugative plasmid) for producing and secreting an Auxin Like Molecule (ALM) which is indole-3-acrylic acid (IA), wherein the plasmid comprises one or more heterologous gene(s) for the biosynthesis of IA, and further comprises a heterologous gene encoding an exporter which is capable of exporting IA out of a bacterium which comprises said plasmid and produces and secretes IA (i.e. out of a recipient bacterium when the plasmid is a conjugative plasmid).

There is provided a host (e.g. a donor) cell (as described elsewhere herein) comprising a plasmid (e.g. a conjugative plasmid) as described herein for producing and secreting an Auxin Like Molecule (ALM) which is indole-3-acrylic acid (IA).

In one embodiment, the bacterium, or the plasmid (e.g. the conjugative plasmid), comprises one or more heterologous gene(s) for the biosynthesis of IA.

In one embodiment, the bacterium, or the plasmid (e.g. the conjugative plasmid), comprises one or more heterologous gene(s) for the biosynthesis of IA from indole.

In one embodiment, the one or more heterologous gene(s) for the biosynthesis of IA comprise (or consists of) a gene for the conversion of indole to tryptophan, a gene for the conversion of tryptophan to IPyA, a gene for the conversion of IPyA to ILA, and a gene for the conversion of ILA to IA.

In one embodiment, the one or more heterologous gene(s) for the biosynthesis of IA comprise (or consists of) a gene for the conversion of indole to tryptophan, and a gene for the conversion of tryptophan to IA.

In one embodiment, the bacterium, or the plasmid (e.g. the conjugative plasmid), comprises one or more heterologous gene(s) for the biosynthesis of IA from tryptophan.

In one embodiment, the one or more heterologous gene(s) for the biosynthesis of IA comprise (or consists of) a gene for the conversion of tryptophan to IPyA, a gene for the conversion of IPyA to ILA, and a gene for the conversion of ILA to IA.

In one embodiment, the one or more heterologous gene(s) for the biosynthesis of IA comprise (or consists of) a gene for the conversion of tryptophan to IA.

In another embodiment, the bacterium, or the plasmid (e.g. the conjugative plasmid) as described herein comprises one or more heterologous gene(s) for the biosynthesis of IA from IPyA.

In one embodiment, the one or more heterologous gene(s) for the biosynthesis of IA comprise (or consists of) a gene for the conversion of IPyA to ILA, and a gene for the conversion of ILA to IA.

In another embodiment, the bacterium, or the plasmid (e.g. the conjugative plasmid) as described herein comprises one or more heterologous gene(s) for the biosynthesis of IA from ILA.

In one embodiment, the one or more heterologous gene(s) for the biosynthesis of IA comprise (or consists of) a gene for the conversion of ILA to IA.

In another embodiment, the bacterium, or the plasmid (e.g. the conjugative plasmid), may comprise genes from more than one of the pathways described hereinabove.

### Production of Indole-3-Carboxylic Acid (ICA)

The tryptophan-derived metabolite and ALM, indole-3-carboxylic acid (ICA) may be therapeutically useful in certain cancer treatments and/or treatment or prevention of HIV. Indole-3-carboxylic acid (ICA) is cytotoxic to A549 human lung and MCF-7 human breast cancer cells (EC₅₀s=4.6 and 12.9 μg/ml, respectively) and inhibits HIV replication in infected H9 lymphocytes (IC₅₀=16.4 μg/ml), see Wu et al., Chem. Pharm. Bull. (Tokyo), 52 (3), 345-349, 2004. Due to these effects, biosynthesis of ICA may be therapeutically useful.

There are various pathways for the production of ICA, which are shown schematically in FIG. 7. Each step of the pathway is governed by one or more heterologous genes. Any of the genes of the pathways (or part pathways, if it is desirable to start production of ICA from an intermediate molecule) shown in FIG. 7 could be inserted into a bacterium or into a plasmid (e.g. into a conjugative plasmid) described herein to produce IA.

There is provided a modified bacterium for producing and secreting an Auxin Like Molecule (ALM) which is indole-3-carboxylic acid (ICA), wherein the bacterium comprises one or more heterologous gene(s) for the biosynthesis of ICA, and further comprises a heterologous gene encoding an exporter which is capable of exporting ICA out of the bacterium.

There is provided a plasmid (e.g. a conjugative plasmid) for producing and secreting an Auxin Like Molecule (ALM) which is indole-3-carboxylic acid (ICA), wherein the plasmid comprises one or more heterologous gene(s) for the biosynthesis of ICA, and further comprises a heterologous gene encoding an exporter which is capable of exporting ICA out of a bacterium which comprises said plasmid and produces and secretes ICA (i.e. out of a recipient bacterium when the plasmid is a conjugative plasmid).

There is provided a host (e.g. a donor) cell (as described elsewhere herein) comprising a plasmid (e.g. a conjugative plasmid) as described herein for producing and secreting an Auxin Like Molecule (ALM) which is indole-3-carboxylic acid (ICA).

In one embodiment, the bacterium, or the plasmid (e.g. the conjugative plasmid), comprises one or more heterologous gene(s) for the biosynthesis of ICA.

In one embodiment, the bacterium, or the plasmid (e.g. the conjugative plasmid), comprises one or more heterologous gene(s) for the biosynthesis of ICA from indole.

In one embodiment, the one or more heterologous gene(s) for the biosynthesis of ICA comprise (or consists of) a gene for the conversion of indole to tryptophan, a gene for the conversion of tryptophan to tryptamine, a gene for the conversion of tryptamine to IAAld, a gene for the conversion of IAAld to IAA, a gene for the conversion of IAA to skatole, a gene for the conversion of skatole to indole-3-methanol (IM), a gene for the conversion of IM to indole-3-carboxaldehyde (IAID), and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to tryptamine, a gene for the conversion of tryptamine to IAAld, a gene for the conversion of IAAld to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IPyA, a gene for the conversion of IPyA to IAAld, a gene for the conversion of IAAld to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IPyA, a gene for the conversion of IPyA to IAAld, a gene for the conversion of IAAld to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IPyA, a gene for the conversion of IPyA to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IPyA, a gene for the conversion of IPyA to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IAM, a gene for the conversion of IAM to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IAM, a gene for the conversion of IAM to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IAOx, a gene for the conversion of IAOx to IAN, a gene for the conversion of IAN to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IAOx, a gene for the conversion of IAOx to IAN, a gene for the conversion of IAN to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IAOx, a gene for the conversion of IAOx to IAN, a gene for the conversion of IAN to IAM, a gene for the conversion of IAM to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IAOx, a gene for the conversion of IAOx to IAN, a gene for the conversion of IAN to IAM, a gene for the conversion of IAM to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of indole to tryptophan, a gene for the conversion of tryptophan to IAOx, a gene for the conversion of IAOx to IAN, a gene for the conversion of IAN to IAID, and a gene for the conversion of IAID to ICA.

In one embodiment, the bacterium, or the plasmid (e.g. the conjugative plasmid), comprises one or more heterologous gene(s) for the biosynthesis of ICA from tryptophan.

In one embodiment, the one or more heterologous gene(s) for the biosynthesis of ICA comprise (or consists of) a gene for the conversion of tryptophan to tryptamine, a gene for the conversion of tryptamine to IAAld, a gene for the conversion of IAAld to IAA, a gene for the conversion of IAA to skatole, a gene for the conversion of skatole to indole-3-methanol (IM), a gene for the conversion of IM to indole-3-carboxaldehyde (IAID), and a gene for the conversion of IAID to ICA.

In one embodiment, the one or more heterologous gene(s) for the biosynthesis of IA comprise (or consists of) a gene for the conversion of tryptophan to tryptamine, a gene for the conversion of tryptamine to IAAld, a gene for the conversion of IAAld to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of tryptophan to IPyA, a gene for the conversion of IPyA to IAAld, a gene for the conversion of IAAld to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of tryptophan to IPyA, a gene for the conversion of IPyA to IAAld, a gene for the conversion of IAAld to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of tryptophan to IPyA, a gene for the conversion of IPyA to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of tryptophan to IPyA, a gene for the conversion of IPyA to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of tryptophan to IAM, a gene for the conversion of IAM to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of tryptophan to IAM, a gene for the conversion of IAM to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of tryptophan to IAOx, a gene for the conversion of IAOx to IAN, a gene for the conversion of IAN to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of tryptophan to IAOx, a gene for the conversion of IAOx to IAN, a gene for the conversion of IAN to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of tryptophan to IAOx, a gene for the conversion of IAOx to IAN, a gene for the conversion of IAN to IAM, a gene for the conversion of IAM to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of tryptophan to IAOx, a gene for the conversion of IAOx to IAN, a gene for the conversion of IAN to IAM, a gene for the conversion of IAM to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of tryptophan to IAOx, a gene for the conversion of IAOx to IAN, a gene for the conversion of IAN to IAID, and a gene for the conversion of IAID to ICA.

In another embodiment, the bacterium, or the plasmid (e.g. the conjugative plasmid) as described herein comprises one or more heterologous gene(s) for the biosynthesis of ICA from tryptamine.

In one embodiment, the one or more heterologous gene(s) for the biosynthesis of IA comprise (or consists of) a gene for the conversion of tryptamine to IAAld, a gene for the conversion of IAAld to IAA, a gene for the conversion of IAA to skatole, a gene for the conversion of skatole to indole-3-methanol (IM), a gene for the conversion of IM to indole-3-carboxaldehyde (IAID), and a gene for the conversion of IAID to ICA.

In one embodiment, the one or more heterologous gene(s) for the biosynthesis of IA comprise (or consists of) a gene for the conversion of tryptamine to IAAld, a gene for the conversion of IAAld to IAA, a gene for the conversion of IAA to skatole, a gene for the conversion of skatole to IAID, and a gene for the conversion of IAID to ICA.

In another embodiment, the bacterium, or the plasmid (e.g. the conjugative plasmid) as described herein comprises one or more heterologous gene(s) for the biosynthesis of ICA from IAAld.

In one embodiment, the one or more heterologous gene(s) for the biosynthesis of IA comprise (or consists of) a gene for the conversion of IAAld to IAA, a gene for the conversion of IAA to skatole, a gene for the conversion of skatole to indole-3-methanol (IM), a gene for the conversion of IM to indole-3-carboxaldehyde (IAID), and a gene for the conversion of IAID to ICA.

In one embodiment, the one or more heterologous gene(s) for the biosynthesis of IA comprise (or consists of) a gene for the conversion of IAAld to IAA, a gene for the conversion of IAA to skatole, a gene for the conversion of skatole to IAID, and a gene for the conversion of IAID to ICA.

In another embodiment, the bacterium, or the plasmid (e.g. the conjugative plasmid) as described herein comprises one or more heterologous gene(s) for the biosynthesis of ICA from IAA.

In one embodiment, the one or more heterologous gene(s) for the biosynthesis of IA comprise (or consists of) a gene for the conversion of IAA to skatole, a gene for the conversion of skatole to indole-3-methanol (IM), a gene for the conversion of IM to indole-3-carboxaldehyde (IAID), and a gene for the conversion of IAID to ICA.

In one embodiment, the one or more heterologous gene(s) for the biosynthesis of IA comprise (or consists of) a gene for the conversion of IAA to skatole, a gene for the conversion of skatole to IAID, and a gene for the conversion of IAID to ICA.

In another embodiment, the bacterium, or the plasmid (e.g. the conjugative plasmid) as described herein comprises one or more heterologous gene(s) for the biosynthesis of ICA from skatole. In one embodiment, the one or more heterologous gene(s) for the biosynthesis of IA comprise (or consists of) a gene for the conversion of skatole to indole-3-methanol (IM), a gene for the conversion of IM to indole-3-carboxaldehyde (IAID), and a gene for the conversion of IAID to ICA.

In one embodiment, the one or more heterologous gene(s) for the biosynthesis of IA comprise (or consists of) a gene for the conversion of skatole to IAID, and a gene for the conversion of IAID to ICA.

In another embodiment, the bacterium, or the plasmid (e.g. the conjugative plasmid) as described herein comprises one or more heterologous gene(s) for the biosynthesis of ICA from IM.

In one embodiment, the one or more heterologous gene(s) for the biosynthesis of IA comprise (or consists of) a gene for the conversion of IM to indole-3-carboxaldehyde (IAID), and a gene for the conversion of IAID to ICA.

In another embodiment, the bacterium, or the plasmid (e.g. the conjugative plasmid) as described herein comprises one or more heterologous gene(s) for the biosynthesis of ICA from IAID.

In one embodiment, the one or more heterologous gene(s) for the biosynthesis of IA comprise (or consists of) a gene for the conversion of IAID to ICA.

In another embodiment, the bacterium, or the plasmid (e.g. the conjugative plasmid) as described herein comprises one or more heterologous gene(s) for the biosynthesis of ICA from IPyA.

In one embodiment, the one or more heterologous gene(s) for the biosynthesis of IA comprise (or consists of) a gene for the conversion of IPyA to IAAld, a gene for the conversion of IAAld to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of IPyA to IAAld, a gene for the conversion of IAAld to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of IPyA to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of IPyA to IAA, a gene for the conversion of IAA to skatole, a gene for the conversion of skatole to IAID, and a gene for the conversion of IAID to ICA.

In another embodiment, the bacterium, or the plasmid (e.g. the conjugative plasmid) as described herein comprises one or more heterologous gene(s) for the biosynthesis of ICA from IAM.

In one embodiment, the one or more heterologous gene(s) for the biosynthesis of IA comprise (or consists of) a gene for the conversion of IAM to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of IAM to IAA, a gene for the conversion of IAA to skatole, a gene for the conversion of skatole to IAID, and a gene for the conversion of IAID to ICA.

In another embodiment, the bacterium, or the plasmid (e.g. the conjugative plasmid) as described herein comprises one or more heterologous gene(s) for the biosynthesis of ICA from IAN.

In one embodiment, the one or more heterologous gene(s) for the biosynthesis of IA comprise (or consists of) a gene for the conversion of IAN to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of IAN to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of IAN to IAM, a gene for the conversion of IAM to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of IAN to IAM, a gene for the conversion of IAM to IAA, a gene for the conversion of IAA to skatole, a gene for the conversion of skatole to IAID, and a gene for the conversion of IAID to ICA

In one embodiment, the one or more heterologous gene(s) for the biosynthesis of IA comprise (or consists of) a gene for the conversion of IAN to IAID, and a gene for the conversion of IAID to ICA.

In another embodiment, the bacterium, or the plasmid (e.g. the conjugative plasmid) as described herein comprises one or more heterologous gene(s) for the biosynthesis of ICA from IAOx.

In one embodiment, the one or more heterologous gene(s) for the biosynthesis of IA comprise (or consists of) a gene for the conversion of IAOx to IAN, a gene for the conversion of IAN to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of IAOx to IAN, a gene for the conversion of IAN to IAA, a gene for the conversion of IAA to skatole, a gene for the conversion of skatole to IAID, and a gene for the conversion of IAID to ICA.

In one embodiment, the one or more heterologous gene(s) for the biosynthesis of IA comprise (or consists of) a gene for the conversion of IAOx to IAN, a gene for the conversion of IAN to IAM, a gene for the conversion of IAM to IAA, a gene for the conversion of IAA to skatole, a gene for the conversion of skatole to IM, a gene for the conversion of IM to IAID, and a gene for the conversion of IAID to ICA.

In one embodiment, the one or more heterologous gene(s) for the biosynthesis of IA comprise (or consists of) a gene for the conversion of IAOx to IAN, a gene for the conversion of IAN to IAM, a gene for the conversion of IAM to IAA, a gene for the conversion of IAA to skatole, a gene for the conversion of skatole to IAID, and a gene for the conversion of IAID to ICA

In one embodiment, the one or more heterologous gene(s) for the biosynthesis of IA comprise (or consists of) a gene for the conversion of IAOx to IAN, a gene for the conversion of IAN to IAID, and a gene for the conversion of IAID to ICA.

In another embodiment, the bacterium, or the plasmid (e.g. the conjugative plasmid), may comprise genes from more than one of the pathways described hereinabove.

### Production of Other Indole-Derived Intermediates

In some embodiments, it may be desirable to engineer a modified bacterium, or a plasmid (e.g. a conjugative plasmid), to produce and secrete any of the molecules which are described as intermediates in one of the other pathways (e.g. as described in any one of FIGS. 1 to 7). In those embodiments, the pathway of genes which are included in the modified bacterium, or in the plasmid (e.g. in the conjugative plasmid), comprise any part of any of the pathways described herein.

### Tryptamine

There is provided a modified bacterium for producing and secreting tryptamine, wherein the bacterium comprises one or more heterologous gene(s) for the biosynthesis of tryptamine, and further comprises a heterologous gene encoding an exporter which is capable of exporting tryptamine out of the bacterium.

There is provided a plasmid (e.g. a conjugative plasmid) for producing and secreting tryptamine, wherein the plasmid comprises one or more heterologous gene(s) for the biosynthesis of tryptamine, and further comprises a heterologous gene encoding an exporter which is capable of exporting tryptamine out of a bacterium which comprises said plasmid and produces and secretes tryptamine (i.e. out of a recipient bacterium when the plasmid is a conjugative plasmid).

There is provided a host (e.g. a donor) cell (as described elsewhere herein) comprising a plasmid (e.g. a conjugative plasmid) as described herein for producing and secreting an tryptamine.

In one embodiment, the bacterium, or the plasmid (e.g. the conjugative plasmid), comprises one or more heterologous gene(s) for the biosynthesis of tryptamine.

In one embodiment, the bacterium, or the plasmid (e.g. the conjugative plasmid), comprises one or more heterologous gene(s) for the biosynthesis of tryptamine from indole.

In one embodiment, the one or more heterologous gene(s) for the biosynthesis of tryptamine comprise (or consists of) a gene for the conversion of indole to tryptophan and a gene for the conversion of tryptophan to tryptamine.

In one embodiment, the bacterium, or the plasmid (e.g. the conjugative plasmid), comprises one or more heterologous gene(s) for the biosynthesis of tryptamine from tryptophan.

In one embodiment, the one or more heterologous gene(s) for the biosynthesis of tryptamine comprise (or consists of) a gene for the conversion of tryptophan to tryptamine.

### Indole-3-Methanol (IM):

Indole-3-methanol is a resulting compound that comes from eating cruciferous vegetables such as Brussel sprouts, cabbage, cauliflower, broccoli, and kale. It is known to stimulate detoxifying enzymes in the gut and liver. Because diets high in these vegetables slow cancer growth in animals, IM is thought to be a good candidate for cancer prevention. Lab studies suggest that I3C may have activity across various tumour types, or enhance activity of some chemotherapy drugs.

As an AhR ligand precursor, IM has also been shown to hepatoprotective during alcoholic liver disease (see Choi et al., J. Nutr. Biochem., 55:12-25, 2018, doi: 10.1016/j.jnutbio.2017.11.011).

One study showed a statistically significant regression of cervical intra-epithelial neoplasia (CIN) in patients treated with orally dosed IM, compared with placebo. The 2/16α-hydroxyestrone ratio changed in a dose-dependent fashion, see Bell et al., Gynecologic Oncology, 78 (2), 123-129, 2000).

IM attenuates colitis primarily through induction of IL-22, see Busbee et al., JCI Insight., 5 (1): e127551, 2020, doi: 10.1172/jci.insight.127551. Due to these effects and others known in the art, biosynthesis of IM may be therapeutically useful.

There is provided a modified bacterium for producing and secreting indole-3-methanol (IM), wherein the bacterium comprises one or more heterologous gene(s) for the biosynthesis of IM, and further comprises a heterologous gene encoding an exporter which is capable of exporting IM out of the bacterium.

There is provided a plasmid (e.g. a conjugative plasmid) for producing and secreting indole-3-methanol (IM), wherein the plasmid comprises one or more heterologous gene(s) for the biosynthesis of IM, and further comprises a heterologous gene encoding an exporter which is capable of exporting IM out of a bacterium which comprises said plasmid and produces and secretes IM (i.e. out of a recipient bacterium when the plasmid is a conjugative plasmid)

There is provided a host (e.g. a donor) cell (as described elsewhere herein) comprising a plasmid (e.g. a conjugative plasmid) as described herein for producing and secreting an indole-3-methanol (IM).

In one embodiment, the bacterium, or the plasmid (e.g. the conjugative plasmid), comprises one or more heterologous gene(s) for the biosynthesis of indole-3-methanol (IM).

In one embodiment, the bacterium, or the plasmid (e.g. the conjugative plasmid), comprises one or more heterologous gene(s) for the biosynthesis of IM from indole.

In one embodiment, the one or more heterologous gene(s) for the biosynthesis of IM comprise (or consists of) a gene for the conversion of indole to tryptophan, a gene for the conversion of tryptophan to tryptamine, a gene for the conversion of tryptamine to IAAld, a gene for the conversion of IAAld to IAA, a gene for the conversion of IAA to skatole, and a gene for the conversion of skatole to indole-3-methanol.

In one embodiment, the one or more heterologous gene(s) for the biosynthesis of IM comprise (or consists of) a gene for the conversion of indole to tryptophan, a gene for the conversion of tryptophan to IPyA, a gene for the conversion of IPyA to IAAld, a gene for the conversion of IAAld to IAA, a gene for the conversion of IAA to skatole, and a gene for the conversion of skatole to indole-3-methanol.

In one embodiment, the one or more heterologous gene(s) for the biosynthesis of IM comprise (or consists of) a gene for the conversion of indole to tryptophan, a gene for the conversion of tryptophan to IPyA, a gene for the conversion of IPyA to IAA, a gene for the conversion of IAA to skatole, and a gene for the conversion of skatole to indole-3-methanol.

In one embodiment, the one or more heterologous gene(s) for the biosynthesis of IM comprise (or consists of) a gene for the conversion of indole to tryptophan, a gene for the conversion of tryptophan to IAM, a gene for the conversion of IAM to IAA, a gene for the conversion of IAA to skatole, and a gene for the conversion of skatole to indole-3-methanol.

In one embodiment, the one or more heterologous gene(s) for the biosynthesis of IM comprise (or consists of) a gene for the conversion of indole to tryptophan, a gene for the conversion of tryptophan to IAOx, a gene for the conversion of IAOx to IAN, a gene for the conversion of IAN to IAA, a gene for the conversion of IAA to skatole, and a gene for the conversion of skatole to indole-3-methanol.

In one embodiment, the one or more heterologous gene(s) for the biosynthesis of IM comprise (or consists of) a gene for the conversion of indole to tryptophan, a gene for the conversion of tryptophan to IAOx, a gene for the conversion of IAOx to IAN, a gene for the conversion of IAN to IAM, a gene for the conversion of IAM to IAA, a gene for the conversion of IAA to skatole, and a gene for the conversion of skatole to indole-3-methanol.

In one embodiment, the bacterium, or the plasmid (e.g. the conjugative plasmid), comprises one or more heterologous gene(s) for the biosynthesis of IM from tryptophan.

In one embodiment, the one or more heterologous gene(s) for the biosynthesis of IM comprise (or consists of) a gene for the conversion of tryptophan to tryptamine, a gene for the conversion of tryptamine to IAAld, a gene for the conversion of IAAld to IAA, a gene for the conversion of IAA to skatole, and a gene for the conversion of skatole to indole-3-methanol.

In one embodiment, the one or more heterologous gene(s) for the biosynthesis of IM comprise (or consists of) a gene for the conversion of tryptophan to IPyA, a gene for the conversion of IPyA to IAAld, a gene for the conversion of IAAld to IAA, a gene for the conversion of IAA to skatole, and a gene for the conversion of skatole to indole-3-methanol.

In one embodiment, the one or more heterologous gene(s) for the biosynthesis of IM comprise (or consists of) a gene for the conversion of tryptophan to IPyA, a gene for the conversion of IPyA to IAA, a gene for the conversion of IAA to skatole, and a gene for the conversion of skatole to indole-3-methanol.

In one embodiment, the one or more heterologous gene(s) for the biosynthesis of IM comprise (or consists of) a gene for the conversion of tryptophan to IAM, a gene for the conversion of IAM to IAA, a gene for the conversion of IAA to skatole, and a gene for the conversion of skatole to indole-3-methanol.

In one embodiment, the one or more heterologous gene(s) for the biosynthesis of IM comprise (or consists of) a gene for the conversion of tryptophan to IAOx, a gene for the conversion of IAOx to IAN, a gene for the conversion of IAN to IAA, a gene for the conversion of IAA to skatole, and a gene for the conversion of skatole to indole-3-methanol.

In one embodiment, the one or more heterologous gene(s) for the biosynthesis of IM comprise (or consists of) a gene for the conversion of tryptophan to IAOx, a gene for the conversion of IAOx to IAN, a gene for the conversion of IAN to IAM, a gene for the conversion of IAM to IAA, a gene for the conversion of IAA to skatole, and a gene for the conversion of skatole to indole-3-methanol.

In one embodiment, the bacterium, or the plasmid (e.g. the conjugative plasmid), comprises one or more heterologous gene(s) for the biosynthesis of IM from skatole. In one embodiment, the one or more heterologous gene(s) for the biosynthesis of IM from skatole comprise (or consists of) the genes described in the pathways above.

In one embodiment, the bacterium, or the plasmid (e.g. the conjugative plasmid), comprises one or more heterologous gene(s) for the biosynthesis of IM from IAA. In one embodiment, the one or more heterologous gene(s) for the biosynthesis of IM from IAA comprise (or consists of) the genes described in the pathways above.

In one embodiment, the bacterium, or the plasmid (e.g. the conjugative plasmid), comprises one or more heterologous gene(s) for the biosynthesis of IM from IAAld. In one embodiment, the one or more heterologous gene(s) for the biosynthesis of IM from IAAld comprise (or consists of) the genes described in the pathways above.

In one embodiment, the bacterium, or the plasmid (e.g. the conjugative plasmid), comprises one or more heterologous gene(s) for the biosynthesis of IM from IPyA. In one embodiment, the one or more heterologous gene(s) for the biosynthesis of IM from IPyA comprise (or consists of) the genes described in the pathways above.

In one embodiment, the bacterium, or the plasmid (e.g. the conjugative plasmid), comprises one or more heterologous gene(s) for the biosynthesis of IM from IAM. In one embodiment, the one or more heterologous gene(s) for the biosynthesis of IM from IAM comprise (or consists of) the genes described in the pathways above.

In one embodiment, the bacterium, or the plasmid (e.g. the conjugative plasmid), comprises one or more heterologous gene(s) for the biosynthesis of IM from IAOx. In one embodiment, the one or more heterologous gene(s) for the biosynthesis of IM from IAOx comprise (or consists of) the genes described in the pathways above.

In one embodiment, the bacterium, or the plasmid (e.g. the conjugative plasmid), comprises one or more heterologous gene(s) for the biosynthesis of IM from IAN. In one embodiment, the one or more heterologous gene(s) for the biosynthesis of IM from IAN comprise (or consists of) the genes described in the pathways above.

In another embodiment, the bacterium, or the plasmid (e.g. the conjugative plasmid), may comprise genes from more than one of the pathways described hereinabove.

### Indole-3-Carboxaldehylde (IAID)

Indole-3-carboxaldehyde (IAID) has been shown to be able to regulate intestinal mucosal homeostasis by acting as a ligand of AhR in a murine model of metabolic syndrome (see Puccetti et al., Int. J. Pharm., 602:120610, 2021, doi: 10.1016/j.ijpharm.2021.120610). It has also been shown to restore gut mucosal integrity and to provide protection from liver fibrosis in another murine model, see D'Onofrio et al., Cells, 10 (7), 1622, 2021, doi: 10.3390/cells10071622. Due to these effects and others known in the art, biosynthesis of IAID may be therapeutically useful.

There is provided a modified bacterium for producing and secreting indole-3-carboxaldehyde (IAID), wherein the bacterium comprises one or more heterologous gene(s) for the biosynthesis of IAID, and further comprises a heterologous gene encoding an exporter which is capable of exporting LAID out of the bacterium.

There is provided a plasmid (e.g. a conjugative plasmid) for producing and secreting indole-3-carboxaldehyde (IAID), wherein the plasmid comprises one or more heterologous gene(s) for the biosynthesis of IAID, and further comprises a heterologous gene encoding an exporter which is capable of exporting IAID out of a bacterium which comprises said plasmid and produces and secretes IAID (i.e. out of a recipient bacterium when the plasmid is a conjugative plasmid).

There is provided a host (e.g. a donor) cell (as described elsewhere herein) comprising a plasmid (e.g. a conjugative plasmid) as described herein for producing and secreting indole-3-carboxaldehyde (IAID).

In one embodiment, the bacterium, or the plasmid (e.g. the conjugative plasmid), comprises one or more heterologous gene(s) for the biosynthesis of indole-3-carboxaldehyde (IAID).

In one embodiment, the bacterium, or the plasmid (e.g. the conjugative plasmid), comprises one or more heterologous gene(s) for the biosynthesis of IAID from indole.

In one embodiment, the one or more heterologous gene(s) for the biosynthesis of IAID comprise (or consists of) a gene for the conversion of indole to tryptophan, a gene for the conversion of tryptophan to tryptamine, a gene for the conversion of tryptamine to IAAld, a gene for the conversion of IAAld to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of indole to tryptophan, a gene for the conversion of tryptophan to tryptamine, a gene for the conversion of tryptamine to IAAld, a gene for the conversion of IAAld to IAA, a gene for the conversion of IAA to skatole, a gene for the conversion of skatole to IAID.

In one embodiment, the one or more heterologous gene(s) for the biosynthesis of IAID comprise (or consists of) a gene for the conversion of indole to tryptophan, a gene for the conversion of tryptophan to IPyA, a gene for the conversion of IPyA to IAAld, a gene for the conversion of IAAld to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of indole to tryptophan, a gene for the conversion of tryptophan to IPyA, a gene for the conversion of IPyA to IAAld, a gene for the conversion of IAAld to IAA, a gene for the conversion of IAA to skatole, a gene for the conversion of skatole to IAID.

In one embodiment, the one or more heterologous gene(s) for the biosynthesis of IAID comprise (or consists of) a gene for the conversion of indole to tryptophan, a gene for the conversion of tryptophan to IPyA, a gene for the conversion of IPyA to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of indole to tryptophan, a gene for the conversion of tryptophan to IPyA, a gene for the conversion of IPyA to IAA, a gene for the conversion of IAA to skatole, a gene for the conversion of skatole to IAID.

In one embodiment, the one or more heterologous gene(s) for the biosynthesis of IAID comprise (or consists of) a gene for the conversion of indole to tryptophan, a gene for the conversion of tryptophan to IAM, a gene for the conversion of IAM to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of indole to tryptophan, a gene for the conversion of tryptophan to IAM, a gene for the conversion of IAM to IAA, a gene for the conversion of IAA to skatole, a gene for the conversion of skatole to IAID.

In one embodiment, the one or more heterologous gene(s) for the biosynthesis of IAID comprise (or consists of) a gene for the conversion of indole to tryptophan, a gene for the conversion of tryptophan to IAOx, a gene for the conversion of IAOx to IAN, a gene for the conversion of IAN to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of indole to tryptophan, a gene for the conversion of tryptophan to IAOx, a gene for the conversion of IAOx to IAN, a gene for the conversion of IAN to IAA, a gene for the conversion of IAA to skatole, a gene for the conversion of skatole to IAID.

In one embodiment, the one or more heterologous gene(s) for the biosynthesis of IAID comprise (or consists of) a gene for the conversion of indole to tryptophan, a gene for the conversion of tryptophan to IAOx, a gene for the conversion of IAOx to IAN, a gene for the conversion of IAN to IAM, a gene for the conversion of IAM to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of indole to tryptophan, a gene for the conversion of tryptophan to IAOx, a gene for the conversion of IAOx to IAN, a gene for the conversion of IAN to IAM, a gene for the conversion of IAM to IAA, a gene for the conversion of IAA to skatole, a gene for the conversion of skatole to IAID.

In one embodiment, the bacterium, or the plasmid (e.g. the conjugative plasmid), comprises one or more heterologous gene(s) for the biosynthesis of IAID from tryptophan.

In one embodiment, the one or more heterologous gene(s) for the biosynthesis of IAID comprise (or consists of) a gene for the conversion of tryptophan to tryptamine, a gene for the conversion of tryptamine to IAAld, a gene for the conversion of IAAld to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of tryptophan to tryptamine, a gene for the conversion of tryptamine to IAAld, a gene for the conversion of IAAld to IAA, a gene for the conversion of IAA to skatole, a gene for the conversion of skatole to IAID.

In one embodiment, the one or more heterologous gene(s) for the biosynthesis of IAID comprise (or consists of) a gene for the conversion of tryptophan to IPyA, a gene for the conversion of IPyA to IAAld, a gene for the conversion of IAAld to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of tryptophan to IPyA, a gene for the conversion of IPyA to IAAld, a gene for the conversion of IAAld to IAA, a gene for the conversion of IAA to skatole, a gene for the conversion of skatole to IAID.

In one embodiment, the one or more heterologous gene(s) for the biosynthesis of IAID comprise (or consists of) a gene for the conversion of tryptophan to IPyA, a gene for the conversion of IPyA to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of tryptophan to IPyA, a gene for the conversion of IPyA to IAA, a gene for the conversion of IAA to skatole, a gene for the conversion of skatole to IAID.

In one embodiment, the one or more heterologous gene(s) for the biosynthesis of IAID comprise (or consists of) a gene for the conversion of tryptophan to IAM, a gene for the conversion of IAM to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of tryptophan to IAM, a gene for the conversion of IAM to IAA, a gene for the conversion of IAA to skatole, a gene for the conversion of skatole to IAID.

In one embodiment, the one or more heterologous gene(s) for the biosynthesis of IAID comprise (or consists of) a gene for the conversion of tryptophan to IAOx, a gene for the conversion of IAOx to IAN, a gene for the conversion of IAN to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of tryptophan to IAOx, a gene for the conversion of IAOx to IAN, a gene for the conversion of IAN to IAA, a gene for the conversion of IAA to skatole, a gene for the conversion of skatole to IAID.

In one embodiment, the one or more heterologous gene(s) for the biosynthesis of IAID comprise (or consists of) a gene for the conversion of tryptophan to IAOx, a gene for the conversion of IAOx to IAN, a gene for the conversion of IAN to IAM, a gene for the conversion of IAM to IAA, a gene for the conversion of IAA to skatole, a gene for the conversion of skatole to IM, a gene for the conversion of IM to IAID.

In one embodiment, the one or more heterologous gene(s) for the biosynthesis of IAID comprise (or consists of) a gene for the conversion of tryptophan to IAOx, a gene for the conversion of IAOx to IAN, a gene for the conversion of IAN to IAM, a gene for the conversion of IAM to IAA, a gene for the conversion of IAA to skatole, a gene for the conversion of skatole to IAID.

In one embodiment, the bacterium, or the plasmid (e.g. the conjugative plasmid), comprises one or more heterologous gene(s) for the biosynthesis of IAID from skatole. In one embodiment, the one or more heterologous gene(s) for the biosynthesis of IAID from skatole comprise (or consists of) the genes described in the pathways above.

In one embodiment, the bacterium, or the plasmid (e.g. the conjugative plasmid), comprises one or more heterologous gene(s) for the biosynthesis of IAID from IAA. In one embodiment, the one or more heterologous gene(s) for the biosynthesis of IAID from IAA comprise (or consists of) the genes described in the pathways above.

In one embodiment, the bacterium, or the plasmid (e.g. the conjugative plasmid), comprises one or more heterologous gene(s) for the biosynthesis of IAID from IAAld. In one embodiment, the one or more heterologous gene(s) for the biosynthesis of IAID from IAAld comprise (or consists of) the genes described in the pathways above.

In one embodiment, the bacterium, or the plasmid (e.g. the conjugative plasmid), comprises one or more heterologous gene(s) for the biosynthesis of IAID from IPyA. In one embodiment, the one or more heterologous gene(s) for the biosynthesis of IAID from IPyA comprise (or consists of) the genes described in the pathways above.

In one embodiment, the bacterium, or the plasmid (e.g. the conjugative plasmid), comprises one or more heterologous gene(s) for the biosynthesis of IAID from IAM. In one embodiment, the one or more heterologous gene(s) for the biosynthesis of IAID from IAM comprise (or consists of) the genes described in the pathways above.

In one embodiment, the bacterium, or the plasmid (e.g. the conjugative plasmid), comprises one or more heterologous gene(s) for the biosynthesis of IAID from IAOx. In one embodiment, the one or more heterologous gene(s) for the biosynthesis of IAID from IAOx comprise (or consists of) the genes described in the pathways above.

In one embodiment, the bacterium, or the plasmid (e.g. the conjugative plasmid), comprises one or more heterologous gene(s) for the biosynthesis of IAID from IAN. In one embodiment, the one or more heterologous gene(s) for the biosynthesis of IAID from IAN comprise (or consists of) the genes described in the pathways above.

In one embodiment, the bacterium, or the plasmid (e.g. the conjugative plasmid), comprises one or more heterologous gene(s) for the biosynthesis of IAID from IM. In one embodiment, the one or more heterologous gene(s) for the biosynthesis of IAID from IM comprise (or consists of) the genes described in the pathways above.

In another embodiment, the bacterium, or the plasmid (e.g. the conjugative plasmid), may comprise genes from more than one of the pathways described hereinabove.

### Exporters

In plants, IAA is an amphipathic weak acid that diffuses through cellular membranes only when protonated (IAAH) and is membrane-impermeant at neutral cytosolic pH. In bacteria which have been engineered to produce IAA, previous attempts have relied on simple diffusion of the IAA across the bacterial membrane(s), which has resulted in sub-optimal levels of IAA being secreted into the local environment.

The inventors have advantageously realised that increased levels of secretion of ALMs can be achieved by modifying bacteria to include various heterologous exporters. As is known in the art, diffusion of molecules through bacterial membrane(s) (both gram-negative and gram-positive bacteria) can limit the amount of a particular molecule which diffuses to the local environment. Without being bound by theory, in general, bacterial membrane(s) are hydrophobic, so the more hydrophilic the small molecule is, the less likely it is to be able to cross the membrane. However, certain molecules which are detrimental to the cell (for example toxins, bacteriocins, etc) are exported more often, along with certain molecules which provide specific extra-cellular functions (such as molecules associated with quorum sensing, iron acquisition, etc). In addition, some molecules are actively transported through the membrane(s) by dedicated exporters (for example, excess amino acids may be exported from the cell to maintain homeostasis, such as the alaE exporter of alanine, and the leuE exporter of leucine both found in E. coli), or exporters which export a certain class of molecule (for example, the setA transporter found in E. coli exports various sugar molecules). However, the majority of ALM transporters to date have been found in plant species. Thus, in many bacteria, secretion of ALMs are limited by the rate of diffusion. The inventors have engineered bacteria to express heterologous exporters which are capable of exporting the ALMs across the membrane(s) of the bacteria. Unexpectedly, heterologous exporters are able to form and function within the bacterial membrane. This leads to an increased secretion of the desired ALMs into the local environment, and contributes to the reduced fitness disadvantage of the expression of ALMs within the bacteria, because (without being bound by theory) the ALMs are removed from the cytoplasm and/or periplasm into the local environment.

In one embodiment, the heterologous gene encoding an exporter which is capable of exporting said one or more ALMs is from a plant species. In one embodiment, the plant species is an Arabidopsis species, e.g. is Arabidopsis thaliana. In one embodiment, the exporter is from a bacterial species. In one embodiment, the bacterial species is a Pantoea species, e.g. is Pantoea agglomerans. In one embodiment, the exporter is not capable of exporting proteins out of a bacterial cell.

In one embodiment, the exporter is not an ABC family protein transporter.

Auxin efflux proteins are a large class of molecules which are predominantly expressed in plants. These molecules facilitate the export of naturally occurring auxins (primarily IAA) from the plant cell, rather than other transporter systems which are used by cells to transport large molecules such as peptides and proteins. In plants, auxin efflux proteins are broadly divided into two classes: PIN (Pin-formed) family protein transporters and ABC (ATP-binding cassette transporters) family protein transporters. The latter class comprises many sub-families, as detailed further below. Despite the differences between the structure of plant cells and bacterial cells, the inventors surprisingly show in the Examples hereinbelow that a PIN family protein transporter from Arabidopsis mediates auxin efflux from bacterial cells without needing additional plant-specific factors.

In one embodiment, the heterologous gene encoding an exporter which is capable of exporting said one or more ALMs encodes an auxin efflux protein. In one embodiment, the auxin efflux protein is from a plant species. In one embodiment, the plant species is an Arabidopsis species, e.g. is Arabidopsis thaliana. In one embodiment, the auxin efflux protein is from a bacterial species. In one embodiment, the bacterial species is a Pantoea species, e.g. is Pantoea agglomerans. In one embodiment, the exporter is a PIN family protein transporter which is capable of exporting said one or more ALMs out of a bacterial cell. In one embodiment, the exporter is an ABC family protein transporter which is capable of exporting said one or more ALMs out of a bacterial cell.

Methods for determining whether any given exporter is capable of exporting any given ALM out of a cell are known to those skilled in the art. For instance, as shown in the examples below, a bacterium producing an ALM can be produced with and without the exporter of interest, and the amount of ALM in the supernatant is measured. Where the exporter is capable of exporting the ALM out of the bacterium, the amount of ALM seen in the supernatant is increased. A test that may be used is the Salkowski colorimetric assay, as described below in Example 4.3.

A specific protein in the bacterium Pantoea agglomerans C1 was recently discovered (see Luziatelli, et al., Microorganisms, 8 (2): 153, 2020, which is incorporated herein by reference in its entirety) and identified as an Auxin Efflux Carrier (AEC) family protein transporter (See Table 4, Contig1: 1779607-1780566, on page 9 of Luziatelli et al, supra) with gene name aec.

Based on predicted functionality from sequence information (e.g. based on the sequence described in Luziatelli et al, supra), GenBank predicts that there are over 1500 “auxin efflux carrier” family protein transporters present in bacteria, many of which remain to be studied and functionality confirmed. Functionality of a predicted Auxin Efflux Carrier (AEC) family protein transporter can be determined using the methods described elsewhere herein. Without being bound by theory, Auxin Efflux Carrier (AEC) family protein transporters of bacterial origin may enable more efficient export of ALMs from bacteria than Auxin Efflux Carrier (AEC) family protein transporters of other origins.

Thus, in one embodiment, the exporter is a Auxin Efflux Carrier (AEC) family protein transporter. In one embodiment, the exporter is a Auxin Efflux Carrier (AEC) family protein transporter from a bacterial species. In one embodiment, the exporter is a Auxin Efflux Carrier (AEC) family protein transporter from a Pantoea species, e.g. is from Pantoea agglomerans. In one embodiment, the exporter is aec from a Pantoea species, e.g. from Pantoea agglomerans. In one embodiment, the exporter comprises the nucleotide sequence of SEQ ID No:2.

### PIN Family Protein Transporters:

The bias of auxin transport is attributed to highly regulated, polar-localized efflux complexes characterized by the PIN-FORMED (PIN) family protein transporters. PINs have been shown to align with the auxin transport vector and to be necessary for normal polarized organ development in plants and auxin movement.

The PIN auxin efflux carriers are distantly related to some fungal transporters with 9-11 transmembrane helices, but are thought to have differentiated into a discrete group early in vascular plant evolution. The PIN nomenclature is derived from the PIN-FORMED inflorescence phenotype associated with loss of PIN1, which is the primary mediator of polar auxin flow functioning in angiosperm development. The Arabidopsis genome contains eight PIN genes, five of which encode full-length PINs (PIN1, 2, 3, 4, and 7) and three of them encode short PINs (PIN5, 6, and 8). Short PIN proteins lack the long central hydrophilic loop found in full-length PINs and are localized to endomembrane structures where they are thought to function in homeostatic auxin compartmentalization, although the motive force maintaining the endomembrane auxin gradient has yet to be defined. Auxin efflux directly mediated by PIN1, 2, 4, 5, and 7 has been demonstrated in multiple heterologous systems (Geisler et al., The Plant Journal, 44, 179-194, 2005; Petrášek et al., Science, 312, 914-918, 2006; Blakeslee et al., Plant Cell., 19 (1): 131-147, 2007 each of which is incorporated herein by reference).

Thus, in one embodiment, the exporter is a PIN family protein transporter. In one embodiment, the exporter is a PIN family protein transporter which is capable of exporting the ALM out of a bacterial cell. In one embodiment, the exporter is a PIN family protein transporter from a plant species. In one embodiment, the exporter is a PIN1 protein transporter, such as a PIN1 protein transporter from a plant species, for example from a species of Arabidopsis, e.g. from Arabidopsis thaliana. In one embodiment, the exporter is a PIN2 protein transporter, such as a PIN2 protein transporter from a plant species, for example from a species of Arabidopsis, e.g. from Arabidopsis thaliana. In one embodiment, the exporter is a PIN3 protein transporter, such as a PIN3 protein transporter from a plant species, for example from a species of Arabidopsis, e.g. from Arabidopsis thaliana. In one embodiment, the exporter is a PIN4 protein transporter, such as a PIN4 protein transporter from a plant species, for example from a species of Arabidopsis, e.g. from Arabidopsis thaliana. In one embodiment, the exporter is a PIN5 protein transporter, such as a PIN5 protein transporter from a plant species, for example from a species of Arabidopsis, e.g. from Arabidopsis thaliana. In one embodiment, the exporter is a PIN6 protein transporter, such as a PIN6 protein transporter from a plant species, for example from a species of Arabidopsis, e.g. from Arabidopsis thaliana. In one embodiment, the exporter is a PIN7 protein transporter, such as a PIN7 protein transporter from a plant species, for example from a species of Arabidopsis, e.g. from Arabidopsis thaliana. In one embodiment, the exporter is a PIN2 or a PIN7 protein transporter, such as a PIN2 or PIN7 protein transporter from a plant species, for example from a species of Arabidopsis, e.g. from Arabidopsis thaliana. In one embodiment, the heterologous gene encoding the exporter comprises the nucleotide sequence of Seq ID No:3. In one embodiment, the exporter is any of the PINs described in Table 1 hereinbelow.

### ABC Proteins:

The ATP-binding cassette (ABC) transporters use ATP to actively pump substrates across membranes in or out of cells, against their electrochemical gradients. Thus, ABC exporters (as opposed to importers) can be used to transport small molecule ALMs out of bacterial cells. ABC proteins possess an ATP binding cassette, also known as the nucleotide-binding domain (NBD). The NBD contains several highly conserved motifs, including the Walker A and Walker B sequences, the ABC signature motif, the H loop and the Q loop. ABC transporters also contain trans-membrane domains (TMDs), each of which comprises several hydrophobic α-helices. The ABC transporter core unit consists of four domains, two NBDs and two TMDs. The two NBDs together bind and hydrolyse ATP (thereby providing the driving force for transport), while the TMDs participate in substrate recognition and translocation across the lipid membrane.

The ABC family protein transporters comprise many sub-families, which are usually classified into seven sub-families ABCA, ABCB, ABCC, ABCD, ABCE, ABCF and ABCG, each of which comprise several sub-designations, such as MDR (multi-drug resistant) or PGP (P-glycoproteins).

For a review of ABC family protein transporters see Rees et al., Nature Reviews Molecular Cell Biology, 10, 218-227, 2009, doi: 10.1038/nrm2646 and Vasiliou et al., Hum Genomics, 3 (3), 281-290, 2009, doi: 10.1186/1479-7364-3-3-281, each of which are incorporated herein by reference in their entirety.

A subclass of particular interest in the present disclosure is the ABCB subfamily of ABC family protein transporters, in particular the ABCB family from plant origins. Plant ABCB exporters have been shown to export IAA (see Geisler et al., FEBS Letters, 580 (4), 1094-102, 2006, doi: 10.1016/j.febslet.2005.11.054, incorporated herein by reference in its entirety). The auxin transport activity of Arabidopsis, maize, and sorghum ABCB1 and Arabidopsis ABCB4 and ABCB19 have been demonstrated in plants and in heterologous systems. In plants, ABCB1 and ABCB19 function primarily in the maintenance of long-distance auxin transport streams and movement of auxin out of apical tissues. In Arabidopsis, ABCB19 functions as a rate-limiting negative regulator of auxin-dependent tropic bending responses. ABCB4 appears to be an inducible auxin efflux transporter, as it mediates auxin import at very low IAA concentrations, but rapidly reverts to a much stronger export activity with increased IAA concentrations. Therefore, ABCBs function in long-distance auxin transport, loading of auxin into these streams, apical dominance, root elongation, and phototropism, but the roles in gravitropism and the root meristem are unclear.

In one embodiment, the exporter is an ABC family protein transporter. In one embodiment, the exporter is an ABC family protein transporter which is capable of exporting an ALM out of a bacterial cell. In one embodiment, the exporter is an ABC family protein transporter which does not export proteins out of a bacterial cell. In one embodiment, the exporter is an ABC family protein transporter which is capable of exporting an ALM out of a bacterial cell, but does not export proteins out of a bacterial cell. In one embodiment, the ACB family protein transporter is from a plant species. In one embodiment, the ACB family protein transporter is from an Arabidopsis species, e.g. from Arabidopsis thaliana.

Thus, in one embodiment, the exporter is an ABCA subfamily protein transporter. In one embodiment, the exporter is an ABCA subfamily protein transporter which is capable of exporting an ALM out of a bacterial cell. In one embodiment, the exporter is an ABCA subfamily protein transporter from a plant species. In one embodiment, the exporter is an ABCA subfamily protein transporter from an Arabidopsis species, e.g. from Arabidopsis thaliana. In one embodiment, the exporter is an ABCA-PGP subfamily protein transporter which is capable of exporting an ALM out of a bacterial cell. In one embodiment, the exporter is an ABCA-PGP subfamily protein transporter from a plant species. In one embodiment, the exporter is an ABCA-PGP subfamily protein transporter from an Arabidopsis species, e.g. from Arabidopsis thaliana.

Thus, in one embodiment, the exporter is an ABCB subfamily protein transporter. In one embodiment, the exporter is an ABCB subfamily protein transporter which is capable of exporting an ALM out of a bacterial cell. In one embodiment, the exporter is an ABCB subfamily protein transporter from a plant species. In one embodiment, the exporter is an ABCB subfamily protein transporter from an Arabidopsis species, e.g. from Arabidopsis thaliana. In one embodiment, the exporter is an ABCB-PGP subfamily protein transporter which is capable of exporting an ALM out of a bacterial cell. In one embodiment, the exporter is an ABCB-PGP subfamily protein transporter from a plant species. In one embodiment, the exporter is an ABCB-PGP subfamily protein transporter from an Arabidopsis species, e.g. from Arabidopsis thaliana.

Thus, in one embodiment, the exporter is an ABCD subfamily protein transporter. In one embodiment, the exporter is an ABCD subfamily protein transporter which is capable of exporting an ALM out of a bacterial cell. In one embodiment, the exporter is an ABCD subfamily protein transporter from a plant species. In one embodiment, the exporter is an ABCD subfamily protein transporter from an Arabidopsis species, e.g. from Arabidopsis thaliana. In one embodiment, the exporter is an ABCD-PGP subfamily protein transporter which is capable of exporting an ALM out of a bacterial cell. In one embodiment, the exporter is an ABCD-PGP subfamily protein transporter from a plant species. In one embodiment, the exporter is an ABCD-PGP subfamily protein transporter from an Arabidopsis species, e.g. from Arabidopsis thaliana.

Thus, in one embodiment, the exporter is an ABCG subfamily protein transporter. In one embodiment, the exporter is an ABCG subfamily protein transporter which is capable of exporting an ALM out of a bacterial cell. In one embodiment, the exporter is an ABCG subfamily protein transporter from a plant species. In one embodiment, the exporter is an ABCG subfamily protein transporter from an Arabidopsis species, e.g. from Arabidopsis thaliana. In one embodiment, the exporter is an ABCG-PGP subfamily protein transporter which is capable of exporting an ALM out of a bacterial cell. In one embodiment, the exporter is an ABCG-PGP subfamily protein transporter from a plant species. In one embodiment, the exporter is an ABCG-PGP subfamily protein transporter from an Arabidopsis species, e.g. from Arabidopsis thaliana.

In one embodiment, the exporter is any of the ABC family protein transporter described in Table 1 hereinbelow.

### Exporters of IBA

As well as the general exporters of ALMs described above, the inventors have identified several exporters which may be suitable for exporting IBA out of a bacterial cell. A further class of auxin efflux proteins known as NTR1 PTR (NPF) family, and specifically the Transporter of IBA1 (TOB1) efflux protein identified in Arabidopsis was shown to export IBA (see Damodaran et al, Front. Plant Sci, 2019, 10:851. doi: 10.3389/fpls. 2019.00851, which is incorporated herein in its entirety). Thus, in one embodiment, the heterologous gene encoding an exporter which is capable of exporting IBA encodes a protein which is TOB1. In one embodiment, the TOB1 is from a plant species. In another embodiment, the TOB1 is from Arabidopsis, e.g. from Arabidopsis thaliana.

In one embodiment, the heterologous gene encoding an exporter which is capable of exporting IBA encodes a protein selected from PXA1/ABCD1, ABCG36, ABCG37, and ABCG36/PDR8/PEN3. In one embodiment, the protein is from a plant species. In another embodiment, the protein is from Arabidopsis, e.g. from Arabidopsis thaliana.

TABLE 1
Bacterial and Plant Auxin Efflux Proteins

| | Exporter | Example | Substrate | |
|---|---|---|---|---|
| Exporter Family | name | species | exported | Reference |
| Auxin efflux carrier | aec | Pantoea | IAA | Luziatelli, et al., |
| (AEC) protein | | agglomerans | | supra |
| PIN-FORMED (PIN) | PIN1 | Arabidopsis | IAA | Petrášek et al., |
| | | | 1- | supra, |
| | | | naphthalene | Blakeslee et al., |
| | | | acetic acid | supra |
| | | | (NAA) | |
| | PIN2 | Arabidopsis | IAA | Geisler et al., |
| | | | | supra |
| | PIN3 | Arabidopsis | IAA | |
| | PIN4 | Arabidopsis | IAA | Petrášek et al., |
| | | | | supra |
| | PIN5 | Arabidopsis | IAA | |
| | PIN6 | Arabidopsis | IAA | Petrášek et al., |
| | | | | supra |
| | PIN7 | Arabidopsis | IAA | Petrášek et al., |
| | | | | supra, |
| | | | | Blakeslee et al., |
| | | | | supra |
| | PIN8 | Arabidopsis | IAA | |
| ABCB-PGP | ABCB1 | Arabidopsis | IAA | |
| | | Maize | | |
| | | Sorghum | | |
| | | Triticum | | |
| | | aestivum | | |
| | ABCB4 | Arabidopsis | IAA | Terasaka et al., |
| | | | | Plant Cell, |
| | | | | 17: 2922-2939, |
| | | | | 2005 |
| | ABCB19 | Arabidopsis | IAA | |
| | ABCB21 | Arabidopsis | IAA | Kamimoto et |
| | | | NAA | al., Plant Cell |
| | | | | Physiology, |
| | | | | 53(12): 2090- |
| | | | | 2100, 2012 |
| ABCB-MDR/PGP | AtPGP1 | Arabidopsis | IAA | Geisler et al., |
| | | | NAA | supra |
| | PGP4 | | | Blakeslee et al. |
| | | | | supra |
| ABCG-pleiotropic drug | ABCG36 | Arabidopsis | IBA | Damodaran et |
| resistance (PDR) | | | | al, supra |
| | ABCG37 | Arabidopsis | IBA | Damodaran et |
| | | | | al, supra |
| | ABCG29 | Arabidopsis | IBA | Damodaran et |
| | | | | al, supra |
| | ABCG33 | Arabidopsis | IBA | Damodaran et |
| | | | | al, supra |
| NTR1 PTR FAMILY | TRANSPORTER | Arabidopsis | IBA | Damodaran et |
| (NPF) | OF IBA1 | | | al, supra |
| | (TOB1) | | | |

### Promoters

Previous attempts to include heterologous genes for the biosynthesis of ALMs on plasmids has required the control of inducible and/or weak promoters. This has been necessitated because, when constitutive promoters are used to control heterologous genes expression of ALMs on a plasmid, it results in plasmid instability and high levels of genetic mutation within the heterologous genes. These mutations and instability ultimately result in gene inactivation and can even result in bacterial cell death. The present inventors unexpectedly realised that the provision of the heterologous genes directly into the chromosome of the bacterium leads to stable expression of the heterologous genes, without the associated plasmid instability and detrimental genetic mutations.

Thus, in one embodiment, the one or more heterologous genes for the biosynthesis of said one or more ALMs (e.g. any of the ALMs described herein, such as IAA) are each or all under the control of one or more constitutive promoter(s) (e.g. any of the constitutive promoters described herein).

In one embodiment, the one or more heterologous genes for the biosynthesis of said one or more ALMs (e.g. any of the ALMs described herein, such as IAA) are comprised within an operon under the control of a single constitutive promoter (e.g. any of the constitutive promoters described herein).

In one embodiment, the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), is under the control of a constitutive promoter (e.g. any of the constitutive promoters described elsewhere herein).

In one embodiment, the one or more heterologous genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), are each or all under the control of one or more constitutive promoter(s) (e.g. any of the constitutive promoters described elsewhere herein).

In one embodiment, the one or more heterologous genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), are all under the control of a single constitutive promoter (e.g. any of the constitutive promoters described elsewhere herein).

In one embodiment, the one or more heterologous genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), are comprised within an operon under the control of a single constitutive promoter (e.g. any of the constitutive promoters described elsewhere herein).

In one embodiment, the promoter is a constitutive promoter which results in stable production of the one or more ALMs for at least 5 days (for example at least 6, at least 7 or at least 8 days) in vitro or in vivo. In one embodiment, the stability is in vivo. Methods for determining in vivo stability are described in Example 2.4 hereinbelow.

In one embodiment, the promoter is a promoter which is based on the sequence of a tac promoter. Tac promoters are based on a combination of promoters from the trp and lac operons, see de Boer, et al., PNAS, 80 (1), 21-25, 1983. doi: 10.1073/pnas.80.1.21, which is incorporated herein in its entirety. Several tac-based promoters have been reported in the art, see e.g. Zhang et al., Microb. Cell Fact, 16:84, 2017, doi: 10.1186/s12934-017-0700-2, which is incorporated herein in its entirety. In one embodiment the promoter is a Pc-tga promoter. In one embodiment, the promoter has a nucleotide sequence of Seq ID No: 1.

In another embodiment, the promoter is a constitutive promoter which results in no mutations in the one or more genes for the biosynthesis of said one or more ALMs (e.g. any of the ALMs described herein, such as IAA) and the gene encoding an exporter which is capable of exporting said one or more ALMs after at least 5 days (for example at least 6, at least 7 or at least 8 days) in vitro or in vivo. In one embodiment, the stability is in vivo. Methods for determining in vivo stability are described in Example 2.4 hereinbelow.

Constitutive promoters and variants are well known in the art and include, but are not limited to, BBa_J23100, a constitutive Escherichia coli cs promoter (e.g., an osmY promoter (International Genetically Engineered Machine (iGEM) Registry of Standard Biological Parts Name BBa_J45992; BBa_J45993)), a constitutive Escherichia coli 02 promoter (e.g. htpG heat shock promoter (BBa_J45504)), a constitutive Escherichia coli 070 promoter (e.g. lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001), M13K07 gene I promoter (BBa_M13101), M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g. promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g. promoter ctc (BBa_K143010) or promoter gsiB (BBa_K143011)), a Salmonella promoter (e.g. Pspv2 from Salmonella (BBa_K112706), Pspv from Salmonella (BBa_K112707)), a bacteriophage 17 promoter (e.g. T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g. SP6 promoter (BBa_J64998)).

One way for measuring the strength of activity is by measuring the Anderson score of any given promoter. The activity of the reporters is measured by the relative fluorescence of the promoter when used in the control plasmid EX-Ptet-S-rbsRFP-P “RFP reporter” (see http://parts.igem.org/Part:BBa_J61002) in strain TG1 grown in LB media to saturation. BBa_J23119 is the “consensus” promoter sequence and the strongest member of the family. The NheI and AvrII restriction sites present within these promoter parts make them a scaffold for further modification. For more information, see http://parts.igem.org/Part:BBa_J23114.

Thus, in one embodiment, the constitutive promoter is a strong constitutive promoter (for example a promoter having an Anderson Score (AS) of AS ≥0.4, such as ≥0.5). In another embodiment, the promoter has an Anderson score of between 0.1 and 0.4 or between 0.1 and 0.5.

TABLE 2
Anderson Promoter Collection

| SEQ ID | | | Measured |
|---|---|---|---|
| NO: | Identifier | Sequenceª | Strength^{b} |
| Seq ID | BBa_J23119 | ttgacagctagctcagtcctaggtataatgctagc | n/a |
| No: 4 | | | |
| Seq ID | BBa_J23100 | ttgacggctagctcagtcctaggtacagtgctagc | 1 |
| No: 5 | | | |
| Seq ID | BBa_J23101 | tttacagctagctcagtcctaggtattatgctagc | 0.7 |
| No: 6 | | | |
| Seq ID | BBa_J23102 | ttgacagctagctcagtcctaggtactgtgctagc | 0.86 |
| No: 7 | | | |
| Seq ID | BBa_J23103 | ctgatagctagctcagtcctagggattatgctagc | 0.01 |
| No: 8 | | | |
| Seq ID | BBa_J23104 | ttgacagctagctcagtcctaggtattgtgctagc | 0.72 |
| No: 9 | | | |
| Seq ID | BBa_J23105 | tttacggctagctcagtcctaggtactatgctagc | 0.24 |
| No: 10 | | | |
| Seq ID | BBa_J23106 | tttacggctagctcagtcctaggtatagtgctagc | 0.47 |
| No: 11 | | | |
| Seq ID | BBa_J23107 | tttacggctagctcagccctaggtattatgctagc | 0.36 |
| No: 12 | | | |
| Seq ID | BBa_J23108 | ctgacagctagctcagtcctaggtataatgctagc | 0.51 |
| No: 13 | | | |
| Seq ID | BBa_J23109 | tttacagctagctcagtcctagggactgtgctagc | 0.04 |
| No: 14 | | | |
| Seq ID | BBa_J23110 | tttacggctagctcagtcctaggtacaatgctagc | 0.33 |
| No: 15 | | | |
| Seq ID | BBa_J23111 | ttgacggctagctcagtcctaggtatagtgctagc | 0.58 |
| No: 16 | | | |
| Seq ID | BBa_J23112 | ctgatagctagctcagtcctagggattatgctagc | 0 |
| No: 17 | | | |
| Seq ID | BBa_J23113 | ctgatggctagctcagtcctagggattatgctagc | 0.01 |
| No: 18 | | | |
| Seq ID | BBa_J23114 | tttatggctagctcagtcctaggtacaatgctagc | 0.1 |
| No: 19 | | | |
| Seq ID | BBa_J23115 | tttatagctagctcagcccttggtacaatgctagc | 0.15 |
| No: 20 | | | |
| Seq ID | BBa_J23116 | ttgacagctagctcagtcctagggactatgctagc | 0.16 |
| No: 21 | | | |
| Seq ID | BBa_J23117 | ttgacagctagctcagtcctagggattgtgctagc | 0.06 |
| No: 22 | | | |
| Seq ID | BBa_J23118 | ttgacggctagctcagtcctaggtattgtgctagc | 0.56 |
| No: 23 | | | |

^{a}also shown in the Anderson Catalog, see http://parts.igem.org/Promoters/Catalog/Anderson
^{b}Strength is the Anderson Score (AS), e.g. a strength of 1 is a AS of 1. Reported activities of the promoters are given as the relative fluorescence of plasmids in strain TG1 grown in LB media to saturation. A suitable plasmid is EX-Ptet-S-rbsRFP-P ″RFP reporter″ as described at http://parts.igem.org/Part:BBa_J61002; insertion of a promoter element between XbaI and SpeI sites results in a RFP reporter.

In some embodiments, it may be desirable to include a promoter which is inducible to produce the one or more ALMs under only certain conditions. For example, the one or more genes for the biosynthesis of said one or more ALMs (e.g. any of the ALMs described herein, such as IAA) could be under the control of a promoter which is active only under certain environmental conditions. For example, the inducible promoter is active under environmental conditions which are specific to the gut of a subject. In one embodiment, the inducible promoter is active under environmental conditions which are specific to the upper gastrointestinal tract of a subject (e.g. bile acids). In one embodiment, the inducible promoter is active under environmental conditions which are specific to the lower gastrointestinal tract of a subject (e.g. anaerobic conditions). In one embodiment, the inducible promoter is active under the low oxygen or anaerobic conditions which are specific to gut (e.g. the upper gastrointestinal tract and/or the lower gastrointestinal tract) of a subject.

In another embodiment, the inducible promoter is a temperature sensitive promoter, such as one which is active under physiological temperatures (e.g. approximately 35 to 39° C., for example approximately 36 to 38° C., such as approximately 37° C.). For a discussion on this type of kill switch, see https://wyss.harvard.edu/news/kill-switches-for-engineered-microbes-gone-rogue/and the “cryodeath” system which is described in more detail in Stirling et al., Mol. Cell, 68, 686-697.e683, 2017, which is incorporated herein in its entirety.

In another embodiment, the promoter is active only in the presence of certain molecules present in the local physiological environment (such as molecules present only in the gut). These inducible promoters may therefore turn on and off production and secretion of the one or more ALMs when in the desired location (such as the gut). Such inducible promoters are described herein.

Other promoters of interest are ones which have been designed to be active when the bacterial cell is in a certain state, for example when it is a “stress-phase active”. Such stress-phase active promoters (SPAs) are described in GB2303409.3, which is incorporated herein in its entirety and for its disclosure relating to SPAs, in particular for any of promoters of SEQ ID Nos: 1 to 10 disclosed therein (disclosed herein as Seq ID Nos: 65 to 74) or any promoters in claims 77 to 86 therein.

Thus, in one embodiment the promoter is a promoter selected from a RelB, BolA, Hya, YiaG and a RpoH promoter. The promoter may be a promoter selected from a RelB promoter sequence, σ70; a BolA promoter sequence, σS, σ70; a Hya promoter sequence, σS, σ70; a YiaG promoter sequence, σS; a RpoH promoter sequence P1, σ70; a RpoH promoter sequence P2, σS; a RpoH promoter sequence P3, σ24; a RpoH promoter sequence P4, σ70; a RpoH promoter sequence P5, σ70; and a RpoH promoter sequence P6, σ54. The promoter may be a promoter having a nucleotide sequence selected from any one of Seq ID Nos: 65 to 74, or a nucleotides sequence having 90% (or 95%) homology thereto.

In one embodiment, the one or more genes for the biosynthesis of said one or more ALMs (e.g. any of the ALMs described herein, such as IAA) are each or all under the control of one or more inducible promoter(s) (e.g. any of the inducible promoters described herein).

In one embodiment, the one or more genes for the biosynthesis of said one or more ALMs (e.g. any of the ALMs described herein, such as IAA) are comprised within an operon under the control of a single inducible promoter (e.g. any of the inducible promoters described herein).

In one embodiment, the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), is under the control of an inducible promoter (e.g. any of the inducible promoters described elsewhere herein).

In one embodiment, the one or more genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), are each or all under the control of one or more constitutive promoter(s) (e.g. any of the constitutive promoters described elsewhere herein).

In one embodiment, the one or more genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), are all under the control of a single inducible promoter (e.g. any of the inducible promoters described elsewhere herein).

In one embodiment, the one or more genes for the biosynthesis of said one or more ALMs (e.g. any of the ALMs described herein, such as IAA) and the gene encoding an exporter which is capable of exporting said one or more ALMs (e.g. any of the ALMs described herein, such as IAA) out of the bacterium, or out of a bacterium which comprises said plasmid and produces and secretes said one or more ALMs (i.e. out of a recipient bacterium when the plasmid is a conjugative plasmid), are comprised within an operon under the control of a single inducible promoter (e.g. any of the inducible promoters described elsewhere herein).

### Bacteria

In any embodiment herein, the modified bacterium, the bacterial host (donor) cell comprising a conjugative plasmid, or the bacterial cell which comprises said plasmid (i.e. the recipient bacterium when the plasmid is a conjugative plasmid) may be a gram-negative bacterium.

In any embodiment herein, the modified bacterium, the bacterial host (donor) cell comprising a conjugative plasmid, or the bacterial cell which comprises said plasmid (i.e. the recipient bacterium when the plasmid is a conjugative plasmid) may be a gram-positive bacterium.

In any embodiment herein, the modified bacterium, the bacterial host (donor) cell comprising a conjugative plasmid or the bacterial cell which comprises said plasmid (i.e. recipient bacterium when the plasmid is a conjugative plasmid) is a strain selected from any of the strains in Table 3.

In any embodiment herein, the modified bacterium, the bacterial host (donor) cell comprising a conjugative plasmid or the bacterial cell which comprises said plasmid (i.e. the recipient bacterium when the plasmid is a conjugative plasmid) may be an E. coli strain. In one embodiment, the E. coli strain is an E. coli strain from phylogroup A. In one embodiment, the E. coli strain is an E. coli strain from phylogroup B1. In one embodiment, the E. coli strain is an E. coli strain from phylogroup E. In one embodiment, the E. coli strain is an E. coli strain which is present in a probiotic product. The probiotic product may be colinfant New Born (e.g. strain A0 34/86). The probiotic product may be symbioflor2 (e.g. strain G1/2, G4/9, G5, G6/7, and G8). The probiotic product may be Mutaflor (e.g. E. coli Nissle).

In any embodiment herein, the modified bacterium, the bacterial host (donor) cell comprising a conjugative plasmid or the bacterial cell which comprises said plasmid (i.e. the recipient bacterium when the plasmid is a conjugative plasmid) may be a strain belonging to a genera selected from Bifidobacterium, Bacteroides, Lactobacillus, Lacticaseibacillus, Lactiplantibacillus, Levilactobacillus, Ligilactobacillus, Limosilactobacillus and Lactococcus.

In any embodiment herein, the modified bacterium, the bacterial host (donor) cell comprising a conjugative plasmid or the bacterial cell which comprises said plasmid (i.e. the recipient bacterium when the plasmid is a conjugative plasmid) may be a strain belonging to a genera selected from a Bifidobacterium genus or a Bacteroides genus. In one embodiment, the genera is a Bacteroides genus. In one embodiment, the genera is a Bifidobacterium genus.

In any embodiment herein, the modified bacterium, the bacterial host (donor) cell comprising a conjugative plasmid or the bacterial cell which comprises said plasmid (i.e. the recipient bacterium when the plasmid is a conjugative plasmid) may be a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacillus brevis, Ligilactobacillus salivarius, Limosilactobacillus reuteri and Lactococcus lactis.

In any embodiment herein, the modified bacterium, the bacterial host (donor) cell comprising a conjugative plasmid or the bacterial cell which comprises said plasmid (i.e. the recipient bacterium when the plasmid is a conjugative plasmid) may be a species which is selected from Bifidobacterium longum, Bifidobacterium breve, Bifidobacterium adolescentis, Bacteroides uniformis, Bacteroides vulgatus and Bacteroides thetaiotaomicron.

In any embodiment herein, the modified bacterium, the bacterial host (donor) cell comprising a conjugative plasmid or the bacterial cell which comprises said plasmid (i.e. the recipient bacterium when the plasmid is a conjugative plasmid) may be a strain belonging to the genus Spirulina.

In any embodiment herein, the modified bacterium, the bacterial host (donor) cell comprising a conjugative plasmid or the bacterial cell which comprises said plasmid (i.e. the recipient bacterium when the plasmid is a conjugative plasmid) may be a strain belonging to the genus Cyanobacteria.

In one embodiment relating to modified bacteria for producing and secreting one or more ALMs, the bacterium may comprise further metabolic pathways, which enable the bacterium to metabolize and selectively grow on exogenously-added compounds (e.g. rare carbohydrates) or may be present only in the local environment (such as in the gut, e.g. lower gastrointestinal tract or upper gastrointestinal tract). These metabolic pathways give a fitness advantage to the modified bacterium, and help it to effectively compete against other, native bacteria in the environment. This results in an increase in the relative abundance of the modified bacterium. Thus, in one embodiment, the modified bacterium may comprise a further metabolic pathway, which enables the bacterium to metabolize and selectively grow on exogenously-added compounds (e.g. rare carbohydrates). In one embodiment, the modified bacterium may comprise a further metabolic pathway, which enables the bacterium to metabolize and selectively grow in the presence of compounds which are present only in the local environment (such as in the gut, e.g. lower gastrointestinal tract or upper gastrointestinal tract).

In one embodiment relating to modified bacteria or donor (host) cells comprising conjugative plasmids for producing and secreting one or more ALMs (in particular IAA), the bacterium is capable of maintaining a stable level of colonisation in a mouse model for at least 5 days, optionally as measured by number of colony-forming units (CFU) in feces. In another embodiment, the bacterium is capable of maintaining a stable level of colonisation in a mouse model for at least 6 days, optionally as measured by number of colony-forming units (CFU) in feces. In another embodiment, the bacterium is capable of maintaining a stable level of colonisation in a mouse model for at least 7 days, optionally as measured by number of colony-forming units (CFU) in feces. In another embodiment, the bacterium is capable of maintaining a stable level of colonisation in a mouse model for at least 8 days, optionally as measured by number of colony-forming units (CFU) in feces.

The level of colonisation may be measured by the level of CFU in the feces. Thus, in one embodiment, the level of colonisation is at least (about) 1×10⁴ CFU/g feces. In another embodiment, the level of colonisation is at least (about) 1×10⁵ CFU/g feces. In another embodiment, the level of colonisation is at least (about) 1×10⁶ CFU/g feces. In another embodiment, the level of colonisation is at least (about) 1×10⁷ CFU/g feces. In another embodiment, the level of colonisation is at least (about) 1×10⁸ CFU/g feces.

In one embodiment relating to modified bacteria or donor (host) cells comprising conjugative plasmids for producing and secreting IAA, the bacterium is capable of producing IAA in a mouse model for at least (about) 24 hours, optionally by measuring IAA levels in feces. In another embodiment, the bacterium is capable of producing IAA in a mouse model for at least (about) 48 hours, optionally by measuring IAA levels in feces. In another embodiment, the bacterium is capable of producing IAA in a mouse model for at least (about) 72 hours, optionally by measuring IAA levels in feces. In another embodiment, the bacterium is capable of producing IAA in a mouse model for at least (about) 96 hours, optionally by measuring IAA levels in feces.

In one embodiment, the IAA levels are at least (about) 10 nmol/g feces. In another embodiment, the IAA levels are at least (about) 15 nmol/g feces. In another embodiment, the IAA levels are at least (about) 20 nmol/g feces.

In any of these embodiments relating to colonisation, or IAA secretion, the levels are measured in an in vivo mouse model. The mouse model may be conducted as described for group 6 in Example 4.2.2 herein.

In one embodiment relating to modified bacteria or donor (host) cells comprising conjugative plasmids for producing and secreting one or more ALMs (in particular IAA), the bacterium or bacterial cell has been engineered to remove some or all (e.g. all) prophage genes present in the bacterium or bacterial cell genome. The modified bacterium or bacterial cell strain may be devoid of some or all (e.g. all) prophage genes.

In one embodiment relating to modified bacteria or donor (host) cells comprising conjugative plasmids for producing and secreting one or more ALMs (in particular IAA), the bacterium or bacterial cell has been engineered to remove any identified pathogenicity factors (such as hlyA, hlyB, hlyC and/or hlyD or any combination thereof) present in the bacterium or bacterial cell genome. The bacterium or bacterial cell strain may be devoid of pathogenicity factors (such as hlyA, hlyB, hlyC and/or hlyD or any combination thereof).

### Conjugative Plasmids

A conjugative plasmid is a plasmid which, when comprised within a bacterial cell (“donor” cell) is able to be transferred to another bacterium (“recipient” cell) through the mechanism of bacterial conjugation. Bacterial conjugation is the unidirectional and horizontal transmission of genetic information from one bacterium to another. Conjugative plasmids generally fall into two classes: mobilizable plasmids and self-transmissible plasmids.

In any embodiment, the conjugative plasmid is capable of being transferred to a recipient bacterial cell. In any embodiment, the conjugative plasmid is transferred to a bacterial cell (i.e. a recipient cell). The recipient cell may be any bacterial cell described elsewhere herein (e.g. a gram-negative bacterial cell).

There is provided a host (donor) cell comprising a conjugative plasmid as described herein. The host (donor) cell may be any bacterial cell described elsewhere herein (e.g. a gram-negative bacterial cell).

Mobilizable plasmids comprise at least an origin of transfer (oriT), a relaxase and other genetic information on the plasmid which is transferred to the recipient cell. They require helper functions provided by e.g. a second plasmid or the chromosome of the donor cell to effect the plasmid transfer. In one embodiment, the conjugative plasmid is a mobilizable plasmid. In one embodiment, the conjugative plasmid is a mobilizable plasmid comprising an origin of transfer (oriT) and a relaxase.

A self-transmissible plasmid, in addition to the genetic information on the plasmid which is transferred to the recipient cell, also contain all the molecular machinery needed for self-transfer (e.g. for pilus formation and initiation of gene transfer) on the same plasmid. In one embodiment, the conjugative plasmid is a self-transmissible plasmid. In one embodiment, the conjugative plasmid is a self-transmissible plasmid which comprises all of the molecular machinery necessary for self-transfer. In one embodiment, the conjugative plasmid comprises an oriT and encodes all proteins required to mobilise the plasmid for conjugative transfer between cells.

Engineered conjugative plasmids are described in more detail in WO2021/037732 (SNIPR Biome ApS), which is incorporated herein in its entirety. The features of such conjugative plasmids and bacterial cells comprising them as described in the claims as filed in WO2021/037732 are also incorporated herein by reference.

Thus, in one embodiment, the conjugative plasmid is devoid of a hypC2 nucleotide sequence, or a homologue thereof, for example a hypC2 nucleotide sequence of Seq ID No:75. The conjugative plasmid may comprise an OriT of an IncX plasmid. The conjugative plasmid may be an IncX plasmid. The conjugative plasmid may be a β10 plasmid.

The conjugative plasmid (or any other plasmid described herein) may be a plasmid based on any plasmid found in a bacterium disclosed herein. For example, the plasmid may be an Enterobacteriaceae plasmid. In one embodiment, the plasmid is an E. coli, Klebsiella, Salmonella, Erwinia, Shigella, Pantoea, Proteus or Citrobacter plasmid. In one embodiment, the plasmid may be from a genera selected from Bifidobacterium, Bacteroides, Lactobacillus, Lacticaseibacillus, Lactiplantibacillus, Levilactobacillus, Ligilactobacillus, Limosilactobacillus and Lactococcus. In one embodiment, the plasmid may be from a strain belonging to a genera selected from a Bifidobacterium genus or a Bacteroides genus. In one embodiment, the genera is a Bacteroides genus. In one embodiment, the genera is a Bifidobacterium genus. In one embodiment, the plasmid may be from a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacillus brevis, Ligilactobacillus salivarius, Limosilactobacillus reuteri and Lactococcus lactis. In one embodiment, the plasmid may be from a species which is selected from Bifidobacterium longum, Bifidobacterium breve, Bifidobacterium adolescentis, Bacteroides uniformis, Bacteroides vulgatus and Bacteroides thetaiotaomicron. In one embodiment, the plasmid may be from a genus or species disclosed in Table 3.

In an example, the conjugative plasmid is capable of replicating in a bacterial cell from any bacterial genus or species described herein. For example, the conjugative plasmid is capable of replicating in an E. coli, Klebsiella, Salmonella, Erwinia, Shigella, Pantoea, Proteus or Citrobacter host (donor) cell. In one embodiment, the conjugative plasmid is capable of replicating in a bacterial cell from a genera selected from Bifidobacterium, Bacteroides, Lactobacillus, Lacticaseibacillus, Lactiplantibacillus, Levilactobacillus, Ligilactobacillus, Limosilactobacillus and Lactococcus. In one embodiment, the conjugative plasmid is capable of replicating in a bacterial cell from a strain belonging to a genera selected from a Bifidobacterium genus or a Bacteroides genus. In one embodiment, the genera is a Bacteroides genus. In one embodiment, the genera is a Bifidobacterium genus. In one embodiment, the conjugative plasmid is capable of replicating in a bacterial cell from a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacillus brevis, Ligilactobacillus salivarius, Limosilactobacillus reuteri and Lactococcus lactis. In one embodiment, the conjugative plasmid is capable of replicating in a bacterial cell from a species which is selected from Bifidobacterium longum, Bifidobacterium breve, Bifidobacterium adolescentis, Bacteroides uniformis, Bacteroides vulgatus and Bacteroides thetaiotaomicron. In one embodiment, the conjugative plasmid is capable of replicating in a bacterial cell from a genus or species disclosed in Table 3.

In one embodiment the conjugative plasmid (or any other plasmid described herein) is capable of being hosted in an Enterobacteriaceae cell. In one embodiment, the plasmid is capable of being hosted in an E. coli, Klebsiella, Salmonella, Erwinia, Shigella, Pantoea, Proteus or Citrobacter cell. In one embodiment, the plasmid is capable of being hosted in a bacterial cell from a genera selected from Bifidobacterium, Bacteroides, Lactobacillus, Lacticaseibacillus, Lactiplantibacillus, Levilactobacillus, Ligilactobacillus, Limosilactobacillus and Lactococcus. In one embodiment, the plasmid is capable of being hosted in a bacterial cell from a strain belonging to a genera selected from a Bifidobacterium genus or a Bacteroides genus. In one embodiment, the genera is a Bacteroides genus. In one embodiment, the genera is a Bifidobacterium genus. In one embodiment, the plasmid is capable of replicating in a bacterial cell from a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacillus brevis, Ligilactobacillus salivarius, Limosilactobacillus reuteri and Lactococcus lactis. In one embodiment, the plasmid is capable of being hosted in a bacterial cell from a species which is selected from Bifidobacterium longum, Bifidobacterium breve, Bifidobacterium adolescentis, Bacteroides uniformis, Bacteroides vulgatus and Bacteroides thetaiotaomicron. In one embodiment, the plasmid is capable of being hosted in a bacterial cell from a genus or species disclosed in Table 3.

In one embodiment, the conjugative plasmid is capable of being conjugatively transferred to an Enterobacteriaceae cell. In one embodiment, the plasmid is capable of being conjugatively transferred to an E. coli, Klebsiella, Salmonella, Erwinia, Shigella, Pantoea, Proteus or Citrobacter cell. In one embodiment, the conjugative plasmid is capable of being conjugatively transferred to a bacterial cell from a genera selected from Bifidobacterium, Bacteroides, Lactobacillus, Lacticaseibacillus, Lactiplantibacillus, Levilactobacillus, Ligilactobacillus, Limosilactobacillus and Lactococcus. In one embodiment, the conjugative plasmid is capable of being conjugatively transferred to in a bacterial cell from a strain belonging to a genera selected from a Bifidobacterium genus or a Bacteroides genus. In one embodiment, the genera is a Bacteroides genus. In one embodiment, the genera is a Bifidobacterium genus. In one embodiment, the conjugative plasmid is capable of being conjugatively transferred to a bacterial cell from a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacillus brevis, Ligilactobacillus salivarius, Limosilactobacillus reuteri and Lactococcus lactis. In one embodiment, the conjugative plasmid is capable of being conjugatively transferred to a bacterial cell from a species which is selected from Bifidobacterium longum, Bifidobacterium breve, Bifidobacterium adolescentis, Bacteroides uniformis, Bacteroides vulgatus and Bacteroides thetaiotaomicron. In one embodiment, the conjugative plasmid is capable of being conjugatively transferred to a bacterial cell from a genus or species disclosed in Table 3.

In one embodiment, the conjugative plasmid is a conjugative plasmid isolated from E. coli. In another embodiment, the conjugative plasmid is a β10 conjugative plasmid from E. coli (e.g. as shown in Example 5 herein).

In one embodiment, the donor (host) cell comprising a conjugative plasmid as described herein is an E. coli (such as a symbioflor E. coli, e.g. G6/7) host cell (e.g. as shown in Example 5 herein). In another embodiment, the donor (host) cell comprising a conjugative plasmid as described herein is an E. hormaechei host cell (e.g. as shown in Example 5 herein).

In one embodiment, the recipient cell is an E. coli recipient strain comprising a conjugative plasmid as described herein. The plasmid is introduced via conjugation from a donor (host) cell described herein (e.g. as shown in Example 5 herein). The E. coli recipient strain may be comprised by a native microbiome, e.g. in the gut of a subject. In another embodiment, the recipient cell is a klebsiella recipient strain comprising a conjugative plasmid as described herein. The plasmid is introduced via conjugation from a donor (host) cell described herein (e.g. as shown in Example 5 herein). The klebsiella recipient strain may be comprised by a native microbiome, e.g. in the gut of a subject.

As an alternative, delivery of the one or more heterologous gene(s) for the biosynthesis of said one or more ALMs and/or the heterologous gene encoding an exporter which is capable of exporting said one or more ALMs are delivered by a phage particle. Thus, there is a provided a phage particle comprising one or more heterologous gene(s) for the biosynthesis of said one or more ALMs (as described elsewhere herein) and/or the heterologous gene encoding an exporter which is capable of exporting said one or more ALMs (as described elsewhere herein) out of a bacterium that has been infected by said phage. In one embodiment, the one or more heterologous gene(s) for the biosynthesis of said one or more ALMs and/or the heterologous gene encoding an exporter which is capable of exporting said one or more ALMs may be comprised by a phagemid within a phage particle. In another embodiment, the one or more heterologous gene(s) for the biosynthesis of said one or more ALMs and/or the heterologous gene encoding an exporter which is capable of exporting said one or more ALMs may be comprised in the chromosome of the phage particle (e.g. in its chromosome). The phage chromosome or the phagemid may further comprise a kill switch as described elsewhere herein.

### Kill Switches

In some embodiments, it may be desirable to include, in either the bacterium or in the plasmid (e.g. in the conjugative plasmid) a kill switch. A kill switch is a biocontainment system which is included in the bacterium or in the plasmid (e.g. conjugative plasmid) and is designed to destroy the bacterium, or in the case of a plasmid (e.g. a conjugative plasmid), either the plasmid itself only, or the plasmid and the bacterium comprising the plasmid together, when no longer contained within its desired environment (e.g. within a microbiome, such as a gut microbiome, within a subject). Such means are well-known in the art, and are regulatable, for example by the addition of non-naturally occurring substances (e.g. synthetic amino acids), temperature and the like. Specific examples of promoters and kill switches (in particular, for removal of plasmids from bacteria) are provided in WO2023/012109A2 (SNIPR Biome, ApS).

Bacteria comprising kill switches have been engineered for in vitro research purposes, e.g. to limit the spread of a biofuel-producing microorganism outside of a laboratory environment. Bacteria engineered for in vivo administration to treat a disease may also be programmed to die at a specific time after the expression and delivery of the ALM(s), or after the subject has experienced the therapeutic effect. For example, in some embodiments, the kill switch is activated to kill the bacterium, remove the plasmid (e.g. the conjugative plasmid) from the bacterium comprising the plasmid, or to kill the bacterium comprising the plasmid (i.e. the recipient bacterium when the plasmid is a conjugative plasmid), after a period of time following oxygen level-dependent expression of the ALM(s). In some embodiments, the kill switch is activated in a delayed fashion following oxygen level dependent expression of the ALM(s). Alternatively, the bacterium, or bacterium comprising the plasmid (i.e. the recipient bacterium when the plasmid is a conjugative plasmid), may be engineered to die after the bacterium has spread outside of a disease site. Specifically, it may be useful to prevent long-term colonization of subjects by the bacterium, or the bacterium comprising the plasmid (i.e. the recipient bacterium when the plasmid is a conjugative plasmid), spread of the bacterium, or the bacterium comprising the plasmid (i.e. the recipient bacterium when the plasmid is a conjugative plasmid) outside the area of interest (for example, outside the gut) within the subject, or spread of the bacterium, or the bacterium comprising the plasmid (i.e. the recipient bacterium when the plasmid is a conjugative plasmid) outside of the subject into the environment (for example, spread to the environment through the stool of the subject).

Kill-switches can be designed such that toxin(s) is/are produced in response to an environmental condition or external signal (e.g. the bacterium is killed in response to an external cue) or, alternatively designed such that a toxin is produced once an environmental condition no longer exists or an external signal is ceased. Examples of such promoters are also described elsewhere herein. The toxin(s) is/are toxic to the bacterium which produces and secretes said one or more ALMs.

The switches that control production of the toxin(s) can be based on, for example, transcriptional activation, translation (riboregulators), or DNA recombination (recombinase-based switches), and can sense environmental stimuli such as anaerobiosis, reactive oxygen species, temperature, bile acids, pH, lactate, caffeine or other biosensors. These switches can be activated by a single environmental factor or may require several activators in AND, OR, NAND and NOR logic configurations to induce cell death. For example, an AND riboregulator switch is activated by tetracycline, isopropyl P-D-I-thiogaiactopyranoside (IPTG), and arabinose to induce the expression of lysins, which permeabilize the cell membrane and kill the cell. IPTG induces the expression of the endolysin and holin mRNAs, which are then derepressed by the addition of arabinose and tetracycline. All three inducers must be present to cause cell death.

Thus, in some embodiments, the bacterium, or the bacterium comprising the plasmid (i.e. the recipient bacterium when the plasmid is a conjugative plasmid) is further programmed to die or specifically degrade the plasmid (e.g. the conjugative plasmid) after sensing an exogenous environmental signal, for example, in a low-oxygen environment.

Kill switches can be permissive, such that the bacterium or recipient bacterium will continue to thrive in its environment, unless a defined condition changes. Cell survival or proliferation may be impeded by the expression of toxins and lysis proteins (e.g. as discussed in Knudsen, et al., Appl. Environ. Microbiol., 57, 85-92, 1991; Callura, et al., Proc. Natl Acad. Sci. USA, 107, 15898-15903, 2010, each of which is incorporated herein by reference in its entirety), cleavage and degradation of the bacterial chromosome by Cas proteins (e.g. as discussed in Caliando, et al., Nat. Commun., 6, 6989, 2015; and Rottinghaus, et al., Nature Comms., 13 (672), 2022, each of which is incorporated herein by reference in its entirety) or the degradation of essential proteins (e.g. as discussed in Chan, et al., Nat. Chem. Biol., 12, 82-86, 2016 which is incorporated herein by reference in its entirety).

Thus, in one embodiment, the kill switch comprises a toxin gene, expression of which is induced in response to an environmental condition(s) and/or signal(s).

Expression of the toxin gene may be provided by an inducible promoter. Such promoters are well-known to those skilled in the art, and may include oxygen level-dependent promoters (e.g. an FNR [fumarate and nitrate reductase regulator]-inducible promoter, the ANR [anaerobic arginine deiminase and nitrate reductase regulator]-inducible promoter, and the DNR [dissimilatory nitrate respiration regulator]-inducible promoter).

The inducible promoter may be a promoter which is induced by inflammation or an inflammatory response (e.g. an RNS and/or ROS-inducible promoter).

The inducible promoter may be a temperature-sensitive promoters (e.g. as described elsewhere herein). Temperature sensitive promoters include TlpA, TcI, TetR (and A89D and I193N mutants thereof), LacI (and A241T and G265D mutants thereof), GrpE, HtpG, Lon, RpoH, Clp and Dnak (which are described in more detail in Piraner et al., Nature Chemical Biology, 13, 75-80, 2016, doi: 10.1038/nchembio.2233; which is incorporated herein in its entirety).

In one embodiment, the kill switch comprises a toxin gene expression of which is induced by a change in temperature. In one embodiment, the kill switch comprises a toxin gene expression of which is induced by an increase in oxygen levels. In one embodiment, the kill switch comprises a toxin gene expression of which is induced by the addition of a molecule which is not usually present in the local environment, such as the gut (e.g. arabinose, sugar alcohol (e.g. sorbitol), tetracycline, IPTG, rhamnose, and non-naturally occurring amino acids).

The inducible promoter may be a promoter induced by a substance that may or may not be naturally present in the local environment (i.e. is exogenously added to the local environment). Examples include, but are not limited to an arabinose-inducible promoter (e.g. a pBAD promoter), tetracycline-inducible promoters, IPTG-inducible promoters, rhamnose-inducible promoters, xylitol-inducible promoters, sorbitol-inducible promoters, and nutritional-inducible promoters.

Examples of such toxins that can be used in kill-switches include, but are not limited to, bacteriocins, lysins, and other molecules that cause cell death by lysing cell membranes, degrading cellular DNA, or other mechanisms. Such toxins can be used individually or in combination.

In one embodiment, the toxin gene is a CRISPR/Cas system which targets and cleaves the chromosome of the bacterium, or the bacterium comprising the plasmid (i.e. the recipient bacterium when the plasmid is a conjugative plasmid) or the plasmid DNA (e.g. the conjugative plasmid DNA). In one embodiment, the toxin gene is a CRISPR/Cas system which targets and cleaves an essential gene in the bacterial or recipient-bacterial chromosome or on the conjugative plasmid. Essential genes are well-known to those in the art. Examples include, but are not limited to DNA synthesis genes such as, thyA), cell wall synthesis genes (such as dapA) and amino acid synthesis genes (such as serA or metA).

In the case of a bacterium for producing and secreting one or more ALMs, the kill switch may be comprised within the chromosome of the bacterium. Alternatively, it may be on a plasmid comprised within the bacterium for producing and secreting one or more ALMs.

### Auxotrophs

In one embodiment relating to modified bacteria, the bacterium may further comprise a mutation which results in the bacterium becoming an auxotroph. In one embodiment, the bacterium includes an auxotrophy.

In one embodiment, the bacterium includes an auxotrophy, such as deletion of thyA.

In one embodiment, the bacterium is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thil auxotroph. In one embodiment, the bacterium or recipient bacterium has more than one auxotrophy, for example, it may be a ΔthyA and ΔdapA auxotroph.

In one embodiment, the bacterium is an auxotroph and also comprises a kill-switch.

In the case of a bacterium for producing and secreting one or more ALMs, the auxotrophy may be comprised within the chromosome of the bacterium. Alternatively, the auxotrophy may be on a plasmid comprised within the bacterium for producing and secreting one or more ALMs.

### Formulations and Compositions Comprising the Bacteria, Conjugative Plasmids or Host Bacteria

There is provided a pharmaceutical composition comprising a bacterium, a plasmid (e.g. conjugative plasmid) or host (e.g. donor) bacterium as described herein, and a pharmaceutically acceptable excipient or carrier.

The bacterium, plasmid (e.g. conjugative plasmid) or host (e.g. donor) bacterium may be formulated in a pharmaceutical composition comprising a diluent, excipient or carrier. The formulation may be comprised within a medical device (such as an ampoule, a syringe, or an inhaler) or is formulated in a tincture, a capsule or a slow-release formulation. The formulation may be an oral tablet, comprised within a blister pack.

In one embodiment, the pharmaceutical composition comprising a bacterium, plasmid (e.g. conjugative plasmid) or host (e.g. donor) bacterium as described herein is formulated for oral or rectal administration. In one embodiment, the pharmaceutical composition is formulated for oral administration. In one embodiment, the pharmaceutical composition is formulated as a capsule or coated tablet.

The formulation comprising the bacterium, plasmid (e.g. conjugative plasmid) or host (e.g. donor) bacterium may be freeze dried prior to encapsulation. Thus the formulation may comprise freeze dried modified bacteria, plasmids (e.g. conjugative plasmids) or host (e.g. donor) bacteria. In one embodiment, the pharmaceutical composition comprising a bacterium, plasmid (e.g. conjugative plasmid) or host (e.g. donor) bacterium as described herein is a lyophilised formulation. In one embodiment, the pharmaceutical composition comprising a bacterium, plasmid (e.g. conjugative plasmid) or host (e.g. donor) bacterium as described herein is an encapsulated formulation to be released in the lower gut of a subject. In one embodiment, the pharmaceutical composition comprising a bacterium, plasmid (e.g. conjugative plasmid) or host (e.g. donor) bacterium as described herein is an encapsulated formulation to be released in the large intestine of a subject. In one particular embodiment, the pharmaceutical composition comprising a bacterium, plasmid (e.g. conjugative plasmid) or host (e.g. donor) bacterium as described herein is an encapsulated formulation to be released in the small intestine of a subject.

Acceptable carriers, excipients, or stabilizers are non-toxic to patients at the dosages and concentrations employed, and include buffers such as phosphate, citrate, and other organic acids; antioxidants including ascorbic acid and methionine; low molecular weight (less than about 10 residues) polypeptides; proteins, such as serum albumin, gelatine, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and metal complexes (e.g. Zn-protein complexes). In limited circumstances, due to stability of the vectors, the formulation may include preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol). A skilled formulator is aware of agents which are compatible with the different modes of delivery of the bacteria, conjugative plasmids or host (e.g. donor) bacteria described herein.

The bacteria, plasmids (e.g. conjugative plasmids) or host (e.g. donor) bacteria can also be formulated in liposomes. Liposomes containing the bacteria, plasmids (e.g. conjugative plasmids) or host (e.g. donor) bacteria are prepared by methods known in the art, such as described in Epstein et al. (1985) Proc. Natl. Acad. Sci. USA 82:3688; Hwang et al. (1980) Proc. Natl. Acad. Sci. USA 77:4030; and U.S. Pat. Nos. 4,485,045 and 4,544,545, each of which is incorporated herein by reference in its entirety. Liposomes with enhanced circulation time are disclosed in U.S. Pat. No. 5,013,556, incorporated herein by reference.

Bacteria, plasmids (e.g. conjugative plasmids) or host (e.g. donor) bacteria described herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1990) Mack Publishing Co., Easton, Pa.

Sustained-release preparations can also be prepared. Suitable examples of sustained-release preparations include semipermeable matrices of solid hydrophobic polymers containing the antagonist, which matrices are in the form of shaped articles, e.g. films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The formulation may be comprised within a medical device, such as an ampoule, a syringe, or an inhaler.

Suitable dosage amounts for the genetically engineered bacteria may range from about 10⁵ to 10¹² bacteria, e.g. approximately 10⁵ bacteria, approximately 10⁶ bacteria, approximately 10⁷ bacteria, approximately 10⁸ bacteria, approximately 10⁹ bacteria, approximately 10¹⁰ bacteria, approximately 10¹¹ bacteria, or approximately 10¹² bacteria. The composition may be administered daily, weekly, or monthly. It may be administered multiple times per day (e.g. twice or three times per day).

Suitable dosage amounts for the genetically engineered bacteria may range from about 1×10⁴ to 1×10¹² colony forming units (CFU)/gram, e.g. approximately 1×10⁴ CFU/gram, approximately 1×10⁵ CFU/gram, approximately 1×10⁶ CFU/gram, approximately 1×10⁷ CFU/gram, approximately 1×10⁸ CFU/gram, approximately 1×10⁹ CFU/gram, approximately 1×10¹⁰ CFU/gram, approximately 1×10¹¹ CFU/gram, or approximately 1×10¹² CFU/gram. In particular, the formulation comprises from about 1×10⁵ to 1×10¹² colony forming units (CFU)/gram. The formulation may comprise from about 1×10⁶ to 1×10¹¹ colony forming units (CFU)/gram. The formulation may comprise from about 1×10⁷ to 1×10¹¹ colony forming units (CFU)/gram. The formulation may comprise from about 1×10⁸ to 1×10¹⁰ colony forming units (CFU)/gram. In particular, the formulation comprises (approximately) 1×10⁹ colony forming units (CFU)/gram of a modified bacterium or host cell as described herein. The composition may be administered daily, weekly, or monthly. It may be administered multiple times per day (e.g. twice or three times per day). In some embodiments, the genetically engineered bacteria are enterically coated for release into the gut or a particular region of the gut, for example, the small or large intestines. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels. In a particular embodiment, the modified bacterium or host cell is formulated as an enteric late release capsule.

Uses of the Methods, Bacteria, Plasmids (e.g. Conjugative Plasmids) and Host Bacteria Disclosed Herein

The methods described herein can be carried out ex vivo. The methods described herein can be carried out in vitro. The methods described herein can be carried out in vivo.

There is provided a bacterium, a plasmid (e.g. conjugative plasmid), a host (e.g. donor) bacterium or a pharmaceutical formulation as described herein for use in a method to treat or prevent a disease or condition mediated in a patient by a lack of, or insufficient amount of an ALM (e.g. of IAA).

There is provided method of producing an ALM (e.g. IAA) in the gut of a subject, comprising administering to said subject a bacterium, a plasmid (e.g. conjugative plasmid), a host (e.g. donor) bacterium or pharmaceutical formulation as described herein.

The patient can be a human or animal subject. The patient can be a mammal such as a non-primate (e.g. cows, pigs, horses, cats, dogs, rats, etc.) or a primate (e.g. monkey and human). The patient may be a rodent, mouse or rat. The patient may be a vertebrate, reptile, bird or fish. In particular, the patient is a human.

There is provided a bacterium, a plasmid (e.g. conjugative plasmid), a host (e.g. donor) bacterium or a pharmaceutical formulation as described herein for use as a medicament or for use in therapy.

There is provided a method of treating a metabolic disease comprising administering to a subject in need thereof a bacterium, a plasmid (e.g. conjugative plasmid), a host (e.g. donor) bacterium or a pharmaceutical formulation as described herein.

There is provided a method of treating a cardiovascular metabolic disease comprising administering to a subject in need thereof a bacterium, a plasmid (e.g. conjugative plasmid), a host (e.g. donor) bacterium or a pharmaceutical formulation as described herein.

There is provided a method of treating a metabolic disease selected from leaky gut, type 1 diabetes, type 2 diabetes (including complications of type 1 and type 2 diabetes, e.g. insulin sensitivity in type 2 diabetes), metabolic syndrome, Bardet-Biedel syndrome, Prader-Willi syndrome, non-alcoholic fatty liver disease, tuberous sclerosis; Albright hereditary osteodystrophy; brain-derived neurotrophic factor (BDNF) deficiency, Single-minded 1 (SIM1) deficiency, leptin deficiency, leptin receptor deficiency, pro-opiomelanocortin (POMC) defects, proprotein convertase subtilisin/kexin type 1 (PCSK1) deficiency, Src homology 2B1 (SH2B1) deficiency, pro-hormone convertase ⅓ deficiency, melanocortin-4-receptor (MC4R) deficiency, Wilms tumor, aniridia, genitourinary anomalies, and mental retardation (WAGR) syndrome, pseudohypoparathyroidism type 1A, Fragile X syndrome, Borjeson-Forsmann-Lehmann syndrome, Alstrom syndrome, Cohen syndrome, and ulnar-mammary syndrome, said method comprising administering to a subject in need thereof a bacterium, a plasmid (e.g. conjugative plasmid), a host (e.g. donor) bacterium or a pharmaceutical formulation as described herein.

There is provided a method of treating a metabolic disease selected from metabolic syndrome, type 2 diabetes (including complications of type 2 diabetes, e.g. insulin sensitivity in type 2 diabetes), and non-alcoholic fatty liver disease, said method comprising administering to a subject in need thereof a bacterium, a plasmid (e.g. conjugative plasmid), a host (e.g. donor) bacterium or a pharmaceutical formulation as described herein.

There is provided a bacterium, a plasmid (e.g. conjugative plasmid), a host (e.g. donor) bacterium or a pharmaceutical formulation as described herein for use in the treatment of a metabolic disease.

There is provided a bacterium, a plasmid (e.g. conjugative plasmid), a host (e.g. donor) bacterium or a pharmaceutical formulation as described herein for use in the treatment of a cardiovascular metabolic disease.

There is provided a bacterium, a plasmid (e.g. conjugative plasmid), a host (e.g. donor) bacterium or a pharmaceutical formulation as described herein for use in the treatment of a metabolic disease selected from leaky gut, type 1 diabetes, type 2 diabetes (including complications of type 1 and type 2 diabetes, e.g. insulin sensitivity in type 2 diabetes), metabolic syndrome, Bardet-Biedel syndrome, Prader-Willi syndrome, non-alcoholic fatty liver disease, tuberous sclerosis; Albright hereditary osteodystrophy; brain-derived neurotrophic factor (BDNF) deficiency, Single-minded 1 (SIM1) deficiency, leptin deficiency, leptin receptor deficiency, pro-opiomelanocortin (POMC) defects, proprotein convertase subtilisin/kexin type 1 (PCSK1) deficiency, Src homology 2B1 (SH2B1) deficiency, pro-hormone convertase ⅓ deficiency, melanocortin-4-receptor (MC4R) deficiency, Wilms tumor, aniridia, genitourinary anomalies, and mental retardation (WAGR) syndrome, pseudohypoparathyroidism type 1A, Fragile X syndrome, Borjeson-Forsmann-Lehmann syndrome, Alstrom syndrome, Cohen syndrome, and ulnar-mammary syndrome.

There is provided a bacterium, a plasmid (e.g. conjugative plasmid), a host (e.g. donor) bacterium or a pharmaceutical formulation as described herein for use in the treatment of a metabolic disease selected from metabolic syndrome, type 2 diabetes (including complications of type 2 diabetes, e.g. insulin sensitivity in type 2 diabetes), and non-alcoholic fatty liver disease.

Metabolic Syndrome affects approximately 20-30% of the middle-aged population, and represents an increased risk to cardiovascular disorders, the leading cause of death in the United States. Obesity, dyslipidemia, hypertension, and type 2 diabetes are described as metabolic syndrome. In some embodiments, the bacteria, conjugative plasmids, host (e.g. donor) cells or pharmaceutical compositions described herein are useful in the treatment, prevention and/or management of metabolic syndrome and/or obesity.

Metabolic syndrome is a clustering of at least three of five of the following medical conditions: abdominal (central) obesity, elevated blood pressure, elevated fasting plasma glucose, high serum triglycerides, and low high-density lipoprotein (HDL) levels.

Metabolic diseases are associated with a variety of physiological changes, including but not limited to elevated glucose levels, elevated triglyceride levels, elevated cholesterol levels, insulin resistance, high blood pressure, hypogonadism, subfertility, infertility, abdominal obesity, pro-thrombotic conditions, and pro-inflammatory conditions.

Cardiovascular disease includes coronary artery diseases (CAD) such as angina and myocardial infarction, stroke, hypertensive heart disease, rheumatic heart disease, cardiomyopathy, heart arrhythmia, congenital heart disease, valvular heart disease, carditis, aortic aneurysms, peripheral artery disease, and venous thrombosis. Coronary artery disease, stroke, and peripheral artery disease involve atherosclerosis, caused inter alia by high blood pressure, smoking, diabetes, lack of exercise, obesity, high blood cholesterol, poor diet, and excessive alcohol consumption, and the like.

Obesity is a common, deadly, and costly disease in developed countries which impacts all age groups, race, and gender. Obesity can be classified as an inflammatory disease because it is associated with immune activation and a chronic, low-grade systemic inflammation. Endotoxemia, a process resulting from translocation of endotoxic compounds (lipopolysaccharides [LPS]) of gram-negative intestinal bacteria. In the last decade, it has become evident that insulin resistance and T2DM are characterized by low-grade inflammation. In this respect, LPS trigger a low-grade inflammatory response, and the process of endotoxemia can therefore result in the development of insulin resistance and other metabolic disorders. Other anti-inflammatory ALMs as described herein may also be useful in the treatment of type 2 diabetes.

In certain embodiments, the bacteria, plasmids (e.g. conjugative plasmids), host (e.g. donor) cells or pharmaceutical compositions as described herein decrease tryptophan levels in the patient, e.g. in the serum and/or in the gut, e.g. for the prevention, treatment, and/or management of obesity.

Metabolic syndrome is an important risk factor for cardiovascular disease incidence and mortality, as well as all-cause mortality. Thus, the detection, prevention, and treatment of the underlying risk factors of the metabolic syndrome are a critical approach to lower the cardiovascular disease incidence in the general population.

The bacteria, plasmids (e.g. conjugative plasmids), host (e.g. donor) cells or pharmaceutical compositions as described herein can be administered to the patient in one or more doses. It is understood that the precise dosage and duration of treatment is a function of the disease being treated and can be determined empirically using known testing protocols or by extrapolation from in vivo or in vitro test data, and taking into account age, weight and sex of the patient. It is to be noted that concentrations and dosage values can also vary with the severity of the condition to be alleviated. It is to be further understood that for any particular patient, specific dosage regimens can be adjusted over time according to the individual need and the professional judgment of the person administering or supervising the administration of the compositions.

Recently, it has been shown that bacterial IAA may enhance the efficacy of chemotherapy in pancreatic cancer patients, see Tintelnot et al., Nature, 615, 2023, 168-174, doi: https://doi.org/10.1038/s41586-023-05728-y and Seo & Wargo, Cell Reports Medicine, 4, 101039, May 16, 2023, doi: https://doi.org/10.1016/j.xcrm.2023.101039, each of which is incorporated herein by reference in its entirety.

Further, it has been shown that IAA is able to activate the Toll-like receptor 4 and c-Jun N-terminal kinase (TLR4-JNK) pathways, as well as decreasing expression of tumour necrosis factor α (TNF-α), which in turn inhibits proliferation of colorectal cancer (CRC) cells, see Tomii et al., Bioscience, Biotechnology, and Biochemistry, 87 (8), 2023, 839-849, which is incorporated herein by reference in its entirety. In that study, IAA did not induce cytotoxicity, suggesting that it regulates cell cycle progression by activating JNK, and consequently inhibiting cell proliferation.

Thus, there is provided a bacterium, a plasmid (e.g. conjugative plasmid), a host (e.g. donor) bacterium or a pharmaceutical formulation as described herein for use in the treatment of cancers, in particular pancreatic cancer, e.g. pancreatic ductal adenocarcinoma (PDAC). There is also provided a method of treating cancers, in particular colorectal cancer (CRC) and/or pancreatic cancer, e.g. pancreatic ductal adenocarcinoma (PDAC) by administration of an effective amount of a bacterium, a plasmid (e.g. a conjugative plasmid), a host (e.g. donor) bacterium or a pharmaceutical formulation as described herein to a patient in need thereof. In this embodiment, the subject may be receiving chemotherapy. The chemotherapy may be provided concurrently, before or after administration of the bacterium, the plasmid (e.g. conjugative plasmid), the host (e.g. donor) bacterium or the pharmaceutical formulation. Chemotherapies are well-known to those skilled in the art.

IAA has been shown to be useful in attenuating hepatic lipogenesis as well as oxidative and inflammatory stress in a mouse model, see Ji et al., Nutrients, 11, 2062, 2019, doi: 10.3390/nu11092062, incorporated herein in its entirety. The authors show that IAA may be useful in treating non-alcoholic fatty liver disease (NAFLD), as well as improving insulin resistance, lipid metabolism, oxidative stress and inflammatory stress. NAFLD includes the subtypes non-alcoholic fatty liver (NAFL) and nonalcoholic steatohepatitis (NASH). Non-alcoholic fatty liver disease (NAFLD) is increasingly being referred to as metabolic dysfunction-associated steatotic liver disease (MASLD), following a review conducted at the end of 2023, see Rinella et al., Hepatology 78 (6), 1966-1986, 2023, doi: 10.1097/HEP.0000000000000520.

Thus, there is provided a bacterium, a plasmid (e.g. conjugative plasmid), a host (e.g. donor) bacterium or a pharmaceutical formulation as described herein for use in the treatment of non-alcoholic fatty liver disease (NAFLD) by administration of an effective amount of a bacterium, a plasmid (e.g. a conjugative plasmid), a host (e.g. donor) bacterium or a pharmaceutical formulation as described herein to a patient in need thereof. There is also provided a bacterium, a plasmid (e.g. conjugative plasmid), a host (e.g. donor) bacterium or a pharmaceutical formulation as described herein for use in improving insulin resistance, lipid metabolism, oxidative stress and/or inflammatory stress by administration of an effective amount of a bacterium, a plasmid (e.g. a conjugative plasmid), a host (e.g. donor) bacterium or a pharmaceutical formulation as described herein to a patient in need thereof.

As used herein, with respect to treatment methods, “prevention” includes a reducing of the risk of contracting the disease. The “treatment or prevention” may be complete or partial treatment or prevention, i.e. a reduction, but not complete reduction of the disease/condition or symptoms thereof; or a reducing of the risk but not total prevention of the disease/condition or a symptom thereof. Similarly, the methods treat or prevent (i.e. reduces the risk of) an undesirable symptom of the disease or condition or the therapy.

The disease or condition may be metabolic syndrome or cardiometabolic disease (e.g. selected from obesity, diabetes, insulin resistance and non-alcoholic fatty liver disease). The disease may be inflammatory bowel disease (e.g. selected from Crohn's disease and Ulcerative Colitis). The condition may be irritable bowel syndrome or leaky gut syndrome.

Where the ALM is ICA, the treatment may be of a cancer or tumour. The treatment may be the treatment or management of HIV.

To improve the ability of any modified bacterium, host cell or plasmid described herein to effectively colonise and produce the one or more ALMs described herein, the subject may be administered a course of antibiotics to provide is a niche in the relevant microbiome which may be colonised by the within one month (for example within 2 weeks, within one week, e.g. within 5, 4 or 3 days, in particular within 34 hours) of receiving a first dose of the modified bacterium, the host cell, or the pharmaceutical composition.

The antibiotic treatment may be an aminoglycoside (e.g. selected from amikacin, liposomal amikacin, gentamicin, plazomicin and tobramycin). The antibiotic may be a β-lactam inhibitor (e.g. selected from ceftolozane and cilastatin). The antibiotic may be a β-lactamase inhibitor (e.g. selected from avibactam, clavulanate, clavulanic acid, salbactam, tazobactam, relebactam and vaborbactam). The antibiotic may be a carbapenem (e.g. selected from doripenem, ertapenem, imipenem, and meropenem). The antibiotic may be a cephalosporin (e.g. selected from cefaclor, cefadroxil, cefazolin, cefdinir, cefditoren, cefepime, cefiderocol, cefixime, cefotaxime, cefotetan, cefoxitin, cefpodoxime, cefprozil, ceftaroline, ceftazidime, ceftibuten, ceftizoxime, ceftriaxone, cefuroxime and cephalexin). The antibiotic may be a fluoroquinolone (e.g. selected from ciprofloxacin, delafloxacin, gemifloxacin, levofloxacin and moxifloxacin). The antibiotic may be a folate pathway inhibitor (e.g. selected from sulfisoxazole, sulfamethoxazole and trimethoprim). The antibiotic may be a fosfomycin (e.g. fosfomycin). The antibiotic may be a glycopeptide (e.g. selected from dalbavancin, oritavancin, telavancin and vancomycin). The antibiotic may be a glycocycline (e.g. tigecycline). The antibiotic may be a ketolide (e.g. telithromycine). The antibiotic may be a lincosamide (e.g. clindamycin). The antibiotic may be a lipopeptide (e.g. daptomycin). The antibiotic may be a macrocycle (e.g. fidaxomicin). The antibiotic may be a macrolide (e.g. selected from azithromycin, clarithromycin and erythromycin). The antibiotic may be a monobactam (e.g. aztreonam). The antibiotic may be a nitrofuran (e.g. nitrofurantoin). The antibiotic may be a nitroimidazole (e.g. selected from metronidazole and tinidazole). The antibiotic may be a nucleoside analog (e.g. selected from molnupiravir and remdesivir). The antibiotic may be an oxazolidinone (e.g. selected from linezolid and tedizolid). The antibiotic may be a penicillin (e.g. selected from amoxicillin, ampicillin, dicloxacillin, nafcillin, oxacillin, penicillin G, penicillin V, piperacillin and ticarcillin). The antibiotic may be a phenicol (e.g. chloramphenicol). The antibiotic may be a polyene (e.g. selected from amphotericin B, liposomal amphotericin B and amphotericin B lipid complex). The antibiotic may be a polymerase acidic endonuclease inhibitor (e.g. baloxavir marboxil). The antibiotic may be a polymyxin (e.g. selected from colistimethate, colistin and polymyxin B). The antibiotic may be a pleuromutilin (e.g. lefamulin). The antibiotic may be a protease inhibitor (e.g. nirmatrelvir). The antibiotic may be rifampin. The antibiotic may be a streprogramin (e.g. selected from quinupristin and dalfopristin). The antibiotic may be a tetracycline (e.g. selected from eravacycline, minocycline, omadacycline and tetracycline). The antibiotic may be any combination of the antibiotics listed above.

TABLE 3
Example Bacteria
Optionally, the modified bacteria or the bacteria to
which the conjugative plasmid is hosted are/or transferred
(i.e. recipient bacteria) are selected from this Table.

Abiotrophia
Abiotrophia defectiva
Acaricomes
Acaricomes phytoseiuli
Acetitomaculum
Acetitomaculum ruminis
Acetivibrio
Acetivibrio cellulolyticus
Acetivibrio ethanolgignens
Acetivibrio multivorans
Acetoanaerobium
Acetoanaerobium noterae
Acetobacter
Acetobacter aceti
Acetobacter cerevisiae
Acetobacter cibinongensis
Acetobacter estunensis
Acetobacter fabarum
Acetobacter ghanensis
Acetobacter indonesiensis
Acetobacter lovaniensis
Acetobacter malorum
Acetobacter nitrogenifigens
Acetobacter oeni
Acetobacter orientalis
Acetobacter orleanensis
Acetobacter pasteurianus
Acetobacter pornorurn
Acetobacter senegalensis
Acetobacter xylinus
Acetobacterium
Acetobacterium bakii
Acetobacterium carbinolicum
Acetobacterium dehalogenans
Acetobacterium fimetarium
Acetobacterium malicum
Acetobacterium paludosum
Acetobacterium tundrae
Acetobacterium wieringae
Acetobacterium woodii
Acetofilamentum
Acetofilamentum rigidum
Acetohalobium
Acetohalobium arabaticum
Acetomicrobium
Acetomicrobium faecale
Acetomicrobium flavidum
Acetonema
Acetonema longum
Acetothermus
Acetothermus paucivorans
Acholeplasma
Acholeplasma axanthum
Acholeplasma brassicae
Acholeplasma cavigenitalium
Acholeplasma equifetale
Acholeplasma granularum
Acholeplasma hippikon
Acholeplasma laidlawii
Acholeplasma modicum
Acholeplasma morum
Acholeplasma multilocale
Acholeplasma oculi
Acholeplasma palmae
Acholeplasma parvum
Acholeplasma pleciae
Acholeplasma vituli
Achromobacter
Achromobacter denitrificans
Achromobacter insolitus
Achromobacter piechaudii
Achromobacter ruhlandii
Achromobacter spanius
Acidaminobacter
Acidaminobacter hydrogenoformans
Acidaminococcus
Acidaminococcus fermentans
Acidaminococcus intestini
Acidicaldus
Acidicaldus organivorans
Acidimicrobium
Acidimicrobium ferrooxidans
Acidiphilium
Acidiphilium acidophilum
Acidiphilium angustum
Acidiphilium cryptum
Acidiphilium multivorum
Acidiphilium organovorum
Acidiphilium rubrum
Acidisoma
Acidisoma sibiricum
Acidisoma tundrae
Acidisphaera
Acidisphaera rubrifaciens
Acidithiobacillus
Acidithiobacillus albertensis
Acidithiobacillus caldus
Acidithiobacillus ferrooxidans
Acidithiobacillus thiooxidans
Acidobacterium
Acidobacterium capsulatum
Acidocella
Acidocella aminolytica
Acidocella facilis
Acidomonas
Acidomonas methanolica
Acidothermus
Acidothermus cellulolyticus
Acidovorax
Acidovorax anthurii
Acidovorax caeni
Acidovorax cattleyae
Acidovorax citrulli
Acidovorax defluvii
Acidovorax delafieldii
Acidovorax facilis
Acidovorax konjaci
Acidovorax temperans
Acidovorax valerianellae
Acinetobacter
Acinetobacter baumannii
Acinetobacter baylyi
Acinetobacter bouvetii
Acinetobacter calcoaceticus
Acinetobacter gerneri
Acinetobacter haemolyticus
Acinetobacter johnsonii
Acinetobacter junii
Acinetobacter lwoffi
Acinetobacter parvus
Acinetobacter radioresistens
Acinetobacter schindleri
Acinetobacter soll
Acinetobacter tandoil
Acinetobacter tjernbergiae
Acinetobacter towneri
Acinetobacter ursingii
Acinetobacter venetianus
Acrocarpospora
Acrocarpospora corrugata
Acrocarpospora macrocephala
Acrocarpospora pleiomorpha
Actibacter
Actibacter sediminis
Actinoalloteichus
Actinoalloteichus cyanogriseus
Actinoalloteichus hymeniacidonis
Actinoalloteichus spitiensis
Actinobaccillus
Actinobacillus capsulatus
Actinobacillus delphinicola
Actinobacillus hominis
Actinobacillus indolicus
Actinobacillus lignieresii
Actinobacillus minor
Actinobacillus muris
Actinobacillus pleuropneumoniae
Actinobacillus porcinus
Actinobacillus rossii
Actinobacillus scotiae
Actinobacillus seminis
Actinobacillus succinogenes
Actinobaccillus suis
Actinobacillus ureae
Actinobaculum
Actinobaculum massiliense
Actinobaculum schaalii
Actinobaculum suis
Actinomyces urinale
Actinocatenispora
Actinocatenispora rupis
Actinocatenispora thailandica
Actinocatenispora sera
Actinocorallia
Actinocorallia aurantiaca
Actinocorallia aurea
Actinocorallia cavernae
Actinocorallia glomerata
Actinocorallia herbida
Actinocorallia libanotica
Actinocorallia longicatena
Actinomadura
Actinomadura alba
Actinomadura atramentaria
Actinomadura bangladeshensis
Actinomadura catellatispora
Actinomadura chibensis
Actinomadura chokoriensis
Actinomadura citrea
Actinomadura coerulea
Actinomadura echinospora
Actinomadura fibrosa
Actinomadura formosensis
Actinomadura hibisca
Actinomadura kijaniata
Actinomadura atina
Actinomadura livida
Actinomadura luteofluorescens
Actinomadura macra
Actinomadura madurae
Actinomadura oligospora
Actinomadura pelletieri
Actinomadura rubrobrunea
Actinomadura rugatobispora
Actinomadura umbrina
Actinomadura verrucosospora
Actinomadura vinacea
Actinomadura viridilutea
Actinomadura viridis
Actinomadura yumaensis
Actinomyces
Actinomyces bovis
Actinomyces denticolens
Actinomyces europaeus
Actinomyces georgiae
Actinomyces gerencseriae
Actinomyces hordeovulneris
Actinomyces howellii
Actinomyces hyovaginalis
Actinomyces israelii
Actinomyces johnsonii
Actinomyces meyeri
Actinomyces naeslundii
Actinomyces neuii
Actinomyces odontolyticus
Actinomyces oris
Actinomyces radingae
Actinomyces slackii
Actinomyces turicensis
Actinomyces viscosus
Actinoplanes
Actinoplanes auranticolor
Actinoplanes brasiliensis
Actinoplanes consettensis
Actinoplanes deccanensis
Actinoplanes derwentensis
Actinoplanes digitatis
Actinoplanes durhamensis
Actinoplanes ferrugineus
Actinoplanes globisporus
Actinoplanes humidus
Actinoplanes italicus
Actinoplanes liguriensis
Actinoplanes lobatus
Actinoplanes missouriensis
Actinoplanes palleronil
Actinoplanes philippinensis
Actinoplanes rectilineatus
Actinoplanes regularis
Actinoplanes teichomyceticus
Actinoplanes utahensis
Actinopolyspora
Actinopolyspora halophila
Actinopolyspora mortivallis
Actinosynnema
Actinosynnema mirum
Actinotalea
Actinotalea fermentans
Aerococcus
Aerococcus sanguinicola
Aerococcus urinae
Aerococcus urinaeequi
Aerococcus urinaehominis
Aerococcus viridans
Aeromicrobium
Aeromicrobium erythreum
Aeromonas
Aeromonas allosaccharophila
Aeromonas bestiarum
Aeromonas caviae
Aeromonas encheleia
Aeromonas enteropelogenes
Aeromonas eucrenophila
Aeromonas ichthiosmia
Aeromonas jandaei
Aeromonas media
Aeromonas popoffii
Aeromonas sobria
Aeromonas veronii
Agrobacterium
Agrobacterium gelatinovorum
Agrococcus
Agrococcus citreus
Agrococcus jenensis
Agromonas
Agromonas oligotrophica
Agromyces
Agromyces fucosus
Agromyces hippuratus
Agromyces luteolus
Agromyces mediolanus
Agromyces ramosus
Agromyces rhizospherae
Akkermansia
Akkermansia muciniphila
Albidiferax
Albidiferax ferrireducens
Albidovulum
Albidovulum inexpectatum
Alcaligenes
Alcaligenes denitrificans
Alcaligenes faecalis
Alcanivorax
Alcanivorax borkumensis
Alcanivorax jadensis
Algicola
Algicola bacteriolytica
Alicyclobacillus
Alicyclobacillus disulfidooxidans
Alicyclobacillus sendaiensis
Alicyclobacillus vulcanalis
Alishewanella
Alishewanella fetalis
Alkalibacillus
Alkalibacillus haloalkaliphilus
Alkalilimnicola
Alkalilimnicola ehrlichii
Alkaliphilus
Alkaliphilus oremlandii
Alkaliphilus transvaalensis
Allochromatium
Allochromatium vinosum
Allolococcus
Alloiococcus otitis
Allokutzneria
Allokutzneria albata
Altererythrobacter
Altererythrobacter ishigakiensis
Altermonas
Altermonas haloplanktis
Altermonas macleodii
Alysiella
Alysiella crassa
Alysiella filiformis
Aminobacter
Aminobacter aganoensis
Aminobacter aminovorans
Aminobacter niigataensis
Aminobacterium
Aminobacterium mobile
Aminomonas
Aminomonas paucivorans
Ammoniphilus
Ammoniphilus oxalaticus
Ammoniphilus oxalivorans
Amphibacillus
Amphibacillus xylanus
Amphritea
Amphritea balenae
Amphritea japonica
Amycolatopsis
Amycolatopsis alba
Amycolatopsis albidoflavus
Amycolatopsis azurea
Amycolatopsis coloradensis
Amycolatopsis lurida
Amycolatopsis mediterranei
Amycolatopsis rifamycinica
Amycolatopsis rubida
Amycolatopsis sulphurea
Amycolatopsis tolypomycina
Anabaena
Anabaena cylindrica
Anabaena flos-aquae
Anabaena variabilis
Anaeroarcus
Anaeroarcus burkinensis
Anaerobaculum
Anaerobaculum mobile
Anaerobiospirillum
Anaerobiospirillum succiniciproducens
Anaerobiospirillum thomasii
Anaerococcus
Anaerococcus hydrogenalis
Anaerococcus lactolyticus
Anaerococcus prevotii
Anaerococcus tetradius
Anaerococcus vaginalis
Anaerofustis
Anaerofustis stercorihominis
Anaeromusa
Anaeromusa acidaminophila
Anaeromyxobacter
Anaeromyxobacter dehalogenans
Anaerorhabdus
Anaerorhabdus furcosa
Anaerosinus
Anaerosinus glycerini
Anaerovirgula
Anaerovirgula multivorans
Ancalomicrobium
Ancalomicrobium adetum
Ancylobacter
Ancylobacter aquaticus
Aneurinibacillus
Aneurinibacillus aneurinilyticus
Aneurinibacillus migulanus
Aneurinibacillus thermoaerophilus
Angiococcus
Angiococcus disciformis
Angulomicrobium
Angulomicrobium tetraedrale
Anoxybacillus
Anoxybacillus pushchinoensis
Aquabacterium
Aquabacterium commune
Aquabacterium parvum
Aquaspirillum
Aquaspirillum polymorphum
Aquaspirillum putridiconchylium
Aquaspirillum serpens
Aquimarina
Aquimarina latercula
Arcanobacterium
Arcanobacterium haemolyticum
Arcanobacterium pyogenes
Archangium
Archangium gephyra
Arcobacter
Arcobacter butzleri
Arcobacter cryaerophilus
Arcobacter halophilus
Arcobacter nitrofigilis
Arcobacter skirrowii
Arhodomonas
Arhodomonas aquaeolei
Arsenophonus
Arsenophonus nasoniae
Arthrobacter
Arthrobacter agilis
Arthrobacter albus
Arthrobacter aurescens
Arthrobacter chlorophenolicus
Arthrobacter citreus
Arthrobacter crystallopoietes
Arthrobacter cumminsii
Arthrobacter globiformis
Arthrobacter histidinolovorans
Arthrobacter ilicis
Arthrobacter luteus
Arthrobacter methylotrophus
Arthrobacter mysorens
Arthrobacter nicotianae
Arthrobacter nicotinovorans
Arthrobacter oxydans
Arthrobacter pascens
Arthrobacter phenanthrenivorans
Arthrobacter polychromogenes
Atrhrobacter protophormiae
Arthrobacter psychrolactophilus
Arthrobacter ramosus
Arthrobacter sulfonivorans
Arthrobacter sulfureus
Arthrobacter uratoxydans
Arthrobacter ureafaciens
Arthrobacter viscosus
Arthrobacter woluwensis
Asaia
Asaia bogorensis
Asanoa
Asanoa ferruginea
Asticcacaulis
Asticcacaulis biprosthecium
Asticcacaulis excentricus
Atopobacter
Atopobacter phocae
Atopobium
Atopobium fossor
Atopobium minutum
Atopobium parvulum
Atopobium rimae
Atopobium vaginae
Aureobacterium
Aureobacterium barkeri
Aurobacterium
Aurobacterium liquefaciens
Avibacterium
Avibacterium avium
Avibacterium gallinarum
Avibacterium paragallinarum
Avibacterium volantium
Azoarcus
Azoarcus indigens
Azoarcus tolulyticus
Azoarcus toluvorans
Azohydromonas
Azohydromonas australica
Azohydromonas lata
Azomonas
Azomonas agilis
Azomonas insignis
Azomonas macrocytogenes
Azorhizobium
Azorhizobium caulinodans
Azorhizophilus
Azorhizophilus paspali
Azospirillum
Azospirillum brasilense
Azospirillum halopraeferens
Azospirillum irakense
Azotobacter
Azotobacter beijerinckii
Azotobacter chroococcum
Azotobacter nigricans
Azotobacter salinestris
Azotobacter vinelandii
Bacteriovorax
Bacteriovorax stolpii
Bacteroides
Bacteroides caccae
Bacteroides coagulans
Bacteroides eggerthii
Bacteroides fragilis
Bacteroides galacturonicus
Bacteroides helcogenes
Bacteroides ovatus
Bacteroides pectinophilus
Bacteroides pyogenes
Bacteroides salyersiae
Bacteroides stercoris
Bacteroides suis
Bacteroides tectus
Bacteroides thetaiotaomicron
Bacteroides uniformis
Bacteroides ureolyticus
Bacteroides vulgatus
Balnearium
Balnearium lithotrophicum
Balneatrix
Balneatrix alpica
Balneola
Balneola vulgaris
Barnesiella
Barnesiella viscericola
Bartonella
Bartonella alsatica
Bartonella bacilliformis
Bartonella clarridgeiae
Bartonella doshiae
Bartonella elizabethae
Bartonella grahamii
Bartonella henselae
Bartonella rochalimae
Bartonella vinsonii
Bavariicoccus
Bavariicoccus seileri
Bdellovibrio
Bdellovibrio bacteriovorus
Bdellovibrio exovorus
Beggiatoa
Beggiatoa alba
Beijerinckia
Beijerinckia derxii
Beijerinckia fluminensis
Beijerinckia indica
Beijerinckia mobilis
Belliella
Belliella baltica
Bellilinea
Bellilinea caldifistulae
Belnapia
Belnapia moabensis
Bergeriella
Bergeriella denitrificans
Beutenbergia
Beutenbergia cavernae
Bibersteinia
Bibersteinia trehalosi
Bifidobacterium
Bifidobacterium adolescentis
Bifidobacterium angulatum
Bifidobacterium animalis
Bifidobacterium asteroides
Bifidobacterium bifidum
Bifidobacterium boum
Bifidobacterium breve
Bifidobacterium catenulatum
Bifidobacterium choerinum
Bifidobacterium coryneforme
Bifidobacterium cuniculi
Bifidobacterium dentium
Bifidobacterium gallicum
Bifidobacterium gallinarum
Bifidobacterium indicum
Bifidobacterium longum
Bifidobacterium magnumBifidobacterium merycicum
Bifidobacterium minimum
Bifidobacterium pseudocatenulatum
Bifidobacterium pseudolongum
Bifidobacterium pullorum
Bifidobacterium ruminantium
Bifidobacterium saeculare
Bifidobacterium subtile
Bifidobacterium thermophilum
Bilophila
Bilophila wadsworthia
Biostraticola
Biostraticola tofi
Bizionia
Bizionia argentinensis
Blastobacter
Blastobacter capsulatus
Blastobacter denitrificans
Blastococcus
Blastococcus aggregatus
Blastococcus saxobsidens
Blastochloris
Blastochloris viridis
Blastomonas
Blastomonas natatoria
Blastopirellula
Blastopirellula marina
Blautia
Blautia coccoides
Blautia hansenii
Blautia producta
Blautia wexlerae
Bogoriella
Bogoriella caseilytica
Bordetella
Bordetella avium
Bordetella bronchiseptica
Bordetella hinzii
Bordetella holmesii
Bordetella parapertussis
Bordetella pertussis
Bordetella petrii
Bordetella trematum
Borrelia
Borrelia afzelii
Borrelia americana
Borrelia burgdorferi
Borrelia carolinensis
Borrelia coriaceae
Borrelia garinii
Borrelia japonica
Bosea
Bosea minatitlanensis
Bosea thiooxidans
Brachybacterium
Brachybacterium alimentarium
Brachybacterium faecium
Brachybacterium paraconglomeratum
Brachybacterium rhamnosum
Brachybacterium tyrofermentans
Brachyspira
Brachyspira alvinipulli
Brachyspira hyodysenteriae
Brachyspira innocens
Brachyspira murdochii
Brachyspira pilosicoli
Bradyrhizobium
Bradyrhizobium canariense
Bradyrhizobium elkanii
Bradyrhizobium japonicum
Bradyrhizobium liaoningense
Brenneria
Brenneria alni
Brenneria nigrifluens
Brenneria quercina
Brenneria quercina
Brenneria salicis
Brevibacillus
Brevibacillus agri
Brevibacillus borstelensis
Brevibacillus brevis
Brevibacillus centrosporus
Brevibacillus choshinensis
Brevibacillus invocatus
Brevibacillus laterosporus
Brevibacillus parabrevis
Brevibacillus reuszeri
Brevibacterium
Brevibacterium abidum
Brevibacterium album
Brevibacterium aurantiacum
Brevibacterium celere
Brevibacterium epidermidis
Brevibacterium frigoritolerans
Brevibacterium halotolerans
Brevibacterium iodinum
Brevibacterium linens
Brevibacterium lyticum
Brevibacterium mcbrellneri
Brevibacterium otitidis
Brevibacterium oxydans
Brevibacterium paucivorans
Brevibacterium stationis
Brevinema
Brevinema andersonii
Brevundimonas
Brevundimonas alba
Brevundimonas aurantiaca
Brevundimonas diminuta
Brevundimonas intermedia
Brevundimonas subvibrioides
Brevundimonas vancanneytil
Brevundimonas variabilis
Brevundimonas vesicularis
Brochothrix
Brochothrix campestris
Brochothrix thermosphacta
Brucella
Brucella canis
Brucella neotomae
Bryobacter
Bryobacter aggregatus
Burkholderia
Burkholderia ambifaria
Burkholderia andropogonis
Burkholderia anthina
Burkholderia caledonica
Burkholderia caryophylli
Burkholderia cenocepacia
Burkholderia cepacia
Burkholderia cocovenenans
Burkholderia dolosa
Burkholderia fungorum
Burkholderia glathei
Burkholderia glumae
Burkholderia graminis
Burkholderia kururiensis
Burkholderia multivorans
Burkholderia phenazinium
Burkholderia plantarii
Burkholderia pyrrocinia
Burkholderia silvatlantica
Burkholderia stabilis
Burkholderia thailandensis
Burkholderia tropica
Burkholderia unamae
Burkholderia vietnamiensis
Buttiauxella
Buttiauxella agrestis
Buttiauxella brennerae
Buttiauxella ferragutiae
Buttiauxella gaviniae
Buttiauxella izardii
Buttiauxella noackiae
Buttiauxella warmboldiae
Butyrivibrio
Butyrivibrio fibrisolvens
Butyrivibrio hungatei
Butyrivibrio proteoclasticus
Bacillus
B. acidiceler
B. acidicola
B. acidiproducens
B. acidocaldarius
B. acidoterrestris
B. aeolius
B. aerius
B. aerophilus
B. agaradhaerens
B. agri
B. aidingensis
B. akibai
B. alcalophilus
B. algicola
B. alginolyticus
B. alkalidiazotrophicus
B. alkalinitrilicus
B. alkalisediminis
B. alkalitelluris
B. altitudinis
B. alveayuensis
B. alvei
B. amyloliquefaciens
B. a. subsp. Amyloliquefaciens
B. a. subsp. Plantarum
B. dipsosauri
B. drentensis
B. edaphicus
B. ehimensis
B. eiseniae
B. enclensis
B. endophyticus
B. endoradicis
B. farraginis
B. fastidiosus
B. fengqiuensis
B. firmus
B. flexus
B. foraminis
B. fordii
B. formosus
B. fortis
B. fumarioli
B. funiculus
B. fusiformis
B. galactophilus
B. galactosidilyticus
B. galliciensis
B. gelatini
B. gibsonii
B. ginsengi
B. ginsengihumi
B. ginsengisoli
B. globisporus (eg, B. g. subsp. Globisporus; or B. g. subsp. Marinus)
B. aminovorans
B. amylolyticus
B. andreesenii
B. aneurinilyticus
B. anthracis
B. aquimaris
B. arenosi
B. arseniciselenatis
B. arsenicus
B. aurantiacus
B. arvi
B. aryabhattai
B. asahii
B. atrophaeus
B. axarquiensis
B. azotofixans
B. azotoformans
B. badius
B. barbaricus
B. bataviensis
B. beijingensis
B. benzoevorans
B. beringensis
B. berkeleyi
B. beveridgei
B. bogoriensis
B. boroniphilus
B. borstelensis
B. brevis Migula
B. butanolivorans
B. canaveralius
B. carboniphilus
B. cecembensis
B. cellulosilyticus
B. centrosporus
B. cereus
B. chagannorensis
B. chitinolyticus
B. chondroitinus
B. choshinensis
B. chungangensis
B. cibi
B. circulans
B. clarkii
B. clausii
B. coagulans
B. coahuilensis
B. cohnii
B. composti
B. curdlanolyticus
B. cycloheptanicus
B. cytotoxicus
B. daliensis
B. decisifrondis
B. decolorationis
B. deserti
B. glucanolyticus
B. gordonae
B. gottheilif
B. graminis
B. halmapalus
B. haloalkaliphilus
B. halochares
B. halodenitrificans
B. halodurans
B. halophilus
B. halosaccharovorans
B. hemicellulosilyticus
B. hemicentroti
B. herbersteinensis
B. horikoshii
B. horneckiae
B. horti
B. huizhouensis
B. humi
B. hwajinpoensis
B. idriensis
B. indicus
B. infantis
B. infernus
B. insolitus
B. invictae
B. iranensis
B. isabellae
B. isronensis
B. jeotgali
B. kaustophilus
B. kobensis
B. kochii
B. kokeshiiformis
B. koreensis
B. korlensis
B. kribbensis
B. krulwichiae
B. laevolacticus
B. larvae
B. laterosporus
B. salexigens
B. saliphilus
B. schlegelii
B. sediminis
B. selenatarsenatis
B. selenitireducens
B. seohaeanensis
B. shacheensis
B. shackletonii
B. siamensis
B. silvestris
B. simplex
B. siralis
B. smithii
B. soli
B. solimangrovi
B. solisalsi
B. songklensis
B. sonorensis
B. sphaericus
B. sporothermodurans
B. stearothermophilus
B. stratosphericus
B. subterraneus
B. subtilis (eg, B. s. subsp. Inaquosorum; or B. s. subsp. Spizizeni;
or B. s. subsp. Subtilis)
B. taeanensis
B. tequilensis
B. thermantarcticus
B. thermoaerophilus
B. thermoamylovorans
B. thermocatenulatus
B. thermocloacae
B. thermocopriae
B. thermodenitrificans
B. thermoglucosidasius
B. thermolactis
B. thermoleovorans
B. thermophilus
B. thermoruber
B. thermosphaericus
B. thiaminolyticus
B. thioparans
B. thuringiensis
B. tianshenii
B. trypoxylicola
B. tusciae
B. validus.
B. vallismortis
B. vedderi
B. velezensis
B. vietnamensis
B. vireti
B. vulcani
B. wakoensis
B. weihenstephanensis
B. xiamenensis
B. xiaoxiensis
B. zhanjiangensis
B. peoriae
B. persepolensis
B. persicus
B. pervagus
B. plakortidis
B. pocheonensis
B. polygoni
B. polymyxa
B. popilliae
B. pseudalcalophilus
B. pseudofirmus
B. pseudomycoides
B. psychrodurans
B. psychrophilus
B. psychrosaccharolyticus
B. psychrotolerans
B. pulvifaciens
B. pumilus
B. purgationiresistens
B. pycnus
B. qingdaonensis
B. qingshengii
B. reuszeri
B. rhizosphaerae
B. rigui
B. ruris
B. safensis
B. salarius
B. lautus
B. lehensis
B. lentimorbus
B. lentus
B. licheniformis
B. ligniniphilus
B. litoralis
B. locisalis
B. luciferensis
B. luteolus
B. luteus
B. macauensis
B. macerans
B. macquariensis
B. macyae
B. malacitensis
B. mannanilyticus
B. marisflavi
B. marismortui
B. marmarensis
B. massiliensis
B. megaterium
B. mesonae
B. methanolicus
B. methylotrophicus
B. migulanus
B. mojavensis
B. mucilaginosus
B. muralis
B. murimartini
B. mycoides
B. naganoensis
B. nanhaiensis
B. nanhaiisediminis
B. nealsonii
B. neidei
B. neizhouensis
B. niabensis
B. niacini
B. novalis
B. oceanisediminis
B. odysseyi
B. okhensis
B. okuhidensis
B. oleronius
B. oryzaecorticis
B. oshimensis
B. pabuli
B. pakistanensis
B. pallidus
B. pallidus
B. panacisoli
B. panaciterrae
B. pantothenticus
B. parabrevis
B. paraflexus
B. pasteurii
B. patagonensis
Caenimonas
Caenononas koreensis
Caldalkalibacillus
Caldalkalibacillus uzonensis
Caldanaerobacter
Caldanaerobacter subterraneus
Caldanaerobius
Caldanaerobius fijiensis
Caldanaerobius polysaccharolyticus
Caldanaerobius zeae
Caldanaerovirga
Caldanaerovirga acetigignens
Caldicellulosiruptor
Caldicellulosiruptor bescii
Caldicellulosiruptor kristjanssonii
Caldicellulosiruptor owensensis
Campylobacter
Campylobacter coli
Campylobacter concisus
Campylobacter curvus
Campylobacter fetus
Campylobacter gracilis
Campylobacter helveticus
Campylobacter hominis
Campylobacter hyointestinalis
Campylobacter jejuni
Campylobacter lari
Campylobacter mucosalis
Campylobacter rectus
Campylobacter showae
Campylobacter sputorum
Campylobacter upsaliensis
Capnocytophaga
Capnocytophaga canimorsus
Capnocytophaga cynodegmi
Capnocytophaga gingivalis
Capnocytophaga granulosa
Capnocytophaga haemolytica
Capnocytophaga ochracea
Capnocytophaga sputigena
Cardiobacterium
Cardiobacterium hominis
Carnimonas
Carnimonas nigrificans
Carnobacterium
Carnobacterium alterfunditum
Carnobacterium divergens
Carnobacterium funditum
Carnobacterium gallinarum
Carnobacterium maltaromaticum
Carnobacterium mobile
Carnobacterium viridans
Caryophanon
Caryophanon latum
Caryophanon tenue
Catellatospora
Catellatospora citrea
Catellatospora methionotrophica
Catenococcus
Catenococcus thiocycli
Catenuloplanes
Catenuloplanes atrovinosus
Catenuloplanes castaneus
Catenuloplanes crispus
Catenuloplanes indicus
Catenuloplanes japonicus
Catenuloplanes nepalensis
Catenuloplanes niger
Chryseobacterium
Chryseobacterium balustinum
Citrobacter
C. amalonaticus
C. braakii
C. diversus
C. farmeri
C. freundii
C. gillenii
C. koseri
C. murliniae
C. pasteurii
C. rodentium
C. sedlakii
C. werkmanii
C. youngae
Coccochloris
Coccochloris elabens
Corynebacterium
Corynebacterium flavescens
Corynebacterium variabile
Curtobacterium
Curtobacterium albidum
Curtobacterium citreus
Clostridium
Clostridium absonum,
Clostridium aceticum,
Clostridium acetireducens,
Clostridium acetobutylicum,
Clostridium acidisoli,
Clostridium aciditolerans,
Clostridium acidurici,
Clostridium aerotolerans,
Clostridium aestuarii,
Clostridium akagii,
Clostridium aldenense,
Clostridium aldrichii,
Clostridium algidicarni,
Clostridium algidixylanolyticum,
Clostridium algifaecis,
Clostridium algoriphilum,
Clostridium alkalicellulosi,
Clostridium aminophilum,
Clostridium aminovalericum,
Clostridium amygdalinum,
Clostridium amylolyticum,
Clostridium arbusti,
Clostridium arcticum,
Clostridium argentinense,
Clostridium asparagiforme,
Clostridium aurantibutyricum,
Clostridium autoethanogenum,
Clostridium baratii,
Clostridium barkeri,
Clostridium bartlettii,
Clostridium beijerinckii,
Clostridium bifermentans,
Clostridium bolteae,
Clostridium bornimense,
Clostridium botulinum,
Clostridium bowmanii,
Clostridium bryantii,
Clostridium butyricum,
Clostridium cadaveris,
Clostridium caenicola,
Clostridium caminithermale,
Clostridium carboxidivorans,
Clostridium carnis,
Clostridium cavendishii,
Clostridium celatum,
Clostridium celerecrescens,
Clostridium cellobioparum,
Clostridium cellulofermentans,
Clostridium cellulolyticum,
Clostridium cellulosi,
Clostridium cellulovorans,
Clostridium chartatabidum,
Clostridium chauvoei,
Clostridium chromiireducens,
Clostridium citroniae,
Clostridium clariflavum,
Clostridium clostridioforme,
Clostridium coccoides,
Clostridium cochlearium,
Clostridium colletant,
Clostridium colicanis,
Clostridium colinum,
Clostridium collagenovorans,
Clostridium cylindrosporum,
Clostridium difficile,
Clostridium diolis,
Clostridium disporicum,
Clostridium drakei,
Clostridium durum,
Clostridium estertheticum,
Clostridium estertheticum
estertheticum, Clostridium
estertheticum laramiense,
Clostridium fallax,
Clostridium felsineum,
Clostridium fervidum,
Clostridium fimetarium,
Clostridium formicaceticum,
Clostridium frigidicarnis,
Clostridium frigoris,
Clostridium ganghwense,
Clostridium gasigenes,
Clostridium ghonii,
Clostridium glycolicum,
Clostridium glycyrrhizinilyticum,
Clostridium grantii,
Clostridium haemolyticum,
Clostridium halophilum,
Clostridium hastiforme,
Clostridium hathewayi,
Clostridium herbivorans,
Clostridium hiranonis,
Clostridium histolyticum,
Clostridium homopropionicum,
Clostridium huakuii,
Clostridium hungatei,
Clostridium hydrogeniformans,
Clostridium hydroxybenzoicum,
Clostridium hylemonae,
Clostridium jejuense,
Clostridium indolis,
Clostridium innocuum,
Clostridium intestinale,
Clostridium irregulare,
Clostridium isatidis,
Clostridium josui,
Clostridium kluyveri,
Clostridium lactatifermentans,
Clostridium lacusfryxellense,
Clostridium laramiense,
Clostridium lavalense,
Clostridium lentocellum,
Clostridium lentoputrescens,
Clostridium leptum,
Clostridium limosum,
Clostridium litorale,
Clostridium lituseburense,
Clostridium ljungdahlii,
Clostridium lortetii,
Clostridium lundense,
Clostridium magnum,
Clostridium malenominatum,
Clostridium mangenotii,
Clostridium mayombei,
Clostridium methoxybenzovorans,
Clostridium methylpentosum,
Clostridium neopropionicum,
Clostridium nexile,
Clostridium nitrophenolicum,
Clostridium novyi,
Clostridium oceanicum,
Clostridium orbiscindens,
Clostridium oroticum,
Clostridium oxalicum,
Clostridium papyrosolvens,
Clostridium paradoxum,
Clostridium paraperfringens
(Alias: C. welchii),
Clostridium paraputrificum,
Clostridium pascui,
Clostridium pasteurianum,
Clostridium peptidivorans,
Clostridium perenne,
Clostridium perfringens,
Clostridium pfennigii,
Clostridium phytofermentans,
Clostridium piliforme,
Clostridium polysaccharolyticum,
Clostridium populeti,
Clostridium propionicum,
Clostridium proteoclasticum,
Clostridium proteolyticum,
Clostridium psychrophilum,
Clostridium puniceum,
Clostridium purinilyticum,
Clostridium putrefaciens,
Clostridium putrificum,
Clostridium quercicolum,
Clostridium quinii,
Clostridium ramosum,
Clostridium rectum,
Clostridium roseum,
Clostridium saccharobutylicum,
Clostridium saccharogumia,
Clostridium saccharolyticum,
Clostridium saccharoperbutylacetonicum
Clostridium sardiniense,
Clostridium sartagoforme,
Clostridium scatologenes,
Clostridium schirmacherense,
Clostridium scindens,
Clostridium septicum,
Clostridium sordellii,
Clostridium sphenoides,
Clostridium spiroforme,
Clostridium sporogenes,
Clostridium sporosphaeroides,
Clostridium stercorarium,
Clostridium stercorarium leptospartum,
Clostridium stercorarium stercorarium,
Clostridium stercorarium thermolacticum,
Clostridium sticklandii,
Clostridium straminisolvens,
Clostridium subterminale,
Clostridium sufflavum,
Clostridium sulfidigenes,
Clostridium symbiosum,
Clostridium tagluense,
Clostridium tepidiprofundi,
Clostridium termitidis,
Clostridium tertium,
Clostridium tetani,
Clostridium tetanomorphum,
Clostridium thermaceticum,
Clostridium thermautotrophicum,
Clostridium thermoalcaliphilum,
Clostridium thermobutyricum,
Clostridium thermocellum,
Clostridium thermocopriae,
Clostridium thermohydrosulfuricum,
Clostridium thermolacticum,
Clostridium thermopalmarium,
Clostridium thermopapyrolyticum,
Clostridium thermosaccharolyticum,
Clostridium thermosuccinogenes,
Clostridium thermosulfurigenes,
Clostridium thiosulfatireducens,
Clostridium tyrobutyricum,
Clostridium uliginosum,
Clostridium ultunense,
Clostridium villosum,
Clostridium vincentii,
Clostridium viride,
Clostridium xylanolyticum,
Clostridium xylanovorans
Dactylosporangium
Dactylosporangium aurantiacum
Dactylosporangium fulvum
Dactylosporangium matsuzakiense
Dactylosporangium roseum
Dactylosporangium thailandense
Dactylosporangium vinaceum
Deinococcus
Deinococcus aerius
Deinococcus apachensis
Deinococcus aquaticus
Deinococcus aquatilis
Deinococcus caeni
Deinococcus radiodurans
Deinococcus radiophilus
Delftia
Delftia acidovorans
Desulfovibrio
Desulfovibrio desulfuricans
Diplococcus
Diplococcus pneumoniae
Echinicola
Echinicola pacifica
Echinicola vietnamensis
Enterobacter
E. aerogenes
E. amnigenus
E. agglomerans
E. arachidis
E. asburiae
E. cancerogenous
E. cloacae
E. cowanii
E. dissolvens
E. gergoviae
E. helveticus
E. hormaechei
E. intermedius
Enterobacter kobei
E. ludwigii
E. mori
E. nimipressuralis
E. oryzae
E. pulveris
E. pyrinus
E. radicincitans
E. taylorae
E. turicensis
E. sakazakii Enterobacter soli
Enterococcus
Enterococcus durans
Enterococcus faecalis
Enterococcus faecium
Erwinia
Erwinia hapontici
Escherichia
Escherichia coli
Faecalibacterium
Faecalibacterium prausnitzii
Fangia
Fangia hongkongensis
Fastidiosipila
Fastidiosipila sanguinis
Fusobacterium
Fusobacterium nucleatum
Flavobacterium
Flavobacterium antarcticum
Flavobacterium aquatile
Flavobacterium aquidurense
Flavobacterium balustinum
Flavobacterium croceum
Flavobacterium cucumis
Flavobacterium daejeonense
Flavobacterium defluvii
Flavobacterium degerlachei
Flavobacterium denitrificans
Flavobacterium filum
Flavobacterium flevense
Flavobacterium frigidarium
Flavobacterium mizutaii
Flavobacterium okeanokoites
Gaetbulibacter
Gaetbulibacter saemankumensis
Gallibacterium
Gallibacterium anatis
Gallicola
Gallicola barnesae
Garciella
Garciella nitratireducens
Geobacillus
Geobacillus thermoglucosidasius
Geobacillus stearothermophilus
Geobacter
Geobacter bemidjiensis
Geobacter bremensis
Geobacter chapellei
Geobacter grbiciae
Geobacter hydrogenophilus
Geobacter lovleyi
Geobacter metallireducens
Geobacter pelophilus
Geobacter pickeringil
Geobacter sulfurreducens
Geodermatophilus
Geodermatophilus obscurus
Gluconacetobacter
Gluconacetobacter xylinus
Gordonia
Gordonia rubripertincta
Haemophilus
Haemophilus aegyptius
Haemophilus aphrophilus
Haemophilus felis
Haemophilus gallinarum
Haemophilus haemolyticus
Haemophilus influenzae
Haemophilus paracuniculus
Haemophilus parahaemolyticus
Haemophilus parainfluenzae
Haemophilus paraphrohaemolyticus
Haemophilus parasuis
Haemophilus pittmaniae
Hafnia
Hafnia alvei
Hahella
Hahella ganghwensis
Halalkalibacillus
Halalkalibacillus halophilus
Helicobacter
Helicobacter pylori
Ideonella
Ideonella azotifigens
Idiomarina
Idiomarina abyssalis
Idiomarina baltica
Idiomarina fontislapidosi
Idiomarina loihiensis
Idiomarina ramblicola
Idiomarina seosinensis
Idiomarina zobellii
Ignatzschineria
Ignatzschineria larvae
Ignavigranum
Ignavigranum ruoffiae
Ilumatobacter
Ilumatobacter fluminis
Ilyobacter
Ilyobacter delafieldii
Ilyobacter insuetus
Ilyobacter polytropus
Ilyobacter tartaricus
Janibacter
Janibacter anophelis
Janibacter corallicola
Janibacter limosus
Janibacter melonis
Janibacter terrae
Jannaschia
Jannaschia cystaugens
Jannaschia helgolandensis
Jannaschia pohangensis
Jannaschia rubra
Janthinobacterium
Janthinobacterium agaricidamnosum
Janthinobacterium lividum
Jejuia
Jejuia pallidilutea
Jeotgalibacillus
Jeotgalibacillus alimentarius
Jeotgalicoccus
Jeotgalicoccus halotolerans
Kaistia
Kaistia adipata
Kaistia soli
Kangiella
Kangiella aquimarina
Kangiella koreensis
Kerstersia
Kerstersia gyiorum
Kiloniella
Kiloniella laminariae
Klebsiella
K. granulomatis
K. oxytoca
K. pneumoniae
K. terrigena
K. variicola
Kluyvera
Kluyvera ascorbata
Kocuria
Kocuria roasea
Kocuria varians
Kurthia
Kurthia zopfii
Labedella
Labedella gwakjiensis
Labrenzia
Labrenzia aggregata
Labrenzia alba
Labrenzia alexandrii
Labrenzia marina
Labrys
Labrys methylaminiphilus
Labrys miyagiensis
Labrys monachus
Labrys okinawensis
Labrys portucalensis
Laceyella
Laceyella putida
Lechevalieria
Lechevalieria aerocolonigenes
Listeria
L. aquatica
L. booriae
L. cornellensis
L. fleischmannii
L. floridensis
L. grandensis
L. grayi
L. innocua
Listeria ivanovii
L. marthii
L. monocytogenes
L. newyorkensis
L. riparia
L. rocourtiae
L. seeligeri
L. weihenstephanensis
L. welshimeri
Listonella
Listonella anguillarum
Macrococcus
Macrococcus bovicus
Marinobacter
Marinobacter algicola
Marinobacter bryozoorum
Marinobacter flavimaris
Meiothermus
Meiothermus ruber
Methylophilus
Methylophilus methylotrophus
Microbacterium
Microbacterium ammoniaphilum
Microbacterium arborescens
Microbacterium liquefaciens
Microbacterium oxydans
Micrococcus
Micrococcus luteus
Micrococcus lylae
Moraxella
Moraxella bovis
Moraxella nonliquefaciens
Moraxella osloensis
Nakamurella
Nakamurella mulipartita
Nannocystis
Nannocystis pusilla
Natranaerobius
Natranaerobius thermophilus
Natranaerobius trueperi
Naxibacter
Naxibacter alkalitolerans
Neisseria
Neisseria cinerea
Neisseria denitrificans
Neisseria gonorrhoeae
Neisseria lactamica
Neisseria mucosa
Neisseria sicca
Neisseria subflava
Neptunomonas
Neptunomonas japonica
Nesterenkonia
Nesterenkonia holobia
Nocardia
Nocardia argentinensis
Nocardia corallina
Nocardia otitidiscaviarum
Lactobacillus
L. acetotolerans
L. acidifarinae
L. acidipiscis
L. acidophilus
Lactobacillus agilis
L. algidus
L. alimentarius
L. amylolyticus
L. amylophilus
L. amylotrophicus
L. amylovorus
L. animalis
L. antri
L. apodemi
L. aviarius
L. bifermentans
L. brevis
L. buchneri
L. camelliae
L. casei
L. kitasatonis
L. kunkeei
L. leichmannii
L. lindneri
L. malefermentans
L. catenaformis
L. ceti
L. coleohominis
L. collinoides
L. composti
L. concavus
L. coryniformis
L. crispatus
L. crustorum
L. curvatus
L. delbrueckii subsp. Bulgaricus
L. delbrueckii subsp. Delbrueckii
L. delbrueckii subsp. Lactis
L. dextrinicus
L. diolivorans
L. equi
L. equigenerosi
L. farraginis
L. farciminis
L. fermentum
L. fornicalis
L. fructivorans
L. frumenti
L. mali
L. manihotivorans
L. mindensis
L. mucosae
L. murinus
L. nagelii
L. namurensis
L. nantensis
L. oligofermentans
L. oris
L. panis
L. pantheris
L. parabrevis
L. parabuchneri
L. paracasei
L. paracollinoides
L. parafarraginis
L. homohiochii
L. iners
L. ingluviei
L. intestinalis
L. fuchuensis
L. gallinarum
L. gasseri
L. parakefiri
L. paralimentarius
L. paraplantarum
L. pentosus
L. perolens
L. plantarum
L. pontis
L. protectus
L. psittaci
L. rennini
L. reuteri
L. rhamnosus
L. rimae
L. rogosae
L. rossiae
L. ruminis
L. saerimneri
L. jensenii
L. johnsonii
L. kalixensis
L. kefiranofaciens
L. kefiri
L. aviarius
L. helveticus
L. hilgardii
L. sakei
L. salivarius
L. sanfranciscensis
L. satsumensis
L. secaliphilus
L. sharpeae
L. siliginis
L. spicheri
L. suebicus
L. thailandensis
L. ultunensis
L. vaccinostercus
L. vaginalis
L. versmoldensis
L. vini
L. vitulinus
L. zeae
L. zymae
L. gastricus
L. ghanensis
L. graminis
L. hammesii
L. hamsteri
L. harbinensis
L. hayakitensis
Legionella
Legionella adelaidensis
Legionella anisa
Legionella beliardensis
Legionella birminghamensis
Legionella bozemanae
Legionella brunensis
Legionella busanensis
Legionella cardiaca
Legionella cherrii
Legionella cincinnatiensis
Legionella clemsonensis
Legionella donaldsonii
Legionella drancourtii
Legionella dresdenensis
Legionella drozanskii
Legionella dumoffii
Legionella erythra
Legionella fairfieldensis
Legionella fallonii
Legionella feeleii
Legionella geestiana
Legionella genomospecies
Legionella gormanii
Legionella gratiana
Legionella gresilensis
Legionella hackeliae
Legionella impletisoli
Legionella israelensis
Legionella jamestowniensis
Candidatus Legionella jeonii
Legionella jordanis
Legionella lansingensis
Legionella londiniensis
Legionella longbeachae
Legionella lytica
Legionella maceachernii
Legionella massiliensis
Legionella micdadei
Legionella monrovica
Legionella moravica
Legionella nagasakiensis
Legionella nautarum
Legionella norrlandica
Legionella oakridgensis
Legionella parisiensis
Legionella pittsburghensis
Legionella pneumophila
Legionella quateirensis
Legionella quinlivanii
Legionella rowbothamii
Legionella rubrilucens
Legionella sainthelensi
Legionella santicrucis
Legionella shakespearei
Legionella spiritensis
Legionella steelei
Legionella steigerwaltii
Legionella taurinensis
Legionella tucsonensis
Legionella tunisiensis
Legionella wadsworthii
Legionella waltersii
Legionella worsleiensis
Legionella yabuuchiae
Oceanibulbus
Oceanibulbus indolifex
Oceanicaulis
Oceanicaulis alexandrii
Oceanicola
Oceanicola batsensis
Oceanicola granulosus
Oceanicola nanhaiensis
Oceanimonas
Oceanimonas baumannii
Oceaniserpentilla
Oceaniserpentilla haliotis
Oceanisphaera
Oceanisphaera donghaensis
Oceanisphaera litoralis
Oceanithermus
Oceanithermus desulfurans
Oceanithermus profundus
Oceanobacillus
Oceanobacillus caeni
Oceanospirillum
Oceanospirillum linum
Paenibacillus
Paenibacillus thiaminolyticus
Pantoea
Pantoea agglomerans
Paracoccus
Paracoccus alcaliphilus
Paucimonas
Paucimonas lemoignei
Pectobacterium
Pectobacterium aroidearum
Pectobacterium atrosepticum
Pectobacterium betavasculorum
Pectobacterium cacticida
Pectobacterium carnegieana
Pectobacterium carotovorum
Pectobacterium chrysanthemi
Pectobacterium cypripedii
Pectobacterium rhapontici
Pectobacterium wasabiae
Planococcus
Planococcus citreus
Planomicrobium
Planomicrobium okeanokoites
Plesiomonas
Plesiomonas shigelloides
Proteus
Proteus vulgaris
Prevotella
Prevotella albensis
Prevotella amnii
Prevotella bergensis
Prevotella bivia
Prevotella brevis
Prevotella bryantii
Prevotella buccae
Prevotella buccalis
Prevotella copri
Prevotella dentalis
Prevotella denticola
Prevotella disiens
Prevotella histicola
Prevotella intermedia
Prevotella maculosa
Prevotella marshii
Prevotella melaninogenica
Prevotella micans
Prevotella multiformis
Prevotella nigrescens
Prevotella oralis
Prevotella oris
Prevotella oulorum
Prevotella pallens
Prevotella salivae
Prevotella stercorea
Prevotella tannerae
Prevotella timonensis
Prevotella veroralis
Providencia
Providencia stuartii
Pseudomonas
Pseudomonas aeruginosa
Pseudomonas alcaligenes
Pseudomonas anguillispetica
Pseudomonas fluorescens
Pseudoalteromonas haloplanktis
Pseudomonas mendocina
Pseudomonas
pseudoalcaligenes
Pseudomonas putida
Pseudomonas tutzeri
Pseudomonas syringae
Psychrobacter
Psychrobacter faecalis
Psychrobacter phenylpyruvicus
Quadrisphaera
Quadrisphaera granulorum
Quatrionicoccus
Quatrionicoccus australiensis
Quinella
Quinella ovalis
Ralstonia
Ralstonia eutropha
Ralstonia insidiosa
Ralstonia mannitolilytica
Ralstonia pickettil
Ralstonia pseudosolanacearum
Ralstonia syzygii
Ralstonia solanacearum
Ramlibacter
Ramlibacter henchirensis
Ramlibacter tataouinensis
Raoultella
Raoultella ornithinolytica
Raoultella planticola
Raoultella terrigena
Rathayibacter
Rathayibacter caricis
Rathayibacter festucae
Rathayibacter iranicus
Rathayibacter rathayi
Rathayibacter toxicus
Rathayibacter tritici
Rhodobacter
Rhodobacter sphaeroides
Ruegeria
Ruegeria gelatinovorans
Saccharococcus
Saccharococcus thermophilus
Saccharomonospora
Saccharomonospora azurea
Saccharomonospora cyanea
Saccharomonospora viridis
Saccharophagus
Saccharophagus degradans
Saccharopolyspora
Saccharopolyspora erythraea
Saccharopolyspora gregorii
Saccharopolyspora hirsuta
Saccharopolyspora hordei
Saccharopolyspora rectivirgula
Saccharopolyspora spinosa
Saccharopolyspora taberi
Saccharothrix
Saccharothrix australiensis
Saccharothrix coeruleofusca
Saccharothrix espanaensis
Saccharothrix longispora
Saccharothrix mutabilis
Saccharothrix syringae
Saccharothrix tangerinus
Saccharothrix texasensis
Sagittula
Sagittula stellata
Salegentibacter
Salegentibacter salegens
Salimicrobium
Salimicrobium album
Salinibacter
Salinibacter ruber
Salinicoccus
Salinicoccus alkaliphilus
Salinicoccus hispanicus
Salinicoccus roseus
Salinispora
Salinispora arenicola
Salinispora tropica
Salinivibrio
Salinivibrio costicola
Salmonella
Salmonella bongori
Salmonella enterica
Salmonella subterranea
Salmonella typhi
Sanguibacter
Sanguibacter keddieii
Sanguibacter suarezii
Saprospira
Saprospira grandis
Sarcina
Sarcina maxima
Sarcina ventriculi
Sebaldella
Sebaldella termitidis
Serratia
Serratia fonticola
Serratia marcescens
Sphaerotilus
Sphaerotilus natans
Sphingobacterium
Sphingobacterium multivorum
Stenotrophomonas
Stenotrophomonas maltophilia
Streptomyces
Streptomyces achromogenes
Streptomyces cesalbus
Streptomyces cescaepitosus
Streptomyces cesdiastaticus
Streptomyces cesexfoliatus
Streptomyces fimbriatus
Streptomyces fradiae
Streptomyces fulvissimus
Streptomyces griseoruber
Streptomyces griseus
Streptomyces lavendulae
Streptomyces phaeochromogenes
Streptomyces thermodiastaticus
Streptomyces tubercidicus
Tatlockia
Tatlockia maceachernii
Tatlockia micdadei
Tenacibaculum
Tenacibaculum amylolyticum
Tenacibaculum discolor
Tenacibaculum gallaicum
Tenacibaculum lutimaris
Tenacibaculum mesophilum
Tenacibaculum skagerrakense
Tepidanaerobacter
Tepidanaerobacter syntrophicus
Tepidibacter
Tepidibacter formicigenes
Tepidibacter thalassicus
Thermus
Thermus aquaticus
Thermus filiformis
Thermus thermophilus
Staphylococcus
S. arlettae
S. agnetis
S. aureus
S. auricularis
S. capitis
S. caprae
S. carnosus
S. caseolyticus
S. chromogenes
S. cohnii
S. condimenti
S. delphini
S. devriesei
S. epidermidis
S. equorum
S. felis
S. fleurettii
S. gallinarum
S. haemolyticus
S. hominis
S. hyicus
S. intermedius
S. kloosii
S. leei
S. lentus
S. lugdunensis
S. lutrae
S. lyticans
S. massiliensis
S. microti
S. muscae
S. nepalensis
S. pasteuri
S. petrasii
S. pettenkoferi
S. piscifermentans
S. pseudintermedius
S. pseudolugdunensis
S. pulvereri
S. rostri
S. saccharolyticus
S. saprophyticus
S. schleiferi
S. sciuri
S. simiae
S. simulans
S. stepanovicii
S. succinus
S. vitulinus
S. warneri
S. xylosus
Streptococcus
Streptococcus agalactiae
Streptococcus anginosus
Streptococcus bovis
Streptococcus canis
Streptococcus constellatus
Streptococcus downei
Streptococcus dysgalactiae
Streptococcus equines
Streptococcus faecalis
Streptococcus ferus
Streptococcus infantarius
Streptococcus iniae
Streptococcus intermedius
Streptococcus lactarius
Streptococcus milleri
Streptococcus mitis
Streptococcus mutans
Streptococcus oralis
Streptococcus tigurinus
Streptococcus orisratti
Streptococcus parasanguinis
Streptococcus peroris
Streptococcus pneumoniae
Streptococcus pseudopneumoniae
Streptococcus pyogenes
Streptococcus ratti
Streptococcus salivariu
Streptococcus thermophilus
Streptococcus sanguinis
Streptococcus sobrinus
Streptococcus suis
Streptococcus uberis
Streptococcus vestibularis
Streptococcus viridans
Streptococcus zooepidemicus
Uliginosibacterium
Uliginosibacterium gangwonense
Ulvibacter
Ulvibacter litoralis
Umezawaea
Umezawaea angerina
Undibacterium
Undibacterium pigrum
Ureaplasma
Ureaplasma urealyticum
Ureibacillus
Ureibacillus composti
Ureibacillus suwonensis
Ureibacillus terrenus
Ureibacillus thermophilus
Ureibacillus thermosphaericus
Vagococcus
Vagococcus carniphilus
Vagococcus elongatus
Vagococcus fessus
Vagococcus fluvialis
Vagococcus lutrae
Vagococcus salmoninarum
Variovorax
Variovorax boronicumulans
Variovorax dokdonensis
Variovorax paradoxus
Variovorax soli
Veillonella
Veillonella atypica
Veillonella caviae
Veillonella criceti
Veillonella dispar
Veillonella montpellierensis
Veillonella parvula
Veillonella ratti
Veillonella rodentium
Venenivibrio
Venenivibrio stagnispumantis
Verminephrobacter
Verminephrobacter eiseniae
Verrucomicrobium
Verrucomicrobium spinosum
Vibrio
Vibrio aerogenes
Vibrio aestuarianus
Vibrio albensis
Vibrio alginolyticus
Vibrio campbellii
Vibrio cholerae
Vibrio cincinnatiensis
Vibrio coralliilyticus
Vibrio cyclitrophicus
Vibrio diazotrophicus
Vibrio fluvialis
Vibrio furnissii
Vibrio gazogenes
Vibrio halioticoli
Vibrio harveyi
Vibrio ichthyoenteri
Vibrio mediterranei
Vibrio metschnikovil
Vibrio mytili
Vibrio natriegens
Vibrio navarrensis
Vibrio nereis
Vibrio nigripulchritudo
Vibrio ordalii
Vibrio orientalis
Vibrio parahaemolyticus
Vibrio pectenicida
Vibrio penaeicida
Vibrio proteolyticus
Vibrio shilonii
Vibrio splendidus
Vibrio tubiashii
Vibrio vulnificus
Virgibacillus
Virgibacillus halodenitrificans
Virgibacillus pantothenticus
Weissella
Weissella cibaria
Weissella confusa
Weissella halotolerans
Weissella hellenica
Weissella kandleri
Weissella koreensis
Weissella minor
Weissella paramesenteroides
Weissella soll
Weissella thailandensis
Weissella viridescens
Williamsia
Williamsia marianensis
Williamsia maris
Williamsia serinedens
Winogradskyella
Winogradskyella thalassocola
Wolbachia
Wolbachia persica
Wolinella
Wolinella succinogenes
Zobellia
Zobellia galactanivorans
Zobellia uliginosa
Zoogloea
Zoogloea ramigera
Zoogloea resiniphila
Xanthobacter
Xanthobacter agilis
Xanthobacter aminoxidans
Xanthobacter autotrophicus
Xanthobacter flavus
Xanthobacter tagetidis
Xanthobacter viscosus
Xanthomonas
Xanthomonas albilineans
Xanthomonas alfalfae
Xanthomonas arboricola
Xanthomonas axonopodis
Xanthomonas campestris
Xanthomonas citri
Xanthomonas codiaei
Xanthomonas cucurbitae
Xanthomonas euvesicatoria
Xanthomonas fragariae
Xanthomonas fuscans
Xanthomonas gardneri
Xanthomonas hortorum
Xanthomonas hyacinthi
Xanthomonas perforans
Xanthomonas phaseoli
Xanthomonas pisi
Xanthomonas populi
Xanthomonas theicola
Xanthomonas translucens
Xanthomonas vesicatoria
Xylella
Xylella fastidiosa
Xylophilus
Xylophilus ampelinus
Xenophilus
Xenophilus azovorans
Xenorhabdus
Xenorhabdus beddingii
Xenorhabdus bovienii
Xenorhabdus cabanillasii
Xenorhabdus doucetiae
Xenorhabdus griffiniae
Xenorhabdus hominickii
Xenorhabdus koppenhoeferi
Xenorhabdus nematophila
Xenorhabdus poinarii
Xylanibacter
Xylanibacter oryzae
Yangia
Yangia pacifica
Yaniella
Yaniella flava
Yaniella halotolerans
Yeosuana
Yeosuana aromativorans
Yersinia
Yersinia aldovae
Yersinia bercovieri
Yersinia enterocolitica
Yersinia entomophaga
Yersinia frederiksenii
Yersinia intermedia
Yersinia kristensenii
Yersinia mollaretii
Yersinia philomiragia
Yersinia pestis
Yersinia pseudotuberculosis
Yersinia rohdei
Yersinia ruckeri
Yokenella
Yokenella regensburgei
Yonghaparkia
Yonghaparkia alkaliphila
Zavarzinia
Zavarzinia compransoris
Zooshikella
Zooshikella ganghwensis
Zunongwangia
Zunongwangia profunda
Zymobacter
Zymobacter palmae
Zymomonas
Zymomonas mobilis
Zymophilus
Zymophilus paucivorans
Zymophilus raffinosivorans
Zobellella
Zobellella denitrificans
Zobellella taiwanensis
Zeaxanthinibacter
Zeaxanthinibacter enoshimensis
Zhihengliuella
Zhihengliuella halotolerans
Xylanibacterium
Xylanibacterium ulmi

The present invention is described in more detail in the following non-limiting Examples.

### EXAMPLES

### Example 1. IAA Biosynthetic Pathway on Plasmid

### 1.1 Cloning of the IAA Biosynthetic Pathway

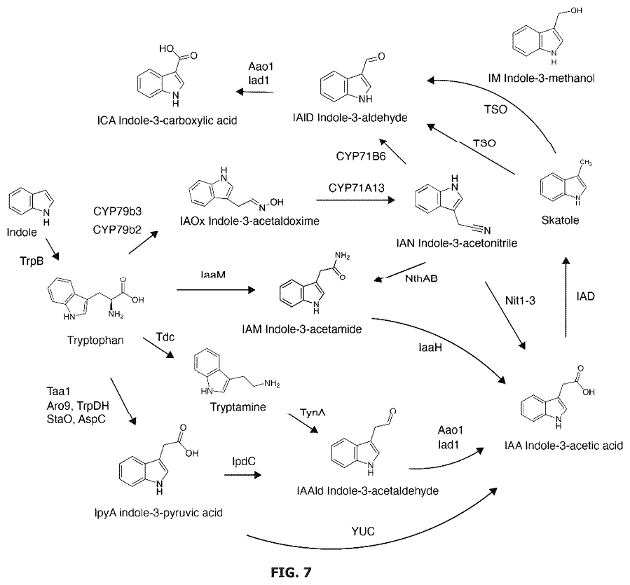

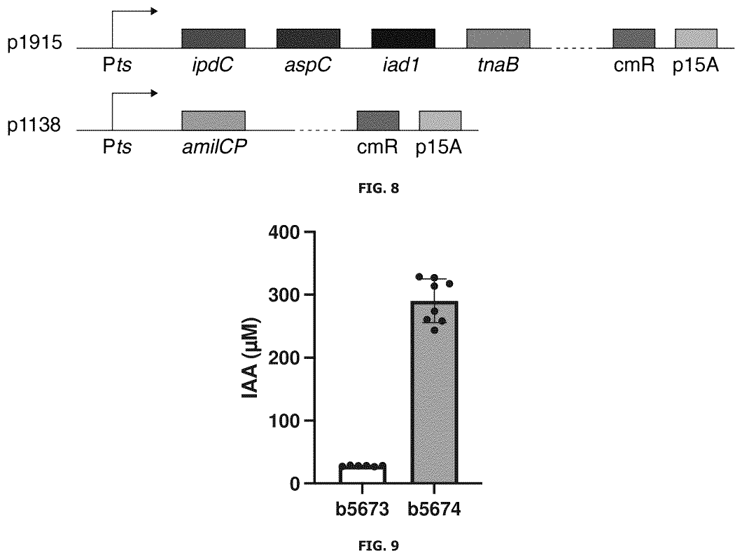

A p15A based plasmid (p1915, Seq ID No: 48) was constructed comprising ipdC from Pantoea agglomerans (Seq ID No: 41), aspC from Escherichia coli (Seq ID No: 39) and iad1 from Ustilago maydis (Seq ID No: 42) under the control of a temperature-inducible promoter (Pts). tnaB permease was also cloned in the vector under the control of the same promoter to improve the uptake of tryptophan (see FIG. 8). Plasmid p1138 carrying a purple pigment, amilCP, under the control of the same temperature-inducible promoter was constructed as a control (see FIG. 8).

### 1.2 Plasmid-Based Production of IAA In Vitro

E. coli MG1655 (b230) was transformed with plasmids p1915 (Seq ID No: 48) and p1138, creating strains b5674 and b5673, respectively. IAA production was demonstrated using Salkowski colorimetric assay (see Example 4.3). As shown in FIG. 9, a significant increase in IAA was detected in the bacterial overnight supernatant of strain b5674 (˜300 UM) in comparison to the empty control vector, in strain b5673.

### 1.3. Pathway Stability of Plasmid-Based Production of IAA

An in vivo study was performed in two groups of 5 mice dosed with the E. coli strain b5673, as described in Example 1.2, control with no IAA production, or strain b5674, as described in Example 1.2, which produces IAA. Full experimental details are provided in Example 4.2. The mice were dosed perorally with a single dose, and faecal samples were collected twice daily up to 54 hours after dosing. The faecal samples were analysed for levels of bacteria (CFU/g faeces). Colonies were recovered from plating of the faecal samples at each timepoint—where possible single colonies for the control strain b5673 and 3 colonies from the production strain b5674. The colonies were then grown in M9 media with added tryptophan and analysed for IAA production using the Salkowski assay (see Example 4.3). This would confirm the capability of the strain to maintain its production abilities after colonization and gastrointestinal passage. As the animals were only dosed once, the analysis would clarify the stability of the construct in the plasmid up to 54 hours after dosing.

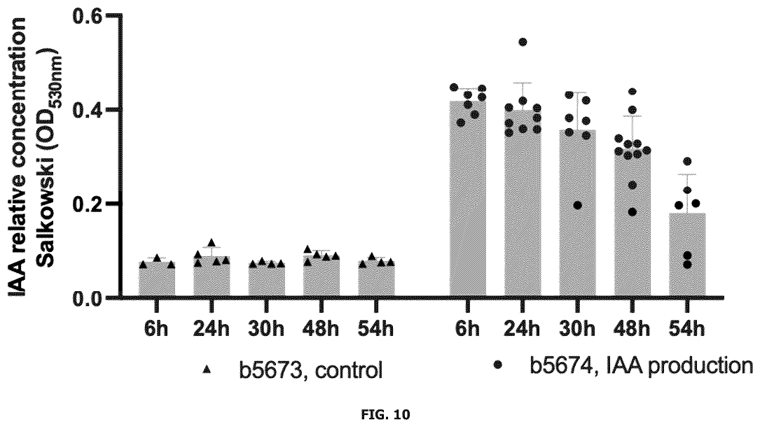

As shown in FIG. 10, the results of the study showed similar colonization capabilities of both strains during the study. It was further confirmed that the colonies isolated from faeces of animals dosed with the production strain b5674 produced IAA. Production by colonies started to decline 30 hours post-dose, and had halved by 54 hours post-dose, although with variability. As shown in FIG. 10 at the 54-hour time-point for the production strain b5674, two of the sampled colonies had levels of IAA production which were comparable to the production levels of the control strain b5673. Thus, these colonies had lost their production capability. Hence, the plasmid-carried IAA production pathway is not stable for continuous IAA production at high level, as seen 6-24 hours after dosing, without repeated dosing of the strain. The use of antibiotic selection confirms that the plasmids are present in the production strain b5674 at all timepoints, even though IAA production is severely reduced. Based on published data, we believe this severe reduction in IAA production is due to mutations introduced into the heterologous IAA biosynthetic genes.

1.4 Cloning IAA Biosynthetic Pathway into Plasmids with Exporters

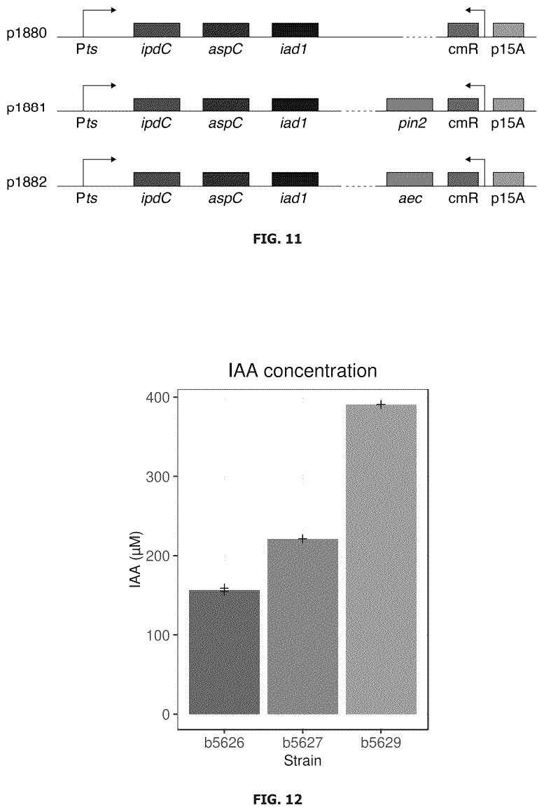

The IAA pathway was cloned together with IAA exporters Pin2 from Arabidopsis thaliana. (Seq ID No: 3) or aec from Pantoea agglomerans (Seq ID No: 2). Three new plasmids (p1880, p1881 and p1882) were cloned using plasmid p1915 (as described in Examples 1.1 and 1.2) as a backbone. tnaB permease was removed and exporters were added to transport IAA out of the cell. The exporters used are pin2 from Arabidopsis thaliana (Seq ID No: 3) and aec from Pantoea agglomerans (Seq ID No: 2). p1880 (Seq ID No: 54), p1881 (Seq ID No: 55) and p1882 (Seq ID No: 56) each contain ipdC from Pantoea agglomerans (Seq ID No: 41), aspC from Escherichia coli (Seq ID No: 39) and iad1 from Ustilago maydis (Seq ID No: 42) under the control of a temperature-inducible promoter (Pts), as well as a chloramphenicol resistance gene and use a p15A origin. In addition, plasmids p1881 and 1882 carry the exporters pin2 and aec respectively, expressed from the promoter that drives expression of the resistance marker. Cloning is performed as described in materials and methods (see Example 4 below), and plasmid maps are shown in FIG. 11.

1.5 Plasmid-Based Production of IAA In Vitro with Exporters

E. coli MG1655 (b230) was transformed with plasmids p1880 (Seq ID No: 54), p1881 (Seq ID No: 55) and p1882 (Seq ID No: 56), creating strains b5626, b5627 and b5629, respectively. IAA production was demonstrated using Salkowski colorimetric assay (see Example 4.3) in duplicate experiments. As shown in FIG. 12, the addition of aec increases the amount of IAA in the supernatant, relative to control which does not comprise any exporter. The addition of pin2 has the same effect, although not to the same level as aec. A further two experiments performed show a less significant effect of pin2, but the trend is still towards increased export with the pin2 exporter.

### Example 2: Pathway Engineering and Stability in the Chromosome

Because of the instability of production of IAA in a plasmid system, the inventors proceeded to optimize IAA production in the modified bacteria by integrating the genes into the chromosome, and placing them under the control of a constitutive promoter. Since the plasmid based IAA production pathway appeared to pick up loss of function mutations, it is reasonable to assume that over-expression of the pathway yields a fitness costs to the cell. The constitutive promoter used to drive expression of the system from the chromosome was therefore optimized to yield high IAA levels without causing detrimental effects.

### 2.1. Optimization of IAA Production & Aec Exporter

Two chromosomal deletions were introduced to Symbioflor G6/7 (b463) to further optimize IAA production in this strain. tnaA (tryptophanase) and the leader peptide tnaC were deleted together, to constitutively express tnaB (importer of tryptophan) and prevent the conversion of tryptophan to indole through the catalytic action of tnaA.

Tryptophan transcriptional repressor (trpR) was deleted with three different effects: (i) to de-repress synthesis of chorismate (thus increasing the amount of chorismate in the cell), (ii) to de-repress trpEDCA, which converts chorismate to indole (thus increasing the amount of indole in the cell), and (iii) to de-repress the mtr proton symporter of tryptophan/indole into the cell (thus increasing the amount of tryptophan/indole in the cell).

The deletions are made using scarless lambda red recombineering (Datsenko & Wanner, PNAS, 97, 6640, 2000, incorporated herein by reference). The resulting strain is designated as b5698. A streptomycin resistant version of this is obtained spontaneously by plating overnight cultures on streptomycin plates and saved as b6570.

To optimize IAA production, E. coli aspC was replaced by a different amino transferase taa1 from Arabidopsis thaliana (Seq ID No: 40), which is more specific for tryptophan and will increase IAA production and at the same time prevent production of by-products. Additionally, the exporter aec from Pantoea agglomerans (Seq ID No: 2) was added to transport IAA out of the cell, expressed from the CmR promoter. Construction was done in a cloDF1 based plasmid, with a medium strength constitutive promoter, Pc-aga (Seq ID No: 24).

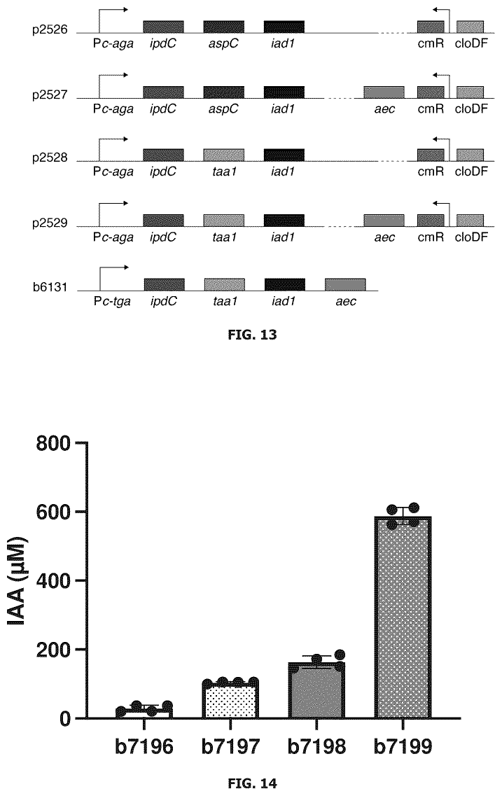

The layouts of the plasmid constructs are shown in FIG. 13. Plasmid p2526 (Seq ID No: 50) contains ipdC from Pantoea agglomerans (Seq ID No: 41), aspC from Escherichia coli (Seq ID No: 39) and iad1 from Ustilago maydis (Seq ID No: 42) under the control of promoter Pc-aga (Seq ID No: 24). Plasmid p2527 (Seq ID No: 51) additionally contains the aec exporter from Pantoea agglomerans (Seq ID No: 2) expressed from the promoter that drives expression of the resistance marker. Plasmid p2528 (Seq ID No: 52) contains ipdC from Pantoea agglomerans (Seq ID No: 41), taa1 from Arabidopsis thaliana (Seq ID No: 40) and iad1 from Ustilago maydis (Seq ID No: 42) under the control of promoter Pc-aga (Seq ID No: 24). Plasmid p2529 (Seq ID No: 53) additionally contains the aec exporter from Pantoea agglomerans (Seq ID No: 2) expressed from the promoter that drives expression of the resistance marker.

Attempts to clone a plasmid comprising the same genes as in plasmid p2526, but using the constitutive promoter pc-tga (Seq ID No: 1) were largely not successful. Without being bound by theory, we believe that this is due to an unsustainably high tryptophan to IAA conversion resulting in an accumulation of mutations in the pathway genes due to the high fitness cost.

Strain b5698 (ΔtnaAΔtrpR) is transformed with these plasmids, creating b7196 (containing plasmid p2526), b7197 (containing plasmid p2527), b7198 (containing plasmid p2528) and b7199 (containing plasmid p2529), as shown in FIG. 13.

### 2.2. IAA Production In Vitro

The four constructs, strain b7196 (aspC), strain b7197 (aspC, aec), strain b7198 (taa1) and strain b7199 (taa1, aec), were subjected to Salkowski analysis (see Example 4.3) to define the impact of the two different aminotransferases (aspC vs taa1) and the aec exporter on IAA secretion. Strain b7199 (taa1, aec) showed a 3-4× increased IAA production in comparison to the other constructs (see FIG. 14) leading to the selection of the taa1 aminotransferase and the addition of the aec exporter as optimized production pathway used for all future engineering.

### 2.3 Chromosomal Integration of IAA Biosynthesis Genes

The optimized pathway was integrated into the chromosome of strain b5698 (ΔtnaAΔtrpR) to increase genetic stability and IAA production further. The heterologous pathway for IAA production from strain b7199 consisting of ipdC from Pantoea agglomerans (Seq ID No: 41), taa1 from Arabidopsis thaliana (Seq ID No: 40), iad1 from Ustilago maydis (Seq ID No: 42) and the aec exporter from Pantoea agglomerans (Seq ID No: 2) under the control of a strong constitutive promoter Pc-tga (Seq ID No: 1) was integrated into the chromosome of strain b5698 (ΔtnaAΔtrpR), creating strain b6089. A streptomycin resistant version of this strain was obtained spontaneously as described above and saved as b6131. The layout of the chromosomal insertion is shown in FIG. 13.

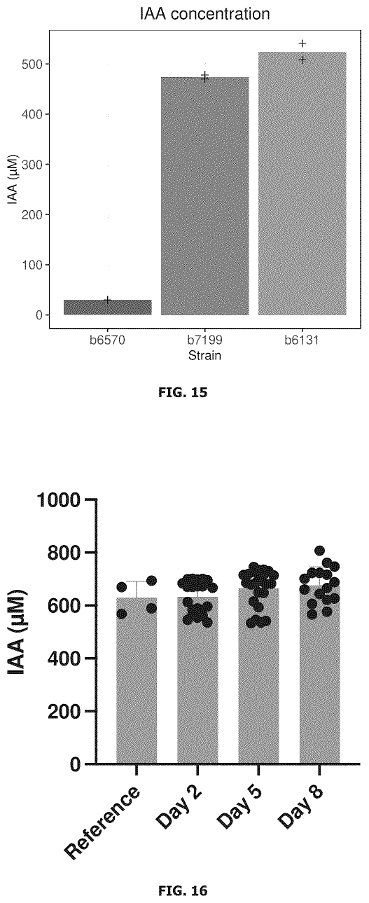

IAA secretion was tested by Salkowski assay (see Example 4.3) comparing plasmid-based expression (from strain b7199) with the new lead construct (strain b6131), see FIG. 15. The backbone strain (b6570, containing no heterologous genes) was added as control.

The results show comparable IAA production levels between genomically-integrated strain b6131 and plasmid-containing strain b7199. We therefore conclude that we can integrate the optimized pathway into the chromosome without detrimental effect on the bacterium.

IAA production was confirmed by liquid chromatography-mass spectrometry (LC-MS). Strain b6131 (genomically integrated) secreted 656.5 UM IAA (see Examples 4.2 and 4.4 for methodology).

### 2.3.1 LC-MS Data for Characterising the Effect of Chassis

The secretion of other metabolites will be additionally assessed by LC-MS. Among others, production of indole-3-butyric acid (IBA) is expected. In addition, production of IAA from indole will be tested. Since strain b6131 has been engineered to increase intracellular tryptophan and indole levels (through the chromosomal deletion of tnaA and trpR having the effects described in section 2.1), we expect to observe IAA production from indole.

LC-MS analyses were performed (A&M Labors, Germany, see Example 4.4 for methodology) to assess the impact of the chassis optimisation steps (deletion of tnaA and trpR) on substrate utilization (indole and tryptophan) and on the production of IAA and potential byproducts (indole-3-pyruvic acid, indole-3-butyric acid, indole-3-glyoxylic acic, indole-3-ethanol and skatole). Indole utilization was tested because indole is a precursor in tryptophan synthesis, and is also abundant in the human colon. Some of the engineering steps were performed to allow indole synthesis, facilitate its uptake, or efficient conversion to tryptophan. Therefore, IAA production was expected in the presence of indole.

Bacterial overnight cultures in LB media are refreshed in M9 media containing Cas amino acids, 0.4% glucose and either tryptophan (1 mM), indole (1 mM) or tryptophan and indole (1 mM each) followed by overnight culture at 37° C. After pelleting of bacterial cells, bacterial supernatants are transferred into fresh Eppendorf tubes containing acetonitrile at a 1:3 ratio. Samples are centrifuged at 2000 g for 10 minutes and the supernatants are stored at −20° C. prior to shipment to A&M Labor (www.am-labor.de) for LCMS analysis.

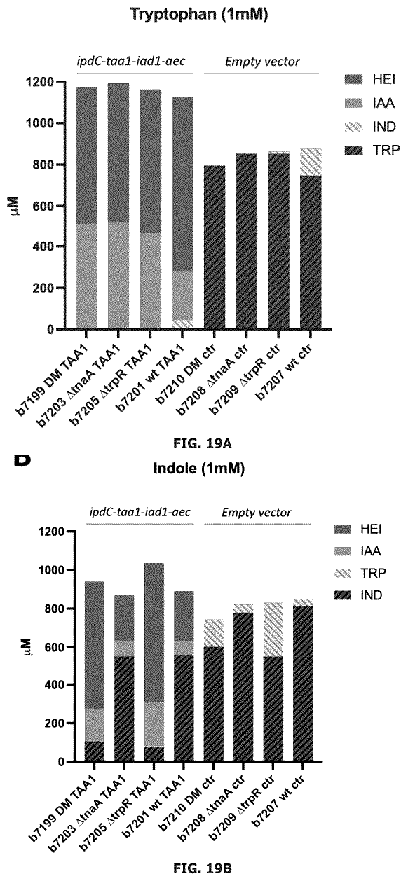

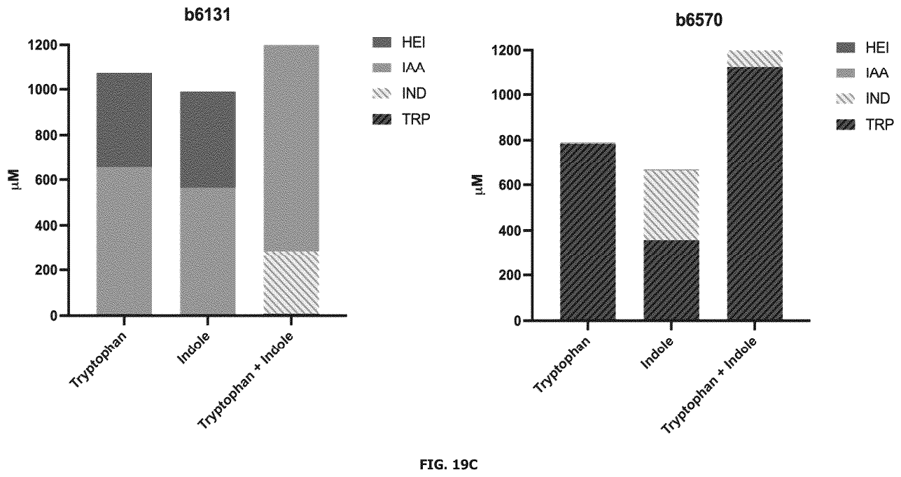

First, the effect of tnaCA and trpR deletions was evaluated using the optimized IAA production/export pathway (ipdC-taa1-iad1-aec) carried on plasmid p2529 (see FIGS. 19A and 19B). Strains carrying the corresponding empty vector, p2564, were used as controls. As expected, deletion of tnaA eliminated the conversion of tryptophan to indole (FIG. 19A, b7208 vs b7207), while deletion of trpR resulted in a lower rate of conversion (FIG. 19A, b7209 vs b7207). The effect of tnaA deletion is also reflected in the presence of the IAA production pathway. In the wild-type strain (b7201), part of the added tryptophan was converted to indole, while all tryptophan was converted to IAA or indole-3-ethanol in the deletion variants (FIG. 19A). The presence of indole-3-ethanol (tryptophol) was not surprising because it is spontaneously formed from indole-3-acetaldehyde (see FIGS. 1 and 4), and it was observed in previous IAA production studies (Romasi et al., J. Microbiol Biotechnol. 2013 December; 23 (12):1726-36).

In the presence of indole, the trpR deletion allowed more efficient conversion of indole to tryptophan (FIG. 19B, b7209 vs b7207), while the tnaA deletion had no effect. The trpR deletion greatly enhanced IAA and Indole-3-ethanol production from indole (FIG. 19B, b7205 vs b7201 and b7199 vs b7203). This may be due to the derepression of the mtr transporter, which primarily facilitates indole uptake.

### 2.3.2 Performance of the Genomically Integrated IAA Production Pathway

IAA production by the lead construct b6131 (chromosomally-integrated ipdC-taa1-iad1-aec operon, tnaA and trpR deletion) was assessed in the presence of different combinations of substrates, i.e. tryptophan, indole, or both (FIG. 19C). The parental strain that carries the tnaA and trpR deletions but not the integrated operon was used as a control (b6570). The added tryptophan was completely converted to IAA and indole-3-ethanol by b6131, and a better IAA to indole-3-ethanol ratio was observed than in the presence of a plasmid borne system (FIG. 19A, b7199). The added indole was also completely converted to IAA and indole-3-ethanol by b6131, with IAA being the primary product. This is a substantial improvement compared to the corresponding plasmid borne construct (FIG. 19B, b7199), where only partial conversion was achieved, and the majority of indole was converted to indole-3-ethanol. When both substrates were added at an equimolar ratio to b6131, complete conversion of tryptophan but only partial conversion of indole was achieved. However, the total IAA produced exceeded the levels obtained in the presence of single substrates.

### 2.3.3 Optimisation of Chromosomal Insertion Site

The IAA production pathway (ipdC-taa1-iad1-aec) was inserted at two new positions, to assess if a chromosomal insertion point closer to the origin of replication would increase IAA production Strains are grown overnight in LB, then refreshed into M9 1 mM tryptophan and incubated for another 24 hr at 37° C. shaking at 300 rpm. Samples are collected by spinning down the cells and harvesting the supernatant. Supernatants are collected and tested for IAA using a Salkowski assay (see Example 4.3). There was no observed increase in IAA production from either b7848 or b7829 as compared to b6131, see FIG. 20.

### 2.3.4 IAA Production Under Anaerobic Conditions

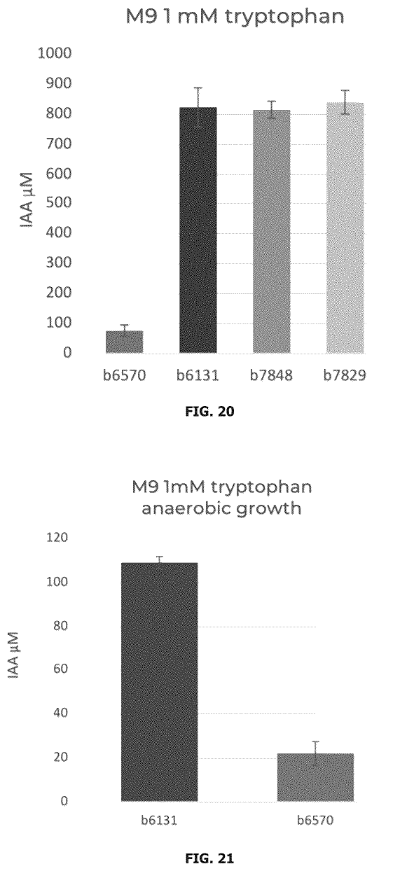

IAA production from b6570 (negative control) and b6131 (IAA producing strain) under anaerobic conditions was quantified using a Salkowski assay (see Example 4.3). Strains are grown anaerobically overnight in LB, then refreshed into reduced M9 1 mM tryptophan and incubated anaerobically for another 24 hr at 37° C. shaking at 300 rpm. Samples are collected by spinning down the cells and harvesting the supernatant. Supernatants are collected and tested for IAA. The level of IAA produced following overnight growth was significantly higher in cultures of b6131 compared to the control strain b6570 as shown in FIG. 21.

### 2.4. In Vivo Stability Data of Chromosomally-Integrated IAA Biosynthesis Genes

An in vivo study was performed in groups of 5 mice perorally dosed with E. coli b6570 (control strain with no IAA production) or lead chromosomal strain b6131 (IAA production, see Example 2.3). The mice were dosed perorally twice daily for 4 days (total of 8 doses), and faecal samples were collected daily up to 4 days after dosing initiation, and on day 7 after dosing. The faecal samples were analysed for levels of bacteria (CFU/g faeces). Single colonies, where possible, were recovered from plating of the faecal samples at 3 timepoints (1, 4 and 7 days after dosing). The colonies were then grown in media with added tryptophan and analysed for IAA production using the Salkowski colorimetric assay (see Example 4.3). This would confirm the capability of the strain to maintain its production abilities after colonization and gastrointestinal passage.

The results of the study showed similar colonization capabilities of both strains. It was further confirmed that the colonies isolated from faeces of animals, dosed with the IAA production strain b6131, produced IAA. Production was stable throughout the study, further supporting stability of the genomically-inserted IAA pathway up to 8 days after dosing initiation, see FIG. 16.

10 colonies isolated from faecal samples from day 4 were submitted for whole genome sequencing. No mutations were found in the pathway. This suggests that not only can high levels of IAA production be maintained over a longer time than the plasmids (as seen in Example 1.3 and FIG. 10), but additionally that the engineered bacteria are long-lasting and the heterologous genes do not appear to come at a significant fitness cost.

### 2.5. IAA Production at 30° C. And 37° C.

The stability of the taa1 pathway in the lead candidate strain b6131 and its control strain b6570 was tested in vitro at temperatures 30° C. and 37° C. The strains were grown overnight at these temperatures, followed by OD measurement.

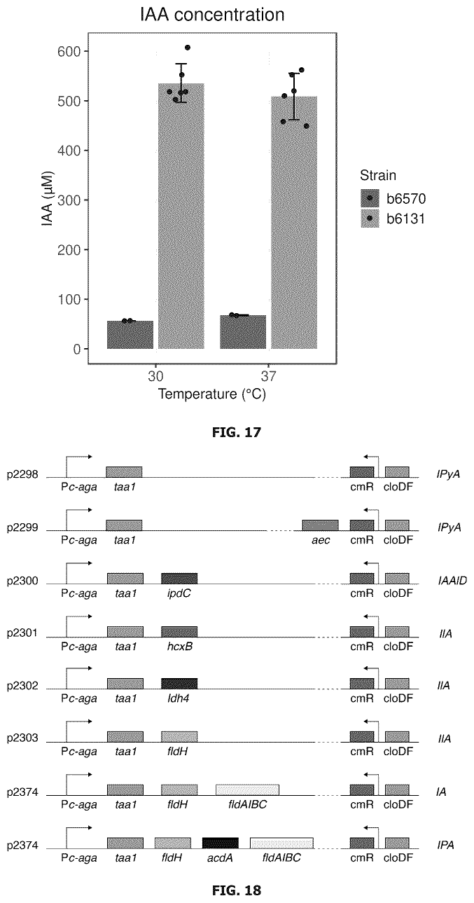

The levels of IAA secreted by the strains grown at 30 and 37° C. were measured using the Salkowski colorimetric assay (see Example 4.3). The results showed IAA production at both temperatures (FIG. 17). With similar growth and similar production levels, it can be concluded that the optimized taa1 based pathway is stable at both 30° C. and 37° C. This data shows that no difference in production is expected at physiological temperature, in contrast to data shown in e.g. FIG. 3 of WO2021/242897 (Synlogic), where a different IAA-producing pathway contained on bacterially-carried plasmids showed a lower average production at 37° C. compared to 30° C., suggesting sub-optimal production at physiological conditions.

### Example 3: Alternative Metabolites

A series of pathways designed to produce various alternative tryptophan metabolites were cloned into the same plasmid backbone as previously used in these Examples, as shown in FIG. 18. The plasmids were cloned as described in the materials and methods section (see Example 4), and Table 7 where the full sequences are listed.

B5698 has been transformed with these plasmids and the resulting strains will be tested for production of the various metabolites using LC-MS at a later time.

### Example 4: Materials and Methods

### 4.1 Engineering

Plasmids are constructed using in-Fusion cloning (Takara) according to manufacturer's protocol (full sequences provided in Table 7) and chromosomal manipulations are done using scarless lambda red mediated recombineering (see e.g. Datsenko & Wanner, supra, and Blank et al. 2011 PloS ONE, incorporated herein by reference in its entirety).

Strain b5698 is made using three rounds of recombineering (Δfix, ΔtrpR, ΔtnaCA) with the primers listed in Table 4. The strain is full genome sequenced to verify correct modifications.

Strain b6089 is made by amplifying the IAA pathway from plasmid p2169 (see Table 5, and Seq ID No: 49) using primers oli1550xoli2893xoli9094 (see Table 7, and Seq ID No: 37, Seq ID No: 26 and Seq ID No: 38 respectively) and recombineering it into the chromosome. The strain is full genome sequenced to verify correct modifications.

Strain b6131 is made by plating overnight cultures of b6089 on LB plates supplemented with streptomycin and picking spontaneous resisters. The strains are full genome sequenced to verify correct modifications.

Strain b6570 are made by plating overnight cultures of b5698 on LB plates supplemented with streptomycin and picking spontaneous resisters. The strain is full genome sequenced to verify correct modifications.

Strains b7196, b7197, b7198 and b7199 are made by introducing plasmids p2526, p2527, p2528, and p2529 (see Table 5 and Seq ID No: 50, Seq ID No: 51, Seq ID No: 52 and Seq ID No: 53 respectively), into b5698 using standard electroporation.

Strains b5673 and b5674 are made by introducing plasmids p1138 and p1915 (Seq ID No: 48) (see Table 5) respectively, into b230 using standard electroporation.

Strains b5626, b5627 and b5629 are made by introducing plasmids p1880 (Seq ID No: 54), p1881 (Seq ID No: 55) and p1882 (Seq ID No: 56) respectively (see Table 5), into b230 using standard electroporation.

TABLE 4
PCR products for engineering b5698

| PCR products | | |
|---|---|---|
| used for | | |
| engineering | | |
| b5698 | 1. step, ZeoR casette | 2. step |
| Δfix | Oli2906xoli2907 | Oli2892xoli2893 |
| | on p1884 | on mg1655 |
| ΔtrpR | Oli2908xoli2909 | Oli2910xoli2911 |
| | on p1884 | on b463 |
| ΔtnaCA | Oli2913xoli2914 | Oli2915xoli2916 |
| | on p1884 | on b463 |

### 4.2 In Vivo Work

### 4.2.1 Study 1

The study on plasmid-based production of IAA in vivo (as described in Example 1.3) was performed in 10 female C57BL/6JRj (Janvier Labs) mice at the age of approximately. 6 weeks upon arrival. The mice were housed in two groups of 5 in IVC cages and were acclimatized for at least 7 days before initiation of the study. The mice had ad libitum access to food and drinking water throughout the study. To ensure tryptophan availability the mice were fed a high-tryptophan diet (A22052401, L-Amino Acid Rodent Diet with 40 gm L-Tryptophan per kg, Research Diets Inc) changed three times per week. On day-4 the animals started 5 g/L streptomycin (Sigma-Aldrich, S6501) treatment through the drinking water, which was substituted by 500 mg/L chloramphenicol (Sigma-Aldrich, C0378) treatment through the drinking water from day-1 and until termination on day 2. The streptomycin treatment enabled engraftment of the various E. coli strains, while the chloramphenicol treatment ensures maintenance of the plasmid in the E. coli strain dosed. On day 0 the animals were dosed approximately 1×10⁷ CFU of our E. coli strain (b5673 (control) or b5674 (IAA-producing)) in 0.2 mL by peroral gavage. Faecal samples were collected from each animal in the morning of day-4 and 0 to evaluate E. coli eradication before inoculation. Two faecal pellets were then collected 6 hours post inoculation, and then morning and afternoon on days 1 and 2. One faecal pellet in PBS was selected for CFU whereas the other pellet was snap frozen and sent for LC-MS analysis to quantify IAA. The faecal pellet in PBS (Biowest, L0615) was spotted on MacConkey agar with streptomycin and chloramphenicol to select for the engineered strain. The colonies were then counted before selection for the Salkowski assay (see Example 4.3). During the study the animals were weighed at arrival and on days −4, −1, 0, 1 and 2. The animals were further observed clinically twice daily by trained personnel and were terminated on day 2 following the last samples.

### 4.2.1 Study 2

The study on in vivo stability as described in Example 2.4 was performed in 30 male C57BL/6JRj (Janvier Labs) mice at the age of approximately 5 weeks upon arrival. The mice had been on 40% fat, 40% carbohydrate and 2% cholesterol diet (D09100310, SSNIFF, Germany) for 40 weeks before study initiation. The mice were single housed during the study. The mice had ad libitum access to food and drinking water throughout the study. The animals were randomized into 6 groups of 5 animals according to bodyweight 1 week before study initiation. Groups 1 and 2 were given normal tap water as drinking water throughout the study, groups 3 and 4 were given 24 hours of 1 g/L streptomycin (Sigma-Aldrich, S6501) in the drinking water from day −2 to −1, and groups 5 and 6 were treated with 1 g/L streptomycin (Sigma-Aldrich, S6501) in the drinking water from day-3 until termination. Groups 1, 3 and 5 were dosed with the control strain b6570 and groups 2, 4 and 6 were dosed with the IAA producing strain b6131. The mice where dosed approximately 1×10⁸ CFU in 0.2 mL twice daily (AM+PM) by peroral gavage starting on day 1 and ending on day 4. Two faecal pellets were collected daily from each animal in the morning from day 1 until termination (day 5 or 8). One faecal pellet in PBS was selected for CFU whereas the other pellet was snap frozen and sent for LC-MS analysis to quantify IAA. The faecal pellet in PBS was spotted on MacConkey agar with streptomycin to select for our strain. The colonies were then counted before selection for the Salkowski assay (see Example 4.3). During the study the animals were weighed daily. The animals were further observed clinically twice daily by trained personnel and were terminated on day 5 or 8 following the last samples.

### 4.2.2 Study 3

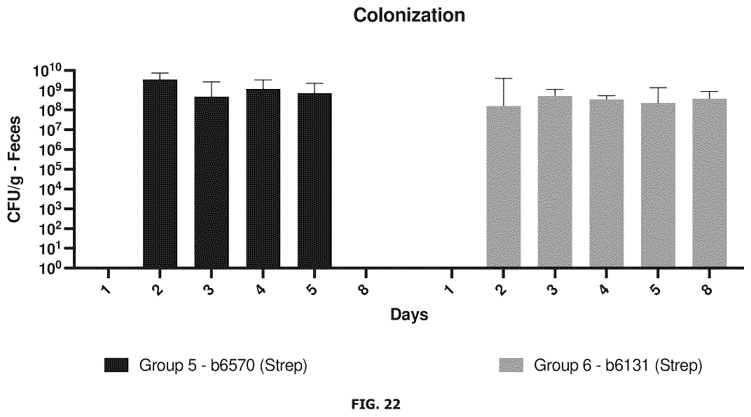

In vivo IAA production by our lead candidate (b6131) was tested. The study was performed in C57BI/6 mice in groups of 5 animals. Groups 5 (control) and 6 (IAA producing) were pre-treated and treated with 1 g/L streptomycin in the drinking water from day-3 until termination. The animals were inoculated with approx. 10⁹ CFU per dose and were dosed twice daily for 4 days. Two fecal samples were collected on day 1 (before first dose) and then daily until termination. One fecal pellet was snap frozen and sent IAA quantification by LC-MS analysis (A&M Labor, Germany, see Example 4.4 below for methodology) and the other fecal pellet was put in PBS for CFU quantification in house. With pre-treatment and continuous streptomycin treatment until termination the CFU counts in feces remained at a stable level around 10⁹ CFU/g feces (FIG. 22).

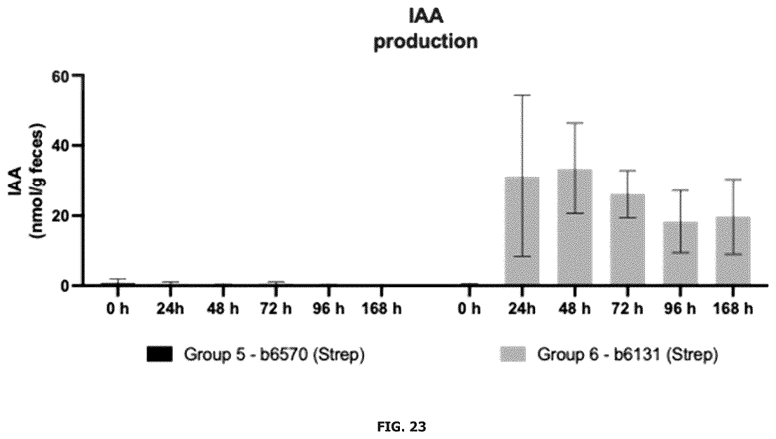

The LC-MS data showed a clear difference in IAA levels between the groups. An increase of 20-30 times relative to controls was observed for the producing strain (FIG. 23). The control (group 5) maintained IAA levels stably below 1 nmol/g feces and the producing strain (group 6), showed an IAA increase to >30 nmol/g feces initially, stabilizing around 20 nmol/g feces by day 8 (day of termination).

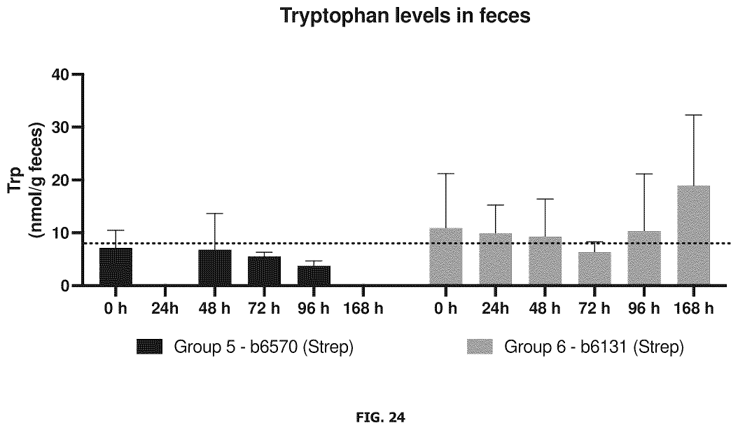

Through further analysis of the feces by LC-MS, it was seen that most kynurenine and skatole levels were below the limit of detection, hence no further analysis could be performed on these data. However, we included tryptophan and indole for analysis as these can be converted into IAA by our strain. There were no visible or statistical change in tryptophan between groups, which indicated that tryptophan levels were not affected by our strain and may not have been the main source for conversion into IAA (FIG. 24).

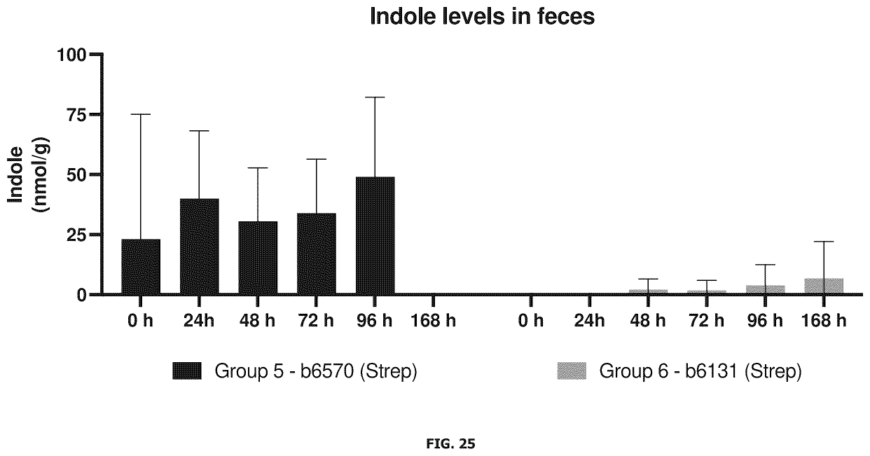

However, analysis of fecal indole levels revealed that the group 6 had continuously low levels close to 0 nmol/g feces throughout the study, whereas the control group (group 5) did have indole levels around 20-50 nmol/g feces throughout the study. This shows a negative correlation between indole usage (FIG. 25) and production of IAA (FIG. 23).

Based on these data we have successfully engineered our strain to produce IAA in the mouse gut.

TABLE 5
Plasmids used in this study

| Plasmid | | |
|---|---|---|
| number | Description | Reference |
| p1138 | Pts- amilCP, chloramphenicol resistance, p15A | Lab collection |
| p1884 | Zeocin resistance cassette flanked by I-sceI site | Blank et al. 2011 |
| | | PloS ONE. |
| P1910 | Laboratory collection, lambda red recombineering system | Lab collection |
| p1915 | Pts- ipdC-aspC-iad1, chloramphenicol resistance, p15A | This work |
| p2169 | taa1-iad1-aec, CloDF13, spectinomycin resistance | This work |
| p2526 | Paga-ipdC-aspC-iad1, CloDF13, chloramphenicol resistance | This work |
| p2527 | Paga- ipdC-aspC-iad1, aec, CloDF13, chloramphenicol | This work |
| | resistance | |
| p2528 | Paga-ipdC-taa1-iad1, CloDF13, chloramphenicol resistance | This work |
| p2529 | Paga-ipdC-taa1-iad1, aec, CloDF13, chloramphenicol | This work |
| | resistance | |
| p2298 | Paga-taa1 CloDF13, chloramphenicol resistance | This work |
| p2299 | Paga-taa1 CloDF13, aec, chloramphenicol resistance | This work |
| p2300 | Paga-taa1-ipdC, CloDF13, chloramphenicol resistance | This work |
| p2301 | Paga-taa1-hcxB, CloDF13, chloramphenicol resistance | This work |
| p2302 | Paga- taa1-Idh4, CloDF13, chloramphenicol resistance | This work |
| p2303 | Paga- taa1-fldH, CloDF13, chloramphenicol resistance | This work |
| p2373 | Paga- taa1-fldH-fldAIBC, CloDF13, chloramphenicol | This work |
| | resistance | |
| p2374 | Paga- taa1-fldH-acdA-fidAIBC, CloDF13, chloramphenicol | This work |
| | resistance | |
| p2564 | Empty vector, Paga CloDF13, chloramphenicol resistance | This work |
| p1880 | Pts- ipdC-aspC-iad1, chloramphenicol resistance, p15A | This work |
| p1881 | Pts-ipdC-aspC-iad1, pin2, chloramphenicol resistance, p15A | This work |
| p1882 | Pts-ipdC-aspC-iad1, aec, chloramphenicol resistance, p15A | This work |

TABLE 6
Strains used in this study

| Strain | |
|---|---|
| number | Description |
| b230 | E. coli K12 MG1655 |
| b5673 | b230 (p1138) |
| b5674 | b230 (p1915) |
| b463 | Symbioflor G6/7 isolated at SnipR Biome |
| b5698 | Symbioflor G6/7 Δfix ΔtnaCA ΔtrpR |
| b6089 | b5698 IAA+ (Ptga-ipdC-taa1-iad1-aec) |
| b6131 | b5698 IAA+ (Ptga-ipdC-taa1-iad1-aec) strR |
| b6570 | b5698 strR |
| b7196 | b5698 p2526 |
| b7197 | b5698 p2527 |
| b7198 | b5698 p2528 |
| b7199 | b5698 p2529 |
| b5626 | b230 p1880 |
| b5627 | b230 p1881 |
| b5629 | b230 p1882 |

### 4.3 Salkowski Assay

The Salkowski assay is a spectrophotometric assay traditionally used for the detection of plant or bacterial derived IAA (Gang et al., Bio-Protocol., 9 (9): e3230, 2019, incorporated herein by reference in its entirety). The assay is based on the detection of a pink colour derived from the reaction of the Salkowski reagents (ferric chloride and perchloric acid) with IAA present in bacterial supernatants. In brief, bacterial overnight cultures in LB media are refreshed in M9 media containing Tryptophan (1 mM), Cas amino acids, 0.4% glucose, cultures are induced after 1.5 h if required (depending on whether the relevant promoter requires induction to express), followed by over-night culture at 37° C. Bacterial cells are pelleted and bacterial supernatants are incubated in the presence of Salkowski reagents for 45 min. The colour change is assessed by measuring absorbance at 530 nm. A standard curve is used to calculate IAA concentrations of each bacterial supernatant. The method has been validated by LC/MS analysis.

### 4.4 LC-MS Analysis

Samples were prepared as follows: Bacterial overnight cultures in LB media are refreshed in M9 media containing Cas amino acids, 0.4% glucose and either tryptophan (1 mM), indole (1 mM) or tryptophan and indole (1 mM each), followed by overnight culture at 37° C. After pelleting of bacterial cells, bacterial supernatants are transferred into fresh Eppendorf tubes containing acetonitrile at a 1:3 ratio. Samples are centrifuged at 2000 g for 10 minutes and the supernatants are stored at −20° C. prior to shipment to A&M Labor for LCMS analysis.

Sample analysis was carried out by A&M Labor using LC-MS substantially as follows. The analysis is carried out using a Thermo Scientific Vanquish LC coupled to Orbitrap Exploris 240 MS (ThermoFisher Scientific). An electrospray ionization interface is used as ionization source. Analysis is performed in positive and negative ionization mode under polarity switching. The UPLC is performed using a slightly modified version of the protocol described by Doneanu et al., 2011 (Water Application note 2011, 720004042en). Peak areas were extracted using Compound Discoverer 3.3 (Thermo Scientific). Identification of compounds were performed at four levels; Level 1: identification by retention times (compared against in-house authentic standards), accurate mass (with an accepted deviation of 3 ppm), and MS/MS spectra, Level 2a: identification by retention times (compared against in-house authentic standards), accurate mass (with an accepted deviation of 3 ppm), Level 2b: identification by accurate mass (with an accepted deviation of 3 ppm), and MS/MS spectra, Level 3: identification by accurate mass alone (with an accepted deviation of 3 ppm).

### Example 5: Conjugative Plasmids Expressing IAA

The IAA pathway together with the AEC exporter can be moved to a conjugative plasmid capable of moving between bacterial cells belonging to certain species in the native microbiome. This will allow native strains to produce IAA, reducing the need for an administered host to colonize.

### 5.1: Materials and Methods

### 5.1.1 Engineering

Non conjugative plasmids are constructed using in-Fusion cloning (Takara) according to manufacturer's protocol and chromosomal manipulations, as well as manipulations of conjugative plasmids are done using scarless lambda red mediated recombineering from plasmid p1910 (Datsenko & Wanner PNAS 97, 6640-45, 2000) (Blank et al. 2011 PLOS ONE)). Primers for chromosomal engineering are listed in Table 8 and sequences can be found in Table 7.

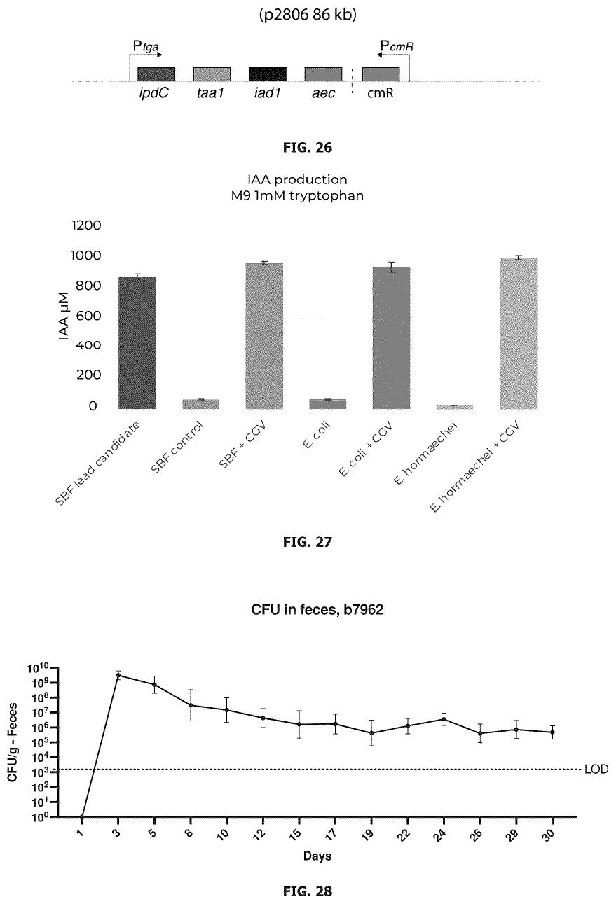

Sequences of the promoter and genes used are as follows: promoter Pc-tga (Seq ID No:1), ipdC (Seq ID No:41), taa1 (Seq ID No:40), iad1 (Seq ID No:42), aec (Seq ID No:2), see FIG. 26.

TABLE 8

| PCR products used for | | |
|---|---|---|
| chromosomal engineering | 1. step, ZeoR cassette | 2. step |
| Δp3 | Oli7154xOli7156 on p1884 | Oli7159xOli7160 on b463 |
| Δhly | Oli8199xOli8200 on p1884 | Oli8201x Oli8197 on b463 |
| I-tev-clbS at csiR | Oli2906xOli2907 on p1884 | Oli8729xOli2893, then |
| | | Oli5622xOli2893. on b6022 |
| ΔdapA | Oli8822xOli8821 on b3104 | |

Conjugative plasmid p2464 is made by amplifying a chloramphenicol cassette from pBAD33 using oli8116 x oli8117 and recombineering it into wt β10 in b3557. This creates strain b6584. The strain is full genome sequenced to verify correct modifications.

Conjugative plasmid p2806 is made by amplifying the IAA pathway and chloramphenicol cassette from p2719 using primers oli8805 x oli8743 and recombineering it into wt β10 in b3557. This creates b7830. The strain is full genome sequenced to verify correct modifications.

Strain b7951 is made based on b463 using three rounds of recombineering (Δp3, Δhly and I-tev-clbS at csiR) with the primers listed in table 1. The strain is full genome sequenced to verify correct modifications.

Strain b8524 is made based on b7951 using one round of recombineering (ΔdapA) with the primers listed in table 1. The strain is full genome sequenced to verify correct modifications.

Strains B7848, b7919 and b7920 are made by conjugating p2806 into b5698, b7396 and b7399 respectively.

Strain B7962 is made by plating overnight cultures of b7919 on LB plates supplemented with streptomycin and picking spontaneous resisters. The strain is full genome sequenced to verify correct modifications.

### 5.1.2 Conjugation Assay

Donor and recipients are inoculated in LB with appropriate supplements and grown to early exponential phase (OD 0.4). Donor and recipients are spotted for CFU and mixed in the ratio of 5:1. For each, 100 μL of mixed cells is placed in the middle of an LB plate and incubated ON at 37° C. The cells are scraped of the plate and resuspended in 300 μL LB. 10-fold dilutions are spotted on non-selective LB plates, as well as plates selective for donor and transconjugants. The plates are incubated ON at 37° C. and counted for CFU.

### 5.1.3 In Vivo Study

A 4-week mouse study was performed testing the strain's ability to produce IAA in the gut of the mice. The mice were pre-treated with 24 hours of streptomycin 1 g/L in the drinking water from day −2 to −1. The mice were then dosed perorally twice daily for 4 weeks, with a dose of approx. 5×10⁸ CFU/dose. The strain dosed was the b7962 which was expected to have a better chance at colonizing in the mouse gut due to its origin in mice. Fecal samples were collected 3 times weekly (Monday, Wednesday, and Friday) and were spotted for determining the CFU/g feces. Further, fecal samples were collected on days 1 (before dosing), 3, 10 and 29/30 (before termination) for quantification of IAA in the feces.

### 5.2 Results

5.2.1 IAA Production from Conjugative Plasmids

A large 80 kb conjugative plasmid (B10) is isolated from a wild type E. coli isolate b3557. A pathway to produce IAA is engineered into this as described in materials and methods (see section 5.1.1). The pathway contains 4 genes (ipdC [Seq ID No:41]-taa1 [Seq ID No:40]-iad1 [Seq ID No:42]-aec [Seq ID No:2]) in that order expressed from a constitutive promoter Pc-tga (Seq ID No:1). A chloramphenicol marker CAM is also inserted next to the pathway. The engineered β10IAA plasmid (p2806) has a total size of 86 kb. A map of the engineered construct is shown in FIG. 26.

This plasmid is moved into:
1) E. coli Symbioflor G6/7 ΔtnaCAΔtrpR (b5698, creating b7848),
2) a wild type t E. coli isolate (b7396, creating b79190), and
3) a wild type E. hormaechei isolate (B7399, creating b7919).

The wild type strains are isolated from mouse feces. IAA production is compared between strains carrying the conjugative vector (CGV) and their wild type counterpart. A strain previously developed to produce IAA (b6131, see Example 4.1 and Table 6) using the same pathway, but integrated into the chromosome together with other modifications, is included as positive control. As seen in FIG. 27, all strains carrying the CGV produces high levels of IAA.

5.2.2 IAA Production from a Conjugative Vector In Vivo

A streptomycin resistant version of b7919 carrying the IAA producing CGV, b7962, went into mice (see Example 5.1.3). The mice had been pre-treated with 1 g/L streptomycin in the drinking water before initiation of dosing perorally twice daily. Dosing twice daily resulted in initial colonization detected at 2×10⁹ CFU/g feces and then slowly declining to 4×10⁵ CFU/g feces by day 19 and remaining at this level until termination (see FIG. 28).

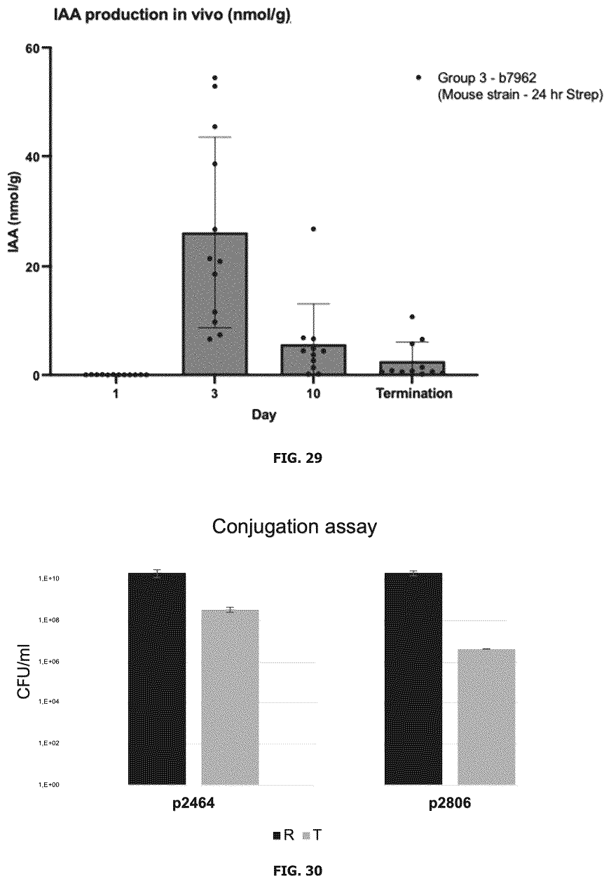

Initially, high levels of IAA production were observed (FIG. 29), yet this declined over time. The IAA levels rose to a mean of 26 nmol/g feces on day 3, which decreased to 5.5 nmol/g feces on day 10 and further to 2.5 nmol/g feces on the day of termination (day 29/30). The decrease in IAA production follows a trend of decreasing numbers of CFU found in feces, which indicates a decreasing level of colonization irrespective of the two times daily dose. Re-isolates from all time points were subjected to full genome sequencing and found to lose or mutate the IAA pathway from the CVG. This suggests that the burden posed on the cell is too high, and that optimization of the expression levels are needed. Transconjugants were isolated from LB, EMB and MacConkey plates supplemented with chloramphenicol to explore whether the CGV moved into the native microbiome. Transconjugants of native E. coli and klebsiella strains were observed starting day 10 and found in increasing numbers over the course of the study. It is speculated that the reason behind the delay in the onset of transconjugation is due to the study design. Large parts of the native microbiome are killed in the beginning of the study using streptomycin to increase colonization of the donor strain, yet this also lowers the number of possible recipients until they have regrown.

### 5.2.3 Conjugation Efficiency of Engineered Vectors

A conjugation assay was performed to evaluate the effect of the IAA pathway on the conjugation efficiency. The donor strains were b8524 (SBF G6/7 ΔdapA) with p2464 (wild-type β10) or p2806 (B10 with IAA pathway). These plasmids were conjugated to b5652 (recipient E. coli strain) following the solid conjugation protocol as described in materials and methods, and the number of transconjugants as well as the total number of recipients were counted (FIG. 30). Plasmid p2806 appear to have the lowest level of transconjugants, indicating that the presence of the IAA pathway possesses a burden to the cell.

### Example 6: Materials and Methods

### 6.1 Engineering

Plasmids are constructed using in-Fusion cloning (Takara) according to manufacturer's protocol (full sequences provided in Table 7) and chromosomal manipulations are done using scarless lambda red mediated recombineering (Datsenko & Wanner PNAS 97, 6640-45, 2000, Blank et al. 2011 PLOS ONE). Primers are listed in Table 9.

TABLE 9
Primers used for strain engineering

| PCR products used | | |
|---|---|---|
| for engineering | 1. step, ZeoR cassette | 2. step |
| Δp3 | Oli7154xoli7156 on p1884* | Oli7159xoli7160 on b463 |
| Δhly | oli8199xoli8200 on p1884* | oli8201xoli8197 on b463 |
| IAA to csiR | Oli2906xoli2907 on p1884* | Oli1550xoli2893 on p2169* |
| ΔtrpR | Oli2908xoli2909 on p1884* | Oli2910xoli2911 on b463 |
| ΔtnaCA | Oli2913xoli2914 on p1884* | Oli2915xoli2916 on b463 |

*see Table 5

Strain b8344 is made based on b463 (see Table 6 above) using five rounds of recombineering (Δp3, Δhly, ΔtnaCA, ΔtrpR and IAA to csiR) with the primers listed in Table 9. The resulting strain is made by plating overnight cultures on LB plates supplemented with streptomycin and picking spontaneous resisters. The strain is full genome sequenced to verify correct modifications. Thus, b8344 is Symbioflor G6/7 ΔtnaCA ΔtrpR Δp3 Δhly IAA+ (Ptga-IpdC-taa1-iad1-aec) strR.

### 6.2 In Vivo Work

6.2.1 In Vivo IAA Production after a Single Peroral Dose

An in vivo IAA production study was performed using strain b8344. The study was performed in five (5) C57BI/6 mice. In the study, the mice were pre-treated for 24 hours with 1 g/L streptomycin in the drinking water from day −2 until day −1. On day −1 and until day 11, the animals were dosed a 5% prebiotic solution in the drinking water. The animals were inoculated with approx. 1×10⁸ CFU given as a single peroral dose by gavage on day 1 (24 hours after streptomycin removal). From day 11 till day 16 (day of termination) the animals were only supplied with normal drinking water. During the study, the level of colonization was estimated through collection of fecal pellets daily excluding days 6, 7 and 14 (further day 8 was excluded). The fecal pellet was placed in PBS and then spotted for CFU determination. Another fecal pellet was collected on days 1, 4, 11 and 16 and snap frozen for IAA quantification by LC-MS analysis (A&M Labor, Germany, see Example 4.4 above for methodology)

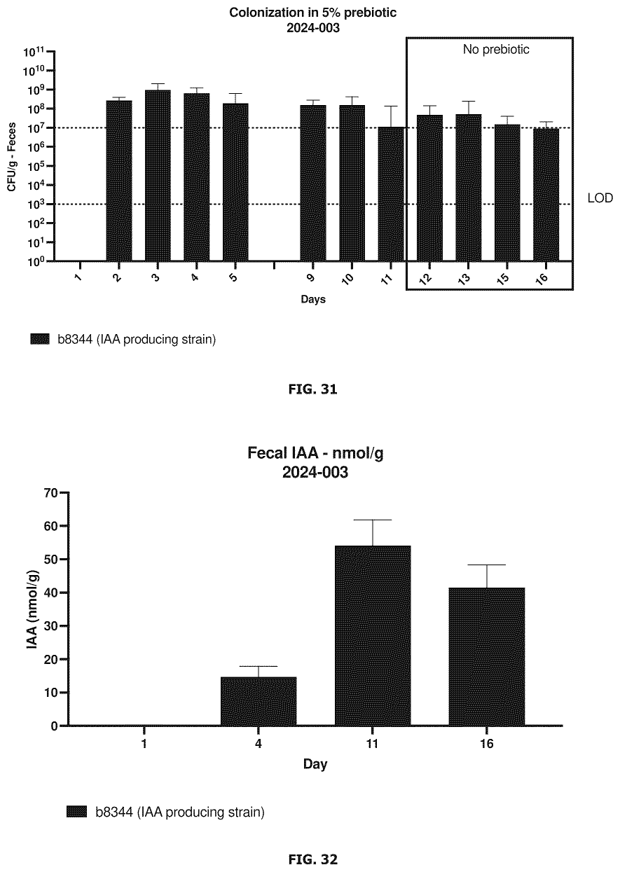

The strain was independent of prebiotic treatment as it maintained a high level of colonization throughout the study with levels stabilizing around approx. 1×10⁷ CFU/g feces from days 11 to 16 (FIG. 31).

The LC-MS data showed than the animals had baseline levels of IAA below 1 nmol/g feces. The levels of IAA increased to a mean of 15 nmol/g feces on day 4 and further to a mean of 54 nmol/g feces on day 11, despite a 2 log CFU decrease of the b8344 strain on day 11. On the day of termination, the animals had an IAA level of 42 nmol/g feces with a stable level of approx. 1×10⁷ CFU/g feces (FIG. 32).

Based on these data, we have successfully engineered our strain to produce IAA in the mouse gut and shown that it can produce IAA up to 15 days after a single peroral dose.

TABLE 7
Sequence listing

| Se- | | |
|---|---|---|
| quence | | |
| number | Description | Sequence |
| Seq ID | Pc-tga | aattcagcgggttcgtgcgaactgttgacaattaatactcggctcgtataatgtgtggaaggctagcaggaggaattca |
| No: 1 | promoter | |
| Seq ID | aec exporter | ATGCCACTGCTGTTTGAAAGCTTAAGCCATCAAATTTACCTGTCAGCCCCGCTGTTTATCCTGATCCTGCTGGGGTACTGCCTGACGCGCCTCGGGA |
| No: 2 | | AATGGCCCGCCAGCATCAGCGAAGGGATGAACCGCTTTGTGTTTAACGTTGCCCTGCCCTGCATGCTGTTTCGGGTGATGAGCACCTTCTCGCAAA |
| | | GTCCGCCGGTGGATGCGCGCCTGTTGCTGGCCTTTTTCGGCAGTTGCCTGCTGGTGTTTATCGTGGGGCGACTGCTGGCAACAAGGCTGTTTAATC |
| | | TGGATGGCGTAGCGGCGTCGGTTTTTGCGCTGGGGGGCATCTTTTCCAATAACGTGATGCTGGGGATCCCAGTGGCCACGGTACTGCTGGGACCAG |
| | | AAGCCCTGCCCCCGGTGGCGCTGGTGCTGGTGTTTAACAGCCTGATCCTCTGGACCTTACTGACCGTTTCGGTCGAGTGGGCAAAACAGGGCAGCT |
| | | TCTCGCTGGAAGGACTCTGGCGCACCCTGCTCGGCGTGCTGAAGAATCCGCTGATCATCGGTATTCTCAGCGGTACAGCCTGGAGCTTTCTGCAAC |
| | | GTCCCTTGCCGCTGCTGGCCGCTGAACCGCTAAAGATGCTGGCCTCACTGGCCGCACCTCTGTCGCTGGTGACGCTGGGCATGAGCCTGGCGCACT |
| | | ATCGGGTGCGGGATGGCTTAAAAGAGAGCTATACCATCTGCCTGCTGAAACTGGTGCTGCAACCGATGACGGTATGGGCTATCGCGTGGCTGACCG |
| | | ATCTGCCGCCGCTGGAGAGTAAAGTGGTGGTGCTGCTGGGATCGATGGCGGTCGGGGTTAACGTCTACCTGATGTCGCAGAAGTTTAATGTTATCA |
| | | CCGGCCCGGCAGCGGCGAGCATGCTCTTTTCTACCGTGTTTGCGGCGGTGACAACGCCAATCTGGATGATGCTGATGACGCTGGGGGGCTATTAAC |
| Seq ID | pin2 | ATGATTACAGGCAAAGATATGTACGACGTGCTGGCAGCTATGGTGCCCCTGTATGTGGCTATGATCCTTGCTTACGGCTCTGTCCGCTGGTGGGGT |
| No: 3 | exporter | ATTTTTACACCCGACCAATGCTCAGGAATTAATCGTTTTGTCGCTGTTTTTGCCGTTCCTTTGTTATCATTTCATTTCATTAGCTCAAATGACCCTTA |
| | | TGCAATGAATTATCATTTTTTAGCCGCTGATTCCTTACAGAAAGTGGTTATCTTAGCAGCCTTGTTCCTGTGGCAAGCCTTCTCTCGTCGCGGGAGT |
| | | TTAGAATGGATGATTACACTTTTTTCCCTTTCTACCTTGCCCAATACGCTGGTAATGGGTATCCCCCTTTTGCGCGCAATGTACGGCGACTTTTCTG |
| | | GTAACCTTATGGTCCAAATCGTCGTCTTACAGAGTATTATTTGGTACACTTTGATGCTGTTTTTGTTCGAGTTTCGCGGCGCTAAACTTCTGATTAG |
| | | TGAACAATTTCCGGAGACTGCGGGGTCAATCACTAGTTTTCGTGTTGATAGTGATGTCATCAGCTTGAATGGGCGCGAGCCATTGCAGACCGACGC |
| | | GGAGATCGGCGATGATGGGAAGCTTCATGTTGTAGTACGCCGTAGCAGTGCTGCATCCAGTATGATTAGTTCATTTAACAAGTCCCATGGAGGGGG |
| | | TCTTAACAGTTCAATGATTACACCACGTGCTAGTAATCTTACTGGGGTTGAGATTTACAGTGTGCAGTCTAGTCGCGAGCCCACCCCGCGTGCATCC |
| | | AGTTTCAACCAAACGGACTTTTACGCTATGTTCAATGCTAGTAAGGCCCCTTCACCGCGCCACGGCTATACTAATAGCTACGGAGGCGCAGGAGCAG |
| | | GCCCAGGTGGTGACGTGTACAGCCTTCAGTCTTCCAAAGGGGTCACACCGCGTACATCTAATTTCGATGAAGAAGTTATGAAAACTGCAAAGAAAGC |
| | | AGGTCGTGGCGGTCGCTCTATGTCAGGGGAATTATACAACAACAATTCGGTGCCAAGTTACCCACCCCCTAACCCGATGTTTACAGGATCAACGAGT |
| | | GGTGCGTCCGGTGTTAAGAAGAAGGAGTCAGGCGGAGGGGGAAGCGGGGGAGGCGTCGGGGTAGGTGGCCAAAACAAGGAAATGAATATGTTCG |
| | | TCTGGTCTAGCAGCGCCTCACCCGTCAGCGAGGCGAACGCAAAGAATGCCATGACGCGCGGATCTAGTACTGACGTGTCAACCGACCCTAAAGTGT |
| | | CCATCCCCACCCCATGACAACTTAGCGACCAAGGCTATGCAGAATCTTATTGAAAATATGTCGCCTGGTCGTAAAGGCCATGTAGAAATGGATCAGGA |
| | | CGGGAACAATGGGGGGAAATCTCCCTACATGGGTAAGAAGGGGTCTGATGTCGAGGATGGAGGTCCCGGACCTCGTAAGCAGCAGATGCCTCCGG |
| | | CGAGTGTAATGACACGCTTGATTCTTATTATGGTCTGGCGTAAGTTAATTCGCAATCCTAACACATACTCAAGCCTTTTTGGTCTGGCCTGGTCTTTA |
| | | GTATCGTTTAAGTGGAATATTAAAATGCCAACTATCATGAGCGGTAGCATTTCCATTTTGAGCGACGGGGGATTGGGCATGGCAATGTTTAGTCTTG |
| | | GCCTGTTCATGGCCTTGCAACCAAAAATTATCGCGTGCGGGAAGTCTGTAGCCGGGTTTGCGATGGCGGTTCGCTTCCTGACTGGACCAGCGGTTA |
| | | TTGCTGCCACCAGTATCGCTATCGGTATTCGCGGGGACCTGTTACACATTGCCATCGTACAGGGGGCACTGCCCCAGGGAATCGTCCCCTTTGTTTT |
| | | TGCCAAAGAATACAATGTGCACCCAGATATTCTTTCTACGGCAGTGATTTTTGGTATGCTTGTGGCGCTGCCGGTTACCGTGTTATATTATGTTTTA |
| | | CTTGGATTATAA |
| Seq ID | BBa_J23119 | ttgacagctagctcagtcctaggtataatgctagc |
| No: 4 | | |
| Seq ID | BBa_J23100 | ttgacggctagctcagtcctaggtacagtgctagc |
| No: 5 | | |
| Seq ID | BBa_J23101 | tttacagctagctcagtcctaggtattatgctagc |
| No: 6 | | |
| Seq ID | BBa_J23102 | ttgacagctagctcagtcctaggtactgtgctagc |
| No: 7 | | |
| Seq ID | BBa_J23103 | ctgatagctagctcagtcctagggattatgctagc |
| No: 8 | | |
| Seq ID | BBa_J23104 | ttgacagctagctcagtcctaggtattgtgctagc |
| No: 9 | | |
| Seq ID | BBa_J23105 | tttacggctagctcagtcctaggtactatgctagc |
| No: 10 | | |
| Seq ID | BBa_J23106 | tttacggctagctcagtcctaggtatagtgctagc |
| No: 11 | | |
| Seq ID | BBa_J23107 | tttacggctagctcagccctaggtattatgctagc |
| No: 12 | | |
| Seq ID | BBa_J23108 | ctgacagctagctcagtcctaggtataatgctagc |
| No: 13 | | |
| Seq ID | BBa_J23109 | tttacagctagctcagtcctagggactgtgctagc |
| No: 14 | | |
| Seq ID | BBa_J23110 | tttacggctagctcagtcctaggtacaatgctagc |
| No: 15 | | |
| Seq ID | BBa_J23111 | ttgacggctagctcagtcctaggtatagtgctagc |
| No: 16 | | |
| Seq ID | BBa_J23112 | ctgatagctagctcagtcctagggattatgctagc |
| No: 17 | | |
| Seq ID | BBa_J23113 | ctgatggctagctcagtcctagggattatgctagc |
| No: 18 | | |
| Seq ID | BBa_J23114 | tttatggctagctcagtcctaggtacaatgctagc |
| No: 19 | | |
| Seq ID | BBa_J23115 | tttatagctagctcagcccttggtacaatgctagc |
| No: 20 | | |
| Seq ID | BBa_J23116 | ttgacagctagctcagtcctagggactatgctagc |
| No: 21 | | |
| Seq ID | BBa_J23117 | ttgacagctagctcagtcctagggattgtgctagc |
| No: 22 | | |
| Seq ID | BBa_J23118 | ttgacggctagctcagtcctaggtattgtgctagc |
| No: 23 | | |
| Seq ID | Pc-aga | aattcagcgggttcgtgcgaactgtagacaattaatactcggctcgtataatgtgtggaaggctagcaggaggaattca |
| No: 24 | promoter | |
| Seq ID | Oli2892 | gcgtcaaggatgctctactc |
| No: 25 | | |
| Seq ID | Oli2893 | ttaattgccagccatcgcct |
| No: 26 | | |
| Seq ID | Oli2906 | gtgatctccaccggcttattagcgatagggattatctgtaccgtgccaattatggctcgcccttagttcctattccgaagttcct |
| No: 27 | | |
| Seq ID | Oli2907 | gtcgaaaatatcgagcagctcctgctctgacatagacgccacccgataccctttttgattcaccaccgccctgccactcatcgcagt |
| No: 28 | | |
| Seq ID | Oli2908 | tccgcacgtttatgatatgctatcgtactctttagcgagtacaaccgggggaggcattttgcttcccttagttcctattccgaagttcct |
| No: 29 | | |
| Seq ID | Oli2909 | cacgccgcatccggcattcggtgcacgatgcctgatgcgccacgtcttatcaggcctacaaaaccgccctgccactcatcgcagtac |
| No: 30 | | |
| Seq ID | Oli2910 | gacgctgccgcgtcttatcat |
| No: 31 | | |
| Seq ID | Oli2911 | tccgcacgtttatgatatgctatcgtactctttagcgagtacaaccgggggaggcattttgcttcttttgtaggcctgataagacgtg |
| No: 32 | | |
| Seq ID | Oli2913 | agaatagacaaaaactctgagtgtaataatgtagcctcgtgtcttgcgaggataagtgcattccttagttcctattccgaagttcct |
| No: 33 | | |
| Seq ID | Oli2914 | accataacaccccaaaatgcagagtgctttttttcagcttgatcagtcatgatgccacctttaccgccctgccactcatcgcagtac |
| No: 34 | | |
| Seq ID | Oli2915 | gtaataccgctgataatgttccaggt |
| No: 35 | | |
| Seq ID | Oli2916 | atagacaaaaactctgagtgtaataatgtagcctcgtgtcttgcgaggataagtgcattggtaaaggwggcatcatgactgatcaagct |
| No: 36 | | |
| Seq ID | Oli1550 | aattcagcgggttcgtgcgagctgtngrcaattaatactcggctcgtataatgtgtggaaggctagcaggaggaattcaatgat |
| No: 37 | | |
| Seq ID | Oli9094 | gttagaagtgatctccaccggcttattagcgatagggattatctgtaccgtgccaattatggctcgctggaaaaagctggtattgtggcaaaaaacacccgttcataatacgcgctgaattcagcgg |
| No: 38 | | gttcgtgcgagctgttgacaattaatactcggctcgtataatgtgt |
| Seq ID | aspC | atgtttgagaacattaccgccgctcctgccgacccgattctgggcctggccgatctgtttcgtgccgatgaacgtcccggcaaaattaacctcgggattggtgtctataaagatgagacgggcaaa |
| No: 39 | (E. coli) | accccggtactgaccagcgtgaaaaaggctgaacagtatctgctcgaaaatgaaaccaccaaaaattacctcggcattgacggcatccctgaatttggtcgctgcactcaggaactgctgtttggt |
| | | aaaggtagcgccctgatcaatgacaaacgtgctcgcacggcacagactccggggggcactggcgcactacgcgtggctgccgatttcctggcaaaaaataccagcgttaagcgtgtgtgggtg |
| | | agcaacccaagctggccgaaccataagagcgtctttaactctgcaggtctggaagttcgtgaatacgcttattatgatgcggaaaatcacactcttgacttcgatgcactgattaacagcctgaatg |
| | | aagctcaggctggcgacgtagtgctgttccatggctgctgccataacccaaccggtatcgaccctacgctggaacaatggcaaacactggcacaactctccgttgagaaaggctggttaccgctg |
| | | tttgacttcgcttaccagggttttgcccgtggtctggaagaagatgctgaaggactgcgcgctttcgcggctatgcataaagagctgattgttgccagttcctactctaaaaactttggcctgtacaac |
| | | gagcgtgttggcgcttgtactctggttgctgccgacagtgaaaccgttgatcgcgcattcagccaaatgaaagcggcgattcgcgctaactactctaacccaccagcacacggcgcttctgttgttg |
| | | ccaccatcctgagcaacgatgcgttacgtgcgatttgggaacaagagctgactgatatgcgccagcgtattcagcgtatgcgtcagttgttcgtcaatacgctgcaggaaaaaggcgcaaaccgc |
| | | gacttcagctttatcatcaaacagaacggcatgttctccttcagtggcctgacaaaagaacaagtgctgcgtctgcgcgaagagtttggcgtatatgcggttgcttctggtcgcgtaaatgtggccg |
| | | ggatgacaccagataacatggctccgctgtgcgaagcgattgtggcagtgctgtaa |
| Seq ID | taa1 | atggttaagttagagaatagtcgcaaacctgagaaaatctcaaataagaatattcctatgagcgacttcgtcgtgaatttagaccacggtgatccgacagcgtacgaagagtactggcgcaaaat |
| No: 40 | (Arabidopsis | gggagaccgctgtaccgtgaccattcgcggttgtgacttgatgagttatttttctgatatgacgaatctgtgttggttcttggagccggaactggaggatgcgattaaggatttacacggtgtcgttg |
| | thaliana) | gcaatgcagctacggaggaccgctatatcgtggtgggaactgggtctacgcagttatgccaggctgcggtccacgcattgtcttctcttgcgcgtagtcaaccagtttccgttgtggctgcggcacc |
| | | attttacagcacgtacgtggaggagaccacctatgttcgcagtgggatgtacaagtgggagggagacgcgtgggggtttgataagaaaggtccttatattgaacttgtgacatcaccgaataacc |
| | | cggacgggacgatccgcgagaccgtggttaatcgcccggatgatgatgaagcaaaggtaatccacgacttcgcttactattggccccattatacacctattacgcgtcgccaggatcacgacatc |
| | | atgctttttacctttagcaaaatcacggggcacgctggtagtcgtattggatgggcgctggtgaaggataaagaggtcgctaagaaaatggttgagtatattatcgtgaacagtatcggcgtttcg |
| | | aaagaatcgcaggtgcgtacagcgaaaattttgaatgtcctgaaggaaacgtgtaaatctgaatccgaatccgagaacttcttcaaatatggtcgtgagatgatgaagaatcgctgggagaagc |
| | | tgcgtgaagtagtcaaggagtcagacgccttcacattgcccaaatatcctgaggctttttgtaattatttcgggaagagtttagaatcatacccggcgtttgcctggctggggacaaaggaagaga |
| | | ccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttaga |
| | | gcgcttagccaacatgaaattgatcaaatctatcgatttgtaa |
| Seq ID | ipdC | atgtcgacttttacagtaggtgactatttattaactcgcttacaagagatcggggtaaagcatttatttggagtacctggtgactataacttacaatttcttgaccgtgtgatcgctcatcccgctattt |
| No: 41 | (Pantoea | catgggtaggctgtgccaacgagttgaacgcagcctatgcggctgatggatatgcacgttgcaatggggccggtgctcttcttacgaccttcggggttggcgagctttccgcgatcaacggcatcgc |
| | agglomerans) | tggttcttatgccgagtatctgccggtcattcatattgtcggggcaccagctacgcaggcgcagttgcaaggggactgcgtgcatcattctttaggagatggggacttccagcattttatccgcatgg |
| | | cgactgaagtatcggctgccacggctcaacttaacgccgacaacgccactgccgaaatcgaccgtgtaatcgtaagtgccctgcaagcacgccgtcccggatatctgtctctggctgtggatgtg |
| | | gcagccatggaagttcaaccaccagctcaaccattgaacagccaccaaagctctagtccggcagcccgtcgcgccttctcagatgctgccgagcgtttattggcgccagcacaacgcgtcagtct |
| | | gcttgcggattttttagccctgcgttggcagcaacaatcagccttagctgcattgcgcgaacaatgcgccatcccgtgcgcttcgctgttaatggggaagggagtcttggatgagcagcagcccgg |
| | | gtatgttggtacttatgctggcgaggcgtctgcgggccaggtttgtgaacagattgaacaagtagacgtcgcaatttgtgtcggggtccgttttacggacaccatcaccgccggatttacacagca |
| | | gtttaatcccgagcgcctgatcgacttgcagccacttagcgctagtgtcggcggagaacgctttgccccactttcaatggcagacgctctttccgaacttttgccgcttttcgagcgctatgggcaac |
| | | agtggcaaccaggggctacgccacctgcagcacaaccggctgaacccgccgccgtcatttcccaacaagcgttttggcaagcaatgcaagccttcttacagccgggcgatcttattttggccgaa |
| | | caaggtactgcggcatttggggctgccgcgcttcgtctgccaagccaggcccagttagtagtacaacctctttgggggtctattggatacaccctgccagcagcttttggtgcgcaaacggctgac |
| | | cctgatcgtcgcgtgatcctgatcattggagatggttcagcgcaattgactatccaagagttaggaagcatgctgcgcgaccaacaacgcttgatccttttcctgctgaacaatgagggctatactg |
| | | ttgaacgtgcgattcacggtgctacgcagcgttataatgacattgcccagtggaattggaccgctctgcctcacgcactttctttgcaaggccaggcacaatcatggcgtatctcggagacggtgca |
| | | attagaggaggtgatggctcgtttgtccgaacccaagtggctgtcattggtcgaggtcgtgatgcagaaggaggacctgcctcctttattacgcaaagtaacagcttgtcttaaccagcgtaatgg |
| | | ctgctaa |
| Seq ID | iad1 | atgccaactctcaacctcgatctgcctaacggcatcaagtcgaccatccaggctgacttgttcatcaacaacaagtttgtgcccgctcttgacggcaagacttttgcgaccatcaacccttccaccgg |
| No: 42 | (Ustilago | taaggagattggtcaggtcgccgaggcttccgccaaggacgttgatctcgctgtcaaggctgcgcgcgaggcattcgagactacttggggcgagaacacacccggtgacgctcgtggcagactt |
| | mayadis) | ctgatcaagctcgccgagctggtcgaggccaacattgacgagcttgctgccatcgagtcgctcgacaacggcaaggccttctcgatcgctaaaagctttgacgtcgctgccgtcgctgccaacttg |
| | | cgatactatggtggctgggccgacaagaaccacggcaaggtcatggaggtcgacaccaagcgcctcaactacacgcgccacgagcccatcggcgtgtgtggtcagatcatcccttggaacttcc |
| | | ccttgctcatgttcgcctggaagctcggccctgcgctcgccacaggtaacacgatcgtcctcaaaaccgccgagcagacaccactgtcagccatcaagatgtgtgagcttatcgtcgaggccggct |
| | | tcccgcctggtgtggtcaacgtcatttccggtttcggtcccgtcgccggtgccgccatctcacagcacatggacattgacaagatcgccttcaccggctcaacactggtgggccgcaacattatgaa |
| | | ggctgctgcttcgaccaacctgaagaaggtgacgctcgagctcggcggcaagtcgcccaacattatctttaaggacgccgacctggaccaggcggttcgatggtcggcgttcggtatcatgttca |
| | | atcacggacaatgctgctgtgcgggctcgcgtgtctatgtggaggaatcgatctatgacgctttcatggagaagatgactgcccactgcaaggcgctccaggtgggtgaccccttttccgccaaca |
| | | cgttccaaggtcctcaggtttcgcagctccagtacgaccgtatcatggagtacatcgagtccggcaagaaggacgctaaccttgcgctcggtggtgtgcgcaagggtaacgaaggctactttatc |
| | | gagcctaccatcttcaccgacgttcctcacgatgccaagattgccaaggaggagatcttcggccctgtcgtggtcgtctccaagttcaaagatgaaaaggacctcattcgcatcgccaacgactcg |
| | | atctacggcctcgctgccgctgtcttcagccgcgacatcagccgtgccatcgagactgcccacaagctcaaggccggcaccgtctgggtcaactgctacaaccagctcatccctcaggttccgttc |
| | | ggcggttacaaggcgtccggtatcggtcgagagctcggcgagtatgcgctcagcaactacacaaacatcaaggcggtgcatgtgaacctcagccagcctgcccctatctaa |
| Seq ID | hcxB | atggaaagtggtcatcgctttgatgctcagacgctgcacagttttattcaggctgtatttcgtcagatgggtagcgaggaacaagaagcgaaattagttgccgatcatttaatcgcggcaaacctg |
| No: 43 | (E. coli) | gcagggcatgattcacatggtattggcatgatcccaagctatgtacgctcctggagtcaggggcacctgcaaattaaccatcatgccaaaaccgttaaagaggggggggggggtcacgctcg |
| | | atggcgatcgcgcatttggtcaggtcgcggcacatgaagcgatggcgctggggattgagaaagcgcatcagcacggtattgccgccgtggcgctacataactcgcatcatatcggccgtatcgg |
| | | ttactgggcggagcagtgtgcagcggcggggtttgtctctatccactttgttagcgtggtcggtattccaatggtcgcgccgttccacggtcgcgacagccgctttggcaccaatccgttctgtgtgg |
| | | ttttccctcgtaaagataatttcccgctgttgcttgattacgccaccagcgccattgcatttggcaaaacccgcgtcgcctggcataaaggcgtccccgtgccgccaggttgcctgattgacgttaacg |
| | | gcgtgccgacgaccaatccggcggtaatgcaggagtcgccgttgggttcgctgttgacctttgccgaacataaaggctacgcccttgcagcgatgtgtgaaattcttggggggcgctttccggc |
| | | ggtaaaacgacgcatcaggaaacgttacaaaccagtcccgatgccattcttaactgcatgaccactatcatcatcaacccggaactcttcggcgcgccggattgtaacgcgcagaccgaagcctt |
| | | tgccgagtgggtgaaagcctcgccgcatgatgatgataagccgattttgctaccgggcgagtgggaagtgaacacgcgtcgcgaacggcagaagcaggggattccactggatgcgggaagct |
| | | ggcaggccatttgtgatgcagcgcggcagattggtatgccggaagagacgttgcaggctttctgtcagcagttagccagctaa |
| Seq ID | Idh4 | atggtgaccatgaatcgtaataaagttgttatcgtgggtacgggccaggtcggcgccaccgctgcgttcggcattgttacgcatggtttgtgcaacgaactggtcttgatcgaccgttccgccgcca |
| No: 44 | (Bifido- | aggcattgggggaggctcgtgaccttgacgacggtagcgaatttcaggaccgtcacgttaaggtccgcgcgggagattacgccgattgcaaagacgctgacattgttgttattactgttgggcgt |
| | bacterium | aagccaccagcgaacagtaaccgtatggcggaacttggttttacggtcggtttggtgggcgaagtagttgataatgttatggctagtggatttgatggagttattgttatggttagcaaccccgtcg |
| | longum) | atgttatggcttggtatgcttggaagcgttcgggactgccccgcacgcaagtccttggaacgggtaccgccttagatacctcccgccttaagacaattatcggagaagaaactggtttagatccac |
| | | gtaacgtaggcggctttgttatgggggaacatggtgattctcagttcactgcttggagcactgtatcgttaggcggtaaaccgtttgctcgctttttagcagacaatcaagatcgcttcgcatcggtat |
| | | caactaccgagatcgaggaaaaaacgcgcacacgtggcgacgaaatcgtagcggccaaggggggaccaattttggcattgcgtcaacagttgccggtatcgtacaaaccatcctgtgggac |
| | | gagcgccgcatcgtaccagtaagcacgctgcttgatggagagtacggtgaacatgacgtatttttgggtgtcccaacggaactgcgtgcaaacggggcgaatgaaatcgtagaattggacttaa |
| | | gcgaagacgagcgcgctaagttacatcattcggccgaattcgtgcgtgaccactgcaaaggattcctttga |
| Seq ID | fldH | atgaaaatcttagcttattgtgtaagacctgatgaaattgattcatttaaaaatttttctgaaaaatatggacatactgttgatttaataccagattcttttggtccaagtgtggctcatttagctaaa |
| No: 45 | (Clostridium | ggatatgatggcatttcaattttgggaaatgatacttgtaacagagaagcattagagaaaattaaagattgtggtataaaatatttagcaaccagaactgctggagtaaataatatagattttgatgctg |
| | sporogenes) | caaaagaatttggtataaatgtagctaatgttccagcttattctcctaattcagtttcagagtttactgtaggactcgcattatccctaacaaggaaaattccttttgcattaaaaagagtagagcttaa |
| | | caattttgcattaggtggattaataggagtagaattaagaaacttaactttaggagttataggaactggaagaataggtttaaaagtaattgaaggcttttcaggctttggtatgaaaaagatgatag |
| | | gttacgatatttttgaaaatgaaaaggctaaagaatatattgaatataaatctttagatgaagtatataaagaagcagacataataacactacatgcacctttaacagatgataattaccatatgata |
| | | gggaaagagagtattgctaaaatgaaagacggtgtattcataataaatgcagcacgaggagcattaattgatagtgaagctttaatagaaggactgaaatctggtaaaatagctggagcagcc |
| | | ttagattcttatgaatatgaacaaggagttttccataacaataaaatgaatgaaatcatgaaagatgatacattagcaagacttaagagttttccaaatgtagttattacacctcatttaggattctata |
| | | cagatgaagctgtatcaaatatgcttgaaattactcttatgaatttacaagagtttgaattaaaaggaacttgtaaaaatcagagggtatgtaagtaa |
| Seq ID | fldAIBC | atggaaaacaatacaaatatgtttagtggagtaaaggttattgaattagcaaattttatagctgctccagcagcaggtagattttttgctgatggtggtgcagaggtaataaaaattgaatcacctg |
| No: 46 | (Clostridium | ctggagatcctttaagatatactgctccttcagaaggaagaccattaagccaagaagaaaatactacttatgatttggaaaatgcaaataaaaaagcaatagtattaaatcttaaaagcgaaaaa |
| | sporogenes) | ggtaaaaagatattacatgaaatgttagcagaagcagatatattattaactaattggagaacaaaggctttagttaaacaaggattagactatgaaacactaaaagaaaaatatcctaaattagtt |
| | | tttgcacaaataactggttatggtgaaaaaggaccagataaagatcttccaggctttgattatactgcatttttcgctagaggcggtgtttcaggtactctttatgaaaaaggaactgtgcctccaaat |
| | | gttgttccaggacttggagaccatcaagctgggatgtttttagcagcgggtatggcaggagctttatataaagcaaaaacaacaggacaaggagataaagtaacagtaagtttaatgcatagtg |
| | | ctatgtatggactaggtattatgatacaagctgctcaatataaagatcatggattagtatatccgataaatcgtaatgaaactccaaatccttttatagtttcatataaatctaaggatgattactttg |
| | | ttcaagtatgtatgccaccatatgatgttttctatgatagatttatgaccgctttaggaagagaagatttagttggagacgaaagatacaataaaatagaaaatttaaaagatggacgtgctaaggaa |
| | | gtatacagtataatcgaacaacaaatggttacaaagacaaaggatgaatgggataaaatatttagagatgcagacattccatttgctatcgcacaaacttgggaagatttattagaagatgaaca |
| | | agcttgggcaaatgattatttgtataagatgaaatatccaacaggaaacgaaagagcattagtaagacttccagtattctttaaagaagcaggattaccagaatataatcaatcaccacaaatagc |
| | | agaaaatactgtagaagttttaaaagaaatgggatatacagaacaagagattgaggaattagaaaaagataaagatataatggtaaggaaggaaaaataatggcagacatttatactatggg |
| | | tgtagacataggttcaactgcatcaaaaacagtagtattaaaaaatggtaaagaaattgtaagtcaagcagtaataagtgtaggggccggaacaagtggccccaagagagctatagattctgta |
| | | ttaaaagatgctaaattatccattgaagatttagactatattgtatccactggatatggaagaaatagtttcgattttgctaacaaacaaatttctgaattaagttgtcatgcaaaaggggtctatttcg |
| | | ataacaataaagctagaacagttattgatataggcggacaagatattaaagtattaaaattagcggatagtggaagacttttaaactttataatgaatgataaatgtgctgcaggaacgggacga |
| | | tttttagatgtaatgtctagagtaatagaagttccagttgatgagttaggaaaaaaagcattagaaagcaaaaatccttgtactattagttctacctgtacagtatttgcagagtcagaagtaatttct |
| | | caacttgcaagaggagttaaaactgaagatttgatagcaggaatttgtaaatctgtagcatcaagagtggctagccttgcaaagagaagtggtatagaagaattagtagttatgagtggaggag |
| | | tagctaaaaatataggtgtagtaaaggcaatggaagcagaattgggaagagacatatatatatctaaaaattctcaattaaatggagcattgggagcaagtctatacgcttatgaaagttttcaaa |
| | | aagaaaggagctaaaaacatgagtgatagaaataaggaagtaaaagaaaaaaaggcaaagcattatcttagagagattactgcaaagcattacaaagaagctctcgaagcaaaagaaagg |
| | | ggagaaaaggttggttggtgtgcatctaacttcccacaagaaatagctacaacattgggggtaaaagttgtttatccagaaaatcatgcagcagctgtagcagctagagggaatggacaaaata |
| | | tgtgtgaacatgctgaggctatgggtttttctaatgatgtatgtggttatgcaagagtaaatttagctgttatggacataggtcatagtgaagatcaaccaatacctatgccagactttgtactttgctg |
| | | taataacatttgtaatcaaatgattaaatggtatgagcatatagcaaaaactttagatataccaatgattcttatagatataccatacaatacagaaaatactgtttcacaagatagaattaaatatat |
| | | tagagcacaatttgatgatgcaataaaacaattggaagaaataacaggcaaaaaatgggatgaaaataaatttgaagaagttatgaaaatatcccaagaaagtgcaaaacaatggttaagag |
| | | cagcatcctatgcaaagtataaaccttcaccatttagcggatttgatttatttaatcatatggctgtagcagtttgtgcaagaggtacacaagaagctgcagatgcatttaagatgttagcagatgaa |
| | | tatgaggagaatgtaaaaactggaaaatccacttataggggagaagaaaaacaacgtatattatttgaagggattgcctgttggccatatttgagacataaattaactaagcttagtgaatatggt |
| | | atgaacgtaactgcaactgtatacgcagaagcctttggtgttatatatgagaatatggatgaattaatggctgcttataataaagttcctaattcaattagttttgaaaacgcattaaaaatgagatta |
| | | aatgctgttacaagcactaatacagaaggtgctgttattcatataaatagaagctgtaaattatggagtggatttttatatgagctagcaagaagattagaaaaggaaacaggaattcctgtagtat |
| | | catttgatggggaccaggcagacccaagaaatttctcagaagctcaatatgatactagaattcaaggacttaatgaagtaatggttgctaaaaaggaggctgaataagatgtcaaattcagataa |
| | | attttttaatgactttaaggatattgtagaaaatcctaaaaaatatataatgaagcatatggaacaaactggacaaaaggctataggatgtatgccattatatactcctgaggaacttgtattagctg |
| | | ctggaatgtttccagtaggggtatggggaagcaatacagaactttcaaaagctaaaacatatttcccagcatttatttgttcaatattacaaacaacattggaaaatgcattaaatggagaatatga |
| | | tatgttatctggtatgatgattacaaattattgtgattcattaaaatgcatgggacaaaattttaaactaaccgttgaaaatattgagtttatcccagtaacagttccacaaaatagaaaaatggaagc |
| | | tggaaaagagtttttaaaaagtcaatataaaatgaatattgagcaattagaaaagatttctggtaataaaataacagatgaatctttagaaaaagctatagaaatatatgatgaacacagaaaag |
| | | taatgaatgacttttcaatgttagcatcaaaatatccaggtataataacaccaactaaacgtaattatgttatgaaatctgcttattatatggataaaaaagaacatactgaaaaagttagacaatta |
| | | atggatgaaattaaagctatagaaccaaaaccatttgaaggaaagagagttataactacaggtataattgcagattcagaagatttacttaaaatattagaagaaaataatatagctatagttggt |
| | | gatgatatagcacatgaatctagacaatatagaacattgactccagaagcgaacacaccaatggataggttagctgagcaatttgctaatagagaatgtagtactttatatgatcctgaaaagaa |
| | | aaggggtcaatatatagtagaaatggctaaagagagaaaagcagatggaattatatttttcatgacaaaattctgtgacccagaggaatatgattatccacaaatgaaaaaggattttgaagaa |
| | | gcaggcattccacatgtactaatagaaactgatatgcaaatgaaaaattatgaacaagctagaactccaattcaggctttttcagaaacactttaa |
| Seq ID | acdA | atgtttttcacagaacaacatgaacttattagaaaattagcaagagattttgcagagcaggaaatagagcctattgcagatgaagtagataaaactgccgagttccctaaagaaattgtgaaaaa |
| No: 47 | (Clostridium | aatggcccaaaatggtttttttggaataaaaatgcctaaagaatatggtggagctggagcagataatagagcctatgtaactataatggaagagatatcaagagcatctggtgtagctggtatat |
| | sporogenes) | atttatcatcaccaaattcattgcttggcacaccgttcctactagttggtacagatgaacaaaaggaaaaatatctaaaaccaatgattagaggagaaaaaacattagcttttgctttaacagaacc |
| | | aggtgcaggttcagacgcaggagcacttgcaacaactgctagagaagaaggggactattatatattaaatggaagaaaaacttttattacaggagctccaatatctgataatatcatagtgttcgc |
| | | aaaaacagatatgagcaaaggaacaaagggaattactacatttatagtagatagtaaacaggaaggtgtatcttttggtaaacctgaagacaagatgggtatgattggttgccctactagtgata |
| | | ttattttagagaatgtaaaggttcataaatctgatatacttggagaagtaaataaaggctttataacagcaatgaagactttaagtgttggtcgtataggagttgcttctcaagctttaggaatagca |
| | | caagctgctgtagatgaagctgttaaatatgcaaaacaaagaaaacaatttaatagacctatagcaaaatttcaagctattcaatttaaattggctaacatggaaacaaaattaaatgcggcaaa |
| | | attattagtgtataatgctgcttataaaatggattgtggagaaaaggcagataaagaagcttctatggcaaaatattttgctgctgaatctgctatacaaatcgtaaatgacgcactacaaatccatg |
| | | gtggttatggatacataaaagattataaaatagaaagattatatagagatgttagagtaattgctatatatgaaggaacttctgaagttcaacaaatggtaatcgcatcaaatcttttaaagtaa |
| Seq ID | p1915 | accgcaccaacaggctccaagccaagctttcctgacggaatgttaattctcgttgaccctgagcaggctgttgagccaggtgatttctgcatagccagacttgggggtgatgagtttaccttcaaga |
| No: 48 | | aactgatcagggatagcggtcaggtgtttttacaaccactaaacccacagtacccaatgatcccatgcaatgagagttgttccgttgtggggaaagttatcgctagtcagtggcctgaagagacgt |
| | | ttggctgatcggcaaggtgttctggtcggcgcatagctgataacaattgagcaagaatcttcatcgaattaggggaattttcactcccctcagaacataacatagtaaatggattgaattatgaaga |
| | | atggtttttatgcgacttaccgcagcaaaaataaagggaaagataagcgctcaataaacctgtctgttttccttaattctctgctggctgataatcatcacctgcaggttggctccaattatttgtata |
| | | ttcataaaatcgatggaaaaacttttctctttaccaaaacaaatgacaagagtctggttcagaagataaatcgctctaaagcttcagttgaagatattaagaacagcctcgcagatgacgaatcattg |
| | | ggattcccatcttttttgtttgttgaaggcgacaccattggttttgccagaactgttttcgggccgaccacatccgatctgacagattttttaatcgggaaaggaatgtcattaagcagtggagagcgc |
| | | gttcagatagagccactgatgaggggaaccaccaaagacgatgttatgcatatgcatttcatcggccgaacaacggtgaaggtagaagccaagctacctgtatttggcgatatattaaaggtctt |
| | | aggggcaacagatattgaaggggagctttttgactcattggatatagtcattaagccaaaatttaaaagggatataaaaaaggttgccaaggatattatttttaacccgtcacctcaattttcagac |
| | | attagcctgcgggcaaaagatgaggccggagatattttaacagaacattatctatcagaaaaaggccatctctcagcgcctctgaacaaggtcaccaatgctgagatagctgaagagatggcat |
| | | attgctacgcaagaatgaaaagtgatatactggaatgttttaaaaggcaggtgggcaaagttaaggattaattatcaggagtaattatgcggaacagaatcatgcctggtgtttacatagtaataa |
| | | ttccttacgttatcgtaagcatttgctatctccttttccgccactacattcctggtgtttctttttcagctcatagagatggtcttggggcgacattgtcatcatatgcaggaaccatgattgcaatcct |
| | | gattgctgccttgacgtttctaatcggaagcagaacgcgccgactggccaagattagagagtatgggtatatgacatcggtagttattgtctatgcccttagttttgttgagcttggagcttttttttct |
| | | gcggcttattgcttctttccagcataagcggctacatgatacccactatcgccatcggcattgcctctgcatcgttcattcatatatgcatccttgttttccaactatataatttgaccagagaacaaga |
| | | ataacccggcctcagcgccgggttttctttgcctcacgatcgcccccaaaacacataaccaattgtatttattgaaaaataaatagatacaactcactaaacatagcaattcagatctctcacctaccaa |
| | | acaatgcccccctgcaaaaaataaattcatataaaaaacatacagataaccatctgcggtgataaattatctctggcggtgttgacataaataccactggcggtgatactgagcacatcagcaggac |
| | | ggtggtggaggtgtcccatgtcgacttttacagtaggtgactatttattaactcgcttacaagagatcggggtaaagcatttatttggagtacctggtgactataacttacaatttcttgaccgtgtgat |
| | | cgctcatcccgctatttcatgggtaggctgtgccaacgagttgaacgcagcctatgcggctgatggatatgcacgttgcaatggggccggtgctcttcttacgaccttcggggttggcgagctttcc |
| | | gcgatcaacggcatcgctggttcttatgccgagtatctgccggtcattcatattgtcggggcaccagctacgcaggcgcagttgcaaggggactgcgtgcatcattctttaggagatggggacttc |
| | | cagcattttatccgcatggcgactgaagtatcggctgccacggctcaacttaacgccgacaacgccactgccgaaatcgaccgtgtaatcgtaagtgccctgcaagcacgccgtcccggatatct |
| | | gtctctggctgtggatgtggcagccatggaagttcaaccaccagctcaaccattgaacagccaccaaagctctagtccggcagcccgtcgcgccttctcagatgctgccgagcgtttattggcgcc |
| | | agcacaacgcgtcagtctgcttgcggattttttagccctgcgttggcagcaacaatcagccttagctgcattgcgcgaacaatgcgccatcccgtgcgcttcgctgttaatggggaagggagtcttg |
| | | gatgagcagcagcccgggtatgttggtacttatgctggcgaggcgtctgcgggccaggtttgtgaacagattgaacaagtagacgtcgcaatttgtgtcggggtccgttttacggacaccatcac |
| | | cgccggatttacacagcagtttaatcccgagcgcctgatcgacttgcagccacttagcgctagtgtcggcggagaacgctttgccccactttcaatggcagacgctctttccgaacttttgccgctttt |
| | | cgagcgctatgggcaacagtggcaaccaggggctacgccacctgcagcacaaccggctgaacccgccgccgtcatttcccaacaagcgttttggcaagcaatgcaagccttcttacagccggg |
| | | cgatcttattttggccgaacaaggtactgcggcatttggggctgccgcgcttcgtctgccaagccaggcccagttagtagtacaacctctttgggggtctattggatacaccctgccagcagcttttg |
| | | gtgcgcaaacggctgaccctgatcgtcgcgtgatcctgatcattggagatggttcagcgcaattgactatccaagagttaggaagcatgctgcgcgaccaacaacgcttgatccttttcctgctgaa |
| | | caatgagggctatactgttgaacgtgcgattcacggtgctacgcagcgttataatgacattgcccagtggaattggaccgctctgcctcacgcactttctttgcaaggccaggcacaatcatggcgt |
| | | atctcggagacggtgcaattagaggaggtgatggctcgtttgtccgaacccaagtggctgtcattggtcgaggtcgtgatgcagaaggaggacctgcctcctttattacgcaaagtaacagcttgt |
| | | cttaaccagcgtaatggctgctaagattcagatagtaagattgataataatctagaccaggcagtcgattaaagaggaggaaagtaatgtttgagaacattaccgccgctcctgccgacccgatt |
| | | ctgggcctggccgatctgtttcgtgccgatgaacgtcccggcaaaattaacctcgggattggtgtctataaagatgagacgggcaaaaccccggtactgaccagcgtgaaaaaggctgaacagt |
| | | atctgctcgaaaatgaaaccaccaaaaattacctcggcattgacggcatccctgaatttggtcgctgcactcaggaactcctgtttggtaaaggtagcgccctgatcaatgacaaacgtgctcgca |
| | | cggcacagactccggggggcactggcgcactacgcgtggctgccgatttcctggcaaaaaataccagcgttaagcgtgtgtgggtgagcaacccaagctggccgaaccataagagcgtcttta |
| | | actctgcaggtctggaagttcgtgaatacgcttattatgatgcggaaaatcacactcttgacttcgatgcactgattaacagcctgaatgaagctcaggctggcgacgtagtgctgttccatggctg |
| | | ctgccataacccaaccggtatcgaccctacgctggaacaatggcaaacactggcacaactctccgttgagaaaggctggttaccgctgtttgacttcgcttaccagggttttgcccgtggtctggaa |
| | | gaagatgctgaaggactgcgcgctttcgcggctatgcataaagagctgattgttgccagttcctactctaaaaactttggcctgtacaacgagcgtgttggcgcttgtactctggttgctgccgaca |
| | | gtgaaaccgttgatcgcgcattcagccaaatgaaagcggcgattcgcgctaactactctaacccaccagcacacggcgcttctgttgttgccaccatcctgagcaacgatgcgttacgtgcgatttg |
| | | ggaacaagagctgactgatatgcgccagcgtattcagcgtatgcgtcagttgttcgtcaatacgctgcaggaaaaaggcgcaaaccgcgacttcagctttatcatcaaacagaacggcatgttct |
| | | ccttcagtggcctgacaaaagaacaagtgctgcgtctgcgcgaagagtttggcgtatatgcggttgcttctggtcgcgtaaatgtggccgggatgacaccagataacatggctccgctgtgcgaa |
| | | gcgattgtggcagtgctgtaaatatcaatgataaaggaggaaacaaatgccaactctcaacctcgatctgcctaacggcatcaagtcgaccatccaggctgacttgttcatcaacaacaagtttgt |
| | | gcccgctcttgacggcaagacttttgcgaccatcaacccttccaccggtaaggagattggtcaggtcgccgaggcttccgccaaggacgttgatctcgctgtcaaggctgcgcgcgaggcattcg |
| | | agactacttggggcgagaacacacccggtgacgctcgtggcagacttctgatcaagctcgccgagctggtcgaggccaacattgacgagcttgctgccatcgagtcgctcgacaacggcaagg |
| | | ccttctcgatcgctaaaagctttgacgtcgctgccgtcgctgccaacttgcgatactatggtggctgggccgacaagaaccacggcaaggtcatggaggtcgacaccaagcgcctcaactacacg |
| | | cgccacgagcccatcggcgtgtgtggtcagatcatcccttggaacttccccttgctcatgttcgcctggaagctcggccctgcgctcgccacaggtaacacgatcgtcctcaaaaccgccgagcag |
| | | acaccactgtcagccatcaagatgtgtgagcttatcgtcgaggccggcttcccgcctggtgtggtcaacgtcatttccggtttcggtcccgtcgccggtgccgccatctcacagcacatggacattg |
| | | acaagatcgccttcaccggctcaacactggtgggccgcaacattatgaaggctgctgcttcgaccaacctgaagaaggtgacgctcgagctcggcggcaagtcgcccaacattatctttaaggac |
| | | gccgacctggaccaggcggttcgatggtcggcgttcggtatcatgttcaatcacggacaatgctgctgtgcgggctcgcgtgtctatgtggaggaatcgatctatgacgctttcatggagaagatg |
| | | actgcccactgcaaggcgctccaggtgggtgaccccttttccgccaacacgttccaaggtcctcaggtttcgcagctccagtacgaccgtatcatggagtacatcgagtccggcaagaaggacgc |
| | | taaccttgcgctcggtggtgtgcgcaagggtaacgaaggctactttatcgagcctaccatcttcaccgacgttcctcacgatgccaagattgccaaggaggagatcttcggccctgtcgtggtcgtc |
| | | tccaagttcaaagatgaaaaggacctcattcgcatcgccaacgactcgatctacggcctcgctgccgctgtcttcagccgcgacatcagccgtgccatcgagactgcccacaagctcaaggccgg |
| | | caccgtctgggtcaactgctacaaccagctcatccctcaggttccgttcggggttacaaggcgtccggtatcggtcgagagctcggcgagtatgcgctcagcaactacacaaacatcaaggcgg |
| | | tgcatgtgaacctcagccagcctgcccctatctaaatccgaactatccgaactggggatcctctagagtcgaccaggaggcaaatcatgactgatcaagctgaaaaaaagcactctgcattttgg |
| | | ggtgttatggttatagcaggtacagtaattggtggaggtatgtttgctttacctgttgatcttgccggtgcctggtttttctggggtgcctttatccttatcattgcctggttttcaatgcttcattccg |
| | | ggttattgttattagaagcaaatttaaattatcccgtcggctccagttttaacaccatcaccaaagatttaatcggtaacacctggaacattatcagcggtattaccgttgccttcgttctctatatcct |
| | | cacttatgcctatatctctgctaatggtgcgatcattagtgaaacgatatcaatgaatttgggttatcacgctaatccacgtattgtcgggatctgcacagccattttcgttgccagcgtattgtggtta |
| | | agttcgttagccgccagtcgtattacctcattgttcctcgggctgaagattatctcctttgtgatcgtgtttggttcttttttcttccaggtcgattactccattctgcgcgacgccaccagctccactg |
| | | cgggaacgtcttacttcccgtatatctttatggctttgccggtgtgtctggcgtcatttggtttccacggcaatattcccagcctgattatttgctatggaaaacgcaaagataagttaatcaaaagcgt |
| | | ggtatttggttcgctgctggcgctggtgatttatctcttctggctctattgcaccatggggaatattccgcgagaaagctttaaggcgattatctcctcaggcggcaacgttgattcgctggtgaaatcg |
| | | ttcctcggcaccaaacagcacggcattatcgagttttgcctgctggtgttctctaacttagctgttgccagttcgttctttggtgtcacgctggggttgttcgattatctggcggacctgtttaagattg |
| | | ataactcccacggcgggcgtttcaaaaccgtgctgttaaccttcctgccacctgcgttgttgtatctgatcttcccgaacggctttatttacgggatcggcggtgccgggctgtgcgccaccatctgggg |
| | | gtcattattcccgcagtgcttgcaatcaaagctcgcaagaagtttcccaatcagatgttcacggtctggggcggcaatcttattccggcgattgtcattctctttggtataaccgtgattttgtgctggt |
| | | tcggcaacgtctttaacgtgttacctaaatttggctaaatccgaactccaattattgaaggccgctaacgcggccttttttttttctggtctccctgaacgctctctactagagtcacactggctcacct |
| | | tcggggggcctttctgcgtttataggtacccgttgaaaagggattataaaagctcacttattcaggcgtagcaccaggcgtttaagggcaccaataactgccttaaaaaaattacgccccgccctgccac |
| | | tcatcgcagtactgttgtaattcattaagcattctgccgacatggaagccatcacagacggcatgatgaacctgaatcgccagcggcatcagcaccttgtcgccttgcgtataatatttgcccatggtga |
| | | aaacgggggcgaagaagttgtccatattggccacgtttaaatcaaaactggtgaaactcacccagggattggctgagacgaaaaacatattctcaataaaccctttagggaaataggccaggttttcacc |
| | | gtaacacgccacatcttgcgaatatatgtgtagaaactgccggaaatcgtcgtggtattcactccagagcgatgaaaacgtttcagtttgctcatggaaaacggtgtaacaagggtgaacactatc |
| | | ccatatcaccagctcaccgtctttcattgccatacggaattccggatgagcattcatcagggggcaagaatgtgaataaaggccggataaaacttgtgcttatttttctttacggtctttaaaaaggc |
| | | cgtaatatccagctgaacggtctggttataggtacattgagcaactgactgaaatgcctcaaaatgttctttacgatgccattgggatatatcaacggtggtatatccagtgatttttttctccatttta |
| | | gcttccttagctcctgaaaatctcgataactcaaaaaatacgcccggtagtgatcttatttcattatggtgaaagttggaacctcttacgtgccgatcaacgtctcattttcgccaaaagttggcccagg |
| | | gcttcccggtatcaacagggacaccaggatttatttattctgcgaagtgatcttccgtcacaggtatttattcggcgcaaagtgcgtcgggtgatgctgccaacttactgatttagtgtatgatggtgtt |
| | | tttgaggtgctccagtggcttctgtttctatcagctgtccctcctgttcagctactgacggggtggtgcgtaacggcaaaagcaccgccggacatcagcgctagcggagtgtatactggcttactatg |
| | | ttggcactgatgagggtgtcagtgaagtgcttcatgtggcaggagaaaaaaggctgcaccggtgcgtcagcagaatatgtgatacaggatatattccgcttcctcgctcactgactcgctacgctc |
| | | ggtcgttcgactgcggcgagcggaaatggcttacgaacggggcggagatttcctggaagatgccaggaagatacttaacagggaagtgagagggccgcggcaaagccgtttttccataggct |
| | | ccgcccccctgacaagcatcacgaaatctgacgctcaaatcagtggtggcgaaacccgacaggactataaagataccaggcgtttccccctggcggctccctcgtgcgctctcctgttcctgccttt |
| | | cggtttaccggtgtcattccgctgttatggccgcgtttgtctcattccacgcctgacactcagttccgggtaggcagttcgctccaagctggactgtatgcacgaaccccccgttcagtccgaccgctg |
| | | cgccttatccggtaactatcgtcttgagtccaacccggaaagacatgcaaaagcaccactggcagcagccactggtaattgatttagaggagttagtcttgaagtcatgcgccggttaaggctaaa |
| | | ctgaaaggacaagttttggtgactgcgctcctccaagccagttacctcggttcaaagagttggtagctcagagaaccttcgaaaaaccgccctgcaaggcggttttttcgttttcagagcaagagat |
| | | tacgcgcagaccaaaacgatctcaagaagatcatcttattaatcagataaaatatttgctcatgagcccgaagtggcgagcccgatcttccccatcggtgatgtcggcgatataggcgccagcaa |
| | | ccgcacctgtggcgccggtgatgccggccacgatgcgtccggcgtagaggatctgctcatgtttgacagcttatcatcgatgcataatgtgcctgtcaaatggacgaagcagggattctgcaaac |
| | | cctatgctactccgtcaagccgtcaattgtctgattcgttaccaagtggaggttgtataatgtatcaccgccagaggtaaaatagtcaacacgcacggtgttagatatttatcccttgcggtgatagat |
| | | ttaacgtatgagcacaaaaaagaaaccattaacacaagagcagcttgaggacgcacgtcgccttaaagcaatttatgaaaaaaagaaaaatgaacttggcttatcccaggaatctgtcgcaga |
| | | caagatggggatggggcagtcaggcgttggtgctttatttaatggcatcaatgcattaaatgcttataacgccgcattgcttgcaaaaattctcaaagttagcgttgaagaatttagcccttcaatcg |
| | | ccagagaaatctacgagatgtatgaagcggttagtatgcagccgtcacttagaagtgagtatgagtaccctgttttttctcatgttcaggcagggatgttctcacctgagcttagaacctttaccaaa |
| | | ggtgatgcggagagatgggtaagcacaaccaaaaaagccagtgattctgcattctgccttgaggttgaaggtaattccatg |
| Seq ID | p2169 | cgatcgtggctggctcgaagatacctgcaagaatgtcattgcgctgccattctccaaattgcagttcgcgcttagctggataacgccacggaatgatgtcgtcgtgcacaacaatggtgacttctac |
| No: 49 | | agcgcggagaatctcgctctctccaggggaagccgaagtttccaaaaggtcgttgatcaaagctcgccgcgttgtttcatcaagccttacggtcaccgtaaccagcaaatcaatatcactgtgtgg |
| | | cttcaggccgccatccactgcggagccgtacaaatgtacggccagcaacgtcggttcgagatggcgctcgatgacgccaactacctctgatagttgagtcgatacttcggcgatcaccgcttccct |
| | | catactcttcctttttcaatattattgaagcatttatcagggttattgtctcatgagcggatacatatttgaatgtatttagaaaaataaacaaatagctagctcactcggtcgctacgctccgggcgtg |
| | | agactgcggcgggcgctgcggacacatacaaagttacccacagattccgtggataagcaggggactaacatgtgaggcaaaacagcagggccgcgccggtggcgtttttccataggctccgcc |
| | | ctcctgccagagttcacataaacagacgcttttccggtgcatctgtgggagccgtgaggctcaaccatgaatctgacagtacgggcgaaacccgacaggacttaaagatccccaccgtttccggc |
| | | gggtcgctccctcttgcgctctcctgttccgaccctgccgtttaccggatacctgttccgcctttctcccttacgggaagtgtggcgctttctcatagctcacacactggtatctcggctcggtgtaggt |
| | | cgttcgctccaagctgggctgtaagcaagaactccccgttcagcccgactgctgcgccttatccggtaactgttcacttgagtccaacccggaaaagcacggtaaaacgccactggcagcagccat |
| | | tggtaactgggagttcgcagaggatttgtttagctaaacacgcggttgctcttgaagtgtgcgccaaagtccggctacactggaaggacagatttggttgctgtgctctgcgaaagccagttacca |
| | | cggttaagcagttccccaactgacttaaccttcgatcaaaccacctccccaggtggttttttcgtttacagggcaaaagattacgcgcagaaaaaaaggatctcaagaagatcctttgatcttttctac |
| | | tgaaccgctctagatttcagtgggctagcaggaggaattcaatgatatgtcgacttttacagtaggtgactatttattaactcgcttacaagagatcggggtaaagcatttatttggagtacctggtg |
| | | actataacttacaatttcttgaccgtgtgatcgctcatcccgctatttcatgggtaggctgtgccaacgagttgaacgcagcctatgcggctgatggatatgcacgttgcaatggggccggtgctcttc |
| | | ttacgaccttcggggttggcgagctttccgcgatcaacggcatcgctggttcttatgccgagtatctgccggtcattcatattgtcggggcaccagctacgcaggcgcagttgcaaggggactgcgt |
| | | gcatcattctttaggagatggggacttccagcattttatccgcatggcgactgaagtatcggctgccacggctcaacttaacgccgacaacgccactgccgaaatcgaccgtgtaatcgtaagtgc |
| | | cctgcaagcacgccgtcccggatatctgtctctggctgtggatgtggcagccatggaagttcaaccaccagctcaaccattgaacagccaccaaagctctagtccggcagcccgtcgcgccttctc |
| | | agatgctgccgagcgtttattggcgccagcacaacgcgtcagtctgcttgcggattttttagccctgcgttggcagcaacaatcagccttagctgcattgcgcgaacaatgcgccatcccgtgcgct |
| | | tcgctgttaatggggaagggagtcttggatgagcagcagcccgggtatgttggtacttatgctggcgaggcgtctgcgggccaggtttgtgaacagattgaacaagtagacgtcgcaatttgtgt |
| | | cggggtccgttttacggacaccatcaccgccggatttacacagcagtttaatcccgagcgcctgatcgacttgcagccacttagcgctagtgtcggcggagaacgctttgccccactttcaatggca |
| | | gacgctctttccgaacttttgccgcttttcgagcgctatgggcaacagtggcaaccaggggctacgccacctgcagcacaaccggctgaacccgccgccgtcatttcccaacaagcgttttggcaa |
| | | gcaatgcaagccttcttacagccgggcgatcttattttggccgaacaaggtactgcggcatttggggctgccgcgcttcgtctgccaagccaggcccagttagtagtacaacctctttgggggtcta |
| | | ttggatacaccctgccagcagcttttggtgcgcaaacggctgaccctgatcgtcgcgtgatcctgatcattggagatggttcagcgcaattgactatccaagagttaggaagcatgctgcgcgacc |
| | | aacaacgcttgatccttttcctgctgaacaatgagggctatactgttgaacgtgcgattcacggtgctacgcagcgttataatgacattgcccagtggaattggaccgctctgcctcacgcactttctt |
| | | tgcaaggccaggcacaatcatggcgtatctcggagacggtgcaattagaggaggtgatggctcgtttgtccgaacccaagtggctgtcattggtcgaggtcgtgatgcagaaggaggacctgc |
| | | ctcctttattacgcaaagtaacagcttgtcttaaccagcgtaatggctgctaagattcagatagtaagattgataataatctagaccaggcagtggattaaagaggaggaaagtaatggttaagtt |
| | | agagaatagtcgcaaacctgagaaaatctcaaataagaatattcctatgagcgacttcgtcgtgaatttagaccacggtgatccgacagcgtacgaagagtactggcgcaaaatgggagaccg |
| | | ctgtaccgtgaccattcgcggttgtgacttgatgagttatttttctgatatgacgaatctgtgttggttcttggagccggaactggaggatgcgattaaggatttacacggtgtcgttggcaatgcagc |
| | | tacggaggaccgctatatcgtggtgggaactgggtctacgcagttatgccaggctgcggtccacgcattgtcttctcttgcgcgtagtcaaccagtttccgttgtggctgcggcaccattttacagca |
| | | cgtacgtggaggagaccacctatgttcgcagtgggatgtacaagtgggagggagacgcgtgggggtttgataagaaaggtccttatattgaacttgtgacatcaccgaataacccggacggga |
| | | cgatccgcgagaccgtggttaatcgcccggatgatgatgaagcaaaggtaatccacgacttcgcttactattggccccattatacacctattacgcgtcgccaggatcacgacatcatgctttttacc |
| | | tttagcaaaatcacggggcacgctggtagtcgtattggatgggcgctggtgaaggataaagaggtcgctaagaaaatggttgagtatattatcgtgaacagtatcggcgtttcgaaagaatcgc |
| | | aggtgcgtacagcgaaaattttgaatgtcctgaaggaaacgtgtaaatctgaatccgaatccgagaacttcttcaaatatggtcgtgagatgatgaagaatcgctgggagaagctgcgtgaagt |
| | | agtcaaggagtcagacgccttcacattgcccaaatatcctgaggctttttgtaattatttcgggaagagtttagaatcatacccggcgtttgcctggctggggacaaaggaagagaccgacttggt |
| | | atcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagcc |
| | | aacatgaaattgatcaaatctatcgatttgtaaatatcaatgataaaggaggaaacaaatgccaactctcaacctcgatctgcctaacggcatcaagtcgaccatccaggctgacttgttcatcaac |
| | | aacaagtigtgcccgctcttgacggcaagacttttgcgaccatcaacccttccaccggtaaggagattggtcaggtcgccgaggcttccgccaaggacgttgatctcgctgtcaaggctgcgcgc |
| | | gaggcattcgagactacttggggcgagaacacacccggtgacgctcgtggcagacttctgatcaagctcgccgagctggtcgaggccaacattgacgagcttgctgccatcgagtcgctcgaca |
| | | acggcaaggccttctcgatcgctaaaagctttgacgtcgctgccgtcgctgccaacttgcgatactatggtggctgggccgacaagaaccacggcaaggtcatggaggtcgacaccaagcgcct |
| | | caactacacgcgccacgagcccatcggcgtgtgtggtcagatcatcccttggaacttccccttgctcatgttcgcctggaagctcggccctgcgctcgccacaggtaacacgatcgtcctcaaaacc |
| | | gccgagcagacaccactgtcagccatcaagatgtgtgagcttatcgtcgaggccggcttcccgcctggtgtggtcaacgtcatttccggtttcggtcccgtcgccggtgccgccatctcacagcaca |
| | | tggacattgacaagatcgccttcaccggctcaacactggtgggccgcaacattatgaaggctgctgcttcgaccaacctgaagaaggtgacgctcgagctcggcggcaagtcgcccaacattat |
| | | ctttaaggacgccgacctggaccaggcggttcgatggtcggcgttcggtatcatgttcaatcacggacaatgctgctgtgcgggctcgcgtgtctatgtggaggaatcgatctatgacgctttcatg |
| | | gagaagatgactgcccactgcaaggcgctccaggtgggtgaccccttttccgccaacacgttccaaggtcctcaggtttcgcagctccagtacgaccgtatcatggagtacatcgagtccggcaa |
| | | gaaggacgctaaccttgcgctcggtggtgtgcgcaagggtaacgaaggctactttatcgagcctaccatcttcaccgacgttcctcacgatgccaagattgccaaggaggagatcttcggccctgt |
| | | cgtggtcgtctccaagttcaaagatgaaaaggacctcattcgcatcgccaacgactcgatctacggcctcgctgccgctgtcttcagccgcgacatcagccgtgccatcgagactgcccacaagct |
| | | caaggccggcaccgtctgggtcaactgctacaaccagctcatccctcaggttccgttcggcggttacaaggcgtccggtatcggtcgagagctcggcgagtatgcgctcagcaactacacaaac |
| | | atcaaggcggtgcatgtgaacctcagccagcctgcccctatctaaaagaagaagtgaatgccactgctgtttgaaagcttaagccatcaaatttacctgtcagccccgtgtttatcctgatcctgct |
| | | ggggtactgcctgacgcgcctcgggaaatggcccgccagcatcagcgaagggatgaaccgctttgtgtttaacgttgccctgccctgcatgctgtttcgggtgatgagcaccttctcgcaaagtcc |
| | | gccggtggatgcgcgcctgttgctggcctttttcggcagttgcctgctggtgtttatcgtggggcgactgctggcaacaaggctgtttaatctggatggcgtagcggcgtcggtttttgcgctgggcg |
| | | gcatcttttccaataacgtgatgctggggatcccagtggccacggtactgctgggaccagaagccctgcccccggtggcgctggtgctggtgtttaacagcctgatcctctggaccttactgaccgt |
| | | ttcggtcgagtgggcaaaacagggcagcttctcgctggaaggactctggcgcaccctgctcggcgtgctgaagaatccgctgatcatcggtattctcagcggtacagcctggagctttctgcaac |
| | | gtcccttgccgctgctggccgctgaaccgctaaagatgctggcctcactggccgcacctctgtcgctggtgacgctgggcatgagcctggcgcactatcgggtgcgggatggcttaaaagagag |
| | | ctataccatctgcctgctgaaactggtgctgcaaccgatgacggtatgggctatcgcgtggctgaccgatctgccgccgctggagagtaaagtggtggtgctgctgggatcgatggcggtcggg |
| | | gttaacgtctacctgatgtcgcagaagtttaatgttatcaccggcccggcagcggcgagcatgctcttttctaccgtgtttgcggcggtgacaacgccaatctggatgatgctgatgacgctggcgg |
| | | gctattaaatcatgatcaggagtcacaccatgaccattacgtctctggatggctatcgctggctgaagaacgatattattcgcggtaattttcaaccggatgaaaaattacgaatgagtttgctgaca |
| | | tcgcgttatgcacttggcgttgggccgttacgggaagctctttcgcaactggtggcggaacggctggtcacggtggtgaatcaaaaagggtatcgggtggcgtctatgtcagagcaggagctgct |
| | | cgatattttcgacgcccgcgccaatatggaagcgatgttagtgagtctggcgattgcccgcggtggcgatgagtgggaggcagacgttctcgcaaaagcgcatctgctgagtaagcttgaggcc |
| | | tgtgacgccagcgagaaaatgcttgatgagtgggatctgcgtcatcaggcgtttcatacggcaattgtggcgggctgtggttctcactatttgctgcaaatgcgtgaacggttgtttgatctggcgg |
| | | cgcgttatcgatttatctggctgcggcgaacggtgctttcggtggaaatgctggaggataaacacgatcagcaccagaccctgactgcggcggtactggcgcgagataccgcgcgcgccagtga |
| | | gttaatgcgccagcatttactgacgccaattcccattatccagcaggcgatggctggcaattaacgctctctactagagtcacactggctcaccttcggggggcctttctgcgggtctcacgctcttg |
| | | atatgcaggtgctgcaggcttaatttgatgcctggcagtttatgggggcgtcctgcccgccaccctccgggccgttgcttcctgcaggcacggcagcggctgcgcagggctcttctcgatcaatat |
| | | cgttcactcatccaatctcactgacggttaggcgtgcctggtaccctgccctgaaccgacgaccgggtcatcgtggccggatcttgcggcccctcggcttgaacgaattgttagacattatttgccga |
| | | ctaccttggtgatctcgcctttcacgtagtggacaaattcttccaactgatctgcgcgcgaggccaagcgatcttcttcttgtccaagataagcctgtctagcttcaagtatgacgggctgatactggg |
| | | ccggcaggcgctccattgcccagtcggcagcgacatccttcggcgcgattttgccggttactgcgctgtaccaaatgcgggacaacgtaagcactacatttcgctcatcgccagcccagtcgggc |
| | | ggcgagttccatagcgttaaggtttcatttagcgcctcaaatagatcctgttcaggaaccggatcaaagagttcctccgccgctggacctaccaaggcaacgctatgttctcttgcttttgtcagcaa |
| | | gatagccagatcaatg |
| Seq ID | p2526 | aaatatcaatgataaaggaggaaacaaatgccaactctcaacctcgatctgcctaacggcatcaagtcgaccatccaggctgacttgttcatcaacaacaagtttgtgcccgctcttgacggcaag |
| No: 50 | | acttttgcgaccatcaacccttccaccggtaaggagattggtcaggtcgccgaggcttccgccaaggacgttgatctcgctgtcaaggctgcgcgcgaggcattcgagactacttggggcgagaa |
| | | cacacccggtgacgctcgtggcagacttctgatcaagctcgccgagctggtcgaggccaacattgacgagcttgctgccatcgagtcgctcgacaacggcaaggccttctcgatcgctaaaagct |
| | | ttgacgtcgctgccgtcgctgccaacttgcgatactatggtggctgggccgacaagaaccacggcaaggtcatggaggtcgacaccaagcgcctcaactacacgcgccacgagcccatcggcg |
| | | tgtgtggtcagatcatcccttggaacttccccttgctcatgttcgcctggaagctcggccctgcgctcgccacaggtaacacgatcgtcctcaaaaccgccgagcagacaccactgtcagccatcaa |
| | | gatgtgtgagcttatcgtcgaggccggcttcccgcctggtgtggtcaacgtcatttccggtttcggtcccgtcgccggtgccgccatctcacagcacatggacattgacaagatcgccttcaccggct |
| | | caacactggtgggccgcaacattatgaaggctgctgcttcgaccaacctgaagaaggtgacgctcgagctcggggcaagtcgcccaacattatctttaaggacgccgacctggaccaggcgg |
| | | ttcgatggtcggcgttcggtatcatgttcaatcacggacaatgctgctgtgcgggctcgcgtgtctatgtggaggaatcgatctatgacgctttcatggagaagatgactgcccactgcaaggcgct |
| | | ccaggtgggtgaccccttttccgccaacacgttccaaggtcctcaggtttcgcagctccagtacgaccgtatcatggagtacatcgagtccggcaagaaggacgctaaccttgcgctcggtggtgt |
| | | gcgcaagggtaacgaaggctactttatcgagcctaccatcttcaccgacgttcctcacgatgccaagattgccaaggaggagatcttcggccctgtcgtggtcgtctccaagttcaaagatgaaaa |
| | | ggacctcattcgcatcgccaacgactcgatctacggcctcgctgccgctgtcttcagccgcgacatcagccgtgccatcgagactgcccacaagctcaaggccggcaccgtctgggtcaactgcta |
| | | caaccagctcatccctcaggttccgttcggcggttacaaggcgtccggtatcggtcgagagctcggcgagtatgcgctcagcaactacacaaacatcaaggcggtgcatgtgaacctcagccag |
| | | cctgcccctatctaaatccgaactccaattattgatgaaaagggattataaaagctcacttattcaggcgtagcacccaggcgtttaagggcaccaataactgccttaaaaaaattacgccccgccc |
| | | tgccactcatcgcagtactgttgtaattcattaagcattctgccgacatggaagccatcacaaacggcatgatgaacctgaatcgccagcggcatcagcaccttgtcgccttgcgtataatatttgcc |
| | | catggtgaaaacgggggcgaagaagttgtccatattggccacgtttaaatcaaaactggtgaaactcacccagggattggctgagacgaaaaacatattctcaataaaccctttagggaaatag |
| | | gccaggttttcaccgtaacacgccacatcttgcgaatatatgtgtagaaactgccggaaatcgtcgtggtattcactccagagcgatgaaaacgtttcagtttgctcatggaaaacggtgtaacaa |
| | | gggtgaacactatcccatatcaccagctcaccgtctttcattgccatacggaattccggatgagcattcatcagggggcaagaatgtgaataaaggccggataaaacttgtgcttatttttctttac |
| | | ggtctttaaaaaggccgtaatatccagctgaacggtctggttataggtacattgagcaactgactgaaatgcctcaaaatgttctttacgatgccattgggatatatcaacggtggtatatccagtga |
| | | tttttttctccattttagcttccttagctcctgaaaatctcgataactcaaaaaatacgcccggtagtgatcttatttcattatggtgaaagttggaacctcttacgtgccgatcaacggtcgctacgct |
| | | ccgggcgtgagactgcggcgggcgctgcggacacatacaaagttacccacagattccgtggataagcaggggactaacatgtgaggcaaaacagcagggccgcgccggtggcgtttttccatag |
| | | gctccgccctcctgccagagttcacataaacagacgcttttccggtgcatctgtgggagccgtgaggctcaaccatgaatctgacagtacgggcgaaacccgacaggacttaaagatccccaccg |
| | | tttccggcgggtcgctccctcttgcgctctcctgttccgaccctgccgtttaccggatacctgttccgcctttctcccttacgggaagtgtggcgctttctcatagctcacacactggtatctcggctcg |
| | | gtgtaggtcgttcgctccaagctgggctgtaagcaagaactccccgttcagcccgactgctgcgccttatccggtaactgttcacttgagtccaacccggaaaagcacggtaaaacgccactggcag |
| | | cagccattcgtaactgggagttcgcagaggatttgtttagctaaacacgcggttgctcttgaagtgtgcgccaaagtccggctacactggaaggacagatttggttgctgtgctctgcgaaagcca |
| | | gttaccacggttaagcagttccccaactgacttaaccttcgatcaaaccacctccccaggtggttttttcgtttacagggcaaaagattacgcgcagaaaaaaaggatctcaagaagatcctttgat |
| | | ctgatgccggccacgatgcgtccggcgtagaggatctgctcatgtttgacagcttatcatcgatgcataatgtgcctgtcaaatggacgaagcagggattctgcaaaccctatgctactccgtcaag |
| | | ccgtcaattgtctgattcgttaccaagtggaggttgtataatgattcagcgggttcgtgcgagctgtagacaattaatactcggctcgtataatgtgtggaaggctagcaggaggaattcaatgata |
| | | tgtcgacttttacagtaggtgactatttattaactcgcttacaagagatcggggtaaagcatttatttggagtacctggtgactataacttacaatttcttgaccgtgtgatcgctcatcccgctatttc |
| | | atgggtaggctgtgccaacgagttgaacgcagcctatgcggctgatggatatgcacgttgcaatggggccggtgctcttcttacgaccttcggggttggcgagctttccgcgatcaacggcatcgctg |
| | | gttcttatgccgagtatctgccggtcattcatattgtcggggcaccagctacgcaggcgcagttgcaaggggactgcgtgcatcattctttaggagatggggacttccagcattttatccgcatggc |
| | | gactgaagtatcggctgccacggctcaacttaacgccgacaacgccactgccgaaatcgaccgtgtaatcgtaagtgccctgcaagcacgccgtcccggatatctgtctctggctgtggatgtgg |
| | | cagccatggaagttcaaccaccagctcaaccattgaacagccaccaaagctctagtccggcagcccgtcgcgccttctcagatgctgccgagcgtttattggcgccagcacaacgcgtcagtctg |
| | | cttgcggattttttagccctgcgttggcagcaacaatcagccttagctgcattgcgcgaacaatgcgccatcccgtgcgcttcgctgttaatggggaagggagtcttggatgagcagcagcccggg |
| | | tatgttggtacttatgctggcgaggcgtctgcgggccaggtttgtgaacagattgaacaagtagacgtcgcaatttgtgtcggggtccgttttacggacaccatcaccgccggatttacacagcagt |
| | | ttaatcccgagcgcctgatcgacttgcagccacttagcgctagtgtcggcggagaacgctttgccccactttcaatggcagacgctctttccgaacttttgccgcttttcgagcgctatgggcaacag |
| | | tggcaaccaggggctacgccacctgcagcacaaccggctgaacccgccgccgtcatttcccaacaagcgttttggcaagcaatgcaagccttcttacagccgggcgatcttattttggccgaaca |
| | | aggtactgcggcatttggggctgccgcgcttcgtctgccaagccaggcccagttagtagtacaacctctttgggggtctattggatacaccctgccagcagcttttggtgcgcaaacggctgaccct |
| | | gatcgtcgcgtgatcctgatcattggagatggttcagcgcaattgactatccaagagttaggaagcatgctgcgcgaccaacaacgcttgatccttttcctgctgaacaatgagggctatactgttg |
| | | aacgtgcgattcacggtgctacgcagcgttataatgacattgcccagtggaattggaccgctctgcctcacgcactttctttgcaaggccaggcacaatcatggcgtatctcggagacggtgcaatt |
| | | agaggaggtgatggctcgtttgtccgaacccaagtggctgtcattggtcgaggtcgtgatgcagaaggaggacctgcctcctttattacgcaaagtaacagcttgtcttaaccagcgtaatggctg |
| | | ctaagattcagatagtaagattgataataatctagaccaggcagtggattaaagaggaggaaagtaatgtttgagaacattaccgccgctcctgccgacccgattctgggcctggccgatctgttt |
| | | cgtgccgatgaacgtcccggcaaaattaacctcgggattggtgtctataaagatgagacgggcaaaaccccggtactgaccagcgtgaaaaaggctgaacagtatctgctcgaaaatgaaacc |
| | | accaaaaattacctcggcattgacggcatccctgaatttggtcgctgcactcaggaactgctgtttggtaaaggtagcgccctgatcaatgacaaacgtgctcgcacggcacagactccgggggg |
| | | cactggcgcactacgcgtggctgccgatttcctggcaaaaaataccagcgttaagcgtgtgtgggtgagcaacccaagctggccgaaccataagagcgtctttaactctgcaggtctggaagttc |
| | | gtgaatacgcttattatgatgcggaaaatcacactcttgacttcgatgcactgattaacagcctgaatgaagctcaggctggcgacgtagtgctgttccatggctgctgccataacccaaccggtat |
| | | cgaccctacgctggaacaatggcaaacactggcacaactctccgttgagaaaggctggttaccgctgtttgacttcgcttaccagggttttgcccgtggtctggaagaagatgctgaaggactgcg |
| | | cgctttcgcggctatgcataaagagctgattgttgccagttcctactctaaaaactttggcctgtacaacgagcgtgttggcgcttgtactctggttgctgccgacagtgaaaccgttgatcgcgcatt |
| | | cagccaaatgaaagcggcgattcgcgctaactactctaacccaccagcacacggcgcttctgttgttgccaccatcctgagcaacgatgcgttacgtgcgatttgggaacaagagctgactgatat |
| | | gcgccagcgtattcagcgtatgcgtcagttgttcgtcaatacgctgcaggaaaaaggcgcaaaccgcgacttcagctttatcatcaaacagaacggcatgttctccttcagtggcctgacaaaaga |
| | | acaagtcctccgtctgcgcgaagagtttggcgtatatgcggttgcttctggtcgcgtaaatgtggcccggatgacaccagataacatggctccgctgtgcgaagcgattgtggcagtgctgt |
| Seq ID | p2527 | tccagagcgatgaaaacgtttcagtttgctcatggaaaacggtgtaacaagggtgaacactatcccatatcaccagctcaccgtctttcattgccatacggaattccggatgagcattcatcaggcg |
| No: 51 | | ggcaagaatgtgaataaaggccggataaaacttgtgcttatttttctttacggtctttaaaaaggccgtaatatccagctgaacggtctggttataggtacattgagcaactgactgaaatgcctcaa |
| | | aatgttctttacgatgccattgggatatatcaacggtggtatatccagtgatttttttctccattttagcttccttagctcctgaaaatctcgataactcaaaaaatacgcccggtagtgatcttattt |
| | | cattatggtgaaagttggaacctcttacgtgccgatcaacggtcgctacgctccgggcgtgagactgcggcgggcgctgcggacacatacaaagttacccacagattccgtggataagcaggggact |
| | | aacatgtgaggcaaaacagcagggccgcgccggtggcgtttttccataggctccgccctcctgccagagttcacataaacagacgcttttccggtgcatctgtgggagccgtgaggctcaaccat |
| | | gaatctgacagtacgggcgaaacccgacaggacttaaagatccccaccgtttccggcgggtcgctccctcttgcgctctcctgttccgaccctgccgtttaccggatacctgttccgcctttctccctt |
| | | acgggaagtgtggcgctttctcatagctcacacactggtatctcggctcggtgtaggtcgttcgctccaagctgggctgtaagcaagaactccccgttcagcccgactgctgcgccttatccggtaa |
| | | ctgttcacttgagtccaacccggaaaagcacggtaaaacgccactggcagcagccattggtaactgggagttcgcagaggatttgtttagctaaacacgcggttgctcttgaagtgtgcgccaaa |
| | | gtccggctacactggaaggacagatttggttgctgtgctctgcgaaagccagttaccacggttaagcagttccccaactgacttaaccttcgatcaaaccacctccccaggtggttttttcgtttacag |
| | | ggcaaaagattacgcgcagaaaaaaaggatctcaagaagatcctttgatctgatgccggccacgatgcgtccggcgtagaggatctgctcatgtttgacagcttatcatcgatgcataatgtgcct |
| | | gtcaaatggacgaagcagggattctgcaaaccctatgctactccgtcaagccgtcaattgtctgattcgttaccaagtggaggttgtataatgattcagcgggttcgtgcgagctgtagacaattaa |
| | | tactcggctcgtataatgtgtggaaggctagcaggaggaattcaatgatatgtcgacttttacagtaggtgactatttattaactcgcttacaagagatcggggtaaagcatttatttggagtacctg |
| | | gtgactataacttacaatttcttgaccgtgtgatcgctcatcccgctatttcatgggtaggctgtgccaacgagttgaacgcagcctatgcggctgatggatatgcacgttgcaatggggccggtgct |
| | | cttcttacgaccttcggggttggcgagctttccgcgatcaacggcatcgctggttcttatgccgagtatctgccggtcattcatattgtcggggcaccagctacgcaggcgcagttgcaaggggact |
| | | gcgtgcatcattctttaggagatggggacttccagcattttatccgcatggcgactgaagtatcggctgccacggctcaacttaacgccgacaacgccactgccgaaatcgaccgtgtaatcgtaa |
| | | gtgccctgcaagcacgccgtcccggatatctgtctctggctgtggatgtggcagccatggaagttcaaccaccagctcaaccattgaacagccaccaaagctctagtccggcagcccgtcgcgcc |
| | | ttctcagatgctgccgagcgtttattggcgccagcacaacgcgtcagtctgcttgcggattttttagccctgcgttggcagcaacaatcagccttagctgcattgcgcgaacaatgcgccatcccgtg |
| | | cgcttcgctgttaatggggaagggagtcttggatgagcagcagcccgggtatgttggtacttatgctggcgaggcgtctgcgggccaggtttgtgaacagattgaacaagtagacgtcgcaattt |
| | | gtgtcggggtccgttttacggacaccatcaccgccggatttacacagcagtttaatcccgagcgcctgatcgacttgcagccacttagcgctagtgtcggcggagaacgctttgccccactttcaat |
| | | ggcagacgctctttccgaacttttgccgcttttcgagcgctatgggcaacagtggcaaccaggggctacgccacctgcagcacaaccggctgaacccgccgccgtcatttcccaacaagcgttttg |
| | | gcaagcaatgcaagccttcttacagccgggcgatcttattttggccgaacaaggtactgcggcatttggggctgccgcgcttcgtctgccaagccaggcccagttagtagtacaacctctttgggg |
| | | gtctattggatacaccctgccagcagcttttggtgcgcaaacggctgaccctgatcgtcgcgtgatcctgatcattggagatggttcagcgcaattgactatccaagagttaggaagcatgctgcgc |
| | | gaccaacaacgcttgatccttttcctgctgaacaatgagggctatactgttgaacgtgcgattcacggtgctacgcagcgttataatgacattgcccagtggaattggaccgctctgcctcacgcact |
| | | ttctttgcaaggccaggcacaatcatggcgtatctcggagacggtgcaattagaggaggtgatggctcgtttgtccgaacccaagtggctgtcattggtcgaggtcgtgatgcagaaggaggac |
| | | ctgcctcctttattacgcaaagtaacagcttgtcttaaccagcgtaatggctgctaagattcagatagtaagattgataataatctagaccaggcagtggattaaagaggaggaaagtaatgtttga |
| | | gaacattaccgccgctcctgccgacccgattctgggcctggccgatctgtttcgtgccgatgaacgtcccggcaaaattaacctcgggattggtgtctataaagatgagacgggcaaaaccccggt |
| | | actgaccagcgtgaaaaaggctgaacagtatctgctcgaaaatgaaaccaccaaaaattacctcggcattgacggcatccctgaatttggtcgctgcactcaggaactgctgtttggtaaaggta |
| | | gcgccctgatcaatgacaaacgtgctcgcacggcacagactccggggggcactggcgcactacgcgtggctgccgatttcctggcaaaaaataccagcgttaagcgtgtgtgggtgagcaacc |
| | | caagctggccgaaccataagagcgtctttaactctgcaggtctggaagttcgtgaatacgcttattatgatgcggaaaatcacactcttgacttcgatgcactgattaacagcctgaatgaagctca |
| | | ggctggcgacgtagtgctgttccatggctgctgccataacccaaccggtatcgaccctacgctggaacaatggcaaacactggcacaactctccgttgagaaaggctggttaccgctgtttgactt |
| | | cgcttaccagggttttgcccgtggtctggaagaagatgctgaaggactgcgcgctttcgcggctatgcataaagagctgattgttgccagttcctactctaaaaactttggcctgtacaacgagcgt |
| | | gttggcgcttgtactctggttgctgccgacagtgaaaccgttgatcgcgcattcagccaaatgaaagcggcgattcgcgctaactactctaacccaccagcacacggcgcttctgttgttgccacca |
| | | tcctgagcaacgatgcgttacgtgcgatttgggaacaagagctgactgatatgcgccagcgtattcagcgtatgcgtcagttgttcgtcaatacgctgcaggaaaaaggcgcaaaccgcgacttc |
| | | agctttatcatcaaacagaacggcatgttctccttcagtggcctgacaaaagaacaagtgctgcgtctgcgcgaagagtttggcgtatatgcggttgcttctggtcgcgtaaatgtggccgggatg |
| | | acaccagataacatggctccgctgtgcgaagcgattgtggcagtgctgtaaatatcaatgataaaggaggaaacaaatgccaactctcaacctcgatctgcctaacggcatcaagtcgaccatcc |
| | | aggctgacttgttcatcaacaacaagtttgtgcccgctcttgacggcaagacttttgcgaccatcaacccttccaccggtaaggagattggtcaggtcgccgaggcttccgccaaggacgttgatct |
| | | cgctgtcaaggctgcgcgcgaggcattcgagactacttggggcgagaacacacccggtgacgctcgtggcagacttctgatcaagctcgccgagctggtcgaggccaacattgacgagcttgct |
| | | gccatcgagtcgctcgacaacggcaaggccttctcgatcgctaaaagctttgacgtcgctgccgtcgctgccaacttgcgatactatggtggctgggccgacaagaaccacggcaaggtcatgg |
| | | aggtcgacaccaagcgcctcaactacacgcgccacgagcccatcggcgtgtgtggtcagatcatcccttggaacttccccttgctcatgttcgcctggaagctcggccctgcgctcgccacaggta |
| | | acacgatcgtcctcaaaaccgccgagcagacaccactgtcagccatcaagatgtgtgagcttatcgtcgaggccggcttcccgcctggtgtggtcaacgtcatttccggtttcggtcccgtcgccg |
| | | gtgccgccatctcacagcacatggacattgacaagatcgccttcaccggctcaacactggtgggccgcaacattatgaaggctgctgcttcgaccaacctgaagaaggtgacgctcgagctcgg |
| | | cggcaagtcgcccaacattatctttaaggacgccgacctggaccaggcggttcgatggtcggcgttcggtatcatgttcaatcacggacaatgctgctgtgcgggctcgcgtgtctatgtggagga |
| | | atcgatctatgacgctttcatggagaagatgactgcccactgcaaggcgctccaggtgggtgaccccttttccgccaacacgttccaaggtcctcaggtttcgcagctccagtacgaccgtatcatg |
| | | gagtacatcgagtccggcaagaaggacgctaaccttgcgctcggtggtgtgcgcaagggtaacgaaggctactttatcgagcctaccatcttcaccgacgttcctcacgatgccaagattgccaa |
| | | ggaggagatcttcggccctgtcgtggtcgtctccaagttcaaagatgaaaaggacctcattcgcatcgccaacgactcgatctacggcctcgctgccgctgtcttcagccgcgacatcagccgtgc |
| | | catcgagactgcccacaagctcaaggccggcaccgtctgggtcaactgctacaaccagctcatccctcaggttccgttcggcggttacaaggcgtccggtatcggtcgagagctcggcgagtatg |
| | | cgctcagcaactacacaaacatcaaggcggtgcatgtgaacctcagccagcctgcccctatctaaatccgaactccaattattgatgaaaagggattataaaagcgttaatagcccgccagcgtc |
| | | atcagcatcatccagattggcgttgtcaccgccgcaaacacggtagaaaagagcatgctcgccgctgccgggccggtgataacattaaacttctgcgacatcaggtagacgttaaccccgaccg |
| | | ccatcgatcccagcagcaccaccactttactctccagcggcggcagatcggtcagccacgcgatagcccataccgtcatcggttgcagcaccagtttcagcaggcagatggtatagctctcttttaa |
| | | gccatcccgcacccgatagtgcgccaggctcatgcccagcgtcaccagcgacagaggtgcggccagtgaggccagcatctttagcggttcagcggccagcagcggcaagggacgttgcagaa |
| | | agctccaggctgtaccgctgagaataccgatgatcagcggattcttcagcacgccgagcagggtgcgccagagtccttccagcgagaagctgccctgttttgcccactcgaccgaaacggtcagt |
| | | aaggtccagaggatcaggctgttaaacaccagcaccagcgccaccgggggcagggcttctggtcccagcagtaccgtggccactgggatccccagcatcacgttattggaaaagatgccgccc |
| | | agcgcaaaaaccgacgccgctacgccatccagattaaacagccttgttgccagcagtcgccccacgataaacaccagcaggcaactgccgaaaaaggccagcaacaggcgcgcatccaccg |
| | | gcggactttgcgagaaggtgctcatcacccgaaacagcatgcagggcagggcaacgttaaacacaaagcggttcatcccttcgctgatgctgggggccatttcccgaggcgcgtcaggcagt |
| | | accccagcaggatcaggataaacagcggggctgacaggtaaatttgatggcttaagctttcaaacagcagtggcattcacttcttcttacgcgccgccctgccactcatcgcagtactgttgtaatt |
| | | cattaagcattctgccgacatggaagccatcacaaacggcatgatgaacctgaatcgccagcggcatcagcaccttgtcgccttgcgtataatatttgcccatggtgaaaacgggggcgaagaa |
| | | gttgtccatattggccacgtttaaatcaaaactggtgaaactcacccagggattggctgagacgaaaaacatattctcaataaaccctttagggaaataggccaggttttcaccgtaacacgccac |
| | | atcttgcgaatatatgtgtagaaactgccggaaatcgtcgtggtattcac |
| Seq ID | p2528 | atatcaatgataaaggaggaaacaaatgccaactctcaacctcgatctgcctaacggcatcaagtcgaccatccaggctgacttgttcatcaacaacaagtttgtgcccgctcttgacggcaagac |
| No: 52 | | ttttgcgaccatcaacccttccaccggtaaggagattggtcaggtcgccgaggcttccgccaaggacgttgatctcgctgtcaaggctgcgcgcgaggcattcgagactacttggggcgagaaca |
| | | cacccggtgacgctcgtggcagacttctgatcaagctcgccgagctggtcgaggccaacattgacgagcttgctgccatcgagtcgctcgacaacggcaaggccttctcgatcgctaaaagcttt |
| | | gacgtcgctgccgtcgctgccaacttgcgatactatggtggctgggccgacaagaaccacggcaaggtcatggaggtcgacaccaagcgcctcaactacacgcgccacgagcccatcggcgtg |
| | | tgtggtcagatcatcccttggaacttccccttgctcatgttcgcctggaagctcggccctgcgctcgccacaggtaacacgatcgtcctcaaaaccgccgagcagacaccactgtcagccatcaag |
| | | atgtgtgagcttatcgtcgaggccggcttcccgcctggtgtggtcaacgtcatttccggtttcggtcccgtcgccggtgccgccatctcacagcacatggacattgacaagatcgccttcaccggctc |
| | | aacactggtgggccgcaacattatgaaggctgctgcttcgaccaacctgaagaaggtgacgctcgagctcggcggcaagtcgcccaacattatctttaaggacgccgacctggaccaggcggtt |
| | | cgatggtcggcgttcggtatcatgttcaatcacggacaatgctgctgtgcgggctcgcgtgtctatgtggaggaatcgatctatgacgctttcatggagaagatgactgcccactgcaaggcgctc |
| | | caggtgggtgaccccttttccgccaacacgttccaaggtcctcaggtttcgcagctccagtacgaccgtatcatggagtacatcgagtccggcaagaaggacgctaaccttgcgctcggtggtgtg |
| | | cgcaagggtaacgaaggctactttatcgagcctaccatcttcaccgacgttcctcacgatgccaagattgccaaggaggagatcttcggccctgtcgtggtcgtctccaagttcaaagatgaaaag |
| | | gacctcattcgcatcgccaacgactcgatctacggcctcgctgccgctgtcttcagccgcgacatcagccgtgccatcgagactgcccacaagctcaaggccggcaccgtctgggtcaactgctac |
| | | aaccagctcatccctcaggttccgttcggcggttacaaggcgtccggtatcggtcgagagctcggcgagtatgcgctcagcaactacacaaacatcaaggggtgcatgtgaacctcagccagc |
| | | ctgcccctatctaaatccgaactccaattattgatgaaaagggattataaaagctcacttattcaggcgtagcacccaggcgtttaagggcaccaataactgccttaaaaaaattacgccccgccct |
| | | gccactcatcgcagtactgttgtaattcattaagcattctgccgacatggaagccatcacaaacggcatgatgaacctgaatcgccagcggcatcagcaccttgtcgccttgcgtataatatttgccc |
| | | atggtgaaaacgggggcgaagaagttgtccatattggccacgtttaaatcaaaactggtgaaactcacccagggattggctgagacgaaaaacatattctcaataaaccctttagggaaatagg |
| | | ccaggttttcaccgtaacacgccacatcttgcgaatatatgtgtagaaactgccggaaatcgtcgtggtattcactccagagcgatgaaaacgtttcagtttgctcatggaaaacggtgtaacaag |
| | | ggtgaacactatcccatatcaccagctcaccgtctttcattgccatacggaattccggatgagcattcatcagggggcaagaatgtgaataaaggccggataaaacttgtgcttatttttctttacg |
| | | gtctttaaaaaggccgtaatatccagctgaacggtctggttataggtacattgagcaactgactgaaatgcctcaaaatgttctttacgatgccattgggatatatcaacggtggtatatccagtgat |
| | | ttttttctccattttagcttccttagctcctgaaaatctcgataactcaaaaaatacgcccggtagtgatcttatttcattatggtgaaagttggaacctcttacgtgccgatcaacggtcgctacgct |
| | | ccgggcgtgagactgcggcgggcgctgcggacacatacaaagttacccacagattccgtggataagcaggggactaacatgtgaggcaaaacagcagggccgcgccggtggcgtttttccatagg |
| | | ctccgccctcctgccagagttcacataaacagacgcttttccggtgcatctgtgggagccgtgaggctcaaccatgaatctgacagtacgggcgaaacccgacaggacttaaagatccccaccgt |
| | | ttccggcgggtcgctccctcttgcgctctcctgttccgaccctgccgtttaccggatacctgttccgcctttctcccttacgggaagtgtggcgctttctcatagctcacacactggtatctcggctcg |
| | | gtgtaggtcgttcgctccaagctgggctgtaagcaagaactccccgttcagcccgactgctgcgccttatccggtaactgttcacttgagtccaacccggaaaagcacggtaaaacgccactggcagc |
| | | agccattggtaactgggagttcgcagaggatttgtttagctaaacacgcggttgctcttgaagtgtgcgccaaagtccggctacactggaaggacagatttggttgctgtgctctgcgaaagccag |
| | | ttaccacggttaagcagttccccaactgacttaaccttcgatcaaaccacctccccaggtggttttttcgtttacagggcaaaagattacgcgcagaaaaaaaggatctcaagaagatcctttgatct |
| | | gatgccggccacgatgcgtccggcgtagaggatctgctcatgtttgacagcttatcatcgatgcataatgtgcctgtcaaatggacgaagcagggattctgcaaaccctatgctactccgtcaagc |
| | | cgtcaattgtctgattcgttaccaagtggaggttgtataatgattcagcgggttcgtgcgagctgtagacaattaatactcggctcgtataatgtgtggaaggctagcaggaggaattcaatgatat |
| | | gtcgacttttacagtaggtgactatttattaactcgcttacaagagatcggggtaaagcatttatttggagtacctggtgactataacttacaatttcttgaccgtgtgatcgctcatcccgctatttca |
| | | tgggtaggctgtgccaacgagttgaacgcagcctatgcggctgatggatatgcacgttgcaatggggccggtgctcttcttacgaccttcggggttggcgagctttccgcgatcaacggcatcgctg |
| | | gttcttatgccgagtatctgccggtcattcatattgtcggggcaccagctacgcaggcgcagttgcaaggggactgcgtgcatcattctttaggagatggggacttccagcattttatccgcatggc |
| | | gactgaagtatcggctgccacggctcaacttaacgccgacaacgccactgccgaaatcgaccgtgtaatcgtaagtgccctgcaagcacgccgtcccggatatctgtctctggctgtggatgtgg |
| | | cagccatggaagttcaaccaccagctcaaccattgaacagccaccaaagctctagtccggcagcccgtcgcgccttctcagatgctgccgagcgtttattggcgccagcacaacgcgtcagtctg |
| | | cttgcggattttttagccctgcgttggcagcaacaatcagccttagctgcattgcgcgaacaatgcgccatcccgtgcgcttcgctgttaatggggaagggagtcttggatgagcagcagcccggg |
| | | tatgttggtacttatgctggcgaggcgtctgcgggccaggtttgtgaacagattgaacaagtagacgtcgcaatttgtgtcggggtccgttttacggacaccatcaccgccggatttacacagcagt |
| | | ttaatcccgagcgcctgatcgacttgcagccacttagcgctagtgtcggcggagaacgctttgccccactttcaatggcagacgctctttccgaacttttgccgcttttcgagcgctatgggcaacag |
| | | tggcaaccaggggctacgccacctgcagcacaaccggctgaacccgccgccgtcatttcccaacaagcgttttggcaagcaatgcaagccttcttacagccgggcgatcttattttggccgaaca |
| | | aggtactgcggcatttggggctgccgcgcttcgtctgccaagccaggcccagttagtagtacaacctctttgggggtctattggatacaccctgccagcagcttttggtgcgcaaacggctgaccct |
| | | gatcgtcgcgtgatcctgatcattggagatggttcagcgcaattgactatccaagagttaggaagcatgctgcgcgaccaacaacgcttgatccttttcctgctgaacaatgagggctatactgttg |
| | | aacgtgcgattcacggtgctacgcagcgttataatgacattgcccagtggaattggaccgctctgcctcacgcactttctttgcaaggccaggcacaatcatggcgtatctcggagacggtgcaatt |
| | | agaggaggtgatggctcgtttgtccgaacccaagtggctgtcattggtcgaggtcgtgatgcagaaggaggacctgcctcctttattacgcaaagtaacagcttgtcttaaccagcgtaatggctg |
| | | ctaagattcagatagtaagattgataataatctagaccaggcagtggattaaagaggaggaaagtaatggttaagttagagaatagtcgcaaacctgagaaaatctcaaataagaatattcctat |
| | | gagcgacttcgtcgtgaatttagaccacggtgatccgacagcgtacgaagagtactggcgcaaaatgggagaccgctgtaccgtgaccattcgcggttgtgacttgatgagttatttttctgatatg |
| | | acgaatctgtgttggttcttggagccggaactggaggatgcgattaaggatttacacggtgtcgttggcaatgcagctacggaggaccgctatatcgtggtgggaactgggtctacgcagttatgc |
| | | caggctgcggtccacgcattgtcttctcttgcgcgtagtcaaccagtttccgttgtggctgcggcaccattttacagcacgtacgtggaggagaccacctatgttcgcagtgggatgtacaagtggg |
| | | agggagacgcgtgggggtttgataagaaaggtccttatattgaacttgtgacatcaccgaataacccggacgggacgatccgcgagaccgtggttaatcgcccggatgatgatgaagcaaagg |
| | | taatccacgacttcgcttactattggccccattatacacctattacgcgtcgccaggatcacgacatcatgctttttacctttagcaaaatcacggggcacgctggtagtcgtattggatgggcgctgg |
| | | tgaaggataaagaggtcgctaagaaaatggttgagtatattatcgtgaacagtatcggcgtttcgaaagaatcgcaggtgcgtacagcgaaaattttgaatgtcctgaaggaaacgtgtaaatct |
| | | gaatccgaatccgagaacttcttcaaatatggtcgtgagatgatgaagaatcgctgggagaagctgcgtgaagtagtcaaggagtcagacgccttcacattgcccaaatatcctgaggctttttgt |
| | | aattatttcgggaagagtttagaatcatacccggcgtttgcctggctggggacaaaggaagagaccgacttggtatcggaattgcgtcgtcacaaggtaatgtcgcgtgcaggcgagcgttgcg |
| | | gttcagataagaagcatgttcgcgtatcaatgctgtcccgcgaggatgttttcaatgtcttcttagagcgcttagccaacatgaaattgatcaaatctatcgatttgtaa |
| Seq ID | p2529 | tccattttagcttccttagctcctgaaaatctcgataactcaaaaaatacgcccggtagtgatcttatttcattatggtgaaagttggaacctcttacgtgccgatcaacggtcgctacgctccgggcgt |
| No: 53 | | gagactgcggcgggcgctgcggacacatacaaagttacccacagattccgtggataagcaggggactaacatgtgaggcaaaacagcagggccgcgccggtggcgtttttccataggctccg |
| | | ccctcctgccagagttcacataaacagacgcttttccggtgcatctgtgggagccgtgaggctcaaccatgaatctgacagtacgggcgaaacccgacaggacttaaagatccccaccgtttccg |
| | | gcgggtcgctccctcttgcgctctcctgttccgaccctgccgtttaccggatacctgttccgcctttctcccttacgggaagtgtggcgctttctcatagctcacacactggtatctcggctcggtgtag |
| | | gtcgttcgctccaagctgggctgtaagcaagaactccccgttcagcccgactgctgcgccttatccggtaactgttcacttgagtccaacccggaaaagcacggtaaaacgccactggcagcagcc |
| | | attggtaactgggagttcgcagaggatttgtttagctaaacacgcggttgctcttgaagtgtgcgccaaagtccggctacactggaaggacagatttggttgctgtgctctgcgaaagccagttac |
| | | cacggttaagcagttccccaactgacttaaccttcgatcaaaccacctccccaggtggttttttcgtttacagggcaaaagattacgcgcagaaaaaaaggatctcaagaagatcctttgatctgat |
| | | gccggccacgatgcgtccggcgtagaggatctgctcatgtttgacagcttatcatcgatgcataatgtgcctgtcaaatggacgaagcagggattctgcaaaccctatgctactccgtcaagccgt |
| | | caattgtctgattcgttaccaagtggaggttgtataatgattcagcgggttcgtgcgagctgtagacaattaatactcggctcgtataatgtgtggaaggctagcaggaggaattcaatgatatgtc |
| | | gacttttacagtaggtgactatttattaactcgcttacaagagatcggggtaaagcatttatttggagtacctggtgactataacttacaatttcttgaccgtgtgatcgctcatcccgctatttcatgg |
| | | gtaggctgtgccaacgagttgaacgcagcctatgcggctgatggatatgcacgttgcaatggggccggtgctcttcttacgaccttcggggttggcgagctttccgcgatcaacggcatcgctggtt |
| | | cttatgccgagtatctgccggtcattcatattgtcggggcaccagctacgcaggcgcagttgcaaggggactgcgtgcatcattctttaggagatggggacttccagcattttatccgcatggcgac |
| | | tgaagtatcggctgccacggctcaacttaacgccgacaacgccactgccgaaatcgaccgtgtaatcgtaagtgccctgcaagcacgccgtcccggatatctgtctctggctgtggatgtggcag |
| | | ccatggaagttcaaccaccagctcaaccattgaacagccaccaaagctctagtccggcagcccgtcgcgccttctcagatgctgccgagcgtttattggcgccagcacaacgcgtcagtctgcttg |
| | | cggattttttagccctgcgttggcagcaacaatcagccttagctgcattgcgcgaacaatgcgccatcccgtgcgcttcgctgttaatggggaagggagtcttggatgagcagcagcccgggtatg |
| | | ttcgtacttatgctggcgaggcgtctgcgggccaggtttgtgaacagattgaacaagtagacgtcgcaatttgtgtcggggtccgttttacggacaccatcaccgccggatttacacagcagtttaa |
| | | tcccgagcgcctgatcgacttgcagccacttagcgctagtgtcggcggagaacgctttgccccactttcaatggcagacgctctttccgaacttttgccgcttttcgagcgctatgggcaacagtggc |
| | | aaccaggggctacgccacctgcagcacaaccggctgaacccgccgccgtcatttcccaacaagcgttttggcaagcaatgcaagccttcttacagccgggcgatcttattttggccgaacaaggt |
| | | actgcggcatttggggctgccgcgcttcgtctgccaagccaggcccagttagtagtacaacctctttgggggtctattggatacaccctgccagcagcttttggtgcgcaaacggctgaccctgatc |
| | | gtcgcgtgatcctgatcattggagatggttcagcgcaattgactatccaagagttaggaagcatgctgcgcgaccaacaacgcttgatccttttcctgctgaacaatgagggctatactgttgaacg |
| | | tgcgattcacggtgctacgcagcgttataatgacattgcccagtggaattggaccgctctgcctcacgcactttctttgcaaggccaggcacaatcatggcgtatctcggagacggtgcaattaga |
| | | ggaggtgatggctcgtttgtccgaacccaagtggctgtcattggtcgaggtcgtgatgcagaaggaggacctgcctcctttattacgcaaagtaacagcttgtcttaaccagcgtaatggctgcta |
| | | agattcagatagtaagattgataataatctagaccaggcagtggattaaagaggaggaaagtaatggttaagttagagaatagtcgcaaacctgagaaaatctcaaataagaatattcctatga |
| | | gcgacttcgtcgtgaatttagaccacggtgatccgacagcgtacgaagagtactggcgcaaaatgggagaccgctgtaccgtgaccattcgcggttgtgacttgatgagttatttttctgatatgac |
| | | gaatctgtgttggttcttggagccggaactggaggatgcgattaaggatttacacggtgtcgttggcaatgcagctacggaggaccgctatatcgtggtgggaactgggtctacgcagttatgcca |
| | | ggctgcggtccacgcattgtcttctcttgcgcgtagtcaaccagtttccgttgtggctgcggcaccattttacagcacgtacgtggaggagaccacctatgttcgcagtgggatgtacaagtgggag |
| | | ggagacgcgtgggggtttgataagaaaggtccttatattgaacttgtgacatcaccgaataacccggacgggacgatccgcgagaccgtggttaatcgcccggatgatgatgaagcaaaggta |
| | | atccacgacttcgcttactattggccccattatacacctattacgcgtcgccaggatcacgacatcatgctttttacctttagcaaaatcacggggcacgctggtagtcgtattggatgggcgctggtg |
| | | aaggataaagaggtcgctaagaaaatggttgagtatattatcgtgaacagtatcggcgtttcgaaagaatcgcaggtgcgtacagcgaaaattttgaatgtcctgaaggaaacgtgtaaatctg |
| | | aatccgaatccgagaacttcttcaaatatggtcgtgagatgatgaagaatcgctgggagaagctgcgtgaagtagtcaaggagtcagacgccttcacattgcccaaatatcctgaggctttttgta |
| | | attatttcgggaagagtttagaatcatacccggcgtttgcctggctggggacaaaggaagagaccgacttggtatcggaattgcgtcgtcacaaggtaatgtcgcgtgcaggcgagcgttgcggt |
| | | tcagataagaagcatgttcgcgtatcaatgctgtcccgcgaggatgttttcaatgtcttcttagagcgcttagccaacatgaaattgatcaaatctatcgatttgtaaatatcaatgataaaggagga |
| | | aacaaatgccaactctcaacctcgatctgcctaacggcatcaagtcgaccatccaggctgacttgttcatcaacaacaagtttgtgcccgctcttgacggcaagacttttgcgaccatcaacccttcc |
| | | accggtaaggagattggtcaggtcgccgaggcttccgccaaggacgttgatctcgctgtcaaggctgcgcgcgaggcattcgagactacttggggcgagaacacacccggtgacgctcgtggc |
| | | agacttctgatcaagctcgccgagctggtcgaggccaacattgacgagcttgctgccatcgagtcgctcgacaacggcaaggccttctcgatcgctaaaagctttgacgtcgctgccgtcgctgcc |
| | | aacttgcgatactatggtggctgggccgacaagaaccacggcaaggtcatggaggtcgacaccaagcgcctcaactacacgcgccacgagcccatcggcgtgtgtggtcagatcatcccttgg |
| | | aacttccccttgctcatgttcgcctggaagctcggccctgcgctcgccacaggtaacacgatcgtcctcaaaaccgccgagcagacaccactgtcagccatcaagatgtgtgagcttatcgtcgag |
| | | gccggcttcccgcctggtgtggtcaacgtcatttccggtttcggtcccgtcgccggtgccgccatctcacagcacatggacattgacaagatcgccttcaccggctcaacactggtgggccgcaac |
| | | attatgaaggctgctgcttcgaccaacctgaagaaggtgacgctcgagctcggcggcaagtcgcccaacattatctttaaggacgccgacctggaccaggcggttcgatggtcggcgttcggtat |
| | | catgttcaatcacggacaatgctgctgtgcgggctcgcgtgtctatgtggaggaatcgatctatgacgctttcatggagaagatgactgcccactgcaaggcgctccaggtgggtgaccccttttcc |
| | | gccaacacgttccaaggtcctcaggtttcgcagctccagtacgaccgtatcatggagtacatcgagtccggcaagaaggacgctaaccttgcgctcggtggtgtgcgcaagggtaacgaaggct |
| | | actttatcgagcctaccatcttcaccgacgttcctcacgatgccaagattcccaaggaggagatcttcggccctgtcgtggtcgtctccaagttcaaagatgaaaaggacctcattcgcatcgccaa |
| | | cgactcgatctacggcctcgctgccgctgtcttcagccgcgacatcagccgtgccatcgagactgcccacaagctcaaggccggcaccgtctgggtcaactgctacaaccagctcatccctcaggt |
| | | tccgttcggcggttacaaggcgtccggtatcggtcgagagctcggcgagtatgcgctcagcaactacacaaacatcaaggggtgcatgtgaacctcagccagcctgcccctatctaaatccgaa |
| | | ctccaattattgatgaaaagggattataaaagcgttaatagcccgccagcgtcatcagcatcatccagattggcgttgtcaccgccgcaaacacggtagaaaagagcatgctcgccgctgccgg |
| | | gccggtgataacattaaacttctgcgacatcaggtagacgttaaccccgaccgccatcgatcccagcagcaccaccactttactctccagcggggcagatcggtcagccacgcgatagcccata |
| | | ccgtcatcggttgcagcaccagtttcagcaggcagatggtatagctctcttttaagccatcccgcacccgatagtgcgccaggctcatgcccagcgtcaccagcgacagaggtgcggccagtgag |
| | | gccagcatctttagcggttcagcggccagcagcggcaagggacgttgcagaaagctccaggctgtaccgctgagaataccgatgatcagcggattcttcagcacgccgagcagggtgcgcca |
| | | gagtccttccagcgagaagctgccctgttttgcccactcgaccgaaacggtcagtaaggtccagaggatcaggctgttaaacaccagcaccagcgccaccgggggcagggcttctggtcccagc |
| | | agtaccgtggccactgggatccccagcatcacgttattggaaaagatgccgcccagcgcaaaaaccgacgccgctacgccatccagattaaacagccttgttgccagcagtcgccccacgataa |
| | | acaccagcaggcaactgccgaaaaaggccagcaacaggcgcgcatccaccggcggactttgcgagaaggtgctcatcacccgaaacagcatgcagggcagggcaacgttaaacacaaagc |
| | | ggttcatcccttcgctgatgctgggggccatttcccgaggcgcgtcaggcagtaccccagcaggatcaggataaacagcggggctgacaggtaaatttgatggcttaagctttcaaacagcagt |
| | | ggcattcacttcttcttacgcgccgccctgccactcatcgcagtactgttgtaattcattaagcattctgccgacatggaagccatcacaaacggcatgatgaacctgaatcgccagcggcatcagc |
| | | accttgtcgccttgcgtataatatttgcccatggtgaaaacgggggcgaagaagttgtccatattggccacgtttaaatcaaaactggtgaaactcacccagggattggctgagacgaaaaacat |
| | | attctcaataaaccctttagggaaataggccaggttttcaccgtaacacgccacatcttgcgaatatatgtgtagaaactgccggaaatcgtcgtggtattcactccagagcgatgaaaacgtttca |
| | | gtttgctcatggaaaacggtgtaacaagggtgaacactatcccatatcaccagctcaccgtctttcattgccatacggaattccggatgagcattcatcagggggcaagaatgtgaataaaggcc |
| | | ggataaaacttgtgcttatttttctttacggtctttaaaaaggccgtaatatccagctgaacggtctggttataggtacattgagcaactgactgaaatgcctcaaaatgttctttacgatgccattggg |
| | | atatatcaacggtggtatatccagtgatttttttc |
| Seq ID | p1880 | ggcgcttctgttgttgccaccatcctgagcaacgatgcgttacgtgcgatttgggaacaagagctgactgatatgcgccagcgtattcagcgtatgcgtcagttgttcgtcaatacgctgcaggaaa |
| No: 54 | | aaggcgcaaaccgcgacttcagctttatcatcaaacagaacggcatgttctccttcagtggcctgacaaaagaacaagtgctgcgtctgcgcgaagagtttggcgtatatgcggttgcttctggtc |
| | | gcgtaaatgtggccgggatgacaccagataacatggctccgctgtgcgaagcgattgtggcagtgctgtaaatatcaatgataaaggaggaaacaaatgccaactctcaacctcgatctgccta |
| | | acggcatcaagtcgaccatccaggctgacttgttcatcaacaacaagtttgtgcccgctcttgacggcaagacttttgcgaccatcaacccttccaccggtaaggagattggtcaggtcgccgagg |
| | | cttccgccaaggacgttgatctcgctgtcaaggctgcgcgcgaggcattcgagactacttggggcgagaacacacccggtgacgctcgtggcagacttctgatcaagctcgccgagctggtcga |
| | | ggccaacattgacgagcttgctgccatcgagtcgctcgacaacggcaaggccttctcgatcgctaaaagctttgacgtcgctgccgtcgctgccaacttgcgatactatggtggctgggccgacaa |
| | | gaaccacggcaaggtcatggaggtcgacaccaagcgcctcaactacacgcgccacgagcccatcggcgtgtgtggtcagatcatcccttggaacttccccttgctcatgttcgcctggaagctcg |
| | | gccctgcgctcgccacaggtaacacgatcgtcctcaaaaccgccgagcagacaccactgtcagccatcaagatgtgtgagcttatcgtcgaggccggcttcccgcctggtgtggtcaacgtcattt |
| | | ccggtttcggtcccgtcgccggtgccgccatctcacagcacatggacattgacaagatcgccttcaccggctcaacactggtgggccgcaacattatgaaggctgctgcttcgaccaacctgaag |
| | | aaggtgacgctcgagctcggcggcaagtcgcccaacattatctttaaggacgccgacctggaccaggcggttcgatggtcggcgttcggtatcatgttcaatcacggacaatgctgctgtgcggg |
| | | ctcgcgtgtctatgtggaggaatcgatctatgacgctttcatggagaagatgactgcccactgcaaggcgctccaggtgggtgaccccttttccgccaacacgttccaaggtcctcaggtttcgcag |
| | | ctccagtacgaccgtatcatggagtacatcgagtccggcaagaaggacgctaaccttgcgctcggtggtgtgcgcaagggtaacgaaggctactttatcgagcctaccatcttcaccgacgttcct |
| | | cacgatgccaagattgccaaggaggagatcttcggccctgtcgtggtcgtctccaagttcaaagatgaaaaggacctcattcgcatcgccaacgactcgatctacggcctcgctgccgctgtcttc |
| | | agccgcgacatcagccgtgccatcgagactgcccacaagctcaaggccggcaccgtctgggtcaactgctacaaccagctcatccctcaggttccgttcggggttacaaggcgtccggtatcgg |
| | | tcgagagctcggcgagtatgcgctcagcaactacacaaacatcaaggcggtgcatgtgaacctcagccagcctgcccctatctaaatccgaactccaattattgaaggccgctaacgcggcctttt |
| | | tttgtttctggtctccctgaacgctctctactagagtcacactggctcaccttcggggggcctttctgcgtttataggtacccgttgaaaagggattataaaagctcacttattcaggcgtagcaccag |
| | | gcgtttaagggcaccaataactgccttaaaaaaattacgccccgccctgccactcatcgcagtactgttgtaattcattaagcattctgccgacatggaagccatcacagacggcatgatgaacctg |
| | | aatcgccagcggcatcagcaccttgtcgccttgcgtataatatttgcccatggtgaaaacgggggcgaagaagttgtccatattggccacgtttaaatcaaaactggtgaaactcacccagggatt |
| | | ggctgagacgaaaaacatattctcaataaaccctttagggaaataggccaggttttcaccgtaacacgccacatcttgcgaatatatgtgtagaaactgccggaaatcgtcgtggtattcactcca |
| | | gagcgatgaaaacgtttcagtttgctcatggaaaacggtgtaacaagggtgaacactatcccatatcaccagctcaccgtctttcattgccatacggaattccggatgagcattcatcaggcgggc |
| | | aagaatgtgaataaaggccggataaaacttgtgcttatttttctttacggtctttaaaaaggccgtaatatccagctgaacggtctggttataggtacattgagcaactgactgaaatgcctcaaaat |
| | | gttctttacgatgccattgggatatatcaacggtggtatatccagtgatttttttctccattttagcttccttagctcctgaaaatctcgataactcaaaaaatacgcccggtagtgatcttatttcatt |
| | | atggtgaaagttggaacctcttacgtgccgatcaacgtctcattttcgccaaaagttggcccagggcttcccggtatcaacagggacaccaggatttatttattctgcgaagtgatcttccgtcacaggt |
| | | atttattcggcgcaaagtgcgtcgggtgatgctgccaacttactgatttagtgtatgatggtgtttttgaggtgctccagtggcttctgtttctatcagctgtccctcctgttcagctactgacggggtg |
| | | gtgcgtaacggcaaaagcaccgccggacatcagcgctagcggagtgtatactggcttactatgttggcactgatgagggtgtcagtgaagtgcttcatgtggcaggagaaaaaaggctgcaccgg |
| | | tgcgtcagcagaatatgtgatacaggatatattccgcttcctcgctcactgactcgctacgctcggtcgttcgactccggcgagcggaaatggcttacgaacggggcggagatttcctggaagatg |
| | | ccaggaagatacttaacagggaagtgagagggccgcggcaaagccgtttttccataggctccgcccccctgacaagcatcacgaaatctgacgctcaaatcagtggtggcgaaacccgacag |
| | | gactataaagataccaggcgtttccccctggcggctccctcgtgcgctctcctgttcctgcctttcggtttaccggtgtcattccgctgttatggccgcgtttgtctcattccacgcctgacactcagtt |
| | | ccgggtaggcagttcgctccaagctggactgtatgcacgaaccccccgttcagtccgaccgctgcgccttatccggtaactatcgtcttgagtccaacccggaaagacatgcaaaagcaccactggc |
| | | agcagccactggtaattgatttagaggagttagtcttgaagtcatgcgccggttaaggctaaactgaaaggacaagttttggtgactgcgctcctccaagccagttacctcggttcaaagagttggt |
| | | agctcagagaaccttcgaaaaaccgccctgcaaggcggttttttcgttttcagagcaagagattacgcgcagaccaaaacgatctcaagaagatcatcttattaatcagataaaatatttgctcatg |
| | | agcccgaagtggcgagcccgatcttccccatcggtgatgtcggcgatataggcgccagcaaccgcacctgtggcgccggtgatgccggccacgatgcgtccggcgtagaggatctgctcatgtt |
| | | tgacagcttatcatcgatgcataatgtgcctgtcaaatggacgaagcagggattctgcaaaccctatgctactccgtcaagccgtcaattgtctgattcgttaccaagtggaggttgtataatgtatc |
| | | accgccagaggtaaaatagtcaacacgcacggtgttagatatttatcccttgcggtgatagatttaacgtatgagcacaaaaaagaaaccattaacacaagagcagcttgaggacgcacgtcgc |
| | | cttaaagcaatttatgaaaaaaagaaaaatgaacttggcttatcccaggaatctgtcgcagacaagatggggatggggcagtcaggcgttggtgctttatttaatggcatcaatgcattaaatgct |
| | | tataacgccgcattgcttgcaaaaattctcaaagttagcgttgaagaatttagcccttcaatcgccagagaaatctacgagatgtatgaagcggttagtatgcagccgtcacttagaagtgagtatg |
| | | agtaccctgttttttctcatgttcaggcagggatgttctcacctgagcttagaacctttaccaaaggtgatgcggagagatgggtaagcacaaccaaaaaagccagtgattctgcattctggcttga |
| | | ggttgaaggtaattccatgaccgcaccaacaggctccaagccaagctttcctgacggaatgttaattctcgttgaccctgagcaggctgttgagccaggtgatttctgcatagccagacttgggggt |
| | | gatgagtttaccttcaagaaactgatcagggatagcggtcaggtgtttttacaaccactaaacccacagtacccaatgatcccatgcaatgagagttgttccgttgtggggaaagttatcgctagtc |
| | | agtggcctgaagagacgtttggctgatcggcaaggtgttctggtcggcgcatagctgataacaattgagcaagaatcttcatcgaattaggggaattttcactcccctcagaacataacatagtaa |
| | | atggattgaattatgaagaatggtttttatgcgacttaccgcagcaaaaataaagggaaagataagcgctcaataaacctgtctgttttccttaattctctgctggctgataatcatcacctgcaggtt |
| | | ggctccaattatttgtatattcataaaatcgatggaaaaacttttctctttaccaaaacaaatgacaagagtctggttcagaagataaatcgctctaaagcttcagttgaagatattaagaacagcctc |
| | | gcagatgacgaatcattgggattcccatcttttttgtttgttgaaggcgacaccattggttttgccagaactgttttcgggccgaccacatccgatctgacagattttttaatcgggaaaggaatgtcat |
| | | taagcagtggagagcgcgttcagatagagccactgatgaggggaaccaccaaagacgatgttatgcatatgcatttcatcggccgaacaacggtgaaggtagaagccaagctacctgtatttg |
| | | gcgatatattaaaggtcttaggggcaacagatattgaaggggagctttttgactcattggatatagtcattaagccaaaatttaaaagggatataaaaaaggttgccaaggatattatttttaaccc |
| | | gtcacctcaattttcagacattagcctgcgggcaaaagatgaggccggagatattttaacagaacattatctatcagaaaaaggccatctctcagcgcctctgaacaaggtcaccaatgctgagat |
| | | agctgaagagatggcatattgctacgcaagaatgaaaagtgatatactggaatgttttaaaaggcaggtgggcaaagttaaggattaattatcaggagtaattatgcggaacagaatcatgcct |
| | | ggtgtttacatagtaataattccttacgttatcgtaagcatttgctatctccttttccgccactacattcctggtgtttctttttcagctcatagagatggtcttggggcgacattgtcatcatatgcag |
| | | gaaccatgattgcaatcctgattgctgccttgacgtttctaatcggaagcagaacgcgccgactggccaagattagagagtatgggtatatgacatcggtagttattgtctatgcccttagttttgttga |
| | | gcttggagctttgtttttctgcgggttattgcttctttccagcataagcggctacatgatacccactatcgccatcggcattgcctctgcatcgttcattcatatatgcatccttgttttccaactatat |
| | | aatttgaccagagaacaagaataacccggcctcagcgccgggttttctttgcctcacgatcgcccccaaaacacataaccaattgtatttattgaaaaataaatagatacaactcactaaacatagcaat |
| | | tcagatctctcacctaccaaacaatgcccccctgcaaaaaataaattcatataaaaaacatacagataaccatctgcggtgataaattatctctggcggtgttgacataaataccactggcggtgatact |
| | | gagcacatcagcaggacggtggtggaggtgtcccatgtcgacttttacagtaggtgactatttattaactcgcttacaagagatcggggtaaagcatttatttggagtacctggtgactataactta |
| | | caatttcttgaccgtgtgatcgctcatcccgctatttcatgggtaggctgtgccaacgagttgaacgcagcctatgcggctgatggatatgcacgttgcaatggggccggtgctcttcttacgaccttc |
| | | ggggttggcgagctttccgcgatcaacggcatcgctggttcttatgccgagtatctgccggtcattcatattgtcggggcaccagctacgcaggcgcagttgcaaggggactgcgtgcatcattctt |
| | | taggagatggggacttccagcattttatccgcatggcgactgaagtatcggctgccacggctcaacttaacgccgacaacgccactgccgaaatcgaccgtgtaatcgtaagtgccctgcaagca |
| | | cgccgtcccggatatctgtctctggctgtggatgtggcagccatggaagttcaaccaccagctcaaccattgaacagccaccaaagctctagtccggcagcccgtcgcgccttctcagatgctgcc |
| | | gagcgtttattggcgccagcacaacgcgtcagtctgcttgcggattttttagccctgcgttggcagcaacaatcagccttagctgcattgcgcgaacaatgcgccatcccgtgcgcttcgctgttaat |
| | | ggggaagggagtcttggatgagcagcagcccgggtatgttggtacttatgctggcgaggcgtctgcgggccaggtttgtgaacagattgaacaagtagacgtcgcaatttgtgtcggggtccgt |
| | | tttacggacaccatcaccgccggatttacacagcagtttaatcccgagcgcctgatcgacttgcagccacttagcgctagtgtcggcggagaacgctttgccccactttcaatggcagacgctctttc |
| | | cgaacttttgccgcttttcgagcgctatgggcaacagtggcaaccaggggctacgccacctgcagcacaaccggctgaacccgccgccgtcatttcccaacaagcgttttggcaagcaatgcaag |
| | | ccttcttacagccgggcgatcttattttggccgaacaaggtactgcggcatttggggctgccgcgcttcgtctgccaagccaggcccagttagtagtacaacctctttgggggtctattggatacacc |
| | | ctgccagcagcttttggtgcgcaaacggctgaccctgatcgtcgcgtgatcctgatcattggagatggttcagcgcaattgactatccaagagttaggaagcatgctgcgcgaccaacaacgcttg |
| | | atccttttcctgctgaacaatgagggctatactgttgaacgtgcgattcacggtgctacgcagcgttataatgacattgcccagtggaattggaccgctctgcctcacgcactttctttgcaaggccag |
| | | gcacaatcatggcgtatctcggagacggtgcaattagaggaggtgatggctcgtttgtccgaacccaagtggctgtcattggtcgaggtcgtgatgcagaaggaggacctgcctcctttattacgc |
| | | aaagtaacagcttgtcttaaccagcgtaatggctgctaaaagattgataataatctagaccaggcagtggattaaagaggaggaaagtaatgtttgagaacattaccgccgctcctgccgacccg |
| | | attctgggcctggccgatctgtttcgtgccgatgaacgtcccggcaaaattaacctcgggattggtgtctataaagatgagacgggcaaaaccccggtactgaccagcgtgaaaaaggctgaac |
| | | agtatctgctcgaaaatgaaaccaccaaaaattacctcggcattgacggcatccctgaatttggtcgctgcactcaggaactgctgtttggtaaaggtagcgccctgatcaatgacaaacgtgctc |
| | | gcacggcacagactccggggggcactggcgcactacgcgtggctgccgatttcctggcaaaaaataccagcgttaagcgtgtgtgggtgagcaacccaagctggccgaaccataagagcgtc |
| | | tttaactctgcaggtctggaagttcgtgaatacgcttattatgatgcggaaaatcacactcttgacttcgatgcactgattaacagcctgaatgaagctcaggctggcgacgtagtgctgttccatgg |
| | | ctgctgccataacccaaccggtatcgaccctacgctggaacaatggcaaacactggcacaactctccgttgagaaaggctggttaccgctgtttgacttcgcttaccagggttttgcccgtggtctg |
| | | gaagaagatgctgaaggactgcgcgctttcgcggctatgcataaagagctgattgttgccagttcctactctaaaaactttggcctgtacaacgagcgtgttggcgcttgtactctggttgctgccg |
| | | acagtgaaaccgttgatcgcgcattcagccaaatgaaagcggcgattcgcgctaactactctaacccaccagcacac |
| Seq ID | p1881 | accgcaccaacaggctccaagccaagctttcctgacggaatgttaattctcgttgaccctgagcaggctgttgagccaggtgatttctgcatagccagacttgggggtgatgagtttaccttcaaga |
| No: 55 | | aactgatcagggatagcggtcaggtgtttttacaaccactaaacccacagtacccaatgatcccatgcaatgagagttgttccgttgtggggaaagttatcgctagtcagtggcctgaagagacgt |
| | | ttggctgatcggcaaggtgttctggtcggcgcatagctgataacaattgagcaagaatcttcatcgaattaggggaattttcactcccctcagaacataacatagtaaatggattgaattatgaaga |
| | | atggtttttatgcgacttaccgcagcaaaaataaagggaaagataagcgctcaataaacctgtctgttttccttaattctctgctggctgataatcatcacctgcaggttggctccaattatttgtatat |
| | | tcataaaatcgatggaaaaacttttctctttaccaaaacaaatgacaagagtctggttcagaagataaatcgctctaaagcttcagttgaagatattaagaacagcctcgcagatgacgaatcattg |
| | | ggattcccatcttttttgtttgttgaaggcgacaccattggttttgccagaactgttttcgggccgaccacatccgatctgacagattttttaatcgggaaaggaatgtcattaagcagtggagagcgc |
| | | gttcagatagagccactgatgaggggaaccaccaaagacgatgttatgcatatgcatttcatcggccgaacaacggtgaaggtagaagccaagctacctgtatttggcgatatattaaaggtctt |
| | | aggggcaacagatattgaaggggagctttttgactcattggatatagtcattaagccaaaatttaaaagggatataaaaaaggttgccaaggatattatttttaacccgtcacctcaattttcagac |
| | | attagcctgcgggcaaaagatgaggccggagatattttaacagaacattatctatcagaaaaaggccatctctcagcgcctctgaacaaggtcaccaatgctgagatagctgaagagatggcat |
| | | attgctacgcaagaatgaaaagtgatatactggaatgttttaaaaggcaggtgggcaaagttaaggattaattatcaggagtaattatgcggaacagaatcatgcctggtgtttacatagtaataa |
| | | ttccttacgttatcgtaagcatttgctatctccttttccgccactacattcctggtgtttctttttcagctcatagagatggtcttggggcgacattgtcatcatatgcaggaaccatgattgcaatcct |
| | | gattgctgccttgacgtttctaatcggaagcagaacgcgccgactggccaagattagagagtatgggtatatgacatcggtagttattgtctatgcccttagttttgttgagcttggagctttgtttttc |
| | | tgcgggttattgcttctttccagcataagcggctacatgatacccactatcgccatcggcattgcctctgcatcgttcattcatatatgcatccttgttttccaactatataatttgaccagagaacaag |
| | | aataacccggcctcagcgccgggttttctttgcctcacgatcgcccccaaaacacataaccaattgtatttattgaaaaataaatagatacaactcactaaacatagcaattcagatctctcacctacca |
| | | aacaatgcccccctgcaaaaaataaattcatataaaaaacatacagataaccatctgcggtgataaattatctctggcggtgttgacataaataccactggcggtgatactgagcacatcagcaggac |
| | | ggtggtggaggtgtcccatgtcgacttttacagtaggtgactatttattaactcgcttacaagagatcggggtaaagcatttatttggagtacctggtgactataacttacaatttcttgaccgtgtgat |
| | | cgctcatcccgctatttcatgggtaggctgtgccaacgagttgaacgcagcctatgcggctgatggatatgcacgttgcaatggggccggtgctcttcttacgaccttcggggttggcgagctttcc |
| | | gcgatcaacggcatcgctggttcttatgccgagtatctgccggtcattcatattgtcggggcaccagctacgcaggcgcagttgcaaggggactgcgtgcatcattctttaggagatggggacttc |
| | | cagcattttatccgcatggcgactgaagtatcggctgccacggctcaacttaacgccgacaacgccactgccgaaatcgaccgtgtaatcgtaagtgccctgcaagcacgccgtcccggatatct |
| | | gtctctggctgtggatgtggcagccatggaagttcaaccaccagctcaaccattgaacagccaccaaagctctagtccggcagcccgtcgcgccttctcagatgctgccgagcgtttattggcgcc |
| | | agcacaacgcgtcagtctgcttgcggattttttagccctgcgttggcagcaacaatcagccttagctgcattgcgcgaacaatgcgccatcccgtgcgcttcgctgttaatggggaagggagtcttg |
| | | gatgagcagcagcccgggtatgttggtacttatgctggcgaggcgtctgcgggccaggtttgtgaacagattgaacaagtagacgtcgcaatttgtgtcggggtccgttttacggacaccatcac |
| | | cgccggatttacacagcagtttaatcccgagcgcctgatcgacttgcagccacttagcgctagtgtcggcggagaacgctttgccccactttcaatggcagacgctctttccgaacttttgccgctttt |
| | | cgagcgctatgggcaacagtggcaaccaggggctacgccacctgcagcacaaccggctgaacccgccgccgtcatttcccaacaagcgttttggcaagcaatgcaagccttcttacagccggg |
| | | cgatcttattttggccgaacaaggtactgcggcatttggggctgccgcgcttcgtctgccaagccaggcccagttagtagtacaacctctttgggggtctattggatacaccctgccagcagcttttg |
| | | gtgcgcaaacggctgaccctgatcgtcgcgtgatcctgatcattggagatggttcagcgcaattgactatccaagagttaggaagcatgctgcgcgaccaacaacgcttgatccttttcctgctgaa |
| | | caatgagggctatactgttgaacgtgcgattcacggtgctacgcagcgttataatgacattgcccagtggaattggaccgctctgcctcacgcactttctttgcaaggccaggcacaatcatggcgt |
| | | atctcggagacggtgcaattagaggaggtgatggctcgtttgtccgaacccaagtggctgtcattggtcgaggtcgtgatgcagaaggaggacctgcctcctttattacgcaaagtaacagcttgt |
| | | cttaaccagcgtaatggctgctaaaagattgataataatctagaccaggcagtggattaaagaggaggaaagtaatgtttgagaacattaccgccgctcctgccgacccgattctgggcctggcc |
| | | gatctgtttcgtgccgatgaacgtcccggcaaaattaacctcgggattggtgtctataaagatgagacgggcaaaaccccggtactgaccagcgtgaaaaaggctgaacagtatctgctcgaaa |
| | | atgaaaccaccaaaaattacctcggcattgacggcatccctgaatttggtcgctgcactcaggaactgctgtttggtaaaggtagcgccctgatcaatgacaaacgtgctcgcacggcacagactc |
| | | cggggggcactggcgcactacgcgtggctgccgatttcctggcaaaaaataccagcgttaagcgtgtgtgggtgagcaacccaagctggccgaaccataagagcgtctttaactctgcaggtct |
| | | ggaagttcgtgaatacgcttattatgatgcggaaaatcacactcttgacttcgatgcactgattaacagcctgaatgaagctcaggctggcgacgtagtgctgttccatggctgctgccataaccca |
| | | accggtatcgaccctacgctggaacaatggcaaacactggcacaactctccgttgagaaaggctggttaccgctgtttgacttcgcttaccagggttttgcccgtggtctggaagaagatgctgaa |
| | | ggactgcgcgctttcgcggctatgcataaagagctgattgttgccagttcctactctaaaaactttggcctgtacaacgagcgtgttggcgcttgtactctggttgctgccgacagtgaaaccgttga |
| | | tcgcgcattcagccaaatgaaagcggcgattcgcgctaactactctaacccaccagcacacggcgcttctgttgttgccaccatcctgagcaacgatgcgttacgtgcgatttgggaacaagagct |
| | | gactgatatgcgccagcgtattcagcgtatgcgtcagttgttcgtcaatacgctgcaggaaaaaggcgcaaaccgcgacttcagctttatcatcaaacagaacggcatgttctccttcagtggcctg |
| | | acaaaagaacaagtgctgcgtctgcgcgaagagtttggcgtatatgcggttgcttctggtcgcgtaaatgtggccgggatgacaccagataacatggctccgctgtgcgaagcgattgtggcagt |
| | | gctgtaaatatcaatgataaaggaggaaacaaatgccaactctcaacctcgatctgcctaacggcatcaagtcgaccatccaggctgacttgttcatcaacaacaagtttgtgcccgctcttgacg |
| | | gcaagacttttgcgaccatcaacccttccaccggtaaggagattggtcaggtcgccgaggcttccgccaaggacgttgatctcgctgtcaaggctgcgcgcgaggcattcgagactacttggggc |
| | | gagaacacacccggtgacgctcgtggcagacttctgatcaagctcgccgagctggtcgaggccaacattgacgagcttgctgccatcgagtcgctcgacaacggcaaggccttctcgatcgcta |
| | | aaagctttgacgtcgctgccgtcgctgccaacttgcgatactatggtggctgggccgacaagaaccacggcaaggtcatggaggtcgacaccaagcgcctcaactacacgcgccacgagccca |
| | | tcggcgtgtgtggtcagatcatcccttggaacttccccttgctcatgttcgcctggaagctcggccctgcgctcgccacaggtaacacgatcgtcctcaaaaccgccgagcagacaccactgtcagc |
| | | catcaagatgtgtgagcttatcgtcgaggccggcttcccgcctggtgtggtcaacgtcatttccggtttcggtcccgtcgccggtgccgccatctcacagcacatggacattgacaagatcgccttca |
| | | ccggctcaacactggtgggccgcaacattatgaaggctgctgcttcgaccaacctgaagaaggtgacgctcgagctcggcggcaagtcgcccaacattatctttaaggacgccgacctggacca |
| | | ggcggttcgatggtcggcgttcggtatcatgttcaatcacggacaatgctgctgtgcgggctcgcgtgtctatgtggaggaatcgatctatgacgctttcatggagaagatgactgcccactgcaa |
| | | ggcgctccaggtgggtgaccccttttccgccaacacgttccaaggtcctcaggtttcgcagctccagtacgaccgtatcatggagtacatcgagtccggcaagaaggacgctaaccttgcgctcg |
| | | gtggtgtgcgcaagggtaacgaaggctactttatcgagcctaccatcttcaccgacgttcctcacgatgccaagattgccaaggaggagatcttcggccctgtcgtggtcgtctccaagttcaaag |
| | | atgaaaaggacctcattcgcatcgccaacgactcgatctacggcctcgctgccgctgtcttcagccgcgacatcagccgtgccatcgagactgcccacaagctcaaggccggcaccgtctgggtc |
| | | aactgctacaaccagctcatccctcaggttccgttcggcggttacaaggcgtccggtatcggtcgagagctcggcgagtatgcgctcagcaactacacaaacatcaaggcggtgcatgtgaacct |
| | | cagccagcctgcccctatctaaatccgaactccaattattgaaggccgctaacgcggcctttttttgtttctggtctccctgaacgctctctactagagtcacactggctcaccttcgggtgggcctttc |
| | | tgcgtttataggtacccgttgaaaagggattataaaagcttataatccaagtaaaacataatataacacggtaaccggcagcgccacaagcataccaaaaatcactgccgtagaaagaatatctg |
| | | ggtgcacattgtattctttggcaaaaacaaaggggacgattccctggggcagtgccgcctgtacgatggcaatgtgtaacaggtccccgcgaataccgatagcgatactggtggcagcaataac |
| | | cgctggtccagtcaggaagcgaaccgccatcgcaaacccggctacagacttcccgcacgcgataatttttggttgcaaggccatgaacaggccaagactaaacattgccatgcccaatcccgcg |
| | | tcgctcaaaatggaaatgctaccgctcatgatagttggcattttaatattccacttaaacgatactaaagaccaggccagaccaaaaaggcttgagtatgtgttaggattgcgaattaacttacgcc |
| | | agaccataataagaatcaagcgtgtcattacactcgccggaggcatctgctgcttacgaggtccgggacctccatcctcgacatcagaccccttcttacccatgtagggagatttcccgccattgttc |
| | | ccgtcctgatccatttctacatggcctttacgaccaggcgacatattttcaataagattctgcatagccttggtcgctaagttgtcatgggggggatggacactttagggtcggttgacacgtcagta |
| | | ctagatccgcgcgtcatggcattctttgcgttcgcctcgctgacgggtgaggcgctgctagaccagacgaacatattcatttccttgttttggccacctaccccgacgcctcccccgcttccccctccgc |
| | | ctgactccttcttcttaacaccggacgcaccactcgttgatcctgtaaacatcgggttaggggggggtaacttggcaccgaattgttgttgtataattcccctgacatagagcgaccgccacgacct |
| | | gctttctttgcagttttcataacttcttcatcgaaattagatgtacgcggtgtgacccctttggaagactgaaggctgtacacgtcaccacctgggcctgctcctgcgcctccgtagctattagtatagc |
| | | cgtggcgcggtgaaggggccttactagcattgaacatagcgtaaaagtccgtttggttgaaactggatgcacgcggggtgggctcgcgactagactgcacactgtaaatctcaaccccagtaag |
| | | attactagcacgtggtgtaatcattgaactgttaagaccccctccatgggacttgttaaatgaactaatcatactggatgcagcactgctacggcgtactacaacatgaagcttcccatcatcgccga |
| | | tctccgcgtcggtctgcaatggctcgcgcccattcaagctgatgacatcactatcaacacgaaaactagtgattgaccccgcagtctccggaaattgttcactaatcagaagtttagcgccgcgaaa |
| | | ctcgaacaaaaacagcatcaaagtgtaccaaataatactctgtaagacgacgatttggaccataaggttaccagaaaagtcgccgtacattgcgcgcaaaagggggatacccattaccagcgta |
| | | ttgggcaaggtagaaagggaaaaaagtgtaatcatccattctaaactcccgcgacgagagaaggcttgccacaggaacaaggctgctaagataaccactttctgtaaggaatcagcggctaaa |
| | | aaatgataattcattgcataagggtcatttgagctaatgaaatgaaatgataacaaaggaacggcaaaaacagcgacaaaacgattaattcctgagcattggtcgggtgtaaaaataccccacc |
| | | agcggacagagccgtaagcaaggatcatagccacatacaggggcaccatagctgccagcacgtcgtacatatctttgcctgtaatcattcacttcttcttacgcgccgccctgccactcatcgcagt |
| | | actgttgtaattcattaagcattctgccgacatggaagccatcacagacggcatgatgaacctgaatcgccagcggcatcagcaccttgtcgccttgcgtataatatttgcccatggtgaaaacggg |
| | | ggcgaagaagttgtccatattggccacgtttaaatcaaaactggtgaaactcacccagggattggctgagacgaaaaacatattctcaataaaccctttagggaaataggccaggttttcaccgta |
| | | acacgccacatcttgcgaatatatgtgtagaaactgccggaaatcgtcgtggtattcactccagagcgatgaaaacgtttcagtttgctcatggaaaacggtgtaacaagggtgaacactatccca |
| | | tatcaccagctcaccgtctttcattgccatacggaattccggatgagcattcatcagggggcaagaatgtgaataaaggccggataaaacttgtgcttatttttctttacggtctttaaaaaggccgt |
| | | aatatccagctgaacggtctggttataggtacattgagcaactgactgaaatgcctcaaaatgttctttacgatgccattgggatatatcaacggtggtatatccagtgatttttttctccattttagct |
| | | tccttagctcctgaaaatctcgataactcaaaaaatacgcccggtagtgatcttatttcattatggtgaaagttggaacctcttacgtgccgatcaacgtctcattttcgccaaaagttggcccagggct |
| | | tcccggtatcaacagggacaccaggatttatttattctgcgaagtgatcttccgtcacaggtatttattcggcgcaaagtgcgtcgggtgatgctgccaacttactgatttagtgtatgatggtgttttt |
| | | gaggtgctccagtggcttctgtttctatcagctgtccctcctgttcagctactgacggggtggtgcgtaacggcaaaagcaccgccggacatcagcgctagcggagtgtatactggcttactatgtt |
| | | ggcactgatgagggtgtcagtgaagtgcttcatgtggcaggagaaaaaaggctgcaccggtgcgtcagcagaatatgtgatacaggatatattccgcttcctcgctcactgactcgctacgctcg |
| | | gtcgttcgactgcggcgagcggaaatggcttacgaacggggcggagatttcctggaagatgccaggaagatacttaacagggaagtgagagggccgcggcaaagccgtttttccataggctc |
| | | cgcccccctgacaagcatcacgaaatctgacgctcaaatcagtggtggcgaaacccgacaggactataaagataccaggcgtttccccctggcggctccctcgtgcgctctcctgttcctgcctttc |
| | | ggtttaccggtgtcattccgctgttatggccgcgtttgtctcattccacgcctgacactcagttccgggtaggcagttcgctccaagctggactgtatgcacgaaccccccgttcagtccgaccgctgc |
| | | gccttatccggtaactatcgtcttgagtccaacccggaaagacatgcaaaagcaccactggcagcagccactggtaattgatttagaggagttagtcttgaagtcatgcgccggttaaggctaaac |
| | | tgaaaggacaagttttggtgactgcgctcctccaagccagttacctcggttcaaagagttggtagctcagagaaccttcgaaaaaccgccctgcaaggcggttttttcgttttcagagcaagagatt |
| | | acgcgcagaccaaaacgatctcaagaagatcatcttattaatcagataaaatatttgctcatgagcccgaagtggcgagcccgatcttccccatcggtgatgtcggcgatataggcgccagcaac |
| | | cgcacctgtggcgccggtgatgccggccacgatgcgtccggcgtagaggatctgctcatgtttgacagcttatcatcgatgcataatgtgcctgtcaaatggacgaagcagggattctgcaaacc |
| | | ctatgctactccgtcaagccgtcaattgtctgattcgttaccaagtggaggttgtataatgtatcaccgccagaggtaaaatagtcaacacgcacggtgttagatatttatcccttgcggtgatagatt |
| | | taacgtatgagcacaaaaaagaaaccattaacacaagagcagcttgaggacgcacgtcgccttaaagcaatttatgaaaaaaagaaaaatgaacttggcttatcccaggaatctgtcgcagac |
| | | aagatggggatggggcagtcaggcgttggtgctttatttaatggcatcaatgcattaaatgcttataacgccgcattgcttgcaaaaattctcaaagttagcgttgaagaatttagcccttcaatcgc |
| | | cagagaaatctacgagatgtatgaagcggttagtatgcagccgtcacttagaagtgagtatgagtaccctgttttttctcatgttcaggcagggatcttctcacctgagcttagaacctttaccaaag |
| | | gtgatgcggagagatgggtaagcacaaccaaaaaagccagtgattctgcattctggcttgaggttgaaggtaattccatg |
| Seq ID | p1882 | accgcaccaacaggctccaagccaagctttcctgacggaatgttaattctcgttgaccctgagcaggctgttgagccaggtgatttctgcatagccagacttgggggtgatgagtttaccttcaaga |
| No: 56 | | aactgatcagggatagcggtcaggtgtttttacaaccactaaacccacagtacccaatgatcccatgcaatgagagttgttccgttgtggggaaagttatcgctagtcagtggcctgaagagacgt |
| | | ttggctgatcggcaaggtgttctggtcggcgcatagctgataacaattgagcaagaatcttcatgaattaggggaattttcactcccctcagaacataacatagtaaatggattgaattatgaaga |
| | | atggtttttatgcgacttaccgcagcaaaaataaagggaaagataagcgctcaataaacctgtctgttttccttaattctctgctggctgataatcatcacctgcaggttggctccaattatttgtatat |
| | | tcataaaatcgatggaaaaacttttctctttaccaaaacaaatgacaagagtctggttcagaagataaatcgctctaaagcttcagttgaagatattaagaacagcctcgcagatgacgaatcattg |
| | | ggattcccatcttttttgtttgttgaaggcgacaccattggttttgccagaactgttttcgggccgaccacatccgatctgacagattttttaatcgggaaaggaatgtcattaagcagtggagagcgc |
| | | gttcagatagagccactgatgaggggaaccaccaaagacgatgttatgcatatgcatttcatcggccgaacaacggtgaaggtagaagccaagctacctgtatttggcgatatattaaaggtctt |
| | | aggggcaacagatattgaaggggagctttttgactcattggatatagtcattaagccaaaatttaaaagggatataaaaaaggttgccaaggatattatttttaacccgtcacctcaattttcagac |
| | | attagcctgcgggcaaaagatgaggccggagatattttaacagaacattatctatcagaaaaaggccatctctcagcgcctctgaacaaggtcaccaatgctgagatagctgaagagatggcat |
| | | attgctacgcaagaatgaaaagtgatatactggaatgttttaaaaggcaggtgggcaaagttaaggattaattatcaggagtaattatgcggaacagaatcatgcctggtgtttacatagtaataa |
| | | ttccttacgttatcgtaagcatttgctatctccttttccgccactacattcctggtgtttctttttcagctcatagagatggtcttggggcgacattgtcatcatatgcaggaaccatgattgcaatcct |
| | | gattgctgccttgacgtttctaatcggaagcagaacgcgccgactggccaagattagagagtatgggtatatgacatcggtagttattgtctatgcccttagttttgttgagcttggagctttgtttttc |
| | | tgcgggttattgcttctttccagcataagcggctacatgatacccactatcgccatcggcattgcctctgcatcgttcattcatatatgcatccttgttttccaactatataatttgaccagagaacaag |
| | | aataacccggcctcagcgccgggttttctttgcctcacgatcgcccccaaaacacataaccaattgtatttattgaaaaataaatagatacaactcactaaacatagcaattcagatctctcacctacca |
| | | aacaatgcccccctgcaaaaaataaattcatataaaaaacatacagataaccatctgcggtgataaattatctctggcggtgttgacataaataccactggcggtgatactgagcacatcagcaggac |
| | | ggtggtggaggtgtcccatgtcgacttttacagtaggtgactatttattaactcgcttacaagagatcggggtaaagcatttatttggagtacctggtgactataacttacaatttcttgaccgtgtgat |
| | | cgctcatcccgctatttcatgggtaggctgtgccaacgagttgaacgcagcctatgcggctgatggatatgcacgttgcaatggggccggtgctcttcttacgaccttcggggttggcgagctttcc |
| | | gcgatcaacggcatcgctggttcttatgccgagtatctgccggtcattcatattgtcggggcaccagctacgcaggcgcagttgcaaggggactgcgtgcatcattctttaggagatggggacttc |
| | | cagcattttatccgcatggcgactgaagtatcggctgccacggctcaacttaacgccgacaacgccactgccgaaatcgaccgtgtaatcgtaagtgccctgcaagcacgccgtcccggatatct |
| | | gtctctggctgtggatgtggcagccatggaagttcaaccaccagctcaaccattgaacagccaccaaagctctagtccggcagcccgtcgcgccttctcagatgctgccgagcgtttattggcgcc |
| | | agcacaacgcgtcagtctgcttgcggattttttagccctgcgttggcagcaacaatcagccttagctgcattgcgcgaacaatgcgccatcccgtgcgcttcgctgttaatggggaagggagtcttg |
| | | gatgagcagcagcccgggtatgttggtacttatgctggcgaggcgtctgcgggccaggtttgtgaacagattgaacaagtagacgtcgcaatttgtgtcggggtccgttttacggacaccatcac |
| | | cgccggatttacacagcagtttaatcccgagcgcctgatcgacttgcagccacttagcgctagtgtcggcggagaacgctttgccccactttcaatggcagacgctctttccgaacttttgccgctttt |
| | | cgagcgctatgggcaacagtggcaaccaggggctacgccacctgcagcacaaccggctgaacccgccgccgtcatttcccaacaagcgttttggcaagcaatgcaagccttcttacagccggg |
| | | cgatcttattttggccgaacaaggtactgcggcatttggggctgccgcgcttcgtctgccaagccaggcccagttagtagtacaacctctttgggggtctattggatacaccctgccagcagcttttg |
| | | gtgcgcaaacggctgaccctgatcgtcgcgtgatcctgatcattggagatggttcagcgcaattgactatccaagagttaggaagcatgctgcgcgaccaacaacgcttgatccttttcctgctgaa |
| | | caatgagggctatactgttgaacgtgcgattcacggtgctacgcagcgttataatgacattgcccagtggaattggaccgctctgcctcacgcactttctttgcaaggccaggcacaatcatggcgt |
| | | atctcggagacggtgcaattagaggaggtgatggctcgtttgtccgaacccaagtggctgtcattggtcgaggtcgtgatgcagaaggaggacctgcctcctttattacgcaaagtaacagcttgt |
| | | cttaaccagcgtaatggctgctaaaagattgataataatctagaccaggcagtggattaaagaggaggaaagtaatgtttgagaacattaccgccgctcctgccgacccgattctgggcctggcc |
| | | gatctgtttcgtgccgatgaacgtcccggcaaaattaacctcgggattcgtgtctataaagatgagacgggcaaaaccccggtactgaccagcgtgaaaaaggctgaacagtatctgctcgaaa |
| | | atgaaaccaccaaaaattacctcggcattgacggcatccctgaatttggtcgctgcactcaggaactgctgtttggtaaaggtagcgccctgatcaatgacaaacgtgctcgcacggcacagactc |
| | | cggggggcactggcgcactacgcgtggctgccgatttcctggcaaaaaataccagcgttaagcgtgtgtgggtgagcaacccaagctggccgaaccataagagcgtctttaactctgcaggtct |
| | | ggaagttcgtgaatacgcttattatgatgcggaaaatcacactcttgacttcgatgcactgattaacagcctgaatgaagctcaggctggcgacgtagtgctgttccatggctgctgccataaccca |
| | | accggtatcgaccctacgctggaacaatggcaaacactggcacaactctccgttgagaaaggctggttaccgctgtttgacttcgcttaccagggttttgcccgtggtctggaagaagatgctgaa |
| | | ggactgcgcgctttcgcggctatgcataaagagctgattgttgccagttcctactctaaaaactttggcctgtacaacgagcgtgttggcgcttgtactctggttgctgccgacagtgaaaccgttga |
| | | tcgcgcattcagccaaatgaaagcggcgattcgcgctaactactctaacccaccagcacacggcgcttctgttgttgccaccatcctgagcaacgatgcgttacgtgcgatttgggaacaagagct |
| | | gactgatatgcgccagcgtattcagcgtatgcgtcagttgttcgtcaatacgctgcaggaaaaaggcgcaaaccgcgacttcagctttatcatcaaacagaacggcatgttctccttcagtggcctg |
| | | acaaaagaacaagtgctgcgtctgcgcgaagagtttggcgtatatgcggttgcttctggtcgcgtaaatgtggccgggatgacaccagataacatggctccgctgtgcgaagcgattgtggcagt |
| | | gctgtaaatatcaatgataaaggaggaaacaaatgccaactctcaacctcgatctgcctaacggcatcaagtcgaccatccaggctgacttgttcatcaacaacaagtttgtgcccgctcttgacg |
| | | gcaagacttttgcgaccatcaacccttccaccggtaaggagattggtcaggtcgccgaggcttccgccaaggacgttgatctcgctgtcaaggctgcgcgcgaggcattcgagactacttggggc |
| | | gagaacacacccggtgacgctcgtggcagacttctgatcaagctcgccgagctggtcgaggccaacattgacgagcttgctgccatcgagtcgctcgacaacggcaaggccttctcgatcgcta |
| | | aaagctttgacgtcgctgccgtcgctgccaacttgcgatactatggtggctgggccgacaagaaccacggcaaggtcatggaggtcgacaccaagcgcctcaactacacgcgccacgagccca |
| | | tcggcgtgtgtggtcagatcatcccttggaacttccccttgctcatgttcgcctggaagctcggccctgcgctcgccacaggtaacacgatcgtcctcaaaaccgccgagcagacaccactgtcagc |
| | | catcaagatgtgtgagcttatcgtcgaggccggcttcccgcctggtgtggtcaacgtcatttccggtttcggtcccgtcgccggtgccgccatctcacagcacatggacattgacaagatcgccttca |
| | | ccggctcaacactggtgggccgcaacattatgaaggctgctgcttcgaccaacctgaagaaggtgacgctcgagctcggggcaagtcgcccaacattatctttaaggacgccgacctggacca |
| | | ggcggttcgatggtcggcgttcggtatcatgttcaatcacggacaatgctgctgtgcgggctcgcgtgtctatgtggaggaatcgatctatgacgctttcatggagaagatgactgcccactgcaa |
| | | ggcgctccaggtgggtgaccccttttccgccaacacgttccaaggtcctcaggtttcgcagctccagtacgaccgtatcatggagtacatcgagtccggcaagaaggacgctaaccttgcgctcg |
| | | gtggtgtgcgcaagggtaacgaaggctactttatcgagcctaccatcttcaccgacgttcctcacgatgccaagattgccaaggaggagatcttcggccctgtcgtggtcgtctccaagttcaaag |
| | | atgaaaaggacctcattcgcatcgccaacgactcgatctacggcctcgctgccgctgtcttcagccgcgacatcagccgtgccatcgagactgcccacaagctcaaggccggcaccgtctgggtc |
| | | aactgctacaaccagctcatccctcaggttccgttcggcggttacaaggcgtccggtatcggtcgagagctcggcgagtatgcgctcagcaactacacaaacatcaaggcggtgcatgtgaacct |
| | | cagccagcctgcccctatctaaatccgaactccaattattgaaggccgctaacgcggccttttttttttctggtctccctgaacgctctctactagagtcacactggctcaccttcgggtgggcctttct |
| | | gcgtttataggtacccgttgaaaagggattataaaagcgttaatagcccgccagcgtcatcagcatcatccagattggcgttgtcaccgccgcaaacacggtagaaaagagcatgctcgccgctg |
| | | ccgggccggtgataacattaaacttctgcgacatcaggtagacgttaaccccgaccgccatcgatcccagcagcaccaccactttactctccagcggcggcagatcggtcagccacgcgatagcc |
| | | cataccgtcatcggttgcagcaccagtttcagcaggcagatggtatagctctcttttaagccatcccgcacccgatagtgcgccaggctcatgcccagcgtcaccagcgacagaggtgcggccag |
| | | tgaggccagcatctttagcggttcagcggccagcagcggcaagggacgttgcagaaagctccaggctgtaccgctgagaataccgatgatcagcggattcttcagcacgccgagcagggtgc |
| | | gccagagtccttccagcgagaagctgccctgttttgcccactcgaccgaaacggtcagtaaggtccagaggatcaggctgttaaacaccagcaccagcgccaccgggggcagggcttctggtcc |
| | | cagcagtaccgtggccactgggatccccagcatcacgttattggaaaagatgccgcccagcgcaaaaaccgacgccgctacgccatccagattaaacagccttgttgccagcagtcgccccacg |
| | | ataaacaccagcaggcaactgccgaaaaaggccagcaacaggcgcgcatccaccggggactttgcgagaaggtgctcatcacccgaaacagcatgcagggcagggcaacgttaaacaca |
| | | aagcggttcatcccttcgctgatgctgggggccatttcccgaggcgcgtcaggcagtaccccagcaggatcaggataaacagcggggctgacaggtaaatttgatggcttaagctttcaaaca |
| | | gcagtggcattcacttcttcttacgcgccgccctgccactcatcgcagtactgttgtaattcattaagcattctgccgacatggaagccatcacagacggcatgatgaacctgaatcgccagcggcat |
| | | cagcaccttgtcgccttgcgtataatatttgcccatggtgaaaacgggggcgaagaagttgtccatattggccacgtttaaatcaaaactggtgaaactcacccagggattggctgagacgaaaa |
| | | acatattctcaataaaccctttagggaaataggccaggttttcaccgtaacacgccacatcttgcgaatatatgtgtagaaactgccggaaatcgtcgtggtattcactccagagcgatgaaaacgt |
| | | ttcagtttgctcatggaaaacggtgtaacaagggtgaacactatcccatatcaccagctcaccgtctttcattgccatacggaattccggatgagcattcatcagggggcaagaatgtgaataaa |
| | | ggccggataaaacttgtgcttatttttctttacggtctttaaaaaggccgtaatatccagctgaacggtctggttataggtacattgagcaactgactgaaatgcctcaaaatgttctttacgatgccat |
| | | tgggatatatcaacggtggtatatccagtgatttttttctccattttagcttccttagctcctgaaaatctcgataactcaaaaaatacgcccggtagtgatcttatttcattatggtgaaagttggaac |
| | | ctcttacgtgccgatcaacgtctcattttcgccaaaagttggcccagggcttcccggtatcaacagggacaccaggatttatttattctgcgaagtgatcttccgtcacaggtatttattcggcgcaaag |
| | | tgcgtcgggtgatgctgccaacttactgatttagtgtatgatggtgtttttgaggtgctccagtggcttctgtttctatcagctgtccctcctgttcagctactgacggggggtgcgtaacggcaaaagc |
| | | accgccggacatcagcgctagcggagtgtatactggcttactatgttggcactgatgagggtgtcagtgaagtgcttcatgtggcaggagaaaaaaggctgcaccggtgcgtcagcagaatatg |
| | | tgatacaggatatattccgcttcctcgctcactgactcgctacgctcggtcgttcgactgcggcgagcggaaatggcttacgaacggggggagatttcctggaagatgccaggaagatacttaac |
| | | agggaagtgagagggccgcggcaaagccgtttttccataggctccgcccccctgacaagcatcacgaaatctgacgctcaaatcagtggtggcgaaacccgacaggactataaagataccag |
| | | gcgtttccccctggcggctccctcgtgcgctctcctgttcctgcctttcggtttaccggtgtcattccgctgttatggccgcgtttgtctcattccacgcctgacactcagttccgggtaggcagttcgc |
| | | tccaagctggactgtatgcacgaaccccccgttcagtccgaccgctgcgccttatccggtaactatcgtcttgagtccaacccggaaagacatgcaaaagcaccactggcagcagccactggtaatt |
| | | gatttagaggagttagtcttgaagtcatgcgccggttaaggctaaactgaaaggacaagttttggtgactgcgctcctccaagccagttacctcggttcaaagagttggtagctcagagaaccttc |
| | | gaaaaaccgccctgcaaggcggttttttcgttttcagagcaagagattacgcgcagaccaaaacgatctcaagaagatcatcttattaatcagataaaatatttgctcatgagcccgaagtggcga |
| | | gcccgatcttccccatcggtgatgtcggcgatataggcgccagcaaccgcacctgtggcgccggtgatgccggccacgatgcgtccggcgtagaggatctgctcatgtttgacagcttatcatcga |
| | | tgcataatgtgcctgtcaaatggacgaagcagggattctgcaaaccctatgctactccgtcaagccgtcaattgtctgattcgttaccaagtggaggttgtataatgtatcaccgccagaggtaaaa |
| | | tagtcaacacgcacggtgttagatatttatcccttgcggtgatagatttaacgtatgagcacaaaaaagaaaccattaacacaagagcagcttgaggacgcacgtcgccttaaagcaatttatgaa |
| | | aaaaagaaaaatgaacttggcttatcccaggaatctgtcgcagacaagatggggatggggcagtcaggcgttggtgctttatttaatggcatcaatgcattaaatgcttataacgccgcattgctt |
| | | gcaaaaattctcaaagttagcgttgaagaatttagcccttcaatcgccagagaaatctacgagatgtatgaagcggttagtatgcagccgtcacttagaagtgagtatgagtaccctgttttttctca |
| | | tgttcaggcagggatgttctcacctgagcttagaacctttaccaaaggtgatgcggagagatgggtaagcacaaccaaaaaagccagtgattctgcattctggcttgaggttgaaggtaattccat |
| | | g |
| Seq ID | p2298 | attcagcgggttcgtgcgagctgtagacaattaatactcggctcgtataatgtgtggaaggctagcaggaggaattcaatgatatggttaagttagagaatagtcgcaaacctgagaaaatctca |
| No: 57 | | aataagaatattcctatgagcgacttcgtcgtgaatttagaccacggtgatccgacagcgtacgaagagtactggcgcaaaatgggagaccgctgtaccgtgaccattcgcggttgtgacttgat |
| | | gagttatttttctgatatgacgaatctgtgttggttcttggagccggaactggaggatgcgattaaggatttacacggtgtcgttggcaatgcagctacggaggaccgctatatcgtggtgggaact |
| | | gggtctacgcagttatgccaggctgcggtccacgcattgtcttctcttgcgcgtagtcaaccagtttccgttgtggctgcggcaccattttacagcacgtacgtggaggagaccacctatgttcgcag |
| | | tgggatgtacaagtgggagggagacgcgtgggggtttgataagaaaggtccttatattgaacttgtgacatcaccgaataacccggacgggacgatccgcgagaccgtggttaatcgcccgga |
| | | tgatgatgaagcaaaggtaatccacgacttcgcttactattggccccattatacacctattacgcgtcgccaggatcacgacatcatgctttttacctttagcaaaatcacggggcacgctggtagtc |
| | | gtattggatgggcgctggtgaaggataaagaggtcgctaagaaaatggttgagtatattatcgtgaacagtatcggcgtttcgaaagaatcgcaggtgcgtacagcgaaaattttgaatgtcctg |
| | | aaggaaacgtgtaaatctgaatccgaatccgagaacttcttcaaatatggtcgtgagatgatgaagaatcgctgggagaagctgcgtgaagtagtcaaggagtcagacgccttcacattgccca |
| | | aatatcctgaggctttttgtaattatttcgggaagagtttagaatcatacccggcgtttgcctggctggggacaaaggaagagaccgacttggtatcggaattgcgtcgtcacaaggtaatgtcgcg |
| | | tgcaggcgagcgttgcggttcagataagaagcatgttcgcgtatcaatgctgtcccgcgaggatgttttcaatgtcttcttagagcgcttagccaacatgaaattgatcaaatctatcgatttgtaaat |
| | | ccgaactccaattattgatgaaaagggattataaaagctcacttattcaggcgtagcacccaggcgtttaagggcaccaataactgccttaaaaaaattacgccccgccctgccactcatcgcagt |
| | | actgttgtaattcattaagcattctgccgacatggaagccatcacagacggcatgatgaacctgaatcgccagcggcatcagcaccttgtcgccttgcgtataatatttgcccatggtgaaaacggg |
| | | ggcgaagaagttgtccatattggccacgtttaaatcaaaactggtgaaactcacccagggattggctgagacgaaaaacatattctcaataaaccctttagggaaataggccaggttttcaccgta |
| | | acacgccacatcttgcgaatatatgtgtagaaactgccggaaatcgtcgtggtattcactccagagcgatgaaaacgtttcagtttgctcatggaaaacggtgtaacaagggtgaacactatccca |
| | | tatcaccagctcaccgtctttcattgccatacggaattccggatgagcattcatcagggggcaagaatgtgaataaaggccggataaaacttgtgcttatttttctttacggtctttaaaaaggccgt |
| | | aatatccagctgaacggtctggttataggtacattgagcaactgactgaaatgcctcaaaatgttctttacgatgccattgggatatatcaacggtggtatatccagtgatttttttctccattttagct |
| | | tccttagctcctgaaaatctcgataactcaaaaaatacgcccggtagtgatcttatttcattatggtgaaagttggaacctcttacgtgccgatcaacggtcgctacgctccgggcgtgagactgcgg |
| | | cgggcgctgcggacacatacaaagttacccacagattccgtggataagcaggggactaacatgtgaggcaaaacagcagggccgcgccggtggcgtttttccataggctccgccctcctgcca |
| | | gagttcacataaacagacgcttttccggtgcatctgtgggagccgtgaggctcaaccatgaatctgacagtacgggcgaaacccgacaggacttaaagatccccaccgtttccgggggtcgctc |
| | | cctcttgcgctctcctgttccgaccctgccgtttaccggatacctgttccgcctttctcccttacgggaagtgtggcgctttctcatagctcacacactggtatctcggctcggtgtaggtcgttcgctc |
| | | caagctgggctgtaagcaagaactccccgttcagcccgactgctgcgccttatccggtaactgttcacttgagtccaacccggaaaagcacggtaaaacgccactggcagcagccattggtaactgg |
| | | gagttcgcagaggatttgtttagctaaacacgcggttgctcttgaagtgtgcgccaaagtccggctacactggaaggacagatttggttgctgtgctctgcgaaagccagttaccacggttaagca |
| | | gttccccaactgacttaaccttcgatcaaaccacctccccaggtggttttttcgtttacagggcaaaagattacgcgcagaaaaaaaggatctcaagaagatcctttgatctgatgccggccacgat |
| | | gcgtccggcgtagaggatctgctcatgtttgacagcttatcatcgatgcataatgtgcctgtcaaatggacgaagcagggattctgcaaaccctatgctactccgtcaagccgtcaattgtctgattc |
| | | gttaccaagtggaggttgtataatg |
| Seq ID | p2299 | attcagcgggttcgtgcgagctgtagacaattaatactcggctcgtataatgtgtggaaggctagcaggaggaattcaatgatatggttaagttagagaatagtcgcaaacctgagaaaatctca |
| No: 58 | | aataagaatattcctatgagcgacttcgtcgtgaatttagaccacggtgatccgacagcgtacgaagagtactggcgcaaaatgggagaccgctgtaccgtgaccattcgcggttgtgacttgat |
| | | gagttatttttctgatatgacgaatctgtgttggttcttggagccggaactggaggatgcgattaaggatttacacggtgtcgttggcaatgcagctacggaggaccgctatatcgtggtgggaact |
| | | gggtctacgcagttatgccaggctgcggtccacgcattgtcttctcttgcgcgtagtcaaccagtttccgttgtggctgcggcaccattttacagcacgtacgtggaggagaccacctatgttcgcag |
| | | tgggatgtacaagtgggagggagacgcgtgggggtttgataagaaaggtccttatattgaacttgtgacatcaccgaataacccggacgggacgatccgcgagaccgtggttaatcgcccgga |
| | | tgatgatgaagcaaaggtaatccacgacttcgcttactattggccccattatacacctattacgcgtcgccaggatcacgacatcatgctttttacctttagcaaaatcacggggcacgctggtagtc |
| | | gtattggatgggcgctggtgaaggataaagaggtcgctaagaaaatggttgagtatattatcgtgaacagtatcggcgtttcgaaagaatcgcaggtgcgtacagcgaaaattttgaatgtcctg |
| | | aaggaaacgtgtaaatctgaatccgaatccgagaacttcttcaaatatggtcgtgagatgatgaagaatcgctgggagaagctgcgtgaagtagtcaaggagtcagacgccttcacattgccca |
| | | aatatcctgaggctttttgtaattatttcgggaagagtttagaatcatacccggcgtttgcctggctggggacaaaggaagagaccgacttggtatcggaattgcgtcgtcacaaggtaatgtcgcg |
| | | tgcaggcgagcgttgcggttcagataagaagcatgttcgcgtatcaatgctgtcccgcgaggatgttttcaatgtcttcttagagcgcttagccaacatgaaattgatcaaatctatcgatttgtaaat |
| | | ccgaactccaattattgaaggccgctaacgcggccttttttttttctggtctccctgaacgctctctactagagtcacactggctcaccttcggggggcctttctgcgtttataggtacccgttgaaaa |
| | | gggattataaaagcgttaatagcccgccagcgtcatcagcatcatccagattggcgttgtcaccgccgcaaacacggtagaaaagagcatgctcgccgctgccgggccggtgataacattaaac |
| | | ttctgcgacatcaggtagacgttaaccccgaccgccatcgatcccagcagcaccaccactttactctccagcggcggcagatcggtcagccacgcgatagcccataccgtcatcggttgcagcac |
| | | cagtttcagcaggcagatggtatagctctcttttaagccatcccgcacccgatagtgcgccaggctcatgcccagcgtcaccagcgacagaggtgcggccagtgaggccagcatctttagcggtt |
| | | cagcggccagcagcggcaagggacgttgcagaaagctccaggctgtaccgctgagaataccgatgatcagcggattcttcagcacgccgagcagggtgcgccagagtccttccagcgagaa |
| | | gctgccctgttttgcccactcgaccgaaacggtcagtaaggtccagaggatcaggctgttaaacaccagcaccagcgccaccgggggcagggcttctggtcccagcagtaccgtggccactgg |
| | | gatccccagcatcacgttattggaaaagatgccgcccagcgcaaaaaccgacgccgctacgccatccagattaaacagccttgttgccagcagtcgccccacgataaacaccagcaggcaact |
| | | gccgaaaaaggccagcaacaggcgcgcatccaccggcggactttgcgagaaggtgctcatcacccgaaacagcatgcagggcagggcaacgttaaacacaaagcggttcatcccttcgctga |
| | | tgctggcgggccatttcccgaggcgcgtcaggcagtaccccagcaggatcaggataaacagcggggctgacaggtaaatttgatggcttaagctttcaaacagcagtggcattcacttcttcttac |
| | | gcgccgccctgccactcatcgcagtactgttgtaattcattaagcattctgccgacatggaagccatcacagacggcatgatgaacctgaatcgccagcggcatcagcaccttgtcgccttgcgtat |
| | | aatatttgcccatggtgaaaacgggggcgaagaagttgtccatattggccacgtttaaatcaaaactggtgaaactcacccagggattggctgagacgaaaaacatattctcaataaacccttta |
| | | gggaaataggccaggttttcaccgtaacacgccacatcttgcgaatatatgtgtagaaactgccggaaatcgtcgtggtattcactccagagcgatgaaaacgtttcagtttgctcatggaaaacg |
| | | gtgtaacaagggtgaacactatcccatatcaccagctcaccgtctttcattgccatacggaattccggatgagcattcatcagggggcaagaatgtgaataaaggccggataaaacttgtgctta |
| | | tttttctttacggtctttaaaaaggccgtaatatccagctgaacggtctggttataggtacattgagcaactgactgaaatgcctcaaaatgttctttacgatgccattgggatatatcaacggtggtat |
| | | atccagtgatttttttctccattttagcttccttagctcctgaaaatctcgataactcaaaaaatacgcccggtagtgatcttatttcattatggtgaaagttggaacctcttacgtgccgatcaacggt |
| | | cgctacgctccgggcgtgagactgcggcgggcgctgcggacacatacaaagttacccacagattccgtggataagcaggggactaacatgtgaggcaaaacagcagggccgcgccggtggcgt |
| | | ttttccataggctccgccctcctgccagagttcacataaacagacgcttttccggtgcatctgtgggagccgtgaggctcaaccatgaatctgacagtacgggcgaaacccgacaggacttaaaga |
| | | tccccaccgtttccggcgggtcgctccctcttgcgctctcctgttccgaccctgccgtttaccggatacctgttccgcctttctcccttacgggaagtgtggcgctttctcatagctcacacactggtat |
| | | ctcggctcggtgtaggtcgttcgctccaagctgggctgtaagcaagaactccccgttcagcccgactgctgcgccttatccggtaactgttcacttgagtccaacccggaaaagcacggtaaaacgcc |
| | | actggcagcagccattggtaactgggagttcgcagaggatttgtttagctaaacacgcggttgctcttgaagtgtgcgccaaagtccggctacactggaaggacagatttggttgctgtgctctgc |
| | | gaaagccagttaccacggttaagcagttccccaactgacttaaccttcgatcaaaccacctccccaggtggttttttcgtttacagggcaaaagattacgcgcagaaaaaaaggatctcaagaag |
| | | atcctttgatctgatgccggccacgatgcgtccggcgtagaggatctgctcatgtttgacagcttatcatcgatgcataatgtgcctgtcaaatggacgaagcagggattctgcaaaccctatgctac |
| | | tccgtcaagccgtcaattgtctgattcgttaccaagtcgaggttgtataatg |
| Seq ID | p2300 | attcagcgggttcgtgcgagctgtagacaattaatactcggctcgtataatgtgtggaaggctagcaggaggaattcaatgatatgtcgacttttacagtaggtgactatttattaactcgcttacaa |
| No: 59 | | gagatcggggtaaagcatttatttggagtacctggtgactataacttacaatttcttgaccgtgtgatcgctcatcccgctatttcatgggtaggctgtgccaacgagttgaacgcagcctatgcggc |
| | | tgatggatatgcacgttgcaatggggccggtgctcttcttacgaccttcggggttggcgagctttccgcgatcaacggcatcgctggttcttatgccgagtatctgccggtcattcatattgtcggggc |
| | | accagctacgcaggcgcagttgcaaggggactgcgtgcatcattctttaggagatggggacttccagcattttatccgcatggcgactgaagtatcggctgccacggctcaacttaacgccgaca |
| | | acgccactgccgaaatcgaccgtgtaatcgtaagtgccctgcaagcacgccgtcccggatatctgtctctggctgtggatgtggcagccatggaagttcaaccaccagctcaaccattgaacagc |
| | | caccaaagctctagtccggcagcccgtcgcgccttctcagatgctgccgagcgtttattggcgccagcacaacgcgtcagtctgcttgcggattttttagccctgcgttggcagcaacaatcagcctt |
| | | agctgcattgcgcgaacaatgcgccatcccgtgcgcttcgctgttaatggggaagggagtcttggatgagcagcagcccgggtatgttggtacttatgctggcgaggcgtctgcgggccaggttt |
| | | gtgaacagattgaacaagtagacgtcgcaatttgtgtcggggtccgttttacggacaccatcaccgccggatttacacagcagtttaatcccgagcgcctgatcgacttgcagccacttagcgcta |
| | | gtgtcggcggagaacgctttgccccactttcaatggcagacgctctttccgaacttttgccgcttttcgagcgctatgggcaacagtggcaaccaggggctacgccacctgcagcacaaccggctg |
| | | aacccgccgccgtcatttcccaacaagcgttttggcaagcaatgcaagccttcttacagccgggcgatcttattttggccgaacaaggtactgcggcatttggggctgccgcgcttcgtctgccaag |
| | | ccaggcccagttagtagtacaacctctttgggggtctattggatacaccctgccagcagcttttggtgcgcaaacggctgaccctgatcgtcgcgtgatcctgatcattggagatggttcagcgcaa |
| | | ttgactatccaagagttaggaagcatgctgcgcgaccaacaacgcttgatccttttcctgctgaacaatgagggctatactgttgaacgtgcgattcacggtgctacgcagcgttataatgacattg |
| | | cccagtggaattggaccgctctgcctcacgcactttctttgcaaggccaggcacaatcatggcgtatctcggagacggtgcaattagaggaggtgatggctcgtttgtccgaacccaagtggctgt |
| | | cattggtcgaggtcgtgatgcagaaggaggacctgcctcctttattacgcaaagtaacagcttgtcttaaccagcgtaatggctgctaaaagattgataataatctagaccaggcagtggattaaa |
| | | gaggaggaaagtaatggttaagttagagaatagtcgcaaacctgagaaaatctcaaataagaatattcctatgagcgacttcgtgtgaatttagaccacggtgatccgacagcgtacgaagag |
| | | tactggcgcaaaatgggagaccgctgtaccgtgaccattcgcggttgtgacttgatgagttatttttctgatatgacgaatctgtgttggttcttggagccggaactggaggatgcgattaaggattt |
| | | acacggtgtcgttggcaatgcagctacggaggaccgctatatcgtggtgggaactgggtctacgcagttatgccaggctgcggtccacgcattgtcttctcttgcgcgtagtcaaccagtttccgtt |
| | | gtggctgcggcaccattttacagcacgtacgtggaggagaccacctatgttcgcagtgggatgtacaagtgggagggagacgcgtgggggtttgataagaaaggtccttatattgaacttgtga |
| | | catcaccgaataacccggacgggacgatccgcgagaccgtggttaatcgcccggatgatgatgaagcaaaggtaatccacgacttcgcttactattggccccattatacacctattacgcgtcgcc |
| | | aggatcacgacatcatgctttttacctttagcaaaatcacggggcacgctggtagtcgtattggatgggcgctggtgaaggataaagaggtcgctaagaaaatggttgagtatattatcgtgaaca |
| | | gtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaat |
| | | cgctgggagaagctgcgtgaagtagtcaaggagtcagacgccttcacattgcccaaatatcctgaggctttttgtaattatttcgggaagagtttagaatcatacccggcgtttgcctggctgggga |
| | | caaaggaagagaccgacttggtatcggaattgcgtcgtcacaaggtaatgtcgcgtgcaggcgagcgttgcggttcagataagaagcatgttcgcgtatcaatgctgtcccgcgaggatgttttc |
| | | aatgtcttcttagagcgcttagccaacatgaaattgatcaaatctatcgatttgtaatcacttcttcttacgcgccgccctgccactcatcgcagtactgttgtaattcattaagcattctgccgacatg |
| | | gaagccatcacagacggcatgatgaacctgaatcgccagcggcatcagcaccttgtcgccttgcgtataatatttgcccatggtgaaaacgggggcgaagaagttgtccatattggccacgtttaa |
| | | atcaaaactggtgaaactcacccagggattggctgagacgaaaaacatattctcaataaaccctttagggaaataggccaggttttcaccgtaacacgccacatcttgcgaatatatgtgtagaa |
| | | actgccggaaatcgtcgtggtattcactccagagcgatgaaaacgtttcagtttgctcatggaaaacggtgtaacaagggtgaacactatcccatatcaccagctcaccgtctttcattgccatacg |
| | | gaattccggatgagcattcatcagggggcaagaatgtgaataaaggccggataaaacttgtgcttatttttctttacggtctttaaaaaggccgtaatatccagctgaacggtctggttataggta |
| | | cattgagcaactgactgaaatgcctcaaaatgttctttacgatgccattgggatatatcaacggtggtatatccagtgatttttttctccattttagcttccttagctcctgaaaatctcgataactcaa |
| | | aaaatacgcccggtagtgatcttatttcattatggtgaaagttggaacctcttacgtgccgatcaacggtcgctacgctccgggcgtgagactgcgggggcgctgcggacacatacaaagttaccca |
| | | cagattccgtggataagcaggggactaacatgtgaggcaaaacagcagggccgcgccggtggcgtttttccataggctccgccctcctgccagagttcacataaacagacgcttttccggtgcat |
| | | ctgtgggagccgtgaggctcaaccatgaatctgacagtacgggcgaaacccgacaggacttaaagatccccaccgtttccgggggtcgctccctcttgcgctctcctgttccgaccctgccgttta |
| | | ccggatacctgttccgcctttctcccttacgggaagtgtggcgctttctcatagctcacacactggtatctcggctcggtgtaggtcgttcgctccaagctgggctgtaagcaagaactccccgttcag |
| | | cccgactgctgcgccttatccggtaactgttcacttgagtccaacccggaaaagcacggtaaaacgccactggcagcagccattggtaactgggagttcgcagaggatttgtttagctaaacacg |
| | | cggttgctcttgaagtgtgcgccaaagtccggctacactggaaggacagatttggttgctgtgctctgcgaaagccagttaccacggttaagcagttccccaactgacttaaccttcgatcaaacca |
| | | cctccccaggtggttttttcgtttacagggcaaaagattacgcgcagaaaaaaaggatctcaagaagatcctttgatctgatgccggccacgatgcgtccggcgtagaggatctgctcatgtttgac |
| | | agcttatcatcgatgcataatgtccctctcaaatggacgaagcagggattctgcaaaccctatgctactccgtcaagccgtcaattgtctgattcgttaccaagtcgaggttgtataatg |
| Seq ID | p2301 | attcagcgggttcgtgcgagctgtagacaattaatactcggctcgtataatgtgtggaaggctagcaggaggaattcaatgatatggttaagttagagaatagtcgcaaacctgagaaaatctca |
| No: 60 | | aataagaatattcctatgagcgacttcgtcgtgaatttagaccacggtgatccgacagcgtacgaagagtactggcgcaaaatgggagaccgctgtaccgtgaccattcgcggttgtgacttgat |
| | | gagttatttttctgatatgacgaatctgtgttggttcttggagccggaactggaggatgcgattaaggatttacacggtgtcgttggcaatgcagctacggaggaccgctatatcgtggtgggaact |
| | | gggtctacgcagttatgccaggctgcggtccacgcattgtcttctcttgcgcgtagtcaaccagtttccgttgtggctgcggcaccattttacagcacgtacgtggaggagaccacctatgttcgcag |
| | | tgggatgtacaagtgggagggagacgcgtgggggtttgataagaaaggtccttatattgaacttgtgacatcaccgaataacccggacgggacgatccgcgagaccgtggttaatcgcccgga |
| | | tgatgatgaagcaaaggtaatccacgacttcgcttactattggccccattatacacctattacgcgtcgccaggatcacgacatcatgctttttacctttagcaaaatcacggggcacgctggtagtc |
| | | gtattggatgggcgctggtgaaggataaagaggtcgctaagaaaatggttgagtatattatcgtgaacagtatcggcgtttcgaaagaatcgcaggtgcgtacagcgaaaattttgaatgtcctg |
| | | aaggaaacgtgtaaatctgaatccgaatccgagaacttcttcaaatatggtcgtgagatgatgaagaatcgctgggagaagctgcgtgaagtagtcaaggagtcagacgccttcacattgccca |
| | | aatatcctgaggctttttgtaattatttcgggaagagtttagaatcatacccggcgtttgcctggctggggacaaaggaagagaccgacttggtatcggaattgcgtcgtcacaaggtaatgtcgcg |
| | | tgcaggcgagcgttgcggttcagataagaagcatgttcgcgtatcaatgctgtcccgcgaggatgttttcaatgtcttcttagagcgcttagccaacatgaaattgatcaaatctatcgatttgtaaat |
| | | atcaatgataaaggaggaaacaaatggaaagtggtcatcgctttgatgctcagacgctgcacagttttattcaggctgtatttcgtcagatgggtagcgaggaacaagaagcgaaattagttgcc |
| | | gatcatttaatcgcggcaaacctggcagggcatgattcacatggtattggcatgatcccaagctatgtacgctcctggagtcaggggcacctgcaaattaaccatcatgccaaaaccgttaaaga |
| | | ggcgggggcggcggtcacgctcgatggcgatcgcgcatttggtcaggtcgcggcacatgaagcgatggcgctggggattgagaaagcgcatcagcacggtattgccgccgtggcgctacata |
| | | actcgcatcatatcggccgtatcggttactgggcggagcagtgtgcagcggcggggtttgtctctatccactttgttagcgtggtcggtattccaatggtcgcgccgttccacggtcgcgacagccg |
| | | ctttggcaccaatccgttctgtgtggttttccctcgtaaagataatttcccgctgttgcttgattacgccaccagcgccattgcatttggcaaaacccgcgtcgcctggcataaaggcgtccccgtgcc |
| | | gccaggttgcctgattgacgttaacggcgtgccgacgaccaatccggcggtaatgcaggagtcgccgttgggttcgctgttgacctttgccgaacataaaggctacgcccttgcagcgatgtgtg |
| | | aaattcttggggggcgctttccggcggtaaaacgacgcatcaggaaacgttacaaaccagtcccgatgccattcttaactgcatgaccactatcatcatcaacccggaactcttcggcgcgccgg |
| | | attgtaacgcgcagaccgaagcctttgccgagtgggtgaaagcctcgccgcatgatgatgataagccgattttgctaccgggcgagtgggaagtgaacacgcgtcgcgaacggcagaagcag |
| | | gggattccactggatgcgggaagctggcaggccatttgtgatgcagcgcggcagattggtatgccggaagagacgttgcaggctttctgtcagcagttagccagctaaatccgaactccaattat |
| | | tgatgaaaagggattataaaagctcacttattcaggcgtagcacccaggcgtttaagggcaccaataactgccttaaaaaaattacgccccgccctgccactcatcgcagtactgttgtaattcatt |
| | | aagcattctgccgacatggaagccatcacagacggcatgatgaacctgaatcgccagcggcatcagcaccttgtcgccttgcgtataatatttgcccatggtgaaaacgggggcgaagaagttg |
| | | tccatattggccacgtttaaatcaaaactggtgaaactcacccagggattggctgagacgaaaaacatattctcaataaaccctttagggaaataggccaggttttcaccgtaacacgccacatctt |
| | | gcgaatatatgtgtagaaactgccggaaatcgtcgtggtattcactccagagcgatgaaaacgtttcagtttgctcatggaaaacggtgtaacaagggtgaacactatcccatatcaccagctcac |
| | | cgtctttcattgccatacggaattccggatgagcattcatcagggggcaagaatgtgaataaaggccggataaaacttgtgcttatttttctttacggtctttaaaaaggccgtaatatccagctga |
| | | acggtctggttataggtacattgagcaactgactgaaatgcctcaaaatcttctttacgatgccattgggatatatcaacggtggtatatccagtgatttttttctccattttagcttccttagctcctg |
| | | aaaatctcgataactcaaaaaatacgcccggtagtgatcttatttcattatggtgaaagttggaacctcttacgtgccgatcaacggtcgctacgctccgggcgtgagactgcgggggcgctgcgga |
| | | cacatacaaagttacccacagattccgtggataagcaggggactaacatgtgaggcaaaacagcagggccgcgccggtggcgtttttccataggctccgccctcctgccagagttcacataaac |
| | | agacgcttttccggtgcatctgtgggagccgtgaggctcaaccatgaatctgacagtacgggcgaaacccgacaggacttaaagatccccaccgtttccgggggtcgctccctcttgcgctctcc |
| | | tgttccgaccctgccgtttaccggatacctgttccgcctttctcccttacgggaagtgtggcgctttctcatagctcacacactggtatctcggctcggtgtaggtcgttcgctccaagctgggctgtaa |
| | | gcaagaactccccgttcagcccgactgctgcgccttatccggtaactgttcacttgagtccaacccggaaaagcacggtaaaacgccactggcagcagccattggtaactgggagttcgcagag |
| | | gatttgtttagctaaacacgcggttgctcttgaagtgtgcgccaaagtccggctacactggaaggacagatttggttgctgtgctctgcgaaagccagttaccacggttaagcagttccccaactga |
| | | cttaaccttcgatcaaaccacctccccaggtggttttttcgtttacagggcaaaagattacgcgcagaaaaaaaggatctcaagaagatcctttgatctgatgccggccacgatgcgtccggcgta |
| | | gaggatctgctcatgtttgacagcttatcatcgatgcataatgtgcctgtcaaatggacgaagcagggattctgcaaaccctatgctactccgtcaagccgtcaattgtctgattcgttaccaagtgg |
| | | aggttgtataatg |
| Seq ID | p2302 | ccgggcgtgagactgcggcgggcgctgcggacacatacaaagttacccacagattccgtggataagcaggggactaacatgtgaggcaaaacagcagggccgcgccggtggcgtttttccat |
| No: 61 | | aggctccgccctcctgccagagttcacataaacagacgcttttccggtgcatctgtgggagccgtgaggctcaaccatgaatctgacagtacgggcgaaacccgacaggacttaaagatcccca |
| | | ccgtttccggcgggtcgctccctcttgcgctctcctgttccgaccctgccgtttaccggatacctgttccgcctttctcccttacgggaagtgtggcgctttctcatagctcacacactggtatctcggc |
| | | tcggtgtaggtcgttcgctccaagctgggctgtaagcaagaactccccgttcagcccgactgctgcgccttatccggtaactgttcacttgagtccaacccggaaaagcacggtaaaacgccactgg |
| | | cagcagccattggtaactgggagttcgcagaggatttgtttagctaaacacgcggttgctcttgaagtgtgcgccaaagtccggctacactggaaggacagatttggttgctgtgctctgcgaaag |
| | | ccagttaccacggttaagcagttccccaactgacttaaccttcgatcaaaccacctccccaggtggttttttcgtttacagggcaaaagattacgcgcagaaaaaaaggatctcaagaagatccttt |
| | | gatctgatgccggccacgatgcgtccggcgtagaggatctgctcatgtttgacagcttatcatcgatgcataatgtgcctgtcaaatggacgaagcagggattctgcaaaccctatgctactccgtc |
| | | aagccgtcaattgtctgattcgttaccaagtggaggttgtataatgattcagcgggttcgtgcgagctgtagacaattaatactcggctcgtataatgtgtggaaggctagcaggaggaattcaatg |
| | | atatggttaagttagagaatagtcgcaaacctgagaaaatctcaaataagaatattcctatgagcgacttcgtgtgaatttagaccacggtgatccgacagcgtacgaagagtactggcgcaaa |
| | | atgggagaccgctgtaccgtgaccattcgcggttgtgacttgatgagttatttttctgatatgacgaatctgtgttggttcttggagccggaactggaggatgcgattaaggatttacacggtgtcgtt |
| | | ggcaatgcagctacggaggaccgctatatcgtggtgggaactgggtctacgcagttatgccaggctgcggtccacgcattgtcttctcttgcgcgtagtcaaccagtttccgttgtggctgcggcac |
| | | cattttacagcacgtacgtggaggagaccacctatgttcgcagtgggatgtacaagtgggagggagacgcgtgggggtttgataagaaaggtccttatattgaacttgtgacatcaccgaataac |
| | | ccggacgggacgatccgcgagaccgtggttaatcgcccggatgatgatgaagcaaaggtaatccacgacttcgcttactattggccccattatacacctattacgcgtcgccaggatcacgacat |
| | | catgctttttacctttagcaaaatcacggggcacgctggtagtcgtattggatgggcgctggtgaaggataaagaggtcgctaagaaaatggttgagtatattatcgtgaacagtatcggcgtttc |
| | | gaaagaatcgcaggtgcgtacagcgaaaattttgaatgtcctgaaggaaacgtgtaaatctgaatccgaatccgagaacttcttcaaatatggtcgtgagatgatgaagaatcgctgggagaag |
| | | ctgcgtgaagtagtcaaggagtcagacgccttcacattgcccaaatatcctgaggctttttgtaattatttcgggaagagtttagaatcatacccggcgtttgcctggctggggacaaaggaagag |
| | | accgacttggtatcggaattgcgtcgtcacaaggtaatgtcgcgtgcaggcgagcgttgcggttcagataagaagcatgttcgcgtatcaatgctgtcccgcgaggatgttttcaatgtcttcttag |
| | | agcgcttagccaacatgaaattgatcaaatctatcgatttgtaaatatcaatgataaaggaggaaacaaatggtgaccatgaatcgtaataaagttgttatcgtgggtacgggccaggtcggcgc |
| | | caccgctgcgttcggcattgttacgcatggtttgtgcaacgaactggtcttgatcgaccgttccgccgccaaggcattgggggaggctcgtgaccttgacgacggtagcgaatttcaggaccgtca |
| | | cgttaaggtccgcgcgggagattacgccgattgcaaagacgctgacattgttgttattactgttgggcgtaagccaccagcgaacagtaaccgtatggcggaacttggttttacggtcggtttggt |
| | | gggcgaagtagttgataatgttatggctagtggatttgatggagttattgttatggttagcaaccccgtcgatgttatggcttggtatgcttggaagcgttcgggactgccccgcacgcaagtccttg |
| | | gaacgggtaccgccttagatacctcccgccttaagacaattatcggagaagaaactggtttagatccacgtaacgtaggcggctttgttatgggggaacatggtgattctcagttcactgcttgga |
| | | gcactgtatcgttaggcggtaaaccgtttgctcgctttttagcagacaatcaagatcgcttcgcatcggtatcaactaccgagatcgaggaaaaaacgcgcacacgtggcgacgaaatcgtagcg |
| | | gccaaggggggaccaattttggcattgcgtcaacagttgccggtatcgtacaaaccatcctgtgggacgagcgccgcatcgtaccagtaagcacgctgcttgatggagagtacggtgaacatg |
| | | acgtatttttgggtgtcccaacggaactgcgtgcaaacggggcgaatgaaatcgtagaattggacttaagcgaagacgagcgcgctaagttacatcattcggccgaattggtgcgtgaccactg |
| | | caaaggattgctttgaatccgaactccaattattgatgaaaagggattataaaagctcacttattcaggcgtagcaccaggcgtttaagggcaccaataactgccttaaaaaaattacgccccgcc |
| | | ctgccactcatcgcagtactgttgtaattcattaagcattctgccgacatggaagccatcacagacggcatgatgaacctgaatcgccagcggcatcagcaccttgtcgccttgcgtataatatttgc |
| | | ccatggtgaaaacgggggcgaagaagttgtccatattggccacgtttaaatcaaaactggtgaaactcacccagggattggctgagacgaaaaacatattctcaataaaccctttagggaaata |
| | | ggccaggttttcaccgtaacacgccacatcttgcgaatatatgtgtagaaactgccggaaatcgtcgtggtattcactccagagcgatgaaaacgtttcagtttgctcatggaaaacggtgtaaca |
| | | agggtgaacactatcccatatcaccagctcaccgtctttcattgccatacggaattccggatgagcattcatcagggggcaagaatgtgaataaaggccggataaaacttgtgcttatttttcttta |
| | | cggtctttaaaaaggccgtaatatccagctgaacggtctggttataggtacattgagcaactgactgaaatgcctcaaaatgttctttacgatgccattgggatatatcaacggtggtatatccagtg |
| | | atttttttctccattttagcttccttagctcctgaaaatctcgataactcaaaaaatacgcccggtagtgatcttatttcattatggtgaaagttggaacctcttacgtgccgatcaacggtcgctacgc |
| Seq ID | p2303 | tattcagcgggttcgtgcgagctgtagacaattaatactcggctcgtataatgtgtggaaggctagcaggaggaattcaatgatatggttaagttagagaatagtcgcaaacctgagaaaatctca |
| No: 62 | | aataagaatattcctatgagcgacttcgtcgtgaatttagaccacggtgatccgacagcgtacgaagagtactggcgcaaaatgggagaccgctgtaccgtgaccattcgcggttgtgacttgat |
| | | gagttatttttctgatatgacgaatctgtgttggttcttggagccggaactggaggatgcgattaaggatttacacggtgtcgttggcaatgcagctacggaggaccgctatatcgtggtgggaact |
| | | gggtctacgcagttatgccaggctgcggtccacgcattgtcttctcttgcgcgtagtcaaccagtttccgttgtggctgcggcaccattttacagcacgtacgtggaggagaccacctatgttcgcag |
| | | tgggatgtacaagtgggagggagacgcgtgggggtttgataagaaaggtccttatattgaacttgtgacatcaccgaataacccggacgggacgatccgcgagaccgtggttaatcgcccgga |
| | | tgatgatgaagcaaaggtaatccacgacttcgcttactattggccccattatacacctattacgcgtcgccaggatcacgacatcatgctttttacctttagcaaaatcacggggcacgctggtagtc |
| | | gtattggatgggcgctggtgaaggataaagaggtcgctaagaaaatggttgagtatattatcgtgaacagtatcggcgtttcgaaagaatcgcaggtgcgtacagcgaaaattttgaatgtcctg |
| | | aaggaaacgtgtaaatctgaatccgaatccgagaacttcttcaaatatggtcgtgagatgatgaagaatcgctgggagaagctgcgtgaagtagtcaaggagtcagacgccttcacattgccca |
| | | aatatcctgaggctttttgtaattatttcgggaagagtttagaatcatacccggcgtttgcctggctggggacaaaggaagagaccgacttggtatcggaattgcgtcgtcacaaggtaatgtcgcg |
| | | tgcaggcgagcgttgcggttcagataagaagcatgttcgcgtatcaatgctgtcccgcgaggatgttttcaatgtcttcttagagcgcttagccaacatgaaattgatcaaatctatcgatttgtaaat |
| | | atcaatgataaaggaggaaacaaatgaaaatcttagcttattgtgtaagacctgatgaaattgattcatttaaaaatttttctgaaaaatatggacatactgttgatttaataccagattcttttggtcc |
| | | aagtgtggctcatttagctaaaggatatgatggcatttcaattttgggaaatgatacttgtaacagagaagcattagagaaaattaaagattgtggtataaaatatttagcaaccagaactgctgg |
| | | agtaaataatatagattttgatgctgcaaaagaatttggtataaatgtagctaatgttccagcttattctcctaattcagtttcagagtttactgtaggactcgcattatccctaacaaggaaaattcct |
| | | tttgcattaaaaagagtagagcttaacaattttgcattaggtggattaataggagtagaattaagaaacttaactttaggagttataggaactggaagaataggtttaaaagtaattgaaggcttttca |
| | | ggctttggtatgaaaaagatgataggttacgatatttttgaaaatgaaaaggctaaagaatatattgaatataaatctttagatgaagtatataaagaagcagacataataacactacatgcacctt |
| | | taacagatgataattaccatatgatagggaaagagagtattgctaaaatgaaagacggtgtattcataataaatgcagcacgaggagcattaattgatagtgaagctttaatagaaggactgaa |
| | | atctggtaaaatagctggagcagccttagattcttatgaatatgaacaaggagttttccataacaataaaatgaatgaaatcatgaaagatgatacattagcaagacttaagagttttccaaatgta |
| | | gttattacacctcatttaggattctatacagatgaagctgtatcaaatatggttgaaattactcttatgaatttacaagagtttgaattaaaaggaacttgtaaaaatcagagggtatgtaagtaaatc |
| | | cgaactccaattattgatgaaaagggattataaaagctcacttattcaggcgtagcacccaggcgtttaagggcaccaataactgccttaaaaaaattacgccccgccctgccactcatcgcagta |
| | | ctgttgtaattcattaagcattctgccgacatggaagccatcacaaacggcatgatgaacctgaatcgccagcggcatcagcaccttgtcgccttgcgtataatatttgcccatggtgaaaacgggg |
| | | gcgaagaagttgtccatattggccacgtttaaatcaaaactggtgaaactcacccagggattggctgagacgaaaaacatattctcaataaaccctttagggaaataggccaggttttcaccgtaa |
| | | cacgccacatcttgcgaatatatgtgtagaaactgccggaaatcgtcgtggtattcactccagagcgatgaaaacgtttcagtttgctcatggaaaacggtgtaacaagggtgaacactatcccat |
| | | atcaccagctcaccgtctttcattgccatacggaattccggatgagcattcatcagggggcaagaatgtgaataaaggccggataaaacttgtgcttatttttctttacggtctttaaaaaggccgt |
| | | aatatccagctgaacggtctggttataggtacattgagcaactgactgaaatgcctcaaaatgttctttacgatgccattgggatatatcaacggtggtatatccagtgatttttttctccattttagct |
| | | tccttagctcctgaaaatctcgataactcaaaaaatacgcccggtagtgatcttatttcattatggtgaaagttggaacctcttacgtgccgatcaacggtcgctacgctccgggcgtgagactgcgg |
| | | cgggcgctgcggacacatacaaagttacccacagattccgtggataagcaggggactaacatgtgaggcaaaacagcagggccgcgccggtggcgtttttccataggctccgccctcctgcca |
| | | gagttcacataaacagacgcttttccggtgcatctgtgggagccgtgaggctcaaccatgaatctgacagtacgggcgaaacccgacaggacttaaagatccccaccgtttccgggggtcgctc |
| | | cctcttgcgctctcctgttccgaccctgccgtttaccggatacctgttccgcctttctcccttacgggaagtgtggcgctttctcatagctcacacactggtatctcggctcggtgtaggtcgttcgctc |
| | | caagctgggctgtaagcaagaactccccgttcagcccgactgctgcgccttatccggtaactgttcacttgagtccaacccggaaaagcacggtaaaacgccactggcagcagccattggtaactgg |
| | | gagttcgcagaggatttgtttagctaaacacgcggttgctcttgaagtgtgcgccaaagtccggctacactggaaggacagatttggttgctgtgctctgcgaaagccagttaccacggttaagca |
| | | gttccccaactgacttaaccttcgatcaaaccacctccccaggtggttttttcgtttacagggcaaaagattacgcgcagaaaaaaaggatctcaagaagatcctttgatctgatgccggccacgat |
| | | gcgtccggcgtagaggatctgctcatgtttgacagcttatcatcgatgcataatgtgcctgtcaaatggacgaagcagggattctgcaaaccctatgctactccgtcaagccgtcaattgtctgattc |
| | | gttaccaagtggagcttgtataatg |
| Seq ID | p2373 | attcagcgggttcgtgcgagctgtagacaattaatactcggctcgtataatgtgtggaaggctagcaggaggaattcaatgatatggttaagttagagaatagtcgcaaacctgagaaaatctca |
| No: 63 | | aataagaatattcctatgagcgacttcgtcgtgaatttagaccacggtgatccgacagcgtacgaagagtactggcgcaaaatgggagaccgctgtaccgtgaccattcgcggttgtgacttgat |
| | | gagttatttttctgatatgacgaatctgtgttggttcttggagccggaactggaggatgcgattaaggatttacacggtgtcgttggcaatgcagctacggaggaccgctatatcgtggtgggaact |
| | | gggtctacgcagttatgccaggctgcggtccacgcattgtcttctcttgcgcgtagtcaaccagtttccgttgtggctgcggcaccattttacagcacgtacgtggaggagaccacctatgttcgcag |
| | | tgggatgtacaagtgggagggagacgcgtgggggtttgataagaaaggtccttatattgaacttgtgacatcaccgaataacccggacgggacgatccgcgagaccgtggttaatcgcccgga |
| | | tgatgatgaagcaaaggtaatccacgacttcgcttactattggccccattatacacctattacgcgtcgccaggatcacgacatcatgctttttacctttagcaaaatcacggggcacgctggtagtc |
| | | gtattggatgggcgctggtgaaggataaagaggtcgctaagaaaatggttgagtatattatcgtgaacagtatcggcgtttcgaaagaatcgcaggtgcgtacagcgaaaattttgaatgtcctg |
| | | aaggaaacgtgtaaatctgaatccgaatccgagaacttcttcaaatatggtcgtgagatgatgaagaatcgctgggagaagctgcgtgaagtagtcaaggagtcagacgccttcacattgccca |
| | | aatatcctgaggctttttgtaattatttcgggaagagtttagaatcatacccggcgtttgcctggctggggacaaaggaagagaccgacttcgtatcggaattgcgtcgtcacaaggtaatgtcgcg |
| | | tgcaggcgagcgttgcggttcagataagaagcatgttcgcgtatcaatgctgtcccgcgaggatgttttcaatgtcttcttagagcgcttagccaacatgaaattgatcaaatctatcgatttgtaaat |
| | | atcaatgataaaggaggaaacaaatgaaaatcttagcttattgtgtaagacctgatgaaattgattcatttaaaaatttttctgaaaaatatggacatactgttgatttaataccagattcttttggtcc |
| | | aagtgtggctcatttagctaaaggatatgatggcatttcaattttgggaaatgatacttgtaacagagaagcattagagaaaattaaagattgtggtataaaatatttagcaaccagaactgctgg |
| | | agtaaataatatagattttgatgctgcaaaagaatttggtataaatgtagctaatgttccagcttattctcctaattcagtttcagagtttactgtaggactcgcattatccctaacaaggaaaattcct |
| | | tttgcattaaaaagagtagagcttaacaattttgcattaggtggattaataggagtagaattaagaaacttaactttaggagttataggaactggaagaataggtttaaaagtaattgaaggcttttca |
| | | ggctttggtatgaaaaagatgataggttacgatatttttgaaaatgaaaaggctaaagaatatattgaatataaatctttagatgaagtatataaagaagcagacataataacactacatgcacctt |
| | | taacagatgataattaccatatgatagggaaagagagtattgctaaaatgaaagacggtgtattcataataaatgcagcacgaggagcattaattgatagtgaagctttaatagaaggactgaa |
| | | atctggtaaaatagctggagcagccttagattcttatgaatatgaacaaggagttttccataacaataaaatgaatgaaatcatgaaagatgatacattagcaagacttaagagttttccaaatgta |
| | | gttattacacctcatttaggattctatacagatgaagctgtatcaaatatggttgaaattactcttatgaatttacaagagtttgaattaaaaggaacttgtaaaaatcagagggtatgtaagtaaaaa |
| | | ggaggaaacaaatggaaaacaatacaaatatgtttagtggagtaaaggttattgaattagcaaattttatagctgctccagcagcaggtagattttttgctgatggtggtgcagaggtaataaaa |
| | | attgaatcacctgctggagatcctttaagatatactgctccttcagaaggaagaccattaagccaagaagaaaatactacttatgatttggaaaatgcaaataaaaaagcaatagtattaaatctta |
| | | aaagcgaaaaaggtaaaaagatattacatgaaatgttagcagaagcagatatattattaactaattggagaacaaaggctttagttaaacaaggattagactatgaaacactaaaagaaaaat |
| | | atcctaaattagtttttgcacaaataactggttatggtgaaaaaggaccagataaagatcttccaggctttgattatactgcatttttcgctagaggcggtgtttcaggtactctttatgaaaaaggaa |
| | | ctgtgcctccaaatgttgttccaggacttggagaccatcaagctgggatgtttttagcagcgggtatggcaggagctttatataaagcaaaaacaacaggacaaggagataaagtaacagtaag |
| | | tttaatgcatagtgctatgtatggactaggtattatgatacaagctgctcaatataaagatcatggattagtatatccgataaatcgtaatgaaactccaaatccttttatagtttcatataaatctaag |
| | | gatgattactttgttcaagtatgtatgccaccatatgatgttttctatgatagatttatgaccgctttaggaagagaagatttagttggagacgaaagatacaataaaatagaaaatttaaaagatgg |
| | | acgtgctaaggaagtatacagtataatcgaacaacaaatggttacaaagacaaaggatgaatgggataaaatatttagagatgcagacattccatttgctatcgcacaaacttgggaagatttat |
| | | tagaagatgaacaagcttgggcaaatgattatttgtataagatgaaatatccaacaggaaacgaaagagcattagtaagacttccagtattctttaaagaagcaggattaccagaatataatcaa |
| | | tcaccacaaatagcagaaaatactgtagaagttttaaaagaaatgggatatacagaacaagagattgaggaattagaaaaagataaagatataatggtaaggaaggaaaaataatggcaga |
| | | catttatactatgggtgtagacataggttcaactgcatcaaaaacagtagtattaaaaaatggtaaagaaattgtaagtcaagcagtaataagtgtaggggccggaacaagtggccccaagaga |
| | | gctatagattctgtattaaaagatgctaaattatccattgaagatttagactatattgtatccactggatatggaagaaatagtttcgattttgctaacaaacaaatttctgaattaagttgtcatgcaa |
| | | aaggggtctatttcgataacaataaagctagaacagttattgatataggcggacaagatattaaagtattaaaattagcggatagtggaagacttttaaactttataatgaatgataaatgtgctgca |
| | | ggaacgggacgatttttagatgtaatgtctagagtaatagaagttccagttgatgagttaggaaaaaaagcattagaaagcaaaaatccttgtactattagttctacctgtacagtatttgcagagt |
| | | cagaagtaatttctcaacttgcaagaggagttaaaactgaagatttgatagcaggaatttgtaaatctgtagcatcaagagtggctagccttgcaaagagaagtggtatagaagaattagtagtt |
| | | atgagtggaggagtagctaaaaatataggtgtagtaaaggcaatggaagcagaattgggaagagacatatatatatctaaaaattctcaattaaatggagcattgggagcaagtctatacgctt |
| | | atgaaagttttcaaaaagaaaggagctaaaaacatgagtgatagaaataaggaagtaaaagaaaaaaaggcaaagcattatcttagagagattactgcaaagcattacaaagaagctctcga |
| | | agcaaaagaaaggggagaaaaggttggttggtgtgcatctaacttcccacaagaaatagctacaacattgggggtaaaagttgtttatccagaaaatcatgcagcagctgtagcagctagagg |
| | | gaatggacaaaatatgtgtgaacatgctgaggctatgggtttttctaatgatgtatgtggttatgcaagagtaaatttagctgttatggacataggtcatagtgaagatcaaccaatacctatgcca |
| | | gactttgtactttgctgtaataacatttgtaatcaaatgattaaatggtatgagcatatagcaaaaactttagatataccaatgattcttatagatataccatacaatacagaaaatactgtttcacaag |
| | | atagaattaaatatattagagcacaatttgatgatgcaataaaacaattggaagaaataacaggcaaaaaatgggatgaaaaaaatttgaagaagttatgaaaatatcccaagaaagtgcaa |
| | | aacaatggttaagagcagcatcctatgcaaagtataaaccttcaccatttagcggatttgatttatttaatcatatggctgtagcagtttgtgcaagaggtacacaagaagctgcagatgcatttaag |
| | | atgttagcagatgaatatgaggagaatgtaaaaactggaaaatccacttataggggagaagaaaaacaacgtatattatttgaagggattgcctgttggccatatttgagacataaattaactaa |
| | | gcttagtgaatatggtatgaacgtaactgcaactgtatacgcagaagcctttggtgttatatatgagaatatggatgaattaatggctgcttataataaagttcctaattcaattagttttgaaaacgc |
| | | attaaaaatgagattaaatgtgttacaagcactaatacagaaggtgctgttattcatataaatagaagctgtaaattatggagtggatttttatatgagctagcaagaagattagaaaaggaaac |
| | | aggaattcctgtagtatcatttgatggggaccaggcagacccaagaaatttctcagaagctcaatatgatactagaattcaaggacttaatgaagtaatggttgctaaaaaggaggctgaataag |
| | | atgtcaaattcagataaattttttaatgactttaaggatattgtagaaaatcctaaaaaatatataatgaagcatatggaacaaactggacaaaaggctataggatgtatgccattatatactcctga |
| | | ggaacttgtattagctgctggaatgtttccagtaggggtatggggaagcaatacagaactttcaaaagctaaaacatatttcccagcatttatttgttcaatattacaaacaacattggaaaatgcat |
| | | taaatggagaatatgatatgttatctggtatgatgattacaaattattgtgattcattaaaatgcatgggacaaaattttaaactaaccgttgaaaatattgagtttatcccagtaacagttccacaaa |
| | | atagaaaaatggaagctggaaaagagtttttaaaaagtcaatataaaatgaatattgagcaattagaaaagatttctggtaataaaataacagatgaatctttagaaaaagctatagaaatatat |
| | | gatgaacacagaaaagtaatgaatgacttttcaatgttagcatcaaaatatccaggtataataacaccaactaaacgtaattatgttatgaaatctgcttattatatggataaaaaagaacatactg |
| | | aaaaagttagacaattaatggatgaaattaaagctatagaaccaaaaccatttgaaggaaagagagttataactacaggtataattgcagattcagaagatttacttaaaatattagaagaaaat |
| | | aatatagctatagttggtgatgatatagcacatgaatctagacaatatagaacattgactccagaagcgaacacaccaatggataggttagctgagcaatttgctaatagagaatgtagtactttat |
| | | atgatcctgaaaagaaaaggggtcaatatatagtagaaatggctaaagagagaaaagcagatggaattatatttttcatgacaaaattctgtgacccagaggaatatgattatccacaaatgaa |
| | | aaaggattttgaagaagcaggcattccacatgtactaatagaaactgatatgcaaatgaaaaattatgaacaagctagaactgcaattcaggctttttcagaaacactttaaatccgaactccaatt |
| | | attgatgaaaagggattataaaagctcacttattcaggcgtagcacccaggcgtttaagggcaccaataactgccttaaaaaaattacgccccgccctgccactcatcgcagtactgttgtaattca |
| | | ttaagcattctgccgacatggaagccatcacaaacggcatgatgaacctgaatcgccagcggcatcagcaccttgtcgccttgcgtataatatttgcccatggtgaaaacgggggcgaagaagtt |
| | | gtccatattggccacgtttaaatcaaaactggtgaaactcacccagggattggctgagacgaaaaacatattctcaataaaccctttagggaaataggccaggttttcaccgtaacacgccacatc |
| | | ttgcgaatatatgtgtagaaactgccggaaatcgtcgtggtattcactccagagcgatgaaaacgtttcagtttgctcatggaaaacggtgtaacaagggtgaacactatcccatatcaccagctc |
| | | accgtctttcattgccatacggaattccggatgagcattcatcaggcgggcaagaatgtgaataaaggccggataaaacttgtgcttatttttctttacggtctttaaaaaggccgtaatatccagct |
| | | gaacggtctggttataggtacattgagcaactgactgaaatgcctcaaaatgttctttacgatgccattgggatatatcaacggtggtatatccagtgatttttttctccattttagcttccttagctcc |
| | | tgaaaatctcgataactcaaaaaatacgcccggtagtgatcttatttcattatggtgaaagttggaacctcttacgtgccgatcaacggtcgctacgctccgggcgtgagactgcggggggctgcg |
| | | gacacatacaaagttacccacagattccgtggataagcaggggactaacatgtgaggcaaaacagcagggccgcgccggtggcgtttttccataggctccgccctcctgccagagttcacataa |
| | | acagacgcttttccggtgcatctgtgggagccgtgaggctcaaccatgaatctgacagtacgggcgaaacccgacaggacttaaagatccccaccgtttccgggggtcgctccctcttgcgctct |
| | | cctgttccgaccctgccgtttaccggatacctgttccgcctttctcccttacgggaagtgtggcgctttctcatagctcacacactggtatctcggctcggtgtaggtcgttcgctccaagctgggctgt |
| | | aagcaagaactccccgttcagcccgactgctgcgccttatccggtaactgttcacttgagtccaacccggaaaagcacggtaaaacgccactggcagcagccattggtaactgggagttcgcaga |
| | | ggatttgtttagctaaacacgcggttgctcttgaagtgtgcgccaaagtccggctacactggaaggacagatttggttgctgtgctctgcgaaagccagttaccacggttaagcagttccccaactg |
| | | acttaaccttcgatcaaaccacctccccaggtggttttttcgtttacagggcaaaagattacgcgcagaaaaaaaggatctcaagaagatcctttgatctgatgccggccacgatgcgtccggcgt |
| | | agaggatctgctcatgtttgacagcttatcatcgatgcataatgtgcctgtcaaatggacgaagcagggattctgcaaaccctatgctactccgtcaagccgtcaattgtctgattcgttaccaagtg |
| | | gaggttgtataatg |
| Seq ID | p2374 | attcagcgggttcgtgcgagctgtagacaattaatactcggctcgtataatgtgtggaaggctagcaggaggaattcaatgatatggttaagttagagaatagtcgcaaacctgagaaaatctca |
| No: 64 | | aataagaatattcctatgagcgacttcgtcgtgaatttagaccacggtgatccgacagcgtacgaagagtactggcgcaaaatgggagaccgctgtaccgtgaccattcgcggttgtgacttgat |
| | | gagttatttttctgatatgacgaatctgtgttggttcttggagccggaactggaggatgcgattaaggatttacacggtgtcgttggcaatgcagctacggaggaccgctatatcgtggtgggaact |
| | | gggtctacgcagttatgccaggctgcggtccacgcattgtcttctcttgcgcgtagtcaaccagtttccgttgtggctgcggcaccattttacagcacgtacgtggaggagaccacctatgttcgcag |
| | | tgggatgtacaagtgggagggagacgcgtgggggtttgataagaaaggtccttatattgaacttgtgacatcaccgaataacccggacgggacgatccgcgagaccgtggttaatcgcccgga |
| | | tgatgatgaagcaaaggtaatccacgacttcgcttactattggccccattatacacctattacgcgtcgccaggatcacgacatcatgctttttacctttagcaaaatcacggggcacgctggtagtc |
| | | gtattggatgggcgctggtgaaggataaagaggtcgctaagaaaatggttgagtatattatcgtgaacagtatcggcgtttcgaaagaatcgcaggtgcgtacagcgaaaattttgaatgtcctg |
| | | aaggaaacgtgtaaatctgaatccgaatccgagaacttcttcaaatatggtcgtgagatgatgaagaatcgctgggagaagctgcgtgaagtagtcaaggagtcagacgccttcacattgccca |
| | | aatatcctgaggctttttgtaattatttcgggaagagtttagaatcatacccggcgtttgcctggctggggacaaaggaagagaccgacttggtatcggaattgcgtcgtcacaaggtaatgtcgcg |
| | | tgcaggcgagcgttgcggttcagataagaagcatgttcgcgtatcaatgctgtcccgcgaggatgttttcaatgtcttcttagagcgcttagccaacatgaaattgatcaaatctatcgatttgtaaat |
| | | atcaatgataaaggaggaaacaaatgaaaatcttagcttattgtgtaagacctgatgaaattgattcatttaaaaatttttctgaaaaatatggacatactgttgatttaataccagattcttttggtcc |
| | | aagtgtggctcatttagctaaaggatatgatggcatttcaattttgggaaatgatacttgtaacagagaagcattagagaaaattaaagattgtggtataaaatatttagcaaccagaactgctgg |
| | | agtaaataatatagattttgatgctgcaaaagaatttggtataaatgtagctaatgttccagcttattctcctaattcagtttcagagtttactgtaggactcgcattatccctaacaaggaaaattcct |
| | | tttgcattaaaaagagtagagcttaacaattttgcattaggtggattaataggagtagaattaagaaacttaactttaggagttataggaactggaagaataggtttaaaagtaattgaaggcttttca |
| | | ggctttggtatgaaaaagatgataggttacgatatttttgaaaatgaaaaggctaaagaatatattgaatataaatctttagatgaagtatataaagaagcagacataataacactacatgcacctt |
| | | taacagatgataattaccatatgatagggaaagagagtattgctaaaatgaaagacggtgtattcataataaatgcagcacgaggagcattaattgatagtgaagctttaatagaaggactgaa |
| | | atctggtaaaatagctggagcagccttagattcttatgaatatgaacaaggagttttccataacaataaaatgaatgaaatcatgaaagatgatacattagcaagacttaagagttttccaaatgta |
| | | gttattacacctcatttaggattctatacagatgaagctgtatcaaatatggttgaaattactcttatgaatttacaagagtttgaattaaaaggaacttgtaaaaatcagagggtatgtaagtaaaaa |
| | | ggaggaaacaaatgtttttcacagaacaacatgaacttattagaaaattagcaagagattttgcagagcaggaaatagagcctattgcagatgaagtagataaaactgccgagttccctaaaga |
| | | aattgtgaaaaaaatggcccaaaatggtttttttggaataaaaatgcctaaagaatatggtggagctggagcagataatagagcctatgtaactataatggaagagatatcaagagcatctggtg |
| | | tagctggtatatatttatcatcaccaaattcattgcttggcacaccgttcctactagttggtacagatgaacaaaaggaaaaatatctaaaaccaatgattagaggagaaaaaacattagcttttgct |
| | | ttaacagaaccaggtgcaggttcagacgcaggagcacttgcaacaactgctagagaagaaggggactattatatattaaatggaagaaaaacttttattacaggagctccaatatctgataatat |
| | | catagtgttcgcaaaaacagatatgagcaaaggaacaaagggaattactacatttatagtagatagtaaacaggaaggtgtatcttttggtaaacctgaagacaagatgggtatgattggttgcc |
| | | ctactagtgatattattttagagaatgtaaaggttcataaatctgatatacttggagaagtaaataaaggctttataacagcaatgaagactttaagtgttggtcgtataggagttgcttctcaagcttt |
| | | aggaatagcacaagctgctgtagatgaagctgttaaatatgcaaaacaaagaaaacaatttaatagacctatagcaaaatttcaagctattcaatttaaattcgctaacatggaaacaaaattaa |
| | | atgcggcaaaattattagtgtataatgctgcttataaaatggattgtggagaaaaggcagataaagaagcttctatggcaaaatattttgctgctgaatctgctatacaaatcgtaaatgacgcact |
| | | acaaatccatggtggttatggatacataaaagattataaaatagaaagattatatagagatgttagagtaattgctatatatgaaggaacttctgaagttcaacaaatggtaatcgcatcaaatcttt |
| | | taaagtaaaaaggaggaaacaaatggaaaacaatacaaatatgtttagtggagtaaaggttattgaattagcaaattttatagctgctccagcagcaggtagattttttgctgatggtggtgcaga |
| | | ggtaataaaaattgaatcacctgctggagatcctttaagatatactgctccttcagaaggaagaccattaagccaagaagaaaatactacttatgatttggaaaatgcaaataaaaaagcaatag |
| | | tattaaatcttaaaagcgaaaaaggtaaaaagatattacatgaaatgttagcagaagcagatatattattaactaattggagaacaaaggctttagttaaacaaggattagactatgaaacacta |
| | | aaagaaaaatatcctaaattagtttttgcacaaataactggttatggtgaaaaaggaccagataaagatcttccaggctttgattatactgcatttttcgctagaggcggtgtttcaggtactctttatg |
| | | aaaaaggaactgtgcctccaaatgttgttccaggacttggagaccatcaagctgggatgtttttagcagcgggtatggcaggagctttatataaagcaaaaacaacaggacaaggagataaag |
| | | taacagtaagtttaatgcatagtgctatgtatggactaggtattatgatacaagctgctcaatataaagatcatggattagtatatccgataaatcgtaatgaaactccaaatccttttatagtttcata |
| | | taaatctaaggatgattactttgttcaagtatgtatgccaccatatgatgttttctatgatagatttatgaccgctttaggaagagaagatttagttggagacgaaagatacaataaaatagaaaattt |
| | | aaaagatggacgtgctaaggaagtatacagtataatcgaacaacaaatggttacaaagacaaaggatgaatgggataaaatatttagagatgcagacattccatttgctatcgcacaaacttgg |
| | | gaagatttattagaagatgaacaagcttgggcaaatgattatttgtataagatgaaatatccaacaggaaacgaaagagcattagtaagacttccagtattctttaaagaagcaggattaccaga |
| | | atataatcaatcaccacaaatagcagaaaatactgtagaagttttaaaagaaatgggatatacagaacaagagattgaggaattagaaaaagataaagatataatggtaaggaaggaaaaat |
| | | aatggcagacatttatactatgggtgtagacataggttcaactgcatcaaaaacagtagtattaaaaaatggtaaagaaattgtaagtcaagcagtaataagtgtaggggccggaacaagtggc |
| | | cccaagagagctatagattctgtattaaaagatgctaaattatccattgaagatttagactatattgtatccactggatatggaagaaatagtttcgattttgctaacaaacaaatttctgaattaagtt |
| | | gtcatgcaaaaggggtctatttcgataacaataaagctagaacagttattgatataggcggacaagatattaaagtattaaaattagcggatagtggaagacttttaaactttataatgaatgataa |
| | | atgtgctgcaggaacgggacgatttttagatgtaatgtctagagtaatagaagttccagttgatgagttaggaaaaaaagcattagaaagcaaaaatccttgtactattagttctacctgtacagta |
| | | tttgcagagtcagaagtaatttctcaacttgcaagaggagttaaaactgaagatttgatagcaggaatttgtaaatctgtagcatcaagagtggctagccttgcaaagagaagtggtatagaaga |
| | | attagtagttatgagtggaggagtagctaaaaatataggtgtagtaaaggcaatggaagcagaattgggaagagacatatatatatctaaaaattctcaattaaatggagcattgggagcaagt |
| | | ctatacgcttatgaaagttttcaaaaagaaaggagctaaaaacatgagtgatagaaataaggaagtaaaagaaaaaaaggcaaagcattatcttagagagattactgcaaagcattacaaag |
| | | aagctctcgaagcaaaagaaaggggagaaaaggttggttggtgtgcatctaacttcccacaagaaatagctacaacattgggggtaaaagttgtttatccagaaaatcatgcagcagctgtagc |
| | | agctagagggaatggacaaaatatgtgtgaacatgctgaggctatgggtttttctaatgatgtatgtggttatgcaagagtaaatttagctgttatggacataggtcatagtgaagatcaaccaata |
| | | cctatgccagactttgtactttgctgtaataacatttgtaatcaaatgattaaatggtatgagcatatagcaaaaactttagatataccaatgattcttatagatataccatacaatacagaaaatactg |
| | | tttcacaagatagaattaaatatattagagcacaatttgatgatgcaataaaacaattggaagaaataacaggcaaaaaatgggatgaaaataaatttgaagaagttatgaaaatatcccaaga |
| | | aagtgcaaaacaatggttaagagcagcatcctatgcaaagtataaaccttcaccatttagcggatttgatttatttaatcatatggctgtagcagtttgtgcaagaggtacacaagaagctgcagat |
| | | gcatttaagatgttagcagatgaatatgaggagaatgtaaaaactggaaaatccacttataggggagaagaaaaacaacgtatattatttgaagggattgcctgttggccatatttgagacataa |
| | | attaactaagcttagtgaatatggtatgaacgtaactgcaactgtatacgcagaagcctttggtgttatatatgagaatatggatgaattaatggctgcttataataaagttcctaattcaattagtttt |
| | | gaaaacgcattaaaaatgagattaaatgctgttacaagcactaatacagaaggtgctgttattcatataaatagaagctgtaaattatggagtggatttttatatgagctagcaagaagattagaa |
| | | aaggaaacaggaattcctgtagtatcatttgatggggaccaggcagacccaagaaatttctcagaagctcaatatgatactagaattcaaggacttaatgaagtaatggttgctaaaaaggagg |
| | | ctgaataagatgtcaaattcagataaattttttaatgactttaaggatattgtagaaaatcctaaaaaatatataatgaagcatatggaacaaactggacaaaaggctataggatgtatgccattat |
| | | atactcctgaggaacttgtattagctgctggaatgtttccagtaggggtatggggaagcaatacagaactttcaaaagctaaaacatatttcccagcatttatttgttcaatattacaaacaacattgg |
| | | aaaatgcattaaatggagaatatgatatgttatctggtatgatgattacaaattattgtgattcattaaaatgcatgggacaaaattttaaactaaccgttgaaaatattgagtttatcccagtaacag |
| | | ttccacaaaatagaaaaatggaagctggaaaagagtttttaaaaagtcaatataaaatgaatattgagcaattagaaaagatttctggtaataaaataacagatgaatctttagaaaaagctata |
| | | gaaatatatgatgaacacagaaaagtaatgaatgacttttcaatgttagcatcaaaatatccaggtataataacaccaactaaacgtaattatgttatgaaatctgcttattatatggataaaaaag |
| | | aacatactgaaaaagttagacaattaatggatgaaattaaagctatagaaccaaaaccatttgaaggaaagagagttataactacaggtataattgcagattcagaagatttacttaaaatatta |
| | | gaagaaaataatatagctatagttggtgatgatatagcacatgaatctagacaatatagaacattgactccagaagcgaacacaccaatggataggttagctgagcaatttgctaatagagaatg |
| | | tagtactttatatgatcctgaaaagaaaaggggtcaatatatagtagaaatggctaaagagagaaaagcagatggaattatatttttcatgacaaaattctgtgacccagaggaatatgattatcc |
| | | acaaatgaaaaaggattttgaagaagcaggcattccacatgtactaatagaaactgatatgcaaatgaaaaattatgaacaagctagaactgcaattcaggctttttcagaaacactttaaatccg |
| | | aactccaattattgatgaaaagggattataaaagctcacttattcaggcgtagcacccaggcgtttaagggcaccaataactgccttaaaaaaattacgccccgccctgccactcatcgcagtact |
| | | gttgtaattcattaagcattctgccgacatggaagccatcacaaacggcatgatgaacctgaatcgccagcggcatcagcaccttgtcgccttgcgtataatatttgcccatggtgaaaacggggg |
| | | cgaagaagtigtccatattggccacgtttaaatcaaaactggtgaaactcacccagggattggctgagacgaaaaacatattctcaataaaccctttagggaaataggccaggttttcaccgtaac |
| | | acgccacatcttgcgaatatatgtgtagaaactgccggaaatcgtcgtggtattcactccagagcgatgaaaacgtttcagtttgctcatggaaaacggtgtaacaagggtgaacactatcccata |
| | | tcaccagctcaccgtctttcattgccatacggaattccggatgagcattcatcaggcgggcaagaatgtgaataaaggccggataaaacttgtgcttatttttctttacggtctttaaaaaggccgta |
| | | atatccagctgaacggtctggttataggtacattgagcaactgactgaaatgcctcaaaatgttctttacgatgccattgggatatatcaacggtggtatatccagtgatttttttctccattttagctt |
| | | ccttagctcctgaaaatctcgataactcaaaaaatacgcccggtagtgatcttatttcattatggtgaaagttggaacctcttacgtgccgatcaacggtcgctacgctccgggcgtgagactgcggc |
| | | gggcgctgcggacacatacaaagttacccacagattccgtggataagcaggggactaacatgtgaggcaaaacagcagggccgcgccggtggcgtttttccataggctccgccctcctgccag |
| | | agttcacataaacagacgcttttccggtgcatctgtgggagccgtgaggctcaaccatgaatctgacagtacgggcgaaacccgacaggacttaaagatccccaccgtttccgggggtcgctcc |
| | | ctcttgcgctctcctgttccgaccctgccgtttaccggatacctgttccgcctttctcccttacgggaagtgtggcgctttctcatagctcacacactggtatctcggctcggtgtaggtcgttcgctcc |
| | | aagctgggctgtaagcaagaactccccgttcagcccgactgctgcgccttatccggtaactgttcacttgagtccaacccggaaaagcacggtaaaacgccactggcagcagccattggtaactggg |
| | | agttcgcagaggatttgtttagctaaacacgcggttgctcttgaagtgtgcgccaaagtccggctacactggaaggacagatttggttgctgtgctctgcgaaagccagttaccacggttaagcag |
| | | ttccccaactgacttaaccttcgatcaaaccacctccccaggtggttttttcgtttacagggcaaaagattacgcgcagaaaaaaaggatctcaagaagatcctttgatctgatgccggccacgatg |
| | | cgtccggcgtagaggatctgctcatgtttgacagcttatcatcgatgcataatgtgcctgtcaaatggacgaagcagggattctgcaaaccctatgctactccgtcaagccgtcaattgtctgattcg |
| | | ttaccaagtcgaggttgtataatg |
| Seq ID | RelB | cgactactttctgacttccttcgtgacttgccctaagcatgttgtagtgcgatacttgtaAtgacatttgtaattacaagaggtg |
| No: 65 | promoter | |
| | sequence, | |
| | σ70 | |
| Seq ID | BclA | aacctaaatatttgttgttaagctgcaatggaaacggtaaaagcggctagtatttaaaggGatggatgacatctcagcgttgtcg |
| No: 66 | promoter | |
| | sequence, | |
| | σS, σ70 | |
| Seq ID | Hya | aaaagataaatccacacagtttgtattgttttgtgcaaaagtttcactacgctttattaaCaatactttctggcgacgtgcgcca |
| No: 67 | promoter | |
| | sequence, | |
| | σS, σ70 | |
| Seq ID | YiaG | agccagagcatgccctgacttcaccccgctgtgtctgcttttcccgactattcttaatgaGcttcgatgcaattcacgatcccgc |
| No: 68 | promoter | |
| | sequence, | |
| | σS | |
| Seq ID | RpoH | acggtacaacatttacgccactttacgcctgaataataaaagcgtgttatactctttcccTgcaatgggttccgtagcagggaaa |
| No: 69 | promoter | |
| | sequence | |
| | P1, σ70 | |
| Seq ID | RpoH | acggtacaacatttacgccactttacgcctgaataataaaagcgtgttatactctttcccTgcaatgggttccgtagcagggaaa |
| No: 70 | promoter | |
| | sequence | |
| | P2, σS | |
| Seq ID | RpoH | tgcgtaatttattcacaagcttgcattgaacttgtggataaaatcacggtctgataaaacAgtgaatgataacctcgttgctctt |
| No: 71 | promoter | |
| | sequence | |
| | P3, σ24 | |
| Seq ID | RpoH | tttattcacaagcttgcattgaacttgtggataaaatcacggtctgataaaacagtgaatGataacctcgttgctcttaagctct |
| No: 72 | promoter | |
| | sequence | |
| | P4, σ70 | |
| Seq ID | RpoH | tgcattgaacttgtggataaaatcacggtctgataaaacagtgaatgataacctcgttgcTcttaagctctggcacagttgttgc |
| No: 73 | promoter | |
| | sequence | |
| | P5, σ70 | |
| Seq ID | RpoH | aacagtgaatgataacctcgttgctcttaagctctggcacagttgttgctaccactgaagCgccagaagatatcgattgagagga |
| No: 74 | promoter | |
| | sequence | |
| | P6, σ54 | |
| Seq ID | hypC2 | ATGCAGAATAAACCTACACCTGAAGAAGTAAAGAATGCGGGGGTTGCGGCAGGTCTTACTCTTAAAGAAGCTGCTGATATTTT |
| No: 75 | sequence | TGGTTATCAACTGAATTCCTGGCAGATGAAAGAAAGTGCAGGTAAGGCCAGTCGTTCTTTATCTATTGGTGAATATCAGTATT |
| | | TATTGTTATTAGCAAATATGCATCCGTCTTACAGGCTGGTAAAAAAATAA |
| Seq ID | Oli7154 | tacggctgcatgcccgaggcagacagcctcaagcacccgcagctattctacagtaaaaactcgcgcttctctagactatattaccctgtt |
| No: 76 | | |
| Seq ID | Oli7156 | agcggtacggaagtttcccccgacaccatgatggcggttagttgcgtcagaaatagttacgtatgcaactaatcagtcctgctcctcggc |
| No: 77 | | |
| Seq ID | Oli8199 | gtggtttgccacaaaacagtgcagtcacacatgacaggagaagatatgagccgggtaccgcggctctgagactcagtcctgctcctcggc |
| No: 78 | | |
| Seq ID | Oli8200 | ggtgcaggaaataaaaataatcatttctttatatgatctttttatcaatggagatagtatcatttatactctagactatattaccctgtt |
| No: 79 | | |
| Seq ID | Oli2906 | gtgatctccaccggcttattagcgatagggattatctgtaccgtgccaattatggctcgcccttagttcctattccgaagttcct |
| No: 80 | | |
| Seq ID | Oli2907 | gtcgaaaatatcgagcagctcctgctctgacatagacgccacccgataccctttttgattcaccaccgccctgccactcatcgcagt |
| No: 81 | | |
| Seq ID | Oli8821 | gtgatcaccagataatgttgc |
| No: 82 | | |
| Seq ID | Oli8822 | gcttcatcaccactgacc |
| No: 83 | | |
| Seq ID | oli8116 | ccggtatcgggttaaagagtgggaaaaagttgccggtgttcctgttgcagaagtattttgactcacttccctgttaagtatcttcct |
| No: 84 | | |
| Seq ID | oli8117 | aaaagaaaaagccggttcagagaaaaccggctgacggattatcgtctaacagttgatagctaccattacgccccgccctgccactca |
| No: 85 | | |
| Seq ID | oli8805 | cgcagacaataaataatgatcatccccagaaatcatgtggagcacaaccaacaactgacgaaaaatactgatgatcggcacgtaagagg |
| No: 86 | | |
| Seq ID | oli8743 | accccggacttagtgaacagggttttacagtctgattgacagatatggtcaccagaaaatatttgtattcctaattcagcgggttcgtgc |
| No: 87 | | |
| Seq ID | oli7159 | cggcacgtaagaggctaacc |
| No: 88 | | |
| Seq ID | oli7160 | ggaagtttcccccgacaccatgatggcggttagttgcgtcagaaatagttacgtatgcaactaaaagcgcgagtttttactgtagaatag |
| No: 89 | | |
| Seq ID | oli8201 | cctgtatttttctgctcaatgg |
| No: 90 | | |
| Seq ID | oli8197 | aaacagtgcagtcacacatgacaggagaagatatgagccgggtaccgcggctctgagactataaatgatactatctccattgataaaaag |
| No: 91 | | |

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A modified bacterium for the biosynthesis of indole-3-acetic acid (IAA), comprising heterologous genes for the biosynthesis of IAA, which heterologous genes encode:
(i) indole-3-pyruvate decarboxylase (ipdC) from Pantoea agglomerans, having the nucleic acid sequence of SEQ ID No:41; or a sequence having at least 90% identity thereto and converts IPyA to IAAld;
(ii) tryptophan-pyruvate aminotransferase 1 (taa1) from Arabidopsis thaliana, having the nucleic acid sequence of SEQ ID No:40; or a sequence having at least 90% identity thereto and converts tryptophan to IPyA; and
(iii) indole-3-acetaldehyde dehydrogenase (iad1) from Ustilago maydis, having the nucleic acid sequence of SEQ ID No:42; or a sequence having at least 90% identity thereto and converts IAAld to IAA;
wherein heterologous genes (i) to (iii) are each or all under the control of one or more constitutive promoter(s) and are all comprised by the chromosome of said modified bacterium, and wherein: the bacterium further comprises a heterologous gene encoding an auxin efflux protein exporter which is capable of exporting IAA out of the bacterium.

2. The modified bacterium according to claim 1, wherein the heterologous gene encoding the exporter which is capable of exporting IAA is under the control of a constitutive promoter.

3. The modified bacterium according to claim 1, wherein the one or more heterologous gene(s) for the biosynthesis of said IAA are comprised within an operon under the control of a single constitutive promoter, and optionally
wherein the one or more heterologous gene(s) for the biosynthesis of IAA comprised within the operon comprise the following genes in downstream order:
(i) indole-3-pyruvate decarboxylase (ipdC) from Pantoea agglomerans, having the nucleic acid sequence of SEQ ID No:41; or a sequence having at least 90% identity thereto and converts IPyA to IAAld;
(ii) tryptophan-pyruvate aminotransferase 1 (taa1) from Arabidopsis thaliana, having the nucleic acid sequence of SEQ ID No:40; or a sequence having at least 90% identity thereto and converts tryptophan to IPyA; and
(iii) indole-3-acetaldehyde dehydrogenase (iad1) from Ustilago maydis, having the nucleic acid sequence of SEQ ID No:42; or a sequence having at least 90% identity thereto and converts IAAld to IAA.

4. The modified bacterium according to claim 3, wherein the one or more heterologous gene(s) for the biosynthesis of IAA and the heterologous gene encoding the exporter which is capable of exporting IAA are comprised within an operon under the control of a single constitutive promoter.

5. The modified bacterium according to claim 1, wherein the constitutive promoter is a strong constitutive promoter having an Anderson score >0.4 (e.g. >0.5), e.g. a tac promoter, for example a Pc-tga promoter having the sequence of SEQ ID No: 1, and optionally
wherein the promoter is a promoter selected from a RelB, BolA, Hya, YiaG and a RpoH promoter, such as a promoter selected from a RelB promoter sequence, σ70; a BolA promoter sequence, σS, σ70; a Hya promoter sequence, σS, σ70; a YiaG promoter sequence, σS; a RpoH promoter sequence P1, σ70; a RpoH promoter sequence P2, σS; a RpoH promoter sequence P3, σ24; a RpoH promoter sequence P4, σ70; a RpoH promoter sequence P5, σ70; and a RpoH promoter sequence P6, σ54, in particular a promoter having a nucleotide sequence selected from any one of Seq ID Nos: 65 to 74, or a nucleotide sequence having 90% (or 95%) homology thereto.

6. The modified bacterium according to claim 4, wherein the one or more heterologous gene(s) for the biosynthesis of IAA and the heterologous gene encoding the exporter which is capable of exporting IAA comprised within the operon comprise the following genes in downstream order:
(i) indole-3-pyruvate decarboxylase (ipdC) from Pantoea agglomerans, having the nucleic acid sequence of SEQ ID No:41; or a sequence having at least 90% identity thereto and converts IPyA to IAAld;
(ii) tryptophan-pyruvate aminotransferase 1 (taa1) from Arabidopsis thaliana, having the nucleic acid sequence of SEQ ID No:40; or a sequence having at least 90% identity thereto and converts tryptophan to IPyA; and
(iii) indole-3-acetaldehyde dehydrogenase (iad1) from Ustilago maydis, having the nucleic acid sequence of SEQ ID No:42; or a sequence having at least 90% identity thereto and converts IAAld to IAA; and
(iv) the heterologous gene encoding the exporter of IAA,
wherein the operon is comprised by the chromosome of the modified bacterium.

7. The modified bacterium according to claim 1, wherein the bacterium further comprises a modification in an endogenous tryptophanase (tnaA) and/or tnaC which reduces expression (or prevents expression) of said tnaA and/or tnaC, e.g. the bacterium further comprises a deletion of one or more nucleotides in an endogenous tnaA and/or tnaC which prevents or reduces (e.g. prevents) transcription or expression of said tnaA and/or tnaC, such as a deletion which comprises at least the nucleotides which, when transcribed, express a TnaC peptide; and/or
wherein the bacterium further comprises a modification in an endogenous tryptophan transcriptional repressor (trpR) gene which reduces expression (or prevents expression) of said trpR, e.g. the bacterium further comprises a deletion of one or more nucleotides in an endogenous trpR gene which prevents or reduces (e.g. prevents) transcription or expression of said trpR.

8. The modified bacterium according to claim 1, wherein the heterologous gene encoding the exporter which is capable of exporting IAA is comprised by the chromosome of said modified bacterium.

9. The modified bacterium according to claim 1, wherein
(a) the bacterium is a gram negative bacterium; and/or
(b) the bacterium is a strain selected from any of the strains in Table 3; and/or
(c) the bacterium is an E. coli strain, for example an E. coli from phylogroup A, B1 and/or E, or an E. coli strain which is present in a probiotic product, such as colinfant New Born (e.g. strain A0 34/86) or symbioflor2 (e.g. strain G1/2, G4/9, G5, G6/7, and G8, in particular strain G6/7), or Mutaflor (e.g. E. coli Nissle); and/or
(d) the bacterium is a strain belonging to a genera selected from Bifidobacterium, Bacteroides, Lactobacillus, Lacticaseibacillus, Lactiplantibacillus, Levilactobacillus, Ligilactobacillus, Limosilactobacillus and Lactococcus, (e.g. a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacillus brevis, Ligilactobacillus salivarius, Limosilactobacillus reuteri and Lactococcus lactis), in particular a strain belonging to a Bifidobacterium genus or a Bacteroides genus (e.g. a species which is selected from Bifidobacterium longum, Bifidobacterium breve, Bifidobacterium adolescentis, Bacteroides uniformis, Bacteroides vulgatus and Bacteroides thetaiotaomicron).

10. The modified bacterium according to claim 9, wherein the bacterium strain has been engineered to remove some or all (e.g. all) prophage genes present in the bacterium genome, or wherein the bacterium is devoid of some or all (e.g. all) prophage genes; and/or
wherein the bacterium strain has been engineered to remove any identified pathogenicity factors (such as hlyA, hlyB, hlyC and/or hlyD or any combination thereof) present in the bacterium genome, or wherein the bacterium strain is devoid of pathogenicity factors (such as hlyA, hlyB, hlyC and/or hlyD or any combination thereof).

11. The modified bacterium according to claim 1, wherein the bacterium does not comprise any heterologous genes for the biosynthesis of tryptophan, for example genes encoding one or more genes selected from trpA, trpB, trpC, trpD and trpE, for example all of trpA, trpC, trpD and trpE; and/or
wherein the bacterium comprises no other heterologous genes other than:
(I) the one or more heterologous gene(s) for the biosynthesis of IAA; and/or
(II) the heterologous gene encoding an exporter which is capable of exporting IAA;
(III) optionally the kill switch(es); and
(IV) optionally any heterologous or modified auxotrophy genes.

12. The modified bacterium according to claim 1, wherein the heterologous gene encoding the exporter is selected from:
(i) an auxin efflux carrier (AEC) family protein transporter (for example an AEC family protein from a bacterial species);
(ii) a PIN family protein transporter (for example a PIN family protein transporter from a plant species); and
(iii) an ABC family protein transporter, such as an ABCD subfamily protein transporter or an ABCB subfamily protein transporter, e.g. an ABCB-PGP sub-family protein transporter (for example where the ABC family protein transporter is from a plant species).

13. The modified bacterium according to claim 12, wherein the exporter is an AEC family protein transporter.

14. The modified bacterium according to claim 1, wherein the auxin efflux protein is from Pantoea agglomerans and comprises the nucleotide sequence of SEQ ID No:2,
or wherein the exporter of IAA is a PIN transporter (e.g. PIN2 or PIN7), or wherein the heterologous gene encoding the exporter comprises the nucleotide sequence of SEQ ID No: 3.

15. The modified bacterium according to claim 6, wherein the heterologous gene encoding the exporter of IAA of (iv) is an auxin efflux protein from Pantoea agglomerans and comprises the nucleotide sequence of SEQ ID No:2; or a sequence having at least 90% identity thereto, and wherein the bacterium further comprises a deletion which comprises at least the nucleotides which, when transcribed, express a TnaC peptide; and a deletion of one or more nucleotides in an endogenous trpR gene which prevents transcription or expression of said trpR.

16. A pharmaceutical composition comprising a modified bacterium as defined in claim 1, and a pharmaceutically acceptable excipient or carrier, optionally wherein the composition:
(a) is formulated for oral or rectal administration, preferably oral administration, for example formulated as a capsule or coated tablet; and/or
(b) is a lyophilised formulation or is an encapsulated formulation to be released in the lower gut of a subject, for example in the small intestine or large intestine of a subject; further optionally which is formulated as an enteric late release capsule; and/or
(c) comprises freeze dried modified bacteria or host cells; and/or
(d) comprises (approximately) 1×10⁹ colony forming units (CFU)/gram of the modified bacterium.

17. A method of producing IAA in the gut of a subject, comprising administering to said subject a modified bacterium as defined in claim 1.

18. A method of treating a disease in a subject in need thereof, the method comprising administering to the subject the modified bacterium of claim 1, wherein the disease is selected from the group consisting of:
(A) a metabolic disease, such as a cardiovascular metabolic disease, optionally selected from leaky gut, type 1 diabetes, type 2 diabetes (including complications of type 1 and type 2 diabetes, e.g. insulin sensitivity in type 2 diabetes), metabolic syndrome, Bardet-Biedel syndrome, Prader-Willi syndrome, non-alcoholic fatty liver disease, tuberous sclerosis; Albright hereditary osteodystrophy; brain-derived neurotrophic factor (BDNF) deficiency, Single-minded 1 (SIM1) deficiency, leptin deficiency, leptin receptor deficiency, pro-opiomelanocortin (POMC) defects, proprotein convertase subtilisin/kexin type 1 (PCSK1) deficiency, Src homology 2B1 (SH2B1) deficiency, pro-hormone convertase ⅓ deficiency, melanocortin-4-receptor (MC4R) deficiency, Wilms tumor, aniridia, genitourinary anomalies, and mental retardation (WAGR) syndrome, pseudohypoparathyroidism type 1A, Fragile X syndrome, Borjeson-Forsmann-Lehmann syndrome, Alstrom syndrome, Cohen syndrome, and ulnar-mammary syndrome (in particular selected from metabolic syndrome, type 2 diabetes (including complications of type 2 diabetes, e.g. insulin sensitivity in type 2 diabetes), and non-alcoholic fatty liver disease);
(B) a cancer, in particular colorectal cancer (CRC) and/or pancreatic cancer, e.g. pancreatic ductal adenocarcinoma (PDAC),
and optionally wherein the subject is receiving a chemotherapy (e.g. concurrently, before or after administration of the bacterium, the plasmid (e.g. conjugative plasmid), the host cell or the pharmaceutical formulation; and
(C) non-alcoholic fatty liver disease (NAFLD).

19. The method according to claim 18, wherein the subject is administered a course of antibiotics within one month (for example within 2 weeks, within one week, e.g. within 5, 4 or 3 days, in particular within 34 hours) of receiving a first dose of the modified bacterium, the host cell, or the pharmaceutical composition, and further optionally
wherein the antibiotic treatment is selected from an aminoglycoside (e.g. amikacin, liposomal amikacin, gentamicin, plazomicin and tobramycin), a β-lactam inhibitor (e.g. ceftolozane and cilastatin), a β-lactamase inhibitor (e.g. avibactam, clavulanate, clavulanic acid, salbactam, tazobactam, relebactam and vaborbactam), a carbapenem (e.g. doripenem, ertapenem, imipenem, and meropenem), a cephalosporin (e.g. cefaclor, cefadroxil, cefazolin, cefdinir, cefditoren, cefepime, cefiderocol, cefixime, cefotaxime, cefotetan, cefoxitin, cefpodoxime, cefprozil, ceftaroline, ceftazidime, ceftibuten, ceftizoxime, ceftriaxone, cefuroxime and cephalexin), a fluoroquinolone (e.g. ciprofloxacin, delafloxacin, gemifloxacin, levofloxacin and moxifloxacin), a folate pathway inhibitor (e.g. sulfisoxazole, sulfamethoxazole and trimethoprim), a fosfomycin (e.g. fosfomycin), a glycopeptide (e.g. dalbavancin, oritavancin, telavancin and vancomycin), a glycocycline (e.g. tigecycline), a ketolide (e.g. telithromycine), a lincosamide (e.g. clindamycin), a lipopeptide (e.g. daptomycin), a macrocyclic (e.g. fidaxomicin) macrolide (e.g. azithromycin, clarithromycin and erythromycin), a monobactam (e.g. aztreonam), a nitrofuran (e.g. nitrofurantoin), a nitroimidazole (e.g. metronidazole and tinidazole), a nucleoside analog (e.g. molnupiravir and remdesivir), an oxazolidinone (e.g. linezolid and tedizolid), penicillin (e.g. amoxicillin, ampicillin, dicloxacillin, nafcillin, oxacillin, penicillin G, penicillin V, piperacillin and ticarcillin), a phenicol (e.g. chloramphenicol), a polyene (e.g. amphotericin B, liposomal amphotericin B and amphotericin B lipid complex), polymerase acidic endonuclease inhibitor (e.g. baloxavir marboxil), a polymyxin (e.g. colistimethate, colistin and polymyxin B), a pleuromutilin (e.g. lefamulin), a protease inhibitor (e.g. nirmatrelvir), rifampin, a streprogramin (e.g. quinupristin and dalfopristin), a tetracycline (e.g. eravacycline, minocycline, omadacycline and tetracycline), or combinations thereof.

20. A method of producing a modified bacterium as defined in claim 8, said method comprising:
using recombineering to introduce the heterologous genes into the chromosome of the bacterium, and
propagating said bacterium,
optionally formulating said bacterium into a pharmaceutical composition, and
optionally packaging said pharmaceutical composition with instructions for use.
